(19) United States
(12) Patent Application Publication
(10) Pub. No.: US 2026/0132134 A1
(43) Pub. Date: May 14, 2026

(54) ORGANIC COMPOUND, LIGHT-EMITTING DEVICE, THIN FILM, LIGHT-EMITTING APPARATUS, ELECTRONIC APPARATUS, AND LIGHTING DEVICE

(71) Applicant: SEMICONDUCTOR ENERGY LABORATORY CO., LTD., Atsugi-shi, Kanagawa-ken (JP)

(72) Inventors: Yuki HAYASHI, Atsugi, Kanagawa (JP); Sachiko KAWAKAMI, Atsugi, Kanagawa (JP); Hiromitsu KIDO, Atsugi, Kanagawa (JP)

(21) Appl. No.: 18/686,206

(22) PCT Filed: Sep. 21, 2022
(86) PCT No.: PCT/IB2022/058898

§ 371 Date: Feb. 23, 2024

(30) Foreign Application Priority Data
Sep. 30, 2021 (JP) 2021-161191

(51) Int. Cl. C07D 487/04; C07B 59/00; C09K 11/02; H10K 50/11; H10K 85/60

(52) U.S. Cl. CPC C07D 487/04; C07B 59/002; C09K 11/02; H10K 85/654; H10K 85/6572; H10K 50/11

(57) ABSTRACT

An organic compound represented by General Formula (G1) is provided. At least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ to Ar³ independently represents a substituted or unsubstituted aryl group having 6 to 30 carbon atoms or a substituted or unsubstituted heteroaryl group having 2 to 30 carbon atoms; α represents a substituted or unsubstituted arylene group having 6 to 30 carbon atoms or a substituted or unsubstituted heteroarylene group having 2 to 30 carbon atoms; n represents an integer of 0 to 2.

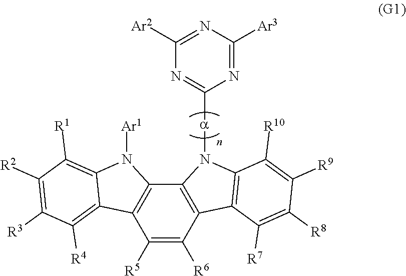

### TECHNICAL FIELD

One embodiment of the present invention relates to an organic compound, a light-emitting device, a light-emitting apparatus, a light-emitting and light-receiving apparatus, a display apparatus, an electronic apparatus, a lighting device, and an electronic device. Note that one embodiment of the present invention is not limited to the above technical field. The technical field of one embodiment of the invention disclosed in this specification and the like relates to an object, a method, or a manufacturing method. Alternatively, one embodiment of the present invention relates to a process, a machine, manufacture, or a composition of matter. Accordingly, more specific examples of the technical field of one embodiment of the present invention disclosed in this specification include a semiconductor device, a display apparatus, a liquid crystal display apparatus, a light-emitting apparatus, a lighting device, a power storage device, a memory device, an imaging device, a driving method thereof, and a manufacturing method thereof.

### BACKGROUND ART

Light-emitting devices including organic compounds and utilizing electroluminescence (EL) (also referred to as organic EL elements or light-emitting elements) have been put into practical use. In the basic structure of such light-emitting devices, an organic compound layer containing a light-emitting material (an EL layer) is sandwiched between a pair of electrodes. Carriers are injected by application of voltage to the device, and recombination energy of the carriers is used, whereby light emission can be obtained from the light-emitting material.

Such light-emitting devices are of self-light-emitting type and thus have higher visibility than liquid crystal displays, and are suitable as pixels of a display. Displays including such light-emitting devices are also highly advantageous in that they require no backlight and fabricated to be thin and lightweight. Moreover, such light-emitting devices also have a feature that the response speed is extremely fast.

Since light-emitting layers of such light-emitting devices can be successively formed two-dimensionally, planar light emission can be achieved. This feature is difficult to realize with point light sources typified by incandescent lamps and LEDs or linear light sources typified by fluorescent lamps; thus, the light-emitting devices also have great potential as planar light sources, which can be applied to lighting and the like.

Displays or lighting devices including light-emitting devices are suitable for a variety of electronic apparatuses as described above, and research and development of light-emitting devices has proceeded for more favorable efficiency and lifetime.

The characteristics of light-emitting devices have been improved considerably, but are still insufficient to satisfy advanced requirements for various characteristics such as efficiency and durability. In particular, to solve a problem such as burn-in, which is an issue peculiar to EL, it is preferable to suppress a reduction in efficiency due to deterioration as much as possible.

Deterioration largely depends on an emission center substance and its surrounding materials; therefore, host materials having favorable characteristics have been actively developed. As host materials, organic compounds having indolocarbazole skeletons are disclosed, for example (Patent Document 1 and Patent Document 2). Organic compounds having indolocarbazole skeletons, which have high glass transition temperature, can offer favorable characteristics when used in light-emitting devices. Meanwhile, materials are being required to have higher heat resistance and a longer lifetime so that deterioration of light-emitting devices is suppressed.

In addition, a technique for substituting deuterium for hydrogen contained in a host material (a deuteration technique) is disclosed (Patent Document 3). Although deuteration of a host material is effective for a longer lifetime of a light-emitting device, a complicated synthesis pathway, necessity of high temperature and high pressure for synthesis, or the like is a problem.

### REFERENCE

### Patent Document

[Patent Document 1] PCT International Publication No. WO2018/198844
[Patent Document 2] PCT International Publication No. WO2018/123783
[Patent Document 3] Japanese Translation of PCT International Application No. 2013-503860

### SUMMARY OF THE INVENTION

### Problems to be Solved by the Invention

An object of one embodiment of the present invention is to provide a novel organic compound. Another object of one embodiment of the present invention is to provide an organic compound with a long lifetime. Another object of one embodiment of the present invention is to provide an organic compound that can be used as a host material. Another object of one embodiment of the present invention is to provide an organic compound that is easily synthesized. Another object of one embodiment of the present invention is to provide a light-emitting device with a long lifetime. Another object of one embodiment of the present invention is to provide a novel light-emitting device. Another object of one embodiment of the present invention is to reduce manufacturing costs of a light-emitting device. Another object of one embodiment of the present invention is to provide a low-power-consumption light-emitting apparatus, electronic apparatus, or lighting device. Another object of one embodiment of the present invention is to provide a low-power-consumption light-emitting apparatus, electronic apparatus, or lighting device.

Another object of one embodiment of the present invention is to provide an organic compound in which a partial structure is selectively deuterated. Another object of one embodiment of the present invention is to provide an organic compound in which a partial structure having an effect of a longer lifetime is selectively deuterated. Another object of one embodiment of the present invention is to perform a molecular design with which the degree of complexity of a synthesis pathway can be reduced and increases in the temperature, pressure, and the like for synthesis can be inhibited.

Note that the description of these objects does not preclude the existence of other objects. One embodiment of the present invention does not have to achieve all of these objects. Note that other objects will be apparent from the description of the specification, the drawings, the claims, and the like, and other objects can be derived from the description of the specification, the drawings, the claims, and the like.

### Means for Solving the Problems

One embodiment of the present invention is an organic compound represented by General Formula (G1).

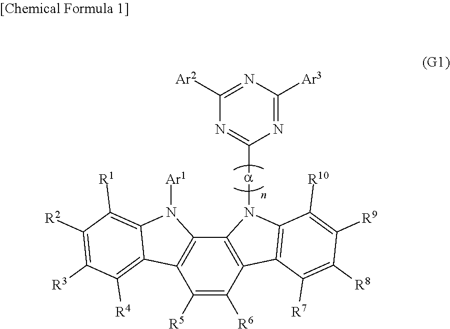

In General Formula (G1), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ to Ar³ independently represents a substituted or unsubstituted aryl group having 6 to 30 carbon atoms or a substituted or unsubstituted heteroaryl group having 2 to 30 carbon atoms; α represents a substituted or unsubstituted arylene group having 6 to 30 carbon atoms or a substituted or unsubstituted heteroarylene group having 2 to 30 carbon atoms; n represents an integer of 0 to 2; and when n is 2, two α may be the same with or different from each other.

Another embodiment of the present invention is an organic compound represented by General Formula (G2).

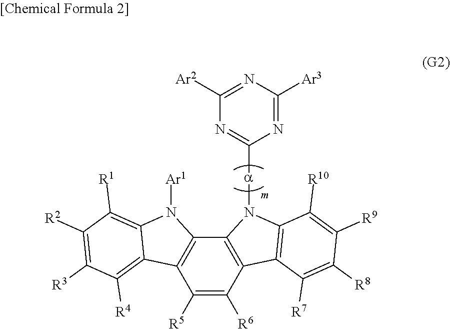

In General Formula (G2), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ to Ar³ independently represents a substituted or unsubstituted aryl group having 6 to 30 carbon atoms or a substituted or unsubstituted heteroaryl group having 2 to 30 carbon atoms; α represents a substituted or unsubstituted arylene group having 6 to 30 carbon atoms or a substituted or unsubstituted heteroarylene group having 2 to 30 carbon atoms; m represents 1 or 2; and when m is 2, two α may be the same with or different from each other.

One embodiment of the present invention is an organic compound with any of the above structures in which each of the arylene group having 6 to 30 carbon atoms and the heteroarylene group having 2 to 30 carbon atoms is independently represented by any one of Formulae (α-1) to (α-20).

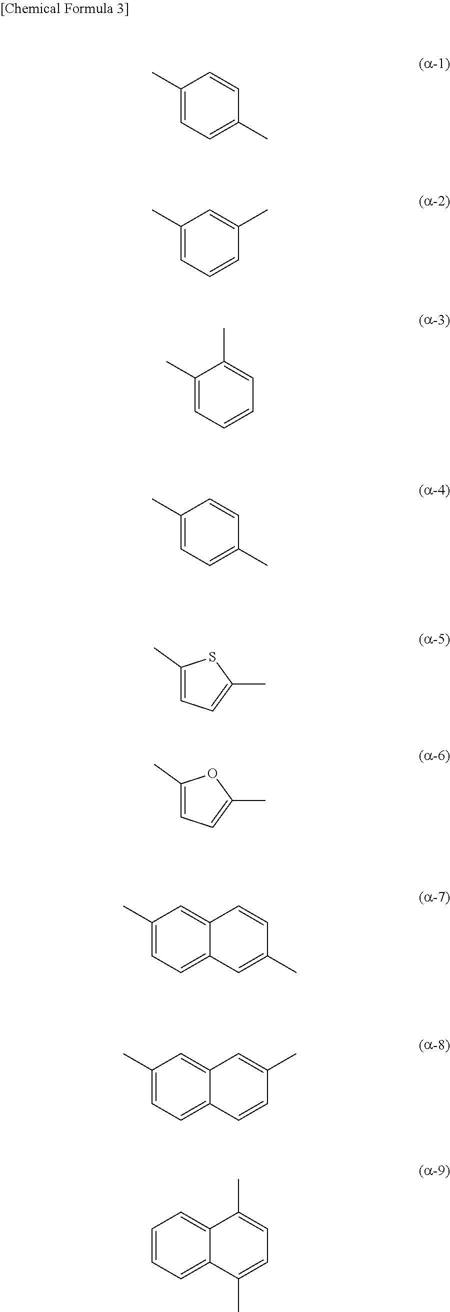

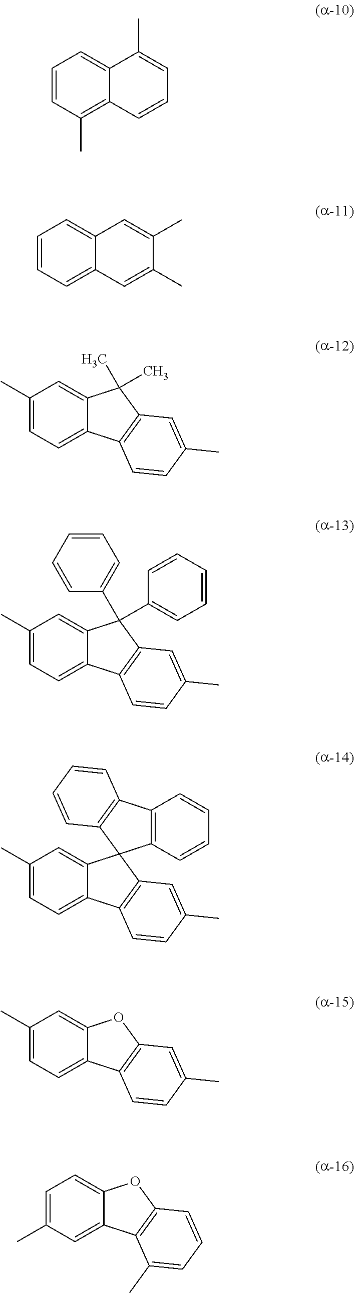

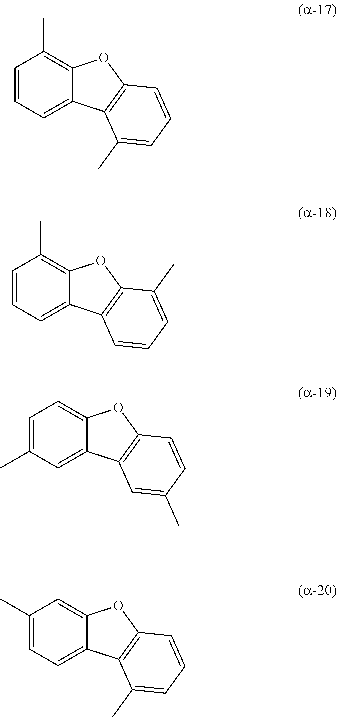

Another embodiment of the present invention is an organic compound represented by General Formula (G3).

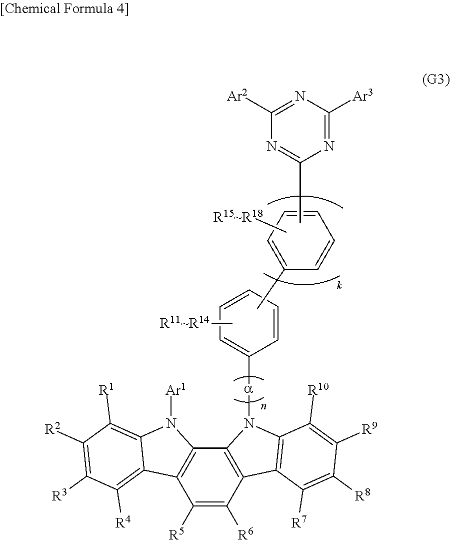

In General Formula (G3), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ to Ar³ independently represents a substituted or unsubstituted aryl group having 6 to 30 carbon atoms or a substituted or unsubstituted heteroaryl group having 2 to 30 carbon atoms; each of R¹¹ to R¹⁸ independently represents hydrogen, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; and k represents 0 or 1.

Another embodiment of the present invention is an organic compound represented by General Formula (G4).

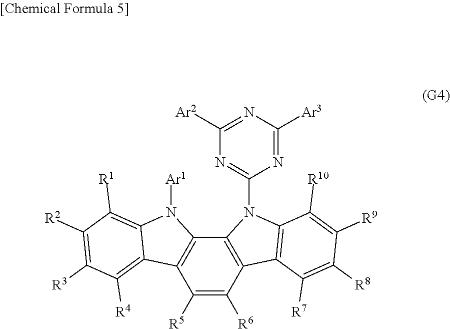

In General Formula (G4), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; and each of Ar¹ to Ar³ independently represents a substituted or unsubstituted aryl group having 6 to 30 carbon atoms or a substituted or unsubstituted heteroaryl group having 2 to 30 carbon atoms.

Another embodiment of the present invention is an organic compound represented by General Formula (G5).

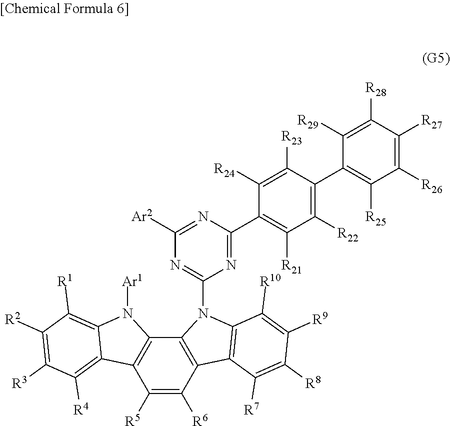

In General Formula (G5), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ and Ar² independently represents a substituted or unsubstituted aryl group having 6 to 30 carbon atoms or a substituted or unsubstituted heteroaryl group having 2 to 30 carbon atoms; and each of R²¹ to R²⁹ independently represents hydrogen, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms.

Another embodiment of the present invention is an organic compound with the above structure in which each of the aryl group having 6 to 30 carbon atoms and the heteroaryl group having 2 to 30 carbon atoms is independently represented by any one of Formulae (Ar-1) to (Ar-80).

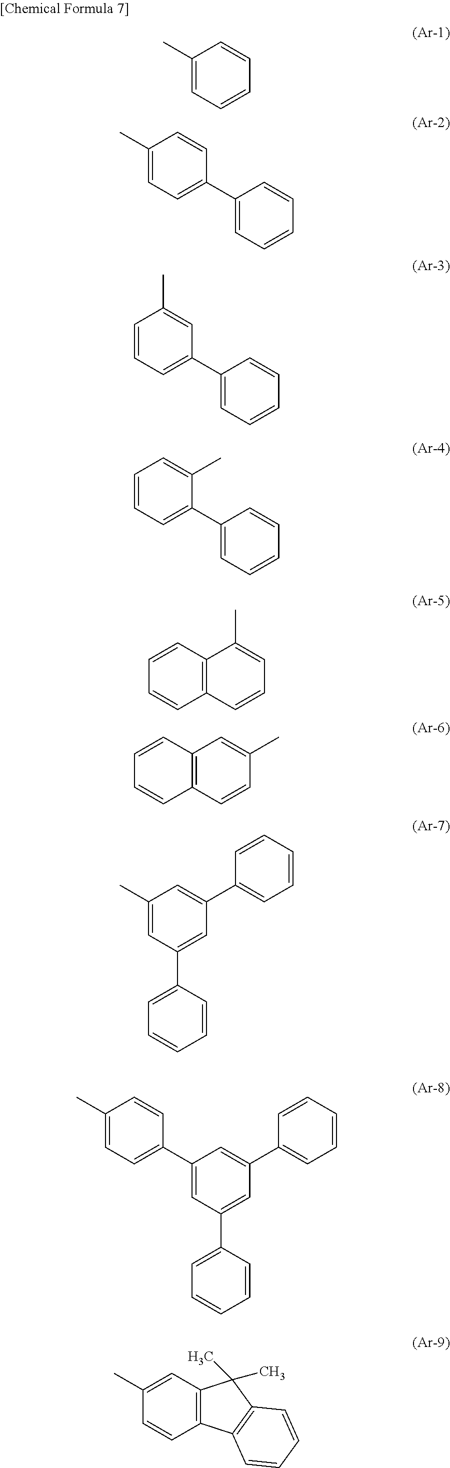

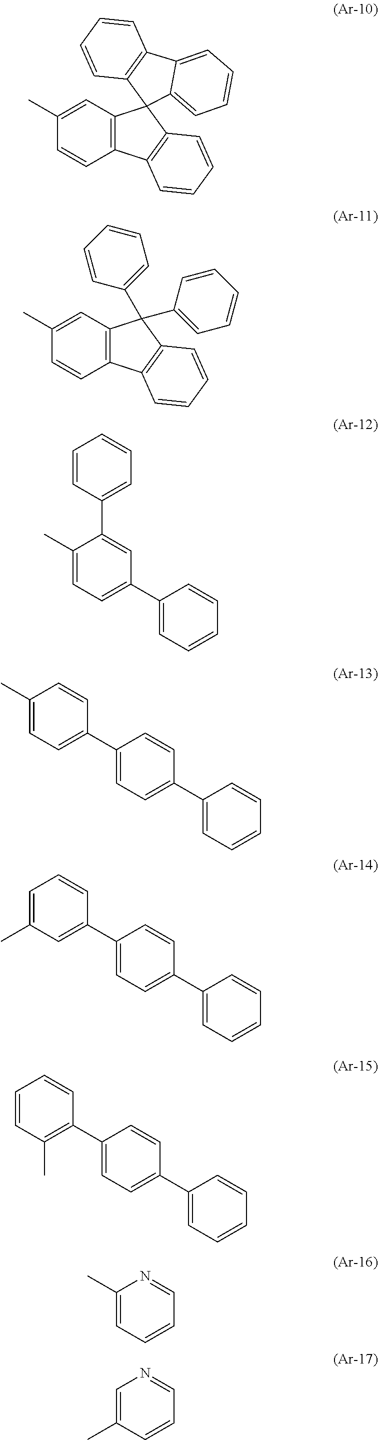

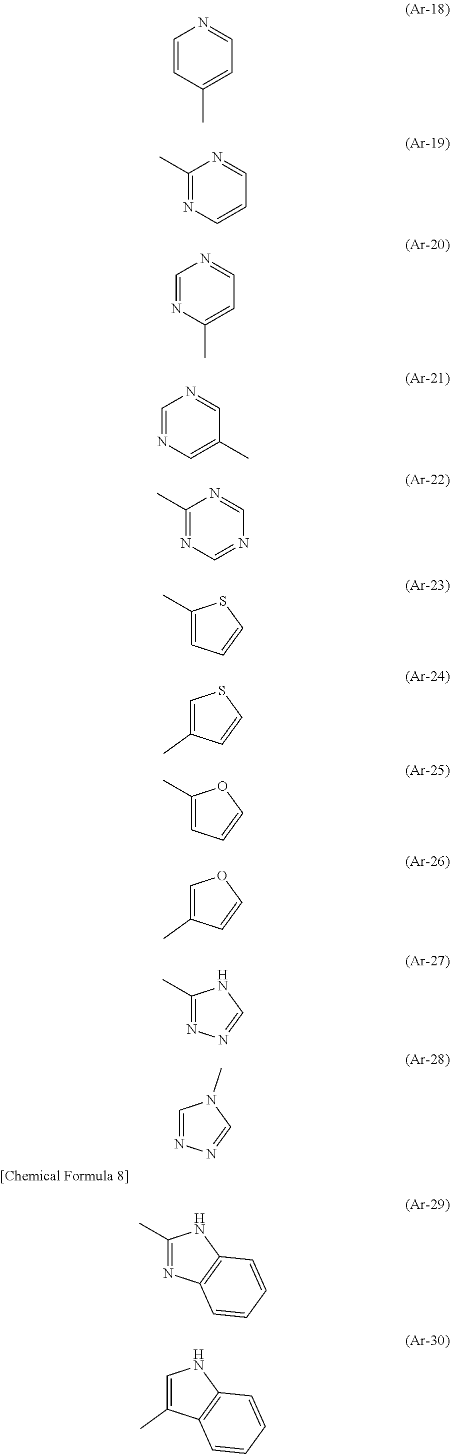

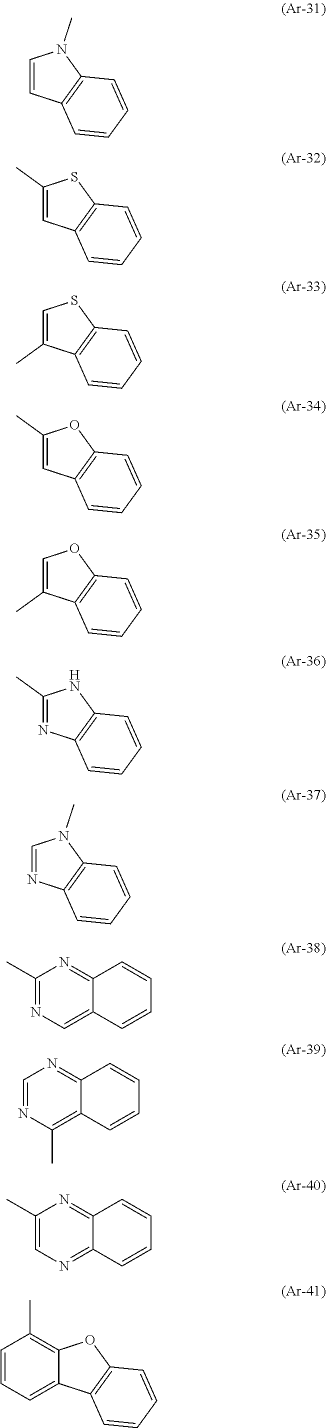

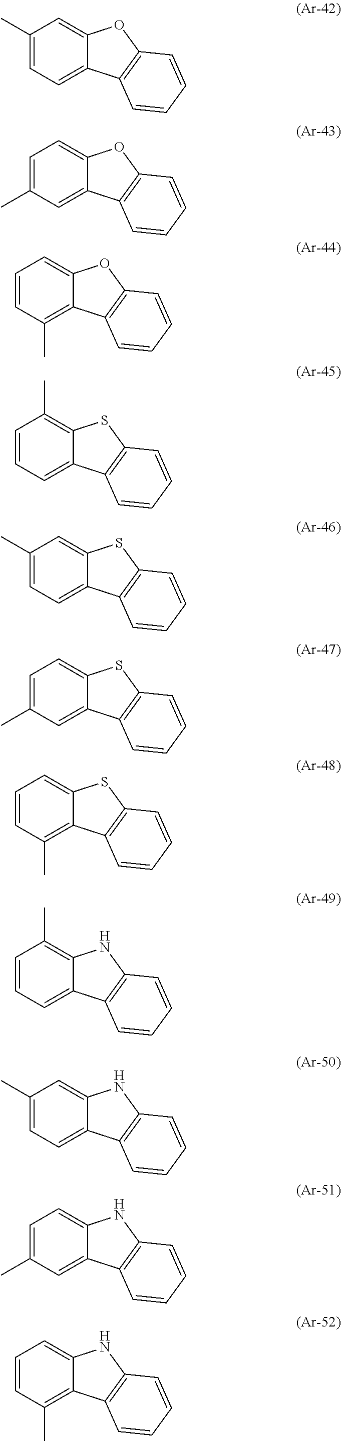

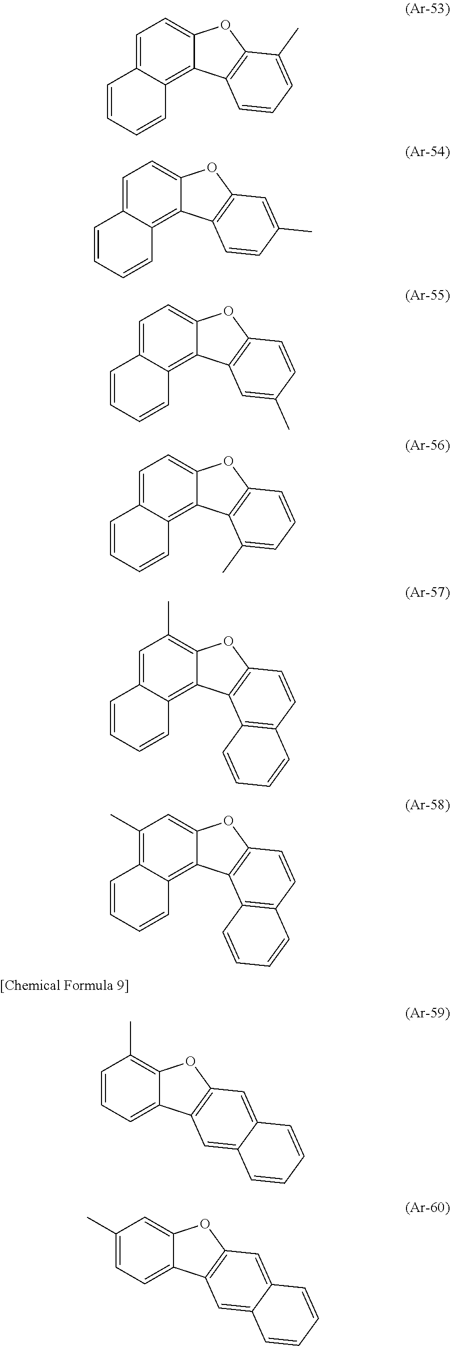

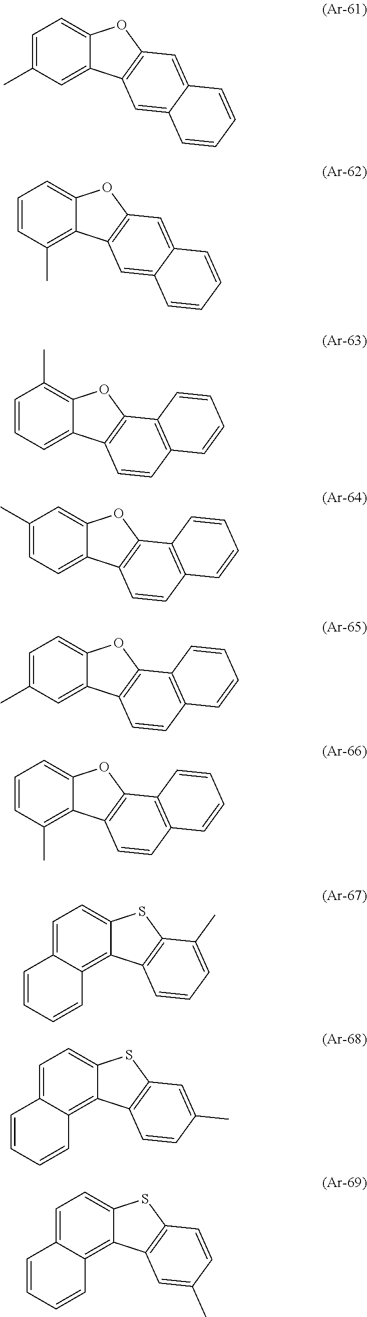

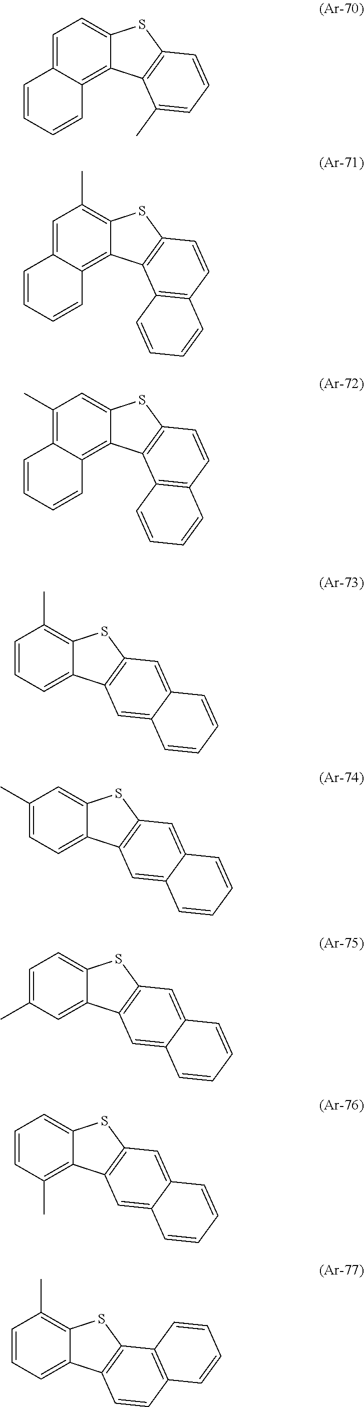

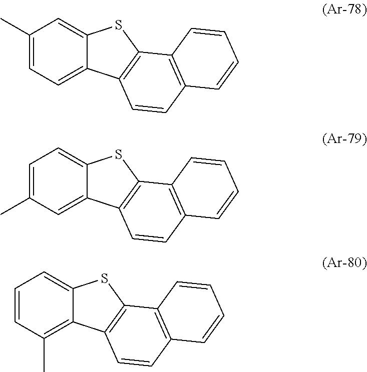

Another embodiment of the present invention is the organic compound with any of the above structures, in which a plurality of or all of R¹ to R¹⁰ are deuterium.

Another embodiment of the present invention is an organic compound represented by Structural Formula (101) or Structural Formula (105).

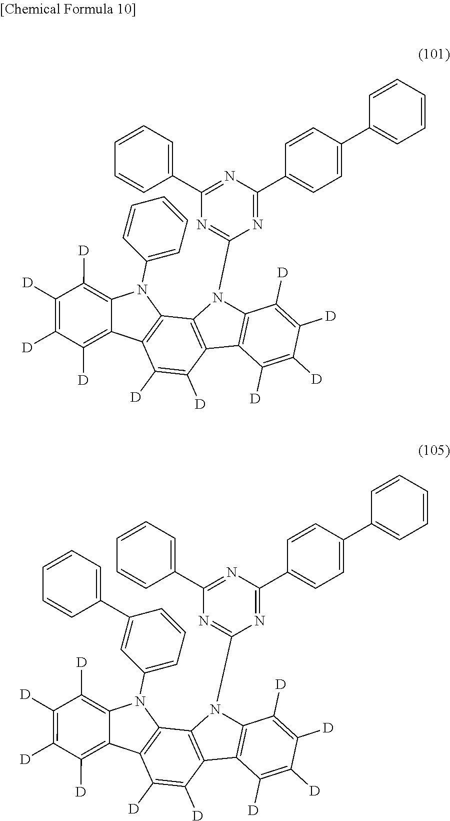 [SMILES: [2H]c1c([2H])c([2H])c2c(c1[2H])c1c([2H])c([2H])c3c4c([2H])c([2H])c([2H])c([2H])c4n(-c4nc(-c5ccccc5)nc(-c5ccc(-c6ccccc6)cc5)n4)c3c1n2-c1ccccc1 ; [2H]c1c([2H])c([2H])c2c(c1[2H])c1c([2H])c([2H])c3c4c([2H])c([2H])c([2H])c([2H])c4n(-c4nc(-c5ccccc5)nc(-c5ccc(-c6ccccc6)cc5)n4)c3c1n2-c1cccc(-c2ccccc2)c1]

Another embodiment of the present invention is an organic compound including an indolocarbazole skeleton and an azine skeleton, in which the indolocarbazole skeleton is selectively deuterated. The azine skeleton is a triazine skeleton, a pyrimidine skeleton, or a pyridine skeleton.

Another embodiment of the present invention is a thin film including the organic compound having any of the above structures.

Another embodiment of the present invention is a light-emitting device containing the organic compound having any of the above structures.

Another embodiment of the present invention is a light-emitting apparatus including the light-emitting device with the above structure, and a transistor or a substrate.

Another embodiment of the present invention is an electronic apparatus including the light-emitting apparatus with the above structure, and a detecting portion, an input portion, or a communication portion.

Another embodiment of the present invention is a lighting device including the light-emitting apparatus with the above structure and a housing.

### Effect of the Invention

One embodiment of the present invention can provide a novel organic compound. One embodiment of the present invention can provide an organic compound with a long lifetime. One embodiment of the present invention can provide an organic compound that can be used as a host material. One embodiment of the present invention can provide an organic compound that is easily synthesized. One embodiment of the present invention can provide a novel light-emitting device. One embodiment of the present invention can provide a light-emitting device with a long lifetime. One embodiment of the present invention can reduce a manufacturing cost of a light-emitting device. One embodiment of the present invention can provide a light-emitting apparatus, an electronic apparatus, or a lighting device with low power consumption. One embodiment of the present invention can provide a light-emitting apparatus, an electronic apparatus, or a lighting device with low power consumption.

One embodiment of the present invention can provide an organic compound in which a partial structure is selectively deuterated. One embodiment of the present invention can provide an organic compound in which a partial structure having an effect of a longer lifetime is selectively deuterated. As a result, the degree of complexity of a synthesis pathway and high temperature and high pressure in the synthesis pathway which are caused in substituting deuterium for all hydrogen of an organic compound can be reduced.

Note that the description of these effects does not preclude the existence of other effects. One embodiment of the present invention does not need to have all of these effects. Other effects will be apparent from the description of the specification, the drawings, the claims, and the like, and other effects can be derived from the description of the specification, the drawings, the claims, and the like.

### BRIEF DESCRIPTION OF THE DRAWINGS

FIG. 1A to FIG. 1E are diagrams illustrating structures of a light-emitting device of Embodiment.

FIG. 2A to FIG. 2D are diagrams illustrating a light-emitting apparatus of Embodiment.

FIG. 3A to FIG. 3C are diagrams illustrating a method of manufacturing a light-emitting apparatus of Embodiment.

FIG. 4A to FIG. 4C are diagrams illustrating a method of manufacturing a light-emitting apparatus of Embodiment.

FIG. 5A to FIG. 5C are diagrams illustrating a method of manufacturing a light-emitting apparatus of Embodiment.

FIG. 6A to FIG. 6D are diagrams illustrating a method of manufacturing a light-emitting apparatus of Embodiment.

FIG. 7A to FIG. 7D are diagrams illustrating a light-emitting apparatus of Embodiment.

FIG. 8A to FIG. 8F are diagrams illustrating an apparatus and pixel arrangement of Embodiment.

FIG. 9A to FIG. 9C are diagrams each illustrating a pixel circuit of Embodiment.

FIG. 10 is a diagram illustrating a light-emitting apparatus of Embodiment.

FIG. 11A to FIG. 11E are diagrams illustrating electronic apparatuses of Embodiment.

FIG. 12A to FIG. 12E are diagrams illustrating electronic apparatuses of Embodiment.

FIG. 13A and FIG. 13B are diagrams illustrating electronic apparatuses of Embodiment.

FIG. 14A and FIG. 14B are diagrams illustrating a lighting device of Embodiment.

FIG. 15 is a diagram illustrating a lighting device of Embodiment.

FIG. 16A to FIG. 16C are diagrams illustrating a light-emitting device and a light-receiving device of Embodiment.

FIG. 17A and FIG. 17B are diagrams each illustrating a light-emitting device and a light-receiving device of Embodiment.

FIG. 18A and FIG. 18B show a ¹HNMR spectrum of BP-Icz(II)Tzn-d10.

FIG. 19A shows a ¹HNMR spectrum of BP-Icz(II)Tzn, and FIG. 19B shows 1HNMR spectra of BP-Icz(II)Tzn-d10 and BP-Icz(II)Tzn.

FIG. 20 shows an absorption spectrum and an emission spectrum of BP-Icz(II)Tzn-d10 in a dichloromethane solution.

FIG. 21 shows an absorption spectrum and an emission spectrum of a thin film of BP-Icz(II)Tzn-d10.

FIG. 22A and FIG. 22B show a ¹HNMR spectrum of BP-mBPIcz(II)Tzn-d10.

FIG. 23 shows an absorption spectrum and an emission spectrum of BP-mBPIcz(II)Tzn-d10 in a dichloromethane solution.

FIG. 24 is a diagram illustrating a structure of a light-emitting device of Example.

FIG. 25 shows luminance-current density characteristics of a light-emitting device 1 and a comparative light-emitting device 2.

FIG. 26 shows current efficiency-luminance characteristics of the light-emitting device 1 and the comparative light-emitting device 2.

FIG. 27 shows luminance-voltage characteristics of the light-emitting device 1 and the comparative light-emitting device 2.

FIG. 28 shows current-voltage characteristics of the light-emitting device 1 and the comparative light-emitting device 2.

FIG. 29 shows external quantum efficiency-luminance characteristics of the light-emitting device 1 and the comparative light-emitting device 2.

FIG. 30 shows emission spectra of the light-emitting device 1 and the comparative light-emitting device 2.

FIG. 31 is a graph showing changes in luminance over driving time of the light-emitting device 1 and the comparative light-emitting device 2.

FIG. 32 is a graph showing changes in luminance over driving time of the light-emitting device 1 and a light-emitting device 1-a to a light-emitting device 1-c.

FIG. 33 is a diagram illustrating HOMO distribution of BP-Icz(II)Tzn-d10.

FIG. 34 is a diagram illustrating HOMO distribution of BP-mBPIcz(II)Tzn-d10.

FIG. 35 shows luminance-current density characteristics of a light-emitting device 3 and a comparative light-emitting device 4.

FIG. 36 shows current efficiency-luminance characteristics of the light-emitting device 3 and the comparative light-emitting device 4.

FIG. 37 shows luminance-voltage characteristics of the light-emitting device 3 and the comparative light-emitting device 4.

FIG. 38 shows current-voltage characteristics of the light-emitting device 3 and the comparative light-emitting device 4.

FIG. 39 shows external quantum efficiency-luminance characteristics of the light-emitting device 3 and the comparative light-emitting device 4.

FIG. 40 shows emission spectra of the light-emitting device 3 and the comparative light-emitting device 4.

FIG. 41 is a graph showing changes in luminance over driving time of the light-emitting device 3 and the comparative light-emitting device 4.

### MODE FOR CARRYING OUT THE INVENTION

### Embodiment 1

In this embodiment, an organic compound and a thin film of one embodiment of the present invention will be described.

One embodiment of the present invention is an organic compound that is a bipolar substance having both a hole-transport skeleton and an electron-transport skeleton, in which the hole-transport skeleton is deuterated. Specifically, one embodiment of the present invention is a bipolar substance having a deuterated indolocarbazole skeleton as a hole-transport skeleton and a triazine skeleton as an electron-transport skeleton. One embodiment of the present invention has both a hole-transport skeleton and an electron-transport skeleton, thereby having both a hole-transport property and an electron-transport property. Accordingly, one embodiment of the present invention can be suitably used as a host material for a light-emitting layer of a light-emitting device, for example. Furthermore, one embodiment of the present invention can be suitably used for a hole-transport layer and an electron-transport layer as a transport layer in contact with a light-emitting layer.

Note that in this specification and the like, deuteration means that deuterium (D) is substituted for at least one hydrogen (H) included in an organic compound, a substituent, or a partial structure of the organic compound. Hydrogen (H) is referred to as light hydrogen in some cases.

The bond dissociation energy of a bond between carbon and deuterium (C-D bond) is higher than the bond dissociation energy of a bond between carbon and hydrogen (light hydrogen) (C—H bond), and thus the C-D bond is stable and not easily cut. Accordingly, in one embodiment of the present invention, when a hole-transport skeleton is deuterated, carbon-hydrogen bond dissociation in a hole-transport skeleton in a ground state or an excited state can be inhibited. Deterioration or a change in quality of an organic compound due to carbon-hydrogen bond dissociation in a hole-transport skeleton can be inhibited.

The organic compound of one embodiment of the present invention has a hole-transport skeleton; therefore, when the organic compound of one embodiment of the present invention is used for a light-emitting device as a host material, for example, the hole-transport skeleton receives a hole in some cases. Carbon-hydrogen bond dissociation easily occurs in hole transfer in some cases; however, a hole-transport skeleton is deuterated in the organic compound of one embodiment of the present invention, and thus carbon-hydrogen bond dissociation can be inhibited.

Note that in synthesis of an organic compound in which the whole structure of a bipolar substance having a hole-transport skeleton and an electron-transport skeleton is deuterated, a complicated synthesis pathway, necessity of high temperature and high pressure, or the like is a problem. Accordingly, one embodiment of the present invention can be easily synthesized by selectively deuterating only a hole-transport skeleton.

In this specification and the like, the deuteration rate of a hole-transport skeleton refers to a rate of substitution of deuterium for hydrogen directly bonded to a hole-transport skeleton. For example, in the case where deuterium is substituted for 10% of hydrogen directly bonded to a hole-transport skeleton, the deuteration rate of the hole-transport skeleton is 10%. Furthermore, in the case where a hole-transport skeleton has a substituent, hydrogen or deuterium of the substituent is not used for the calculation of the deuteration rate of the hole-transport skeleton. In the case where only deuterium and a phenyl group directly bond to a hole-transport skeleton, for example, the deuteration rate of the hole-transport skeleton is 100%, regardless of the proportion of hydrogen and deuterium in the phenyl group.

That is, one embodiment of the present invention is an organic compound represented by General Formula (G1).

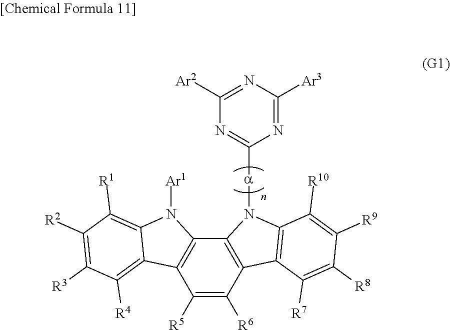

In General Formula (G1), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ to Ar³ independently represents a substituted or unsubstituted aryl group having 6 to 30 carbon atoms or a substituted or unsubstituted heteroaryl group having 2 to 30 carbon atoms; α represents a substituted or unsubstituted arylene group having 6 to 30 carbon atoms or a substituted or unsubstituted heteroarylene group having 2 to 30 carbon atoms; n represents an integer of 0 to 2; and when n is 2, two α may be the same with or different from each other.

Note that in General Formula (G1) above, when n is 0, the highest occupied molecular orbital (HOMO) level tends to be deep; when n is 1 or 2, the HOMO level tends to be shallow. In such a manner, the HOMO level of the organic compound can be changed by changing n. When n is 1 or 2, the molecular weight is larger than that when n is 0, which is preferable because the heat resistance is improved. Meanwhile, when n is 0, a TADF (Thermally Activated Delayed Fluorescence) compound is preferably used as a host, in which case a light-emitting device with high efficiency can be provided, the sublimation property can be increased because the molecular weight is not too large, and a light-emitting layer with a high purity can be provided because decomposition at evaporation can be prevented. As a result, a device with high reliability can be provided.

Another embodiment of the present invention is an organic compound represented by General Formula (G2).

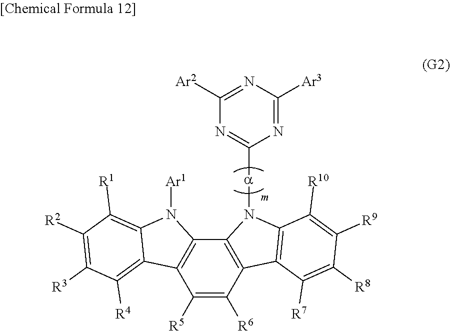

In General Formula (G2), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ to Ar³ independently represents a substituted or unsubstituted aryl group having 6 to 30 carbon atoms or a substituted or unsubstituted heteroaryl group having 2 to 30 carbon atoms; α represents a substituted or unsubstituted arylene group having 6 to 30 carbon atoms or a substituted or unsubstituted heteroarylene group having 2 to 30 carbon atoms; m represents 1 or 2; and when m is 2, two α may be the same with or different from each other.

The organic compound represented by General Formula (G2) includes a substituted or unsubstituted arylene group having 6 to 30 carbon atoms or a substituted or unsubstituted heteroarylene group having 2 to 30 carbon atoms between an indolocarbazole skeleton and a triazine skeleton. This is preferable because the HOMO level can be shallower than that in the case where m is 0, and thus a host material with a HOMO level suitable for device design can be provided. In addition, the heat resistance can be higher than that in the case where m is 0.

In General Formula (G1) and General Formula (G2) above, it is preferable that each of an arylene group having 6 to 30 carbon atoms and a heteroarylene group having 2 to 30 carbon atoms be independently represented by any one of Structural Formulae (α-1) to (α-20).

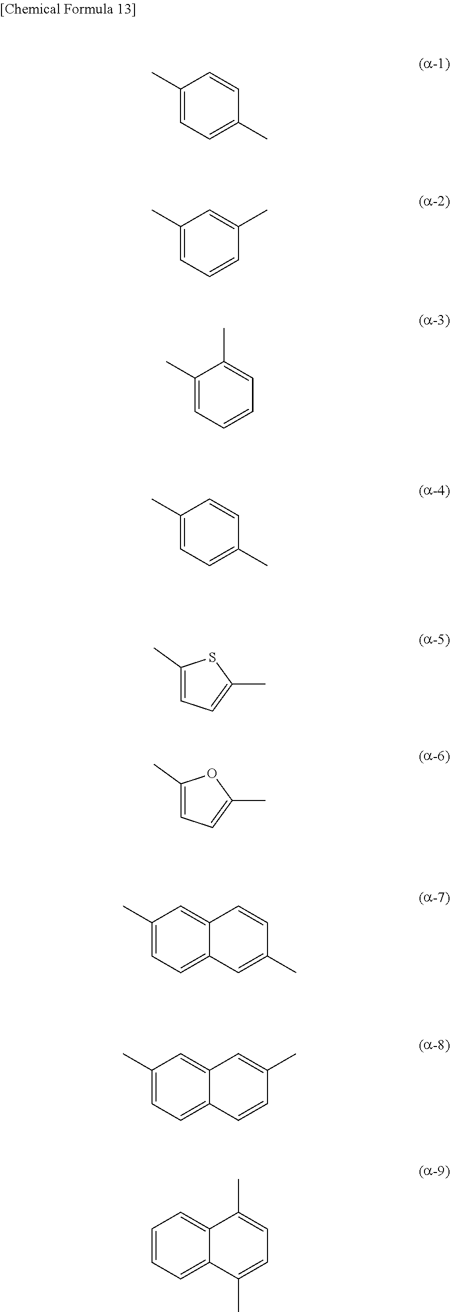

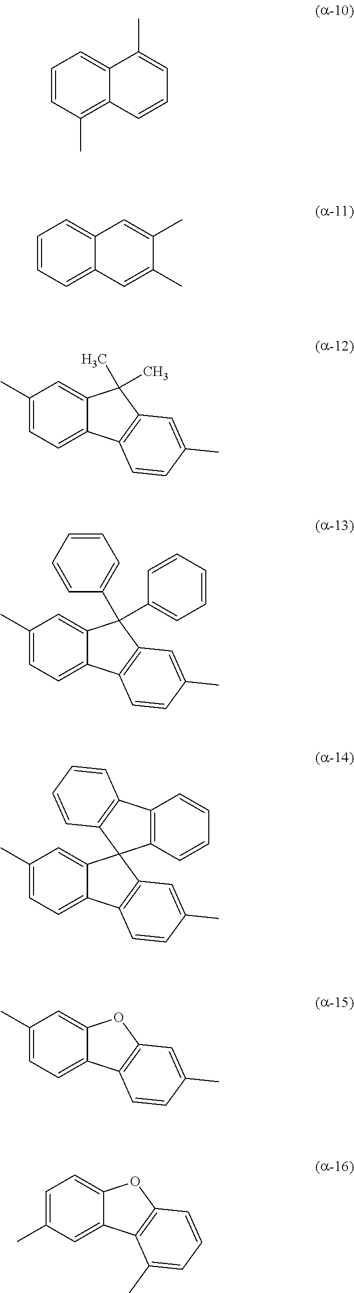

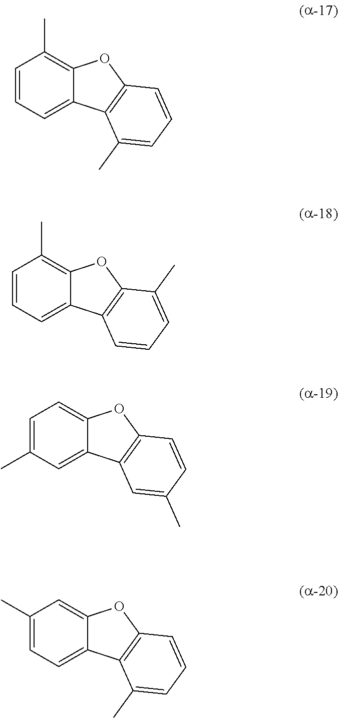

Note that the substituents represented by Structural Formulae (α-1) to (α-20) are examples of the arylene group having 6 to 30 carbon atoms and the heteroarylene group having 2 to 30 carbon atoms; however, the arylene group having 6 to 30 carbon atoms and the heteroarylene group having 2 to 30 carbon atoms which can be used in General Formula (G1) and General Formula (G2) above are not limited to these. When an arylene group or a heteroarylene group is included as a substituent, carrier balance can be adjusted by changing the HOMO level or heat resistance can be improved.

In General Formula (G1) and General Formula (G2) above, in the case where the arylene group having 6 to 30 carbon atoms and the heteroarylene group having 2 to 30 carbon atoms each include a substituent, the substituent is an alkyl group having 1 to 6 carbon atoms or an aryl group having 6 to 13 carbon atoms. When an alkyl group is included as a substituent, the refractive index can be low. When an aryl group is included, the heat resistance can be improved.

Another embodiment of the present invention is an organic compound represented by General Formula (G3).

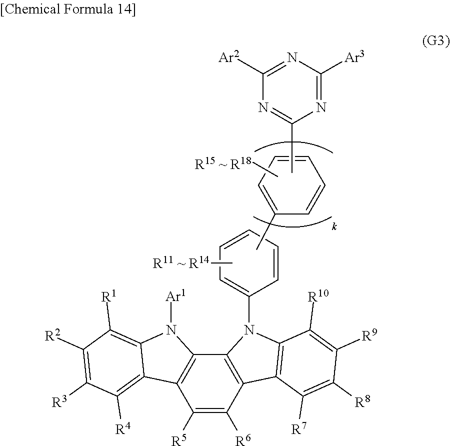

In General Formula (G3), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ to Ar³ independently represents a substituted or unsubstituted aryl group having 6 to 30 carbon atoms or a substituted or unsubstituted heteroaryl group having 2 to 30 carbon atoms; each of R¹¹ to R¹⁸ independently represents hydrogen, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; and k represents 0 or 1.

The organic compound represented by General Formula (G3) necessarily includes a substituted or unsubstituted phenylene group or biphenylene group between an indolocarbazole skeleton and a triazine skeleton. This is preferable because the HOMO level can be shallow and heat resistance can be improved as compared with the case where the organic compound does not include them; thus, a host material having a HOMO level suitable for device design can be provided.

Another embodiment of the present invention is an organic compound represented by General Formula (G4).

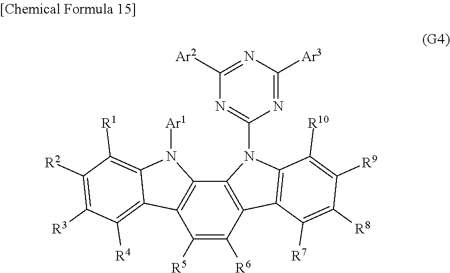

In General Formula (G4), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; and each of Ar¹ to Ar³ independently represents a substituted or unsubstituted aryl group having 6 to 30 carbon atoms or a substituted or unsubstituted heteroaryl group having 2 to 30 carbon atoms.

In the organic compound represented by General Formula (G4), an indolocarbazole skeleton and a triazine skeleton are directly bonded to each other. This is preferable because the HOMO level can be deeper than that in the case where an indolocarbazole skeleton and a triazine skeleton are not directly bonded to each other, and thus a host material with a HOMO level suitable for device design can be provided. In addition, the organic compound has high possibility of having a TADF property, which is preferable because a light-emitting device with high efficiency can be provided when the organic compound is used as a host. Furthermore, the sublimation property can be improved, which is preferable because decomposition at evaporation can be prevented and thus a light-emitting layer with a high purity can be provided. As a result, a device with high reliability can be provided.

Another embodiment of the present invention is an organic compound represented by General Formula (G5).

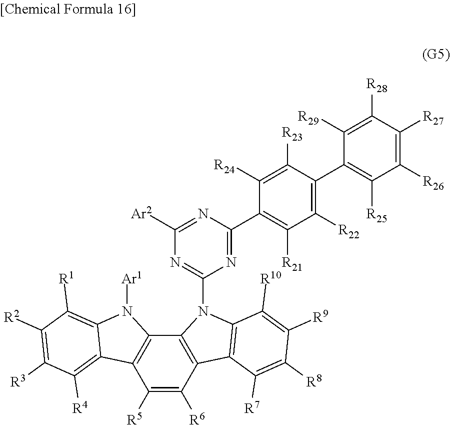

In General Formula (G5), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ and Ar² independently represents a substituted or unsubstituted aryl group having 6 to 30 carbon atoms or a substituted or unsubstituted heteroaryl group having 2 to 30 carbon atoms; and each of R²¹ to R²⁹ independently represents hydrogen, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms.

In General Formula (G1) to General Formula (G5) above, it is preferable that each of an aryl group having 6 to 30 carbon atoms and a heteroaryl group having 2 to 30 carbon atoms be independently represented by any one of Formulae (Ar-1) to (Ar-80).

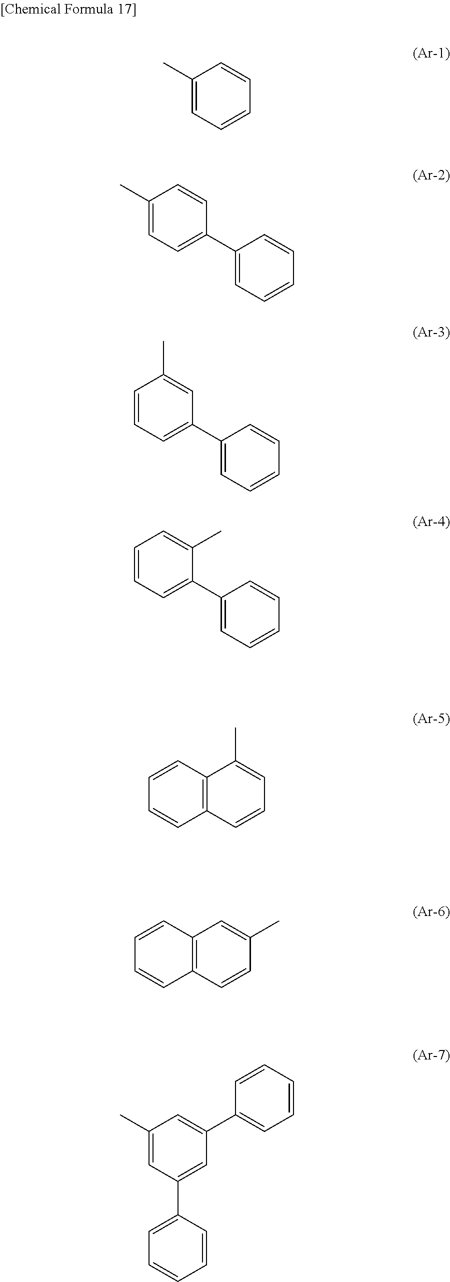

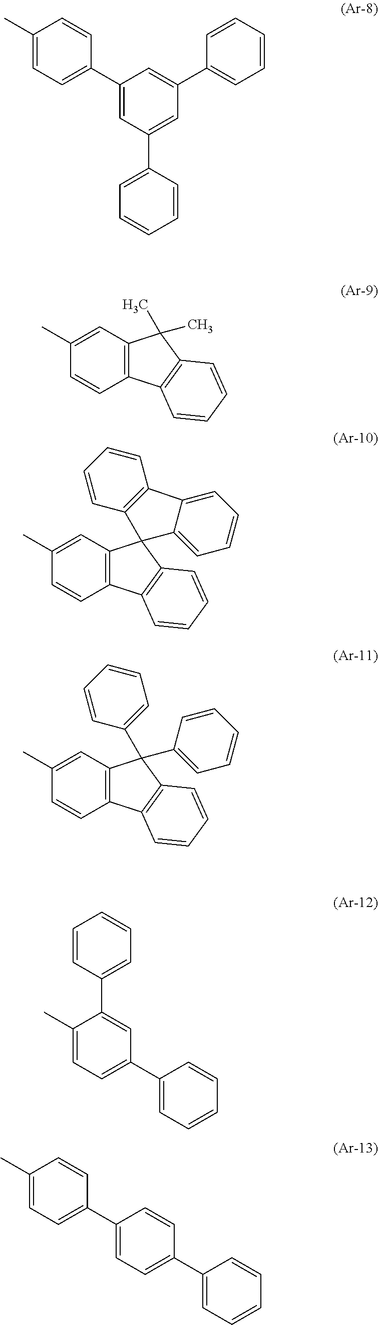

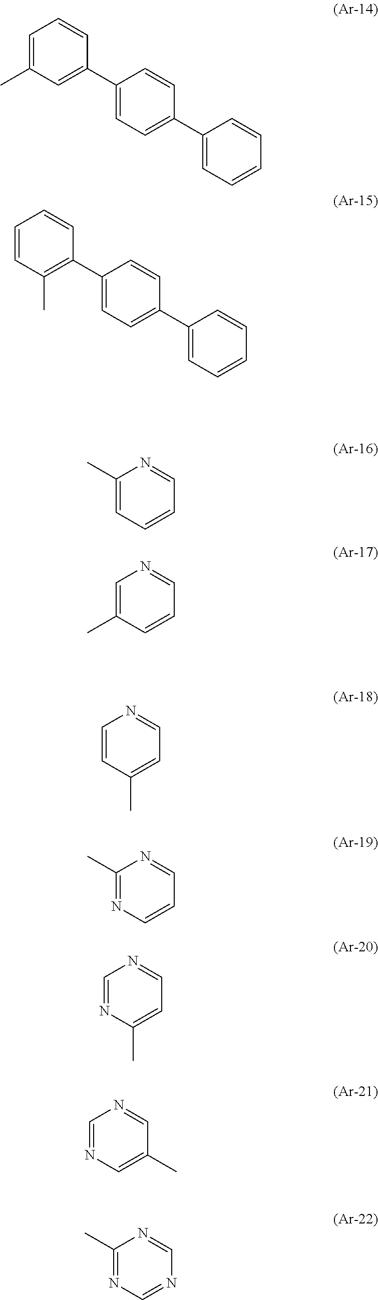

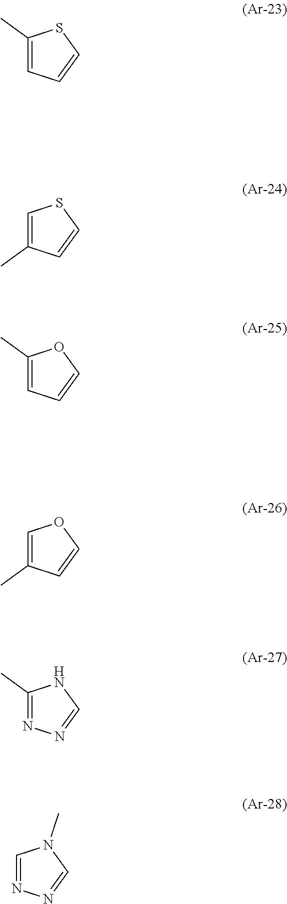

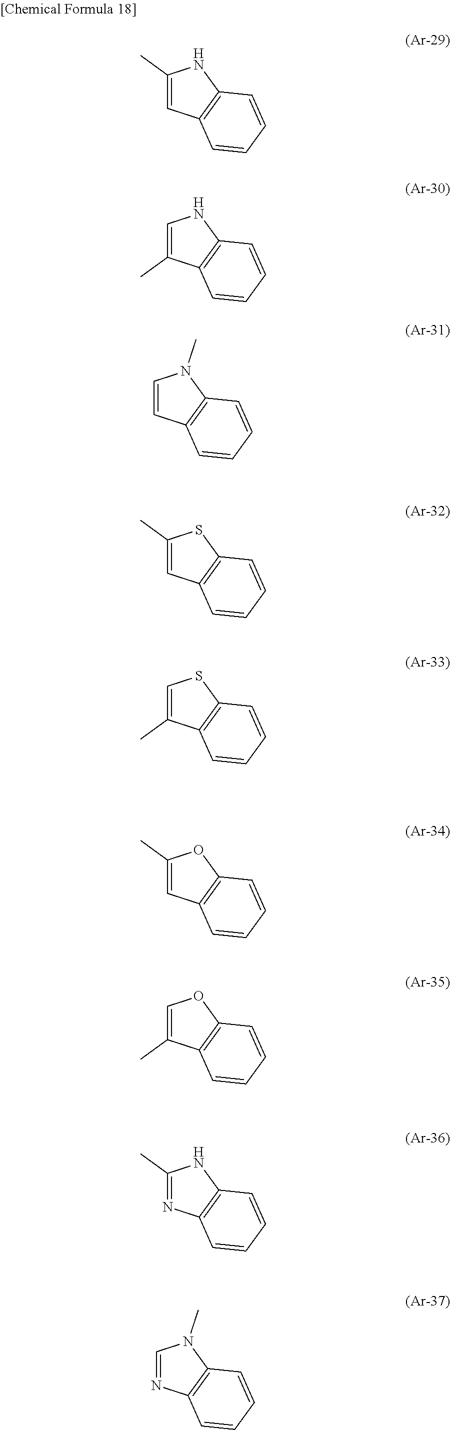

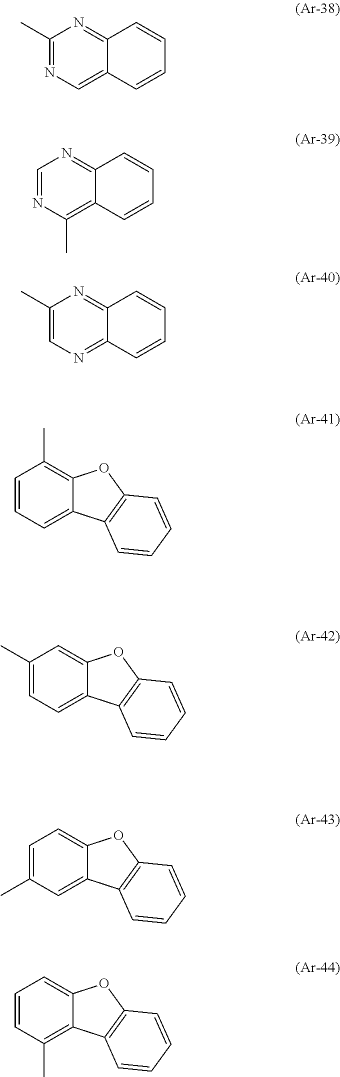

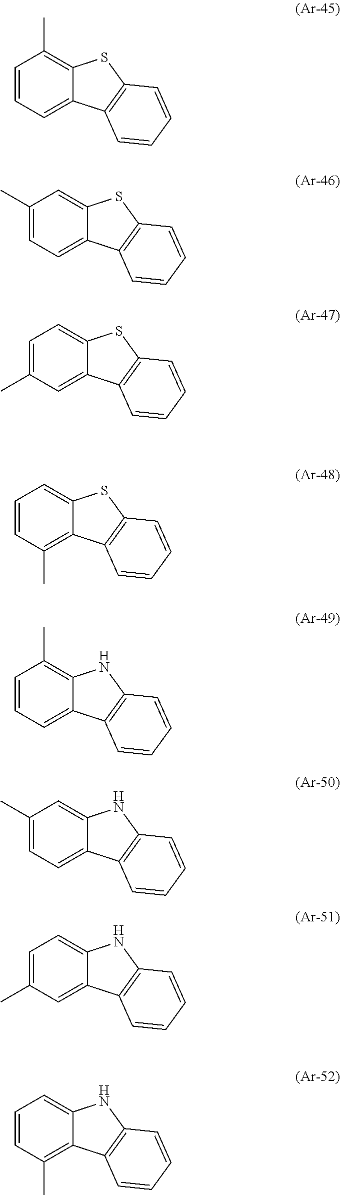

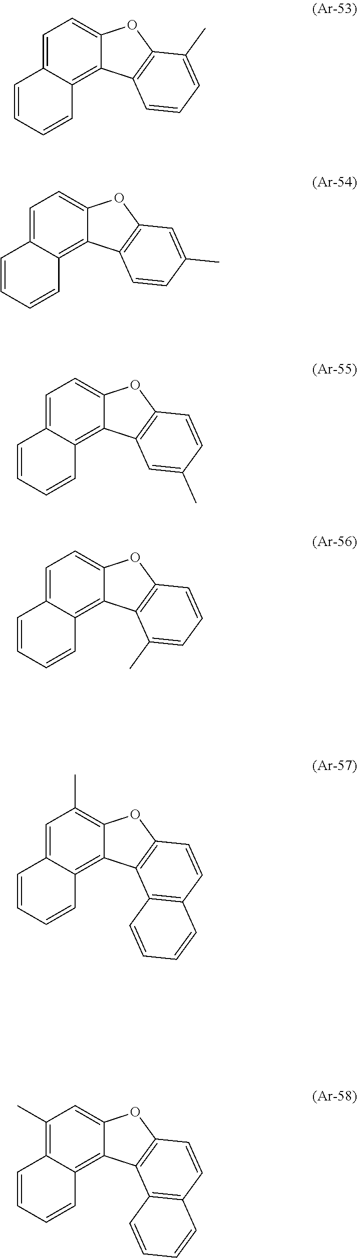

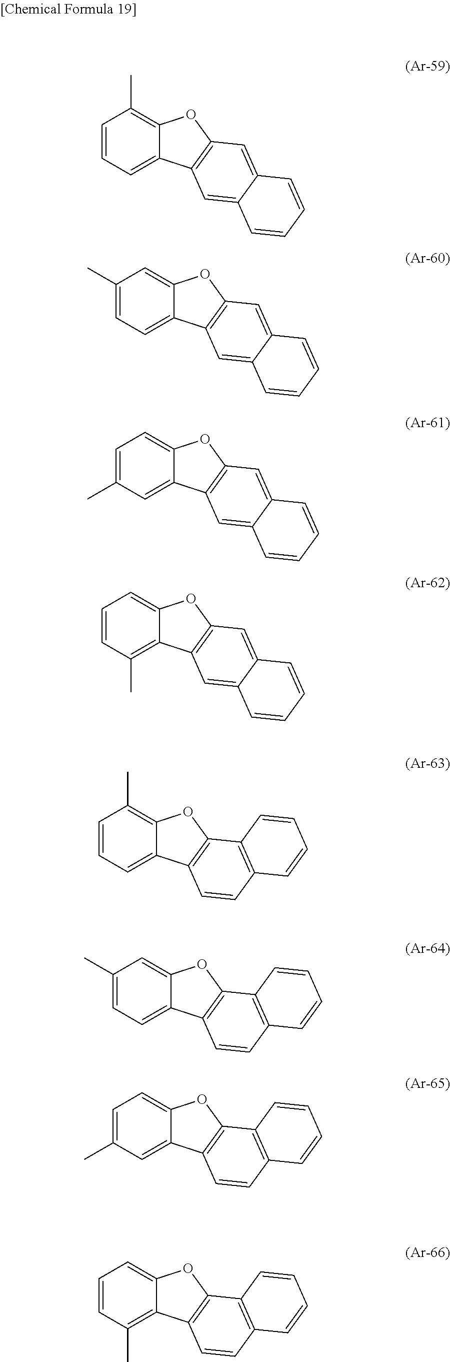

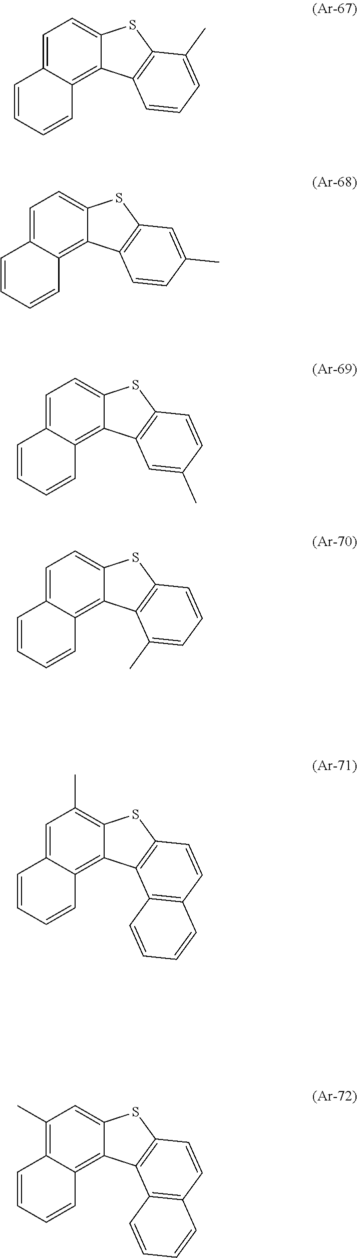

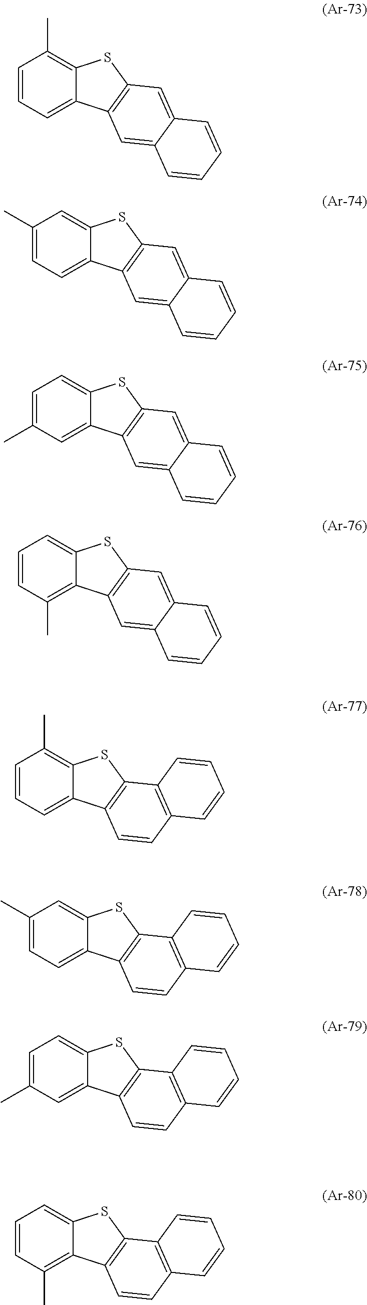

Note that the substituents represented by Structural Formulae (Ar-1) to (Ar-80) are examples of the aryl group having 6 to 30 carbon atoms and the heteroaryl group having 2 to 30 carbon atoms; however, the aryl group having 6 to 30 carbon atoms and the heteroaryl group having 2 to 30 carbon atoms which can be used in General Formula (G1) to General Formula (G5) above are not limited to these.

In General Formula (G1) to General Formula (G5) above, in the case where the aryl group having 6 to 30 carbon atoms and the heteroaryl group having 2 to 30 carbon atoms each include a substituent, the substituent is an alkyl group having 1 to 6 carbon atoms or an aryl group having 6 to 13 carbon atoms.

In General Formula (G1) to General Formula (G5) above, when any one or more of R¹ to R¹⁰ are a substituted or unsubstituted alkyl group having 1 to 6 carbon atoms, a substituted or unsubstituted aryl group having 6 to 30 carbon atoms, a substituted or unsubstituted heteroaryl group having 2 to 30 carbon atoms, these groups may be deuterated or not.

In General Formula (G1) to General Formula (G5) above, at least one of R¹ to R¹⁰ is deuterium, whereby carbon-hydrogen bond dissociation can be inhibited.

Furthermore, in General Formula (G1) to General Formula (G5) above, it is further preferable that some or all of R¹ to R¹⁰ be deuterium. In particular, when all of R¹ to R¹⁰ are deuterium, dissociation of all carbon-hydrogen bonds in a hole-transport skeleton can be prevented.

Note that in this specification and the like, the deuteration rate of the indolocarbazole skeleton in the hole-transport skeleton, that is, in the indolocarbazole skeleton in General Formula (G1) to General Formula (G5) above, refers to the proportion of hydrogen which is directly bonded to the indolocarbazole skeleton and is substituted by deuterium. For example, in the case where all R¹ to R¹⁰ are deuterium, the deuteration rate of the indolocarbazole skeleton is 100%. Furthermore, in the case where some of R¹ to R¹⁰ are not hydrogen or deuterium, that is, in the case where some of R¹ to R¹⁰ are substituents such as a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms, hydrogen or deuterium included in the substituents is not used to calculate the deuteration rate of the indolocarbazole skeleton. For example, in the case where 50% of R¹ to R⁸ is hydrogen, 50% is deuterium, and R⁹ and R¹⁰ each represent a phenyl group, the deuteration rate of the indolocarbazole skeleton is 50% regardless of the proportion of hydrogen and deuterium in the phenyl groups.

In General Formula (G1) to General Formula (G5) above, the deuteration rate of the indolocarbazole skeleton is preferably greater than or equal to 50% and less than or equal to 100%. Note that the deuteration rate of the indolocarbazole skeleton is preferably greater than or equal to 60%, further preferably greater than or equal to 70%, still further preferably greater than or equal to 80%, yet still further preferably greater than or equal to 90%.

Note that even when the organic compound represented by any of General Formula (G1) to General Formula (G5) is a mixture of a compound in which a substituent is deuterium and a compound in which a substituent is hydrogen, an effect can be obtained in some cases. For example, a mixture in which compounds whose R¹ in General Formula (G1) to General Formula (G5) is deuterium occupies 50% and compounds whose R¹ is hydrogen occupies 50% can also have an effect. The same applies to R² to R¹⁰.

In General Formula (G1) to General Formula (G5) above, specific examples of the alkyl group having 1 to 6 carbon atoms include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, and a 2,3-dimethylbutyl group.

In the case where the alkyl group having 1 to 6 carbon atoms includes a substituent, examples of the substituent are an alkyl group having 1 to 6 carbon atoms and an aryl group having 6 to 13 carbon atoms.

In General Formula (G1) to General Formula (G5) above, examples of the aryl group having 6 to 13 carbon atoms include a phenyl group, a tolyl group, a xylyl group, a mesityl group, a biphenyl group, a naphthyl group, and a fluorenyl group.

In the case where the organic compound of one embodiment of the present invention represented by any of General Formulae (G1) to (G5) above is used for a light-emitting device, the organic compound is preferably formed into a thin film (also referred to as an organic compound layer). A thin film including the organic compound of one embodiment of the present invention can be suitably used for a light-emitting layer, a hole-transport layer, an electron-transport layer, or a cap layer in a light-emitting device 100. In addition, the organic compound of one embodiment of the present invention can be used also for a non-light-emitting device. As the non-light-emitting device, a device such as a light-receiving device is given, for example.

Note that the structure in the case of using the organic compound of one embodiment of the present invention for a light-emitting layer, a hole-transport layer, an electron-transport layer, or a cap layer in a light-emitting device, or in the case of using the organic compound of one embodiment of the present invention for a light-receiving device is described in detail in Embodiment 2.

Next, specific examples of the organic compounds of embodiments of the present invention having the above structures represented by General Formulae (G1) to (G5) above are shown below.

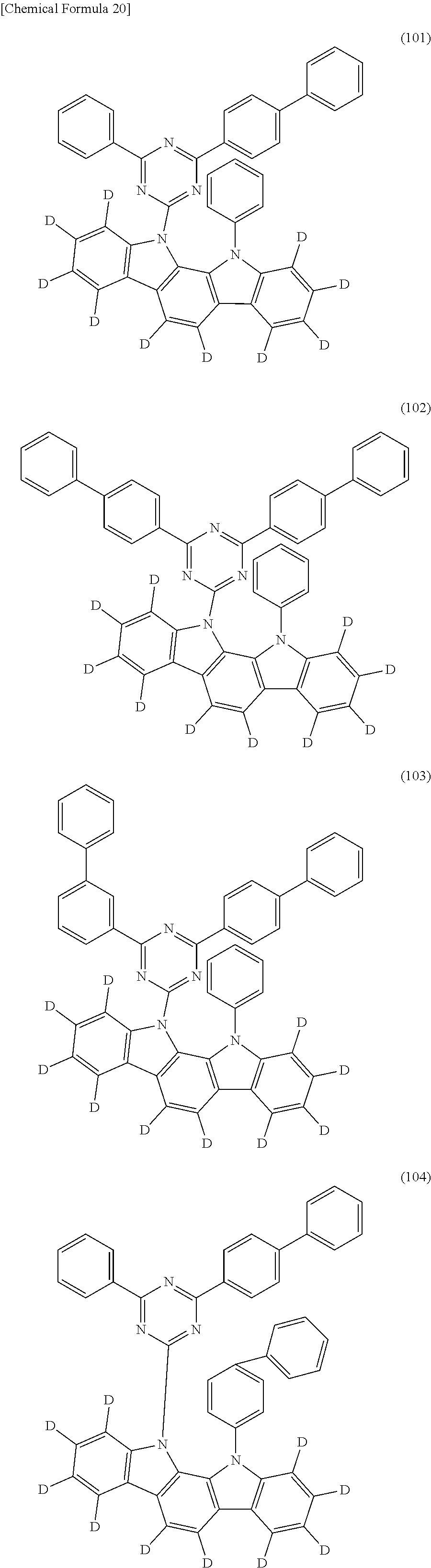

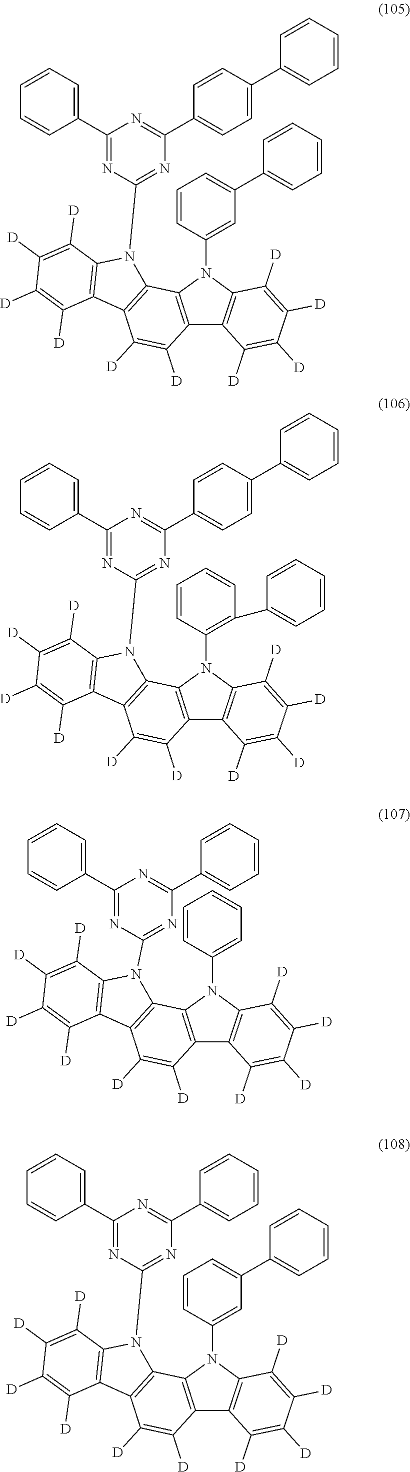

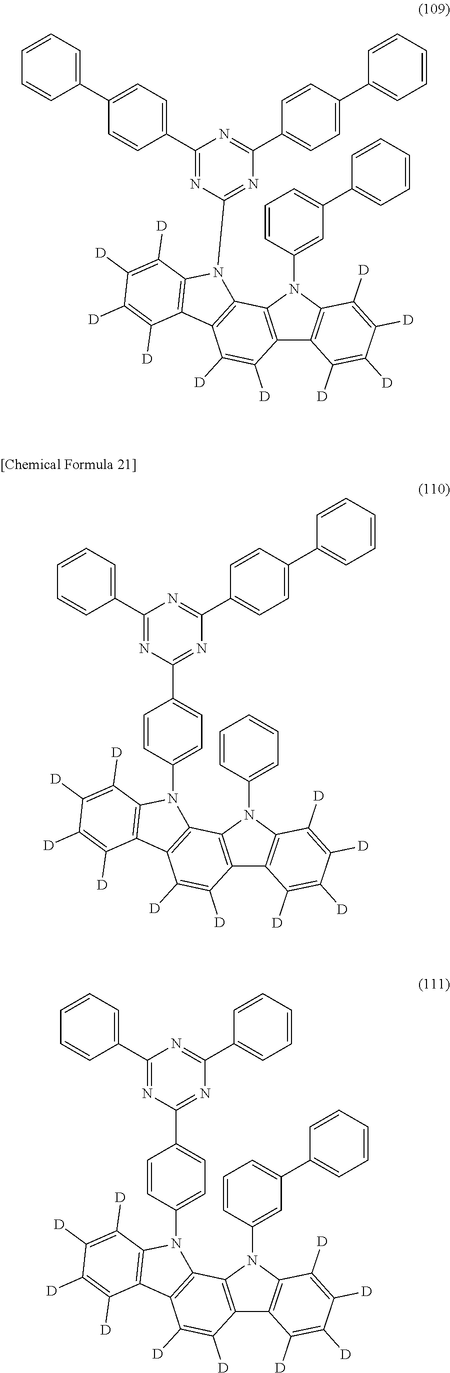

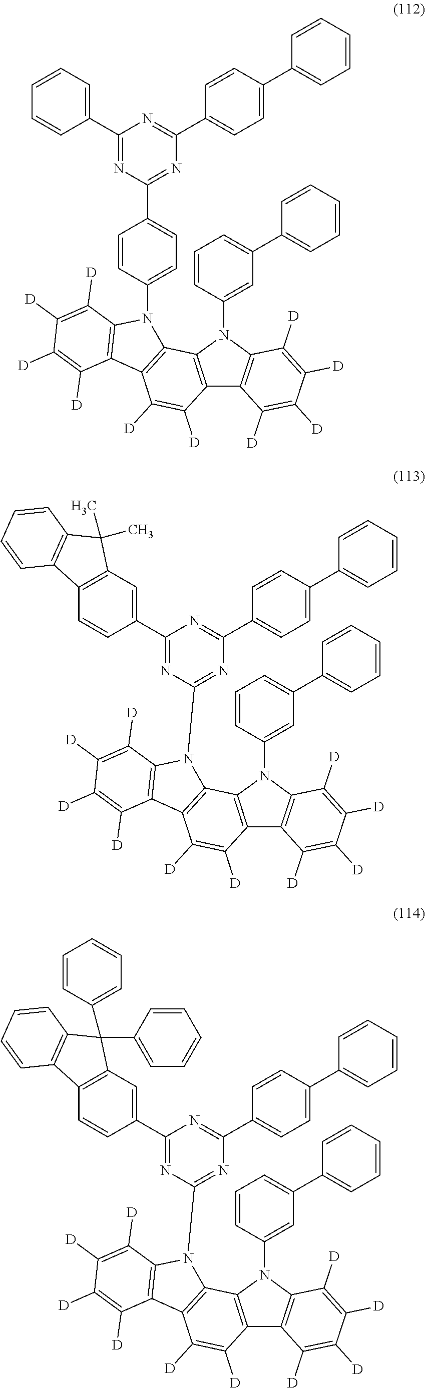

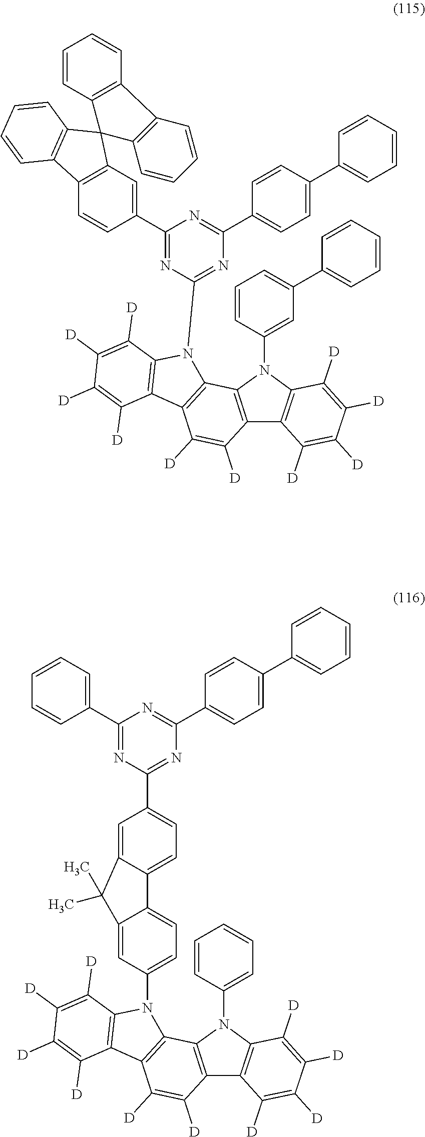

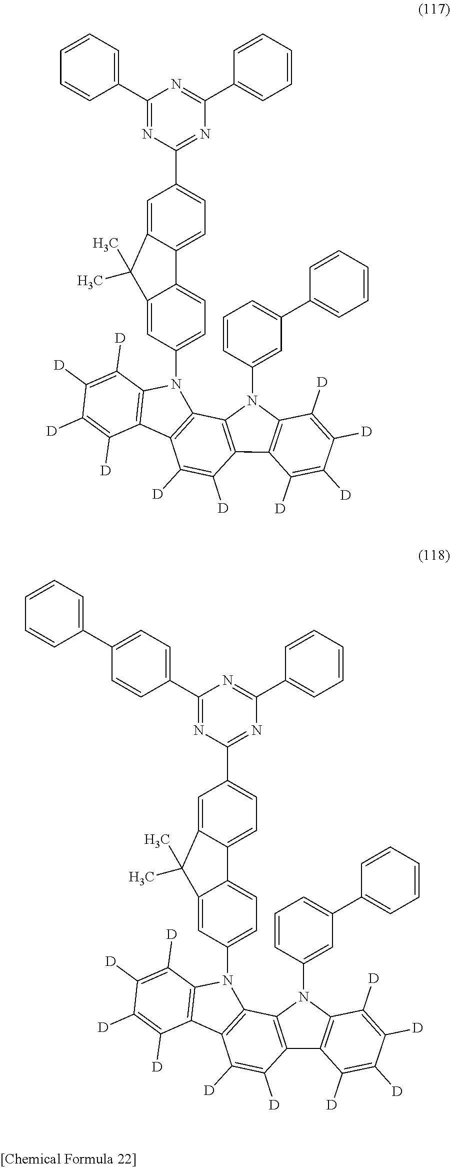

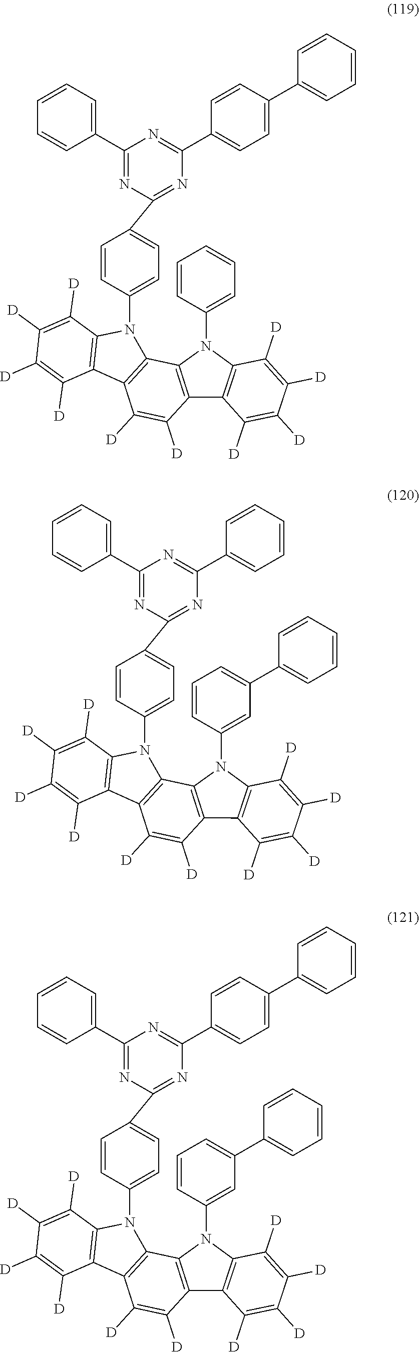

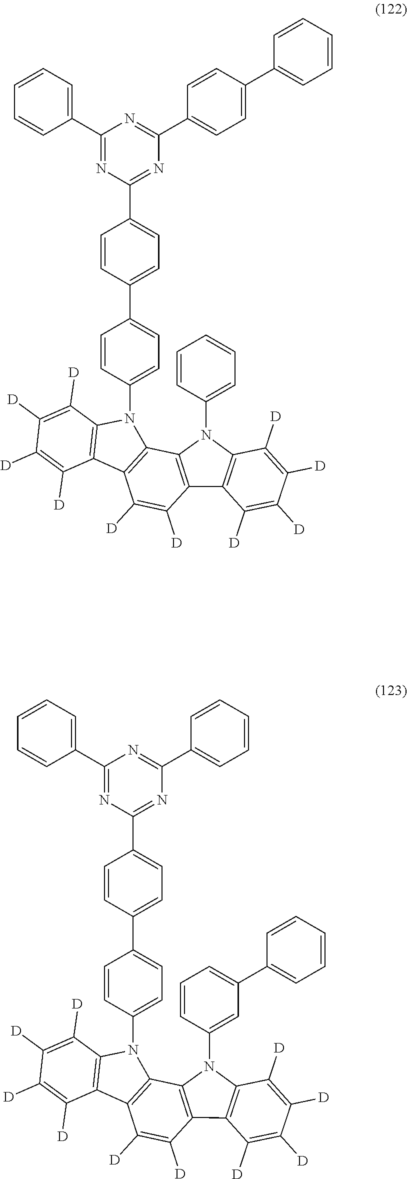

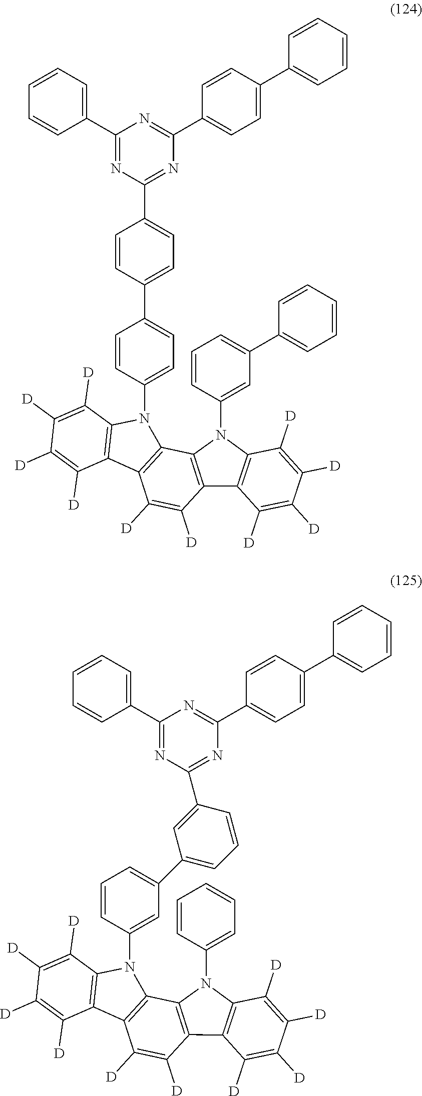

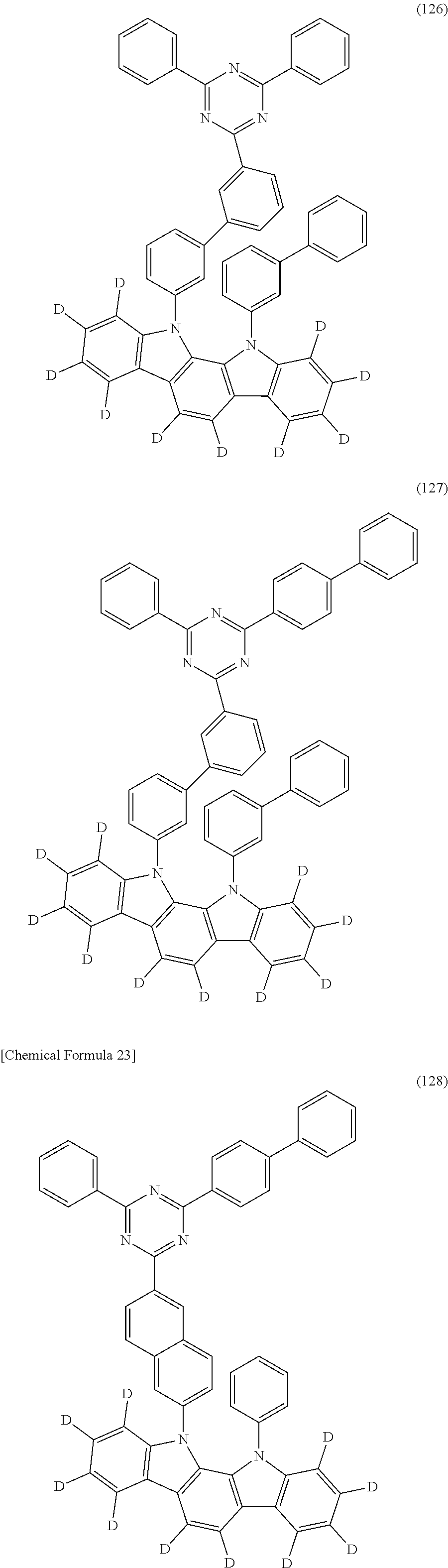

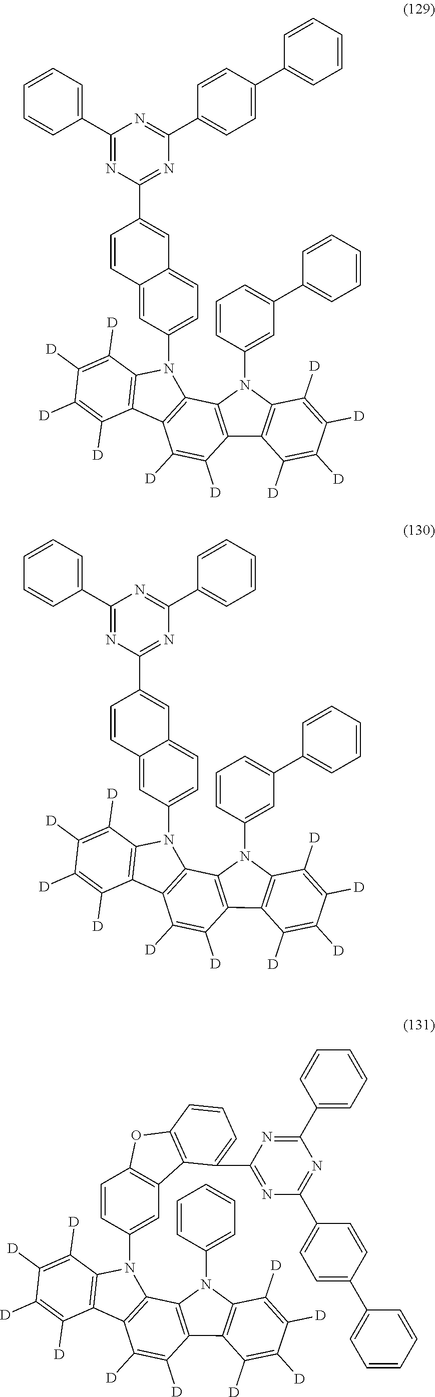

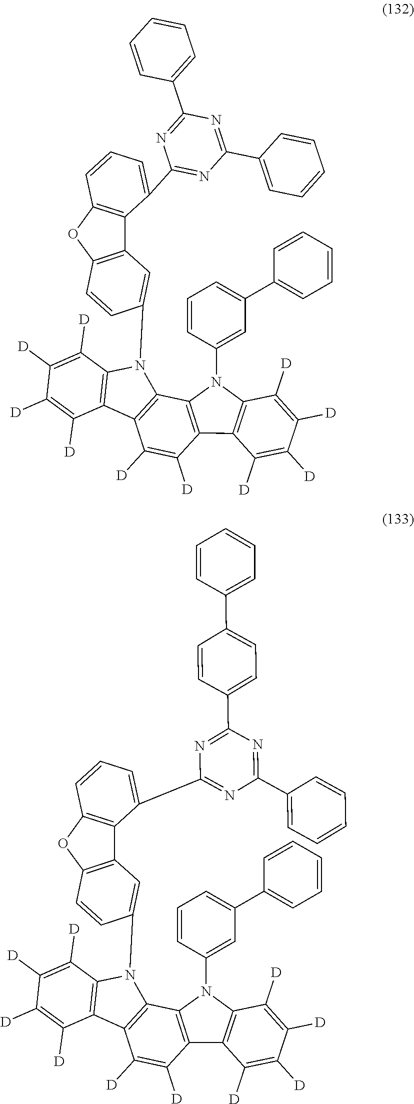

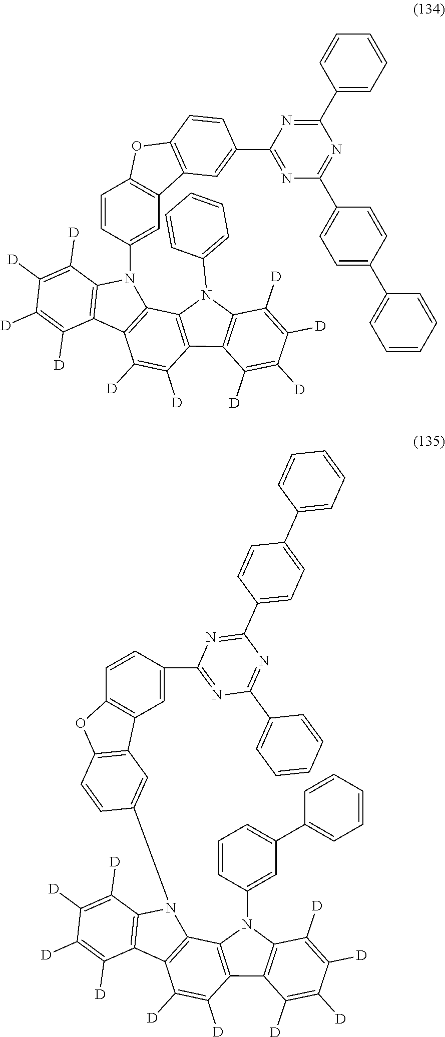

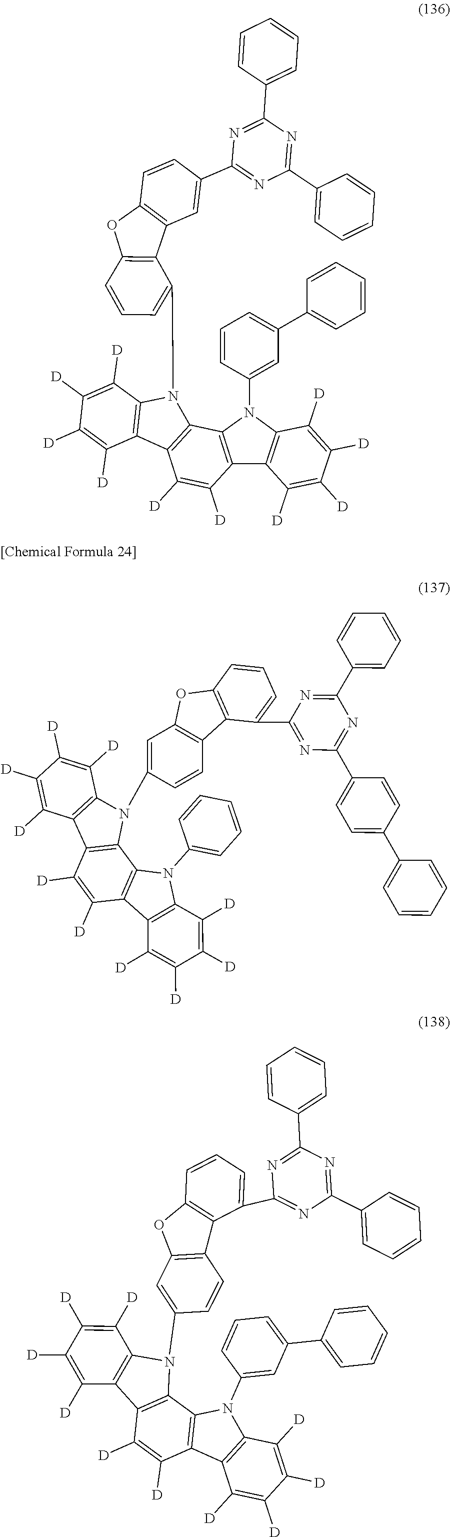

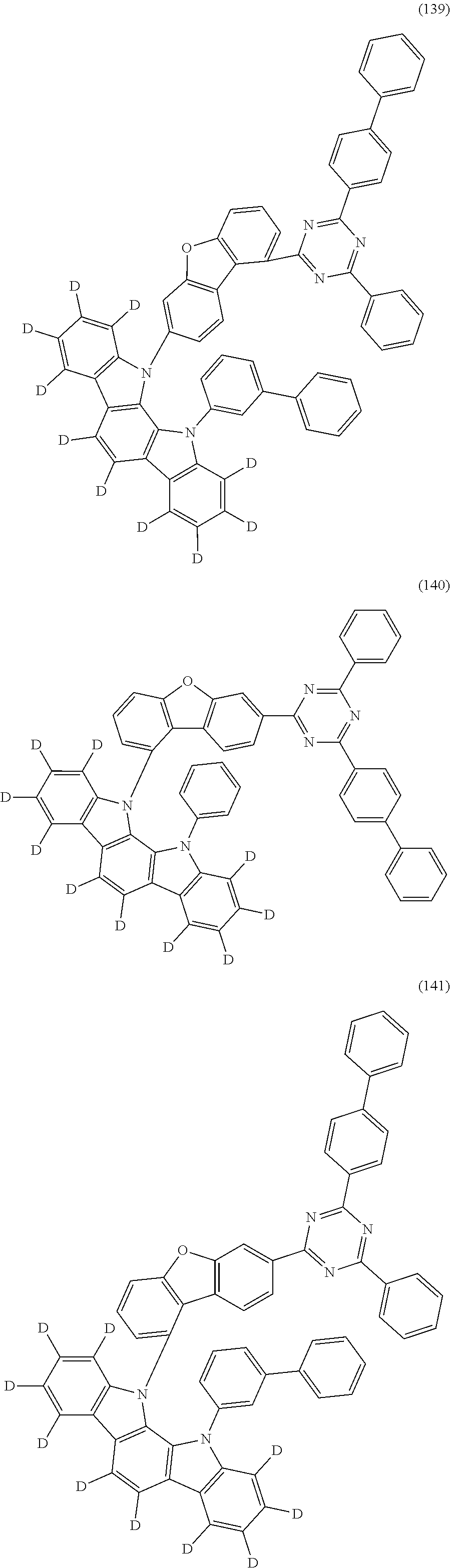

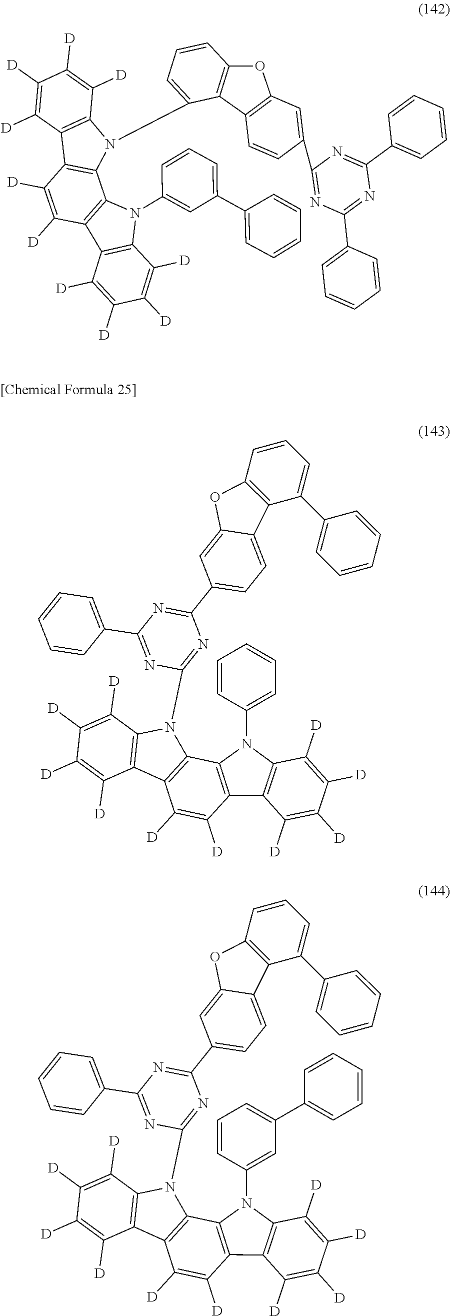

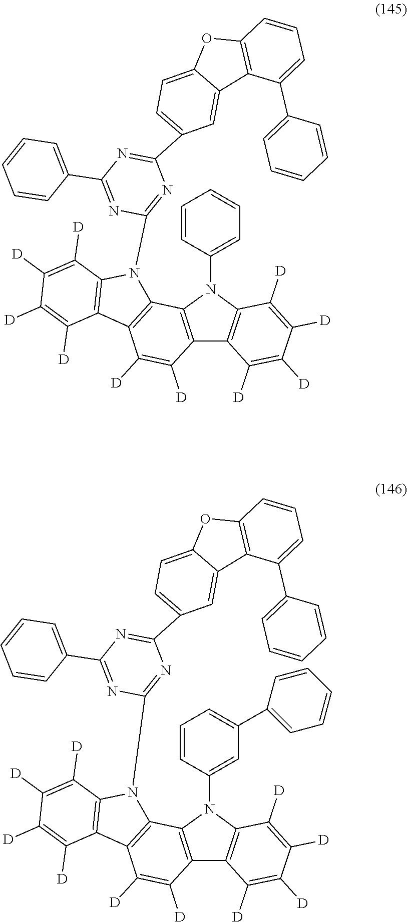

Although the organic compounds represented by Structural Formulae (101) to (146) shown above are examples of the organic compounds represented by General Formulae (G1) to (G5) shown above, the organic compound of one embodiment of the present invention is not limited thereto.

Next, a method of synthesizing the organic compound represented by General Formula (G1) is described. Note that a variety of reactions can be applied to the method of synthesizing the organic compound of one embodiment of the present invention. Accordingly, the method of synthesizing the organic compound of one embodiment of the present invention is not limited to the synthesis methods below.

In this embodiment, a method of synthesizing the organic compound represented by General Formula (G1) below is described.

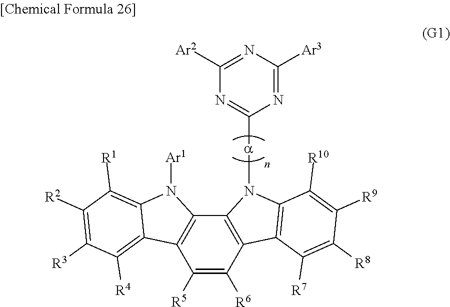

In General Formula (G1), at least one of R¹ to R¹⁰ represents deuterium; each of the others of R¹ to R¹⁰ independently represents hydrogen, deuterium, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms; each of Ar¹ to Ar³ independently represents a substituted or unsubstituted aryl group having 6 to 30 carbon atoms or a substituted or unsubstituted heteroaryl group having 2 to 30 carbon atoms; α represents a substituted or unsubstituted arylene group having 6 to 30 carbon atoms or a substituted or unsubstituted heteroarylene group having 2 to 30 carbon atoms; n represents an integer of 0 to 2; and when n is 2, two α may be the same with or different from each other.

In General Formula (G1), when any one or more of R¹ to R¹⁰ are a substituted or unsubstituted alkyl group having 1 to 6 carbon atoms, a substituted or unsubstituted aryl group having 6 to 30 carbon atoms, a substituted or unsubstituted heteroaryl group having 2 to 30 carbon atoms, these groups may be deuterated or not.

In General Formula (G1), the deuteration rate of the indolocarbazole skeleton is preferably greater than or equal to 50% and less than or equal to 100%. Note that the deuteration rate of the indolocarbazole skeleton is preferably greater than or equal to 60%, further preferably greater than or equal to 70%, still further preferably greater than or equal to 80%, yet still further preferably greater than or equal to 90%.

Note that even when the organic compound represented by any of General Formula (G1) is a mixture of a compound in which a substituent is deuterium and a compound in which a substituent is hydrogen, an effect can be obtained in some cases. For example, a mixture in which compounds whose R¹ in General Formula (G1) to General Formula (G5) is deuterium occupies 50% and compounds whose R¹ is hydrogen occupies 50% can also have an effect. The same applies to R² to R¹⁰.

The organic compound of the present invention represented by General Formula (G1) can be synthesized by Synthesis Schemes (a-1) to (a-3) below.

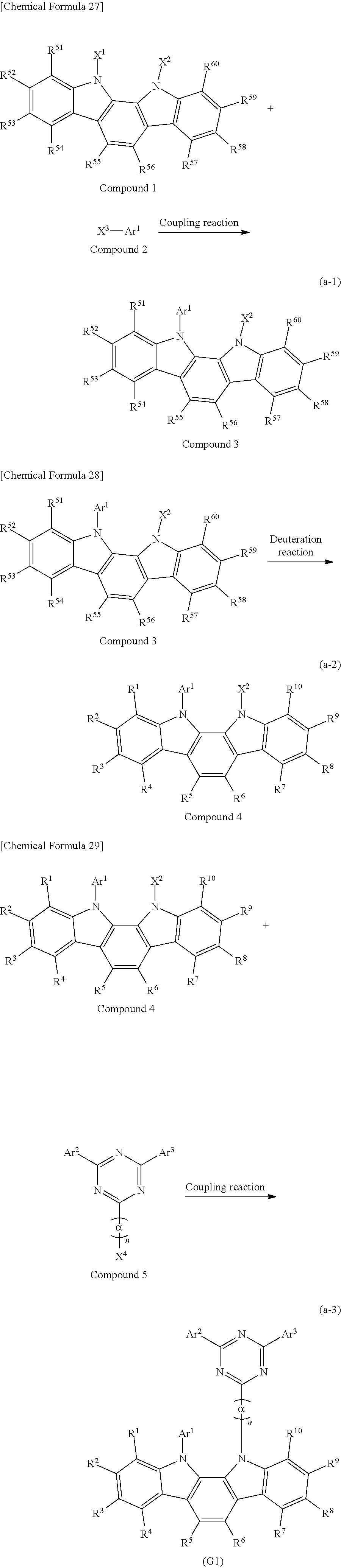

First, the indolocarbazole compound (Compound 1) and a compound (Compound 2) including Ar¹ are coupled in accordance with Reaction Formula (α-1), whereby an indolocarbazole compound (Compound 3) can be obtained. Next, the indolocarbazole compound (Compound 3) is selectively deuterated in accordance with Reaction Formula (a-2), whereby a deuterated indolocarbazole compound (Compound 4) can be obtained. Then, the deuterated indolocarbazole compound (Compound 4) and an azine compound (Compound 5) are coupled in accordance with Reaction Formula (a-3), whereby a target deuterated indolocarbazole compound (G1) can be obtained. Synthesis Schemes (a-1) to (a-3) are shown below.

In Synthesis Schemes (a-1) to (a-3), the above description applies to Ar¹ to Ar³ and R¹ to R¹⁰. In Synthesis Schemes (a-1) and (a-2), each of R⁵¹ to R⁶⁰ independently represents hydrogen, a substituted or unsubstituted alkyl group having 1 to 6 carbon atoms, a substituted or unsubstituted aryl group having 6 to 30 carbon atoms, or a substituted or unsubstituted heteroaryl group having 2 to 30 carbon atoms.

In Synthesis Schemes (a-1) to (a-3), X¹ to X² each represent hydrogen. Furthermore, X³ to X⁴ each represent a halogen group (including chlorine, for example). Note that one embodiment of the present invention is not limited thereto, and each of X¹ to X⁴ may independently represent hydrogen, chlorine, bromine, iodine, a triflate group, an organoboron group, a boronic acid, an organotin group, or the like.

In the coupling reaction in Synthesis Schemes (a-1) and (a-3), Buchwald-Hartwig reaction using a palladium catalyst is performed. In the Buchwald-Hartwig reaction, a palladium compound such as bis(dibenzylideneacetone)palladium(0), palladium(II) acetate, [1,1-bis(diphenylphosphino)ferrocene]palladium(II) dichloride, or tetrakis(triphenylphosphine)palladium(0) and a ligand such as tri(tert-butyl)phosphine, tri(n-hexyl)phosphine, tricyclohexylphosphine, di(1-adamantyl)-n-butylphosphine, 2-dicyclohexylphosphino-2′,6′-dimethoxybiphenyl, or tri(ortho-tolyl)phosphine can be used. In the reaction, an organic base such as sodium tert-butoxide, an inorganic base such as potassium carbonate, cesium carbonate, sodium carbonate, or sodium hydride, or the like can be used. In the reaction, toluene, xylene, mesitylene, diethylene glycol dimethyl ether (diglyme), benzene, tetrahydrofuran, dioxane, N,N′-dimethylformamide (DMF), dimethylsulfoxide (DMSO), ethanol, methanol, water, or the like can be used as the solvent. Reagents that can be used in the reaction are not limited to the above-described reagents.

Synthesis Schemes (a-1) and (a-3) can also be performed without using a palladium catalyst. For example, the use of a strong base such as sodium hydride and a solvent such as DMF can promote the coupling reaction.

A reaction using copper or a copper compound can be used for Synthesis Schemes (a-1) and (a-3).

Examples of the solvent that can be used in a deuteration reaction in Synthesis Scheme (a-2) include benzene-d6, toluene-d8, xylene-d10, and heavy water. Note that the solvent that can be used is not limited thereto. Examples of the catalyst that can be used include molybdenum(V) chloride, tungsten(VI) chloride, niobium(V) chloride, tantalum(V) chloride, aluminum(III) chloride, titanium(IV) chloride, and tin(IV) chloride. Note that the catalyst that can be used is not limited thereto. With the use of such a solvent and a catalyst, R¹ to R¹⁰ of the indolocarbazole compound can be selectively deuterated.

The case where Ar¹ represents an aryl group is more effective in preventing deuterium of Ar¹ than the case where Ar¹ represents a heteroaryl group. Thus, the effect of selective deuteration of R¹ to R¹⁰ can be increased.

The deuteration reaction may be performed before Synthesis Scheme (a-1). In that case, after substituents other than X¹ and X² in Compound 1 are selectively deuterated, an X¹—Ar¹ coupling reaction may be performed in accordance with (α-1). Note that when an N—H structure of a pyrrole ring exists in Compound 1, hydrochloride might be generated in a deuteration reaction using an acidic reagent (e.g., molybdenum(V) chloride). When the hydrochloride is generated, the possibility that the solubility in an organic solvent is decreased and reaction is less likely to proceed is considered. In particular, two NH structures of pyrrole rings exist in one molecule, hydrochloride might be likely to be generated and the solubility in an organic solvent might be significantly decreased. Therefore, Synthesis Scheme (a-1) is performed to set the number of NH structures in a molecular structure to be 1 and then Synthesis Scheme (a-2) is performed, which is preferable because deuteration of the indolocarbazole skeleton (Compound 3) can easily proceed.

Note that the deuteration reaction may be performed after Synthesis Scheme (a-3). Also in that case, a deuteration reaction of hydrogen bonded to the indolocarbazole skeleton proceeds faster than a deuteration reaction of hydrogen bonded to carbon in Ar¹; thus, R¹ to R¹⁰ can be selectively deuterated.

Note that the method of synthesizing the organic compound represented by General Formula (G1) is not limited to the order of Synthesis Schemes (a-1) to (a-3). For example, the target deuterated indolocarbazole compound (G1) can be obtained in the following manner: an indolocarbazole compound and an azine compound are coupled to synthesize an indolocarbazole compound, the indolocarbazole compound is subjected to a deuteration reaction to give a deuterated indolocarbazole compound, and finally the deuterated indolocarbazole compound is coupled with a compound including Ar¹.

Synthesis Scheme (a-3) may be divided into multiple stages and performed separately. For example, the synthesis may be performed through reactions represented by Reaction Formulae (a-4) to (a-5) below. Specifically, an indolocarbazole compound (Compound 4) and an aryl compound (Compound 6) are coupled to give an indolocarbazole compound (Compound 7) and then coupling reaction of Compound 7 and an azine compound (Compound 8) is performed, whereby the target compound (G1) can be obtained. Note that each of X⁵ and X⁶ in Reaction Formulae (α-1) to (a-5) independently represents chlorine, bromine, iodine, a triflate group, an organic boron group, a boronic acid, an organotin group, or the like, and X² and X⁴, R¹ to R¹⁰, Ar¹ to Ar³, and α are the same as those in the above description and thus their description is omitted.

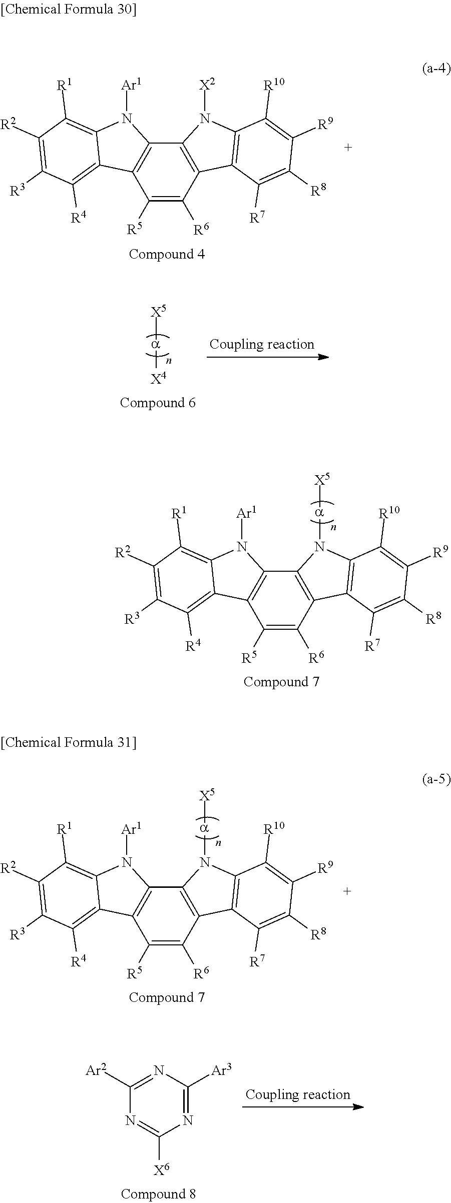

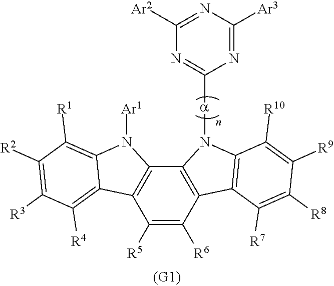

The structures described in this embodiment can be used in an appropriate combination with any of the structures described in the other embodiments.

### Embodiment 2

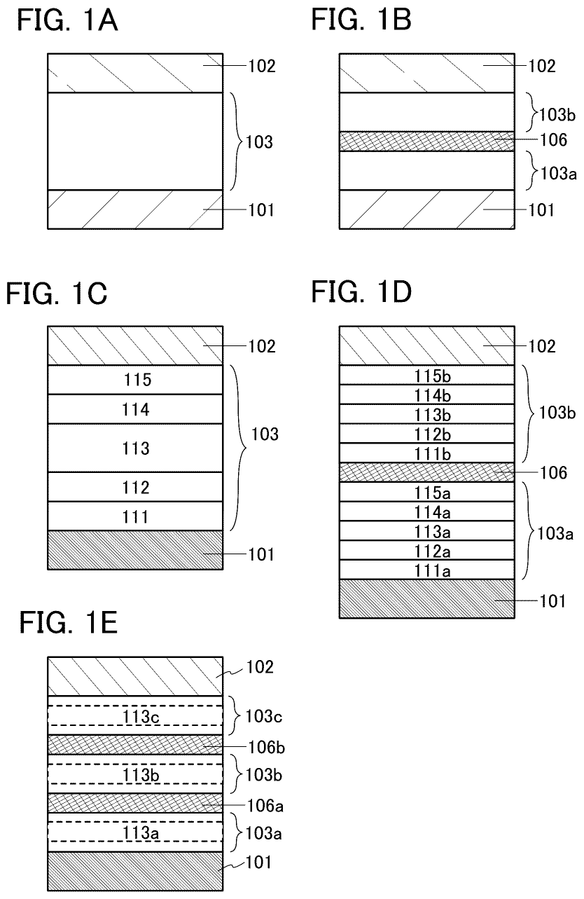

In this embodiment, a structure of a light-emitting apparatus including the organic compound described in Embodiment 1 is described with reference to FIG. 1A to FIG. 1E.

### <<Basic Structure of Light-Emitting Device>>

A basic structure of a light-emitting device is described. FIG. 1A illustrates a light-emitting device in which an EL layer including a light-emitting layer is provided between a pair of electrodes. Specifically, the light-emitting device has a structure in which an EL layer 103 is sandwiched between the first electrode 101 and the second electrode 102.

FIG. 1B illustrates a light-emitting device with a stacked-layer structure (a tandem structure) in which a plurality of EL layers (103a and 103b, two layers in FIG. 1B) are provided between a pair of electrodes and a charge-generation layer 106 is provided between the EL layers. With a light-emitting device having a tandem structure, a light-emitting apparatus with high efficiency can be achieved without changing the current amount.

The charge-generation layer 106 has a function of injecting electrons to one of the EL layers (103a or 103b) and injecting holes to the other of the EL layers (103b or 103a) when a potential difference is generated between the first electrode 101 and the second electrode 102. Thus, when voltage is applied such that the potential of the first electrode 101 is higher than that of the second electrode 102 in FIG. 1B, electrons are injected to the EL layer 103a and holes are injected to the EL layer 103b from the charge-generation layer 106.

Note that in terms of light extraction efficiency, it is preferable that the charge-generation layer 106 have a light-transmitting property with respect to visible light (specifically, the visible light transmittance with respect to the charge-generation layer 106 is preferably 40% or higher). Furthermore, the charge-generation layer 106 functions even when having lower conductivity than the first electrode 101 or the second electrode 102.

FIG. 1C illustrates a stacked-layer structure of the EL layer 103 in the light-emitting device of one embodiment of the present invention. Note that in this case, the first electrode 101 functions as an anode and the second electrode 102 functions as a cathode. The EL layer 103 has a structure in which the hole-injection layer 111, the hole-transport layer 112, a light-emitting layer 113, the electron-transport layer 114, and the electron-injection layer 115 are stacked sequentially over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can achieve higher reliability than a single-layer structure in some cases. Even in the case where a plurality of EL layers are provided as in the tandem structure illustrated in FIG. 1B, the EL layers are sequentially stacked from the anode side as described above. When the first electrode 101 is a cathode and the second electrode 102 is an anode, the stacking order in the EL layer 103 is reversed. Specifically, the layer 111 over the first electrode 101 serving as the cathode denotes an electron-injection layer; the layer 112 denotes an electron-transport layer; the layer 113 denotes a light-emitting layer; the layer 114 denotes a hole-transport layer; and the layer 115 denotes a hole-injection layer.

The light-emitting layers 113 included in the EL layers (103, 103a, and 103b) each contain an appropriate combination of a light-emitting substance and a plurality of substances, so that fluorescent light or phosphorescent light of a desired emission color can be obtained. Furthermore, the light-emitting layer 113 may have a stacked-layer structure having different emission colors. In that case, light-emitting substances and other substances are different between the stacked light-emitting layers. Furthermore, a structure in which different emission colors can be obtained from the plurality of EL layers (103a and 103b) illustrated in FIG. 1B may be employed. Also in that case, light-emitting substances and other substances are different between the stacked light-emitting layers.

In addition, the light-emitting device of one embodiment of the present invention can have an optical micro resonator (microcavity) structure with the first electrode 101 being a reflective electrode and the second electrode 102 being a transflective electrode in FIG. 1C, for example, and light emission obtained from the light-emitting layer 113 in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (a transparent conductive film), optical adjustment can be performed by adjusting the thickness of the transparent conductive film. Specifically, when the wavelength of light obtained from the light-emitting layer 113 is λ, the optical path length (the product of the film thickness and the refractive index) between the first electrode 101 and the second electrode 102 is preferably adjusted to mλ/2 (m is an integer of 1 or larger) or the vicinity thereof.

To amplify desired light (wavelength: A) obtained from the light-emitting layer 113, the optical path length from the first electrode 101 to a region where the desired light is obtained in the light-emitting layer 113 (a light-emitting region) and the optical path length from the second electrode 102 to the region where the desired light is obtained in the light-emitting layer 113 (the light-emitting region) are preferably adjusted to (2m′+1)λ/4 (m′ is an integer of 1 or larger) or the vicinity thereof. Here, the light-emitting region refers to a region where holes and electrons are recombined in the light-emitting layer 113.

By performing such optical adjustment, the spectrum of specific monochromatic light obtained from the light-emitting layer 113 can be narrowed and light emission with high color purity can be obtained.

Note that in the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained with given positions in the first electrode 101 and the second electrode 102 being supposed to be reflective regions. Furthermore, the optical path length between the first electrode 101 and the light-emitting layer from which the desired light is obtained is, to be exact, the optical path length between the reflective region in the first electrode 101 and the light-emitting region in the light-emitting layer from which the desired light is obtained. However, it is difficult to precisely determine the reflective region in the first electrode 101 and the light-emitting region in the light-emitting layer from which the desired light is obtained; thus, it is assumed that the above effect can be sufficiently obtained with a given position in the first electrode 101 being supposed to be the reflective region and a given position in the light-emitting layer from which the desired light is obtained being supposed to be the light-emitting region.

The light-emitting device illustrated in FIG. 1D is a light-emitting device having a tandem structure. Owing to a microcavity structure of the light-emitting device, light (monochromatic light) with different wavelengths from the EL layers (103a and 103b) can be extracted. Thus, side-by-side patterning for obtaining different emission colors (e.g., RGB) is not necessary. Therefore, higher definition can be easily achieved. In addition, a combination with coloring layers (color filters) is also possible. Furthermore, the emission intensity of light with a specific wavelength in the front direction can be increased, whereby power consumption can be reduced.

A light-emitting device illustrated in FIG. 1E is an example of the light-emitting device with the tandem structure illustrated in FIG. 1B, and includes three EL layers (103a, 103b, and 103c) stacked with charge-generation layers (106a and 106b) therebetween, as illustrated in the drawing. Note that the three EL layers (103a, 103b, and 103c) include respective light-emitting layers (113a, 113b, and 113c) and the emission colors of the respective light-emitting layers can be combined freely. For example, the light-emitting layer 113a can emit blue light, the light-emitting layer 113b can emit red, green, or yellow light, and the light-emitting layer 113c can emit blue light; for another example, the light-emitting layer 113a can emit red light, the light-emitting layer 113b can emit blue, green, or yellow light, and the light-emitting layer 113c can emit red light.

In the above light-emitting device of one embodiment of the present invention, at least one of the first electrode 101 and the second electrode 102 is a light-transmitting electrode (a transparent electrode, a transflective electrode, or the like). In the case where the light-transmitting electrode is a transparent electrode, the visible light transmittance of the transparent electrode is 40% or higher. In the case where the light-transmitting electrode is a transflective electrode, the visible light reflectance of the transflective electrode is higher than or equal to 20% and lower than or equal to 80%, preferably higher than or equal to 40% and lower than or equal to 70%. The resistivity of these electrodes is preferably 1×10⁻² Ωcm or lower.

In the case where one of the first electrode 101 and the second electrode 102 is an electrode having a reflecting property (a reflective electrode) in the above light-emitting device of one embodiment of the present invention, the visible light reflectance of the electrode having a reflecting property is higher than or equal to 40% and lower than or equal to 100%, preferably higher than or equal to 70% and lower than or equal to 100%. The resistivity of this electrode is preferably 1×10⁻² Ωcm or lower.

### <<Specific Structure of Light-Emitting Device>>

Next, a specific structure of the light-emitting device of one embodiment of the present invention will be described. Here, description is made using FIG. 1D illustrating the tandem structure. Note that the structure of the EL layer applies also to the light-emitting devices having a single structure in FIG. 1A and FIG. 1C. In the case where the light-emitting device illustrated in FIG. 1D has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the EL layer 103b, with the use of a material selected as appropriate.

### <First Electrode and Second Electrode>

As materials for forming the first electrode 101 and the second electrode 102, any of the following materials can be used in an appropriate combination as long as the functions of the both electrodes described above can be fulfilled. For example, a metal, an alloy, an electrically conductive compound, and a mixture of these can be used as appropriate. Specifically, an In—Sn oxide (also referred to as ITO), an In—Si—Sn oxide (also referred to as ITSO), an In—Zn oxide, and an In—W—Zn oxide are given. In addition, it is also possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use an element belonging to Group 1 or Group 2 in the periodic table, which is not listed above as an example (for example, lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device illustrated in FIG. 1D, when the first electrode 101 is an anode, a hole-injection layer 111a and a hole-transport layer 112a of the EL layer 103a are sequentially stacked over the first electrode 101 by a vacuum evaporation method. After the EL layer 103a and the charge-generation layer 106 are formed, a hole-injection layer 111b and a hole-transport layer 112b of the EL layer 103b are sequentially stacked over the charge-generation layer 106 in a similar manner.

### <Hole-Injection Layer>

The hole-injection layers (111, 111a, and 111b) are each a layer that injects holes from the first electrode 101 which is an anode or from the charge-generation layers (106, 106a, and 106b) to the EL layers (103, 103a, and 103b) and contains an organic acceptor material and a material with a high hole-injection property.

The organic acceptor material is a material that allows holes to be generated in another organic compound whose HOMO level value is close to the LUMO (lowest unoccupied molecular orbital) level value of the organic acceptor material when charge separation is caused between the organic acceptor material and the organic compound. Thus, as the organic acceptor material, a compound having an electron-withdrawing group (a halogen group or a cyano group), such as a quinodimethane derivative, a chloranil derivative, or a hexaazatriphenylene derivative, can be used. For example, it is possible to use 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylen-1,3,4,5,6,8,9,10-octafluoro-7H-pyren-2-ylidene) malononitrile. Note that among organic acceptor materials, a compound in which electron-withdrawing groups are bonded to condensed aromatic rings each having a plurality of heteroatoms, such as HAT-CN, is particularly preferred because it has a high acceptor property and stable film quality against heat. Alternatively, a [3] radialene derivative having an electron-withdrawing group (in particular, a cyano group or a halogen group such as a fluoro group) has a very high electron-accepting property and thus is preferable. Specifically, α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], α,α′,α″-1,2,3-cyclopropanetriylidenetris[2,3,4,5,6-pentafluorobenzeneacetonitrile], or the like can be used.

As the material having a high hole-injection property, an oxide of a metal belonging to Group 4 to Group 8 in the periodic table (e.g., a transition metal oxide such as a molybdenum oxide, a vanadium oxide, a ruthenium oxide, a tungsten oxide, or a manganese oxide) can be used. As specific examples, a molybdenum oxide, a vanadium oxide, a niobium oxide, a tantalum oxide, a chromium oxide, a tungsten oxide, a manganese oxide, and a rhenium oxide are given. In particular, a molybdenum oxide is preferable because it is stable in the air, has a low hygroscopic property, and is easily handled. It is also possible to use phthalocyanine (abbreviation: H₂Pc), a phthalocyanine-based compound such as copper phthalocyanine (abbreviation: CuPc), or the like.

In addition to the above materials, it is also possible to use an aromatic amine compound, which is a low molecular compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N-bis{4-[bis(3-methylphenyl)amino]phenyl}-N,N-diphenyl-(1,1′-biphenyl)-4,4′-diamine (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), or 3-[N-(1-naphthyl)-N-(9-phenylcarbazol-3-yl)amino]-9-phenylcarbazole (abbreviation: PCzPCN1).

It is also possible to use a high molecular compound (an oligomer, a dendrimer, a polymer, or the like) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl) methacrylamide] (abbreviation: PTPDMA), or poly[N,N-bis(4-butylphenyl)-N,N-bis(phenyl)benzidine] (abbreviation: Poly-TPD). It is also possible to use a high molecular compound to which acid such as poly(3,4-ethylenedioxythiophene)/polystyrenesulfonic acid (abbreviation: PEDOT/PSS) or polyaniline/polystyrenesulfonic acid (abbreviation: PAni/PSS) is added.

As the material with a high hole-injection property, a mixed material containing a hole-transport material and the above-described organic acceptor material (an electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed as a single layer of a mixed material containing the hole-transport material and the organic acceptor material (an electron-accepting material), or may be formed by stacking a layer containing the hole-transport material and a layer containing the organic acceptor material (the electron-accepting material).

The hole-transport material is preferably a substance having a hole mobility higher than or equal to 1×10⁻⁶ cm²/Vs in the case where the square root of the electric field strength [V/cm] is 600. Note that other substances can be used as long as they have a property of transporting more holes than electrons.

As the hole-transport material, a material having a high hole-transport property such as a compound having a π-electron rich heteroaromatic ring (e.g., a carbazole derivative, a furan derivative, or a thiophene derivative) or an aromatic amine (an organic compound having an aromatic amine skeleton), is preferable. The compound in Embodiment 1 has a hole-transport property and thus can be used as a hole-transport material.

Examples of the above carbazole derivative (an organic compound having a carbazole ring) include a bicarbazole derivative (e.g., a 3,3′-bicarbazole derivative) and an aromatic amine having a carbazolyl group.

Specific examples of the above bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(1,1′-biphenyl-3-yl)-3,3′-bi-9H-carbazole (abbreviation: BismBPCz), 9-(1,1′-biphenyl-3-yl)-9′-(1,1′-biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: BNCCP).

Specific examples of the above aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9′-carbazol-3-amine (abbreviation: PCBiF), N-(1,1′-biphenyl-4-yl)-N-[4-(9-phenyl-9H-carbazol-3-yl)phenyl]-9,9-dimethyl-9H-fluoren-2-amine (abbreviation: PCBBiF), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N-bis(9-phenylcarbazol-3-yl)-N,N-diphenylbenzene-1,3-diamine (abbreviation: PCA2B), N,N,N′-triphenyl-N,N,N′-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]spiro-9,9′-bifluorene (abbreviation: PCASF), N-[4-(9H-carbazol-9-yl)phenyl]-N-(4-phenyl)phenylaniline (abbreviation: YGA1BP), N,N-bis[4-(carbazol-9-yl)phenyl]-N,N-diphenyl-9,9-dimethylfluorene-2,7-diamine (abbreviation: YGA2F), and 4,4′,4″-tris(carbazol-9-yl)triphenylamine (abbreviation: TCTA).

In addition to the above, other examples of the carbazole derivative include 3-[4-(9-phenanthryl)-phenyl]-9-phenyl-9H-carbazole (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the above furan derivative (an organic compound having a furan ring) include 4,4′,4″-(benzene-1,3,5-triyl)tri(dibenzofuran)(abbreviation: DBF3P-II), and 4-{3-[3-(9-phenyl-9H-fluoren-9-yl)phenyl]phenyl}dibenzofuran (abbreviation: mmDBFFLBi-II).

Specific examples of the above thiophene derivative (an organic compound having a thiophene ring) include 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the above aromatic amine include 4,4′-bis[N-(1-naphthyl)-N-phenylamino]biphenyl (abbreviation: NPB or α-NPD), N,N-bis(3-methylphenyl)-N,N-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]spiro-9,9′-bifluorene (abbreviation: DPASF), 2,7-bis[N-(4-diphenylaminophenyl)-N-phenylamino]-spiro-9,9′-bifluorene (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N-di(p-tolyl)-N,N-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenylamino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yl)triphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis([1,1′-biphenyl]-4-yl)-9,9′-spirobi[9H-fluoren]-2-amine (abbreviation: BBASF), N,N-bis([1,1′-biphenyl]-4-yl)-9,9′-spirobi[9H-fluoren]-4-amine (abbreviation: BBASF(4)), N-(1,1′-biphenyl-2-yl)-N-(9,9-dimethyl-9H-fluoren-2-yl)-9,9′-spirobi[9H-fluoren]-4-amine (abbreviation: oFBiSF), N-(4-biphenyl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Alternatively, it is also possible to use, as the hole-transport material, a high molecular compound (e.g., an oligomer, a dendrimer, or a polymer) such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N-phenylamino}phenyl)methacrylamide](abbreviation: PTPDMA), poly[N,N-bis(4-butylphenyl)-N,N-bis(phenyl)benzidine](abbreviation: Poly-TPD), or the like. Alternatively, it is also possible to use a high molecular compound to which acid such as poly(3,4-ethylenedioxythiophene)/polystyrenesulfonic acid (abbreviation: PEDOT/PSS), and polyaniline/polystyrenesulfonic acid (abbreviation: PAni/PSS) is added.

Note that the hole-transport material is not limited to the above, and one of or a combination of various known materials may be used as the hole-transport material.

Note that the hole-injection layers (111, 111a, and 111b) can be formed by any of various known deposition methods, and can be formed by a vacuum evaporation method, for example.

### <Hole-Transport Layer>

The hole-transport layers (112, 112a, and 112b) are each a layer that transports the holes, which are injected from the first electrode 101 by the hole-injection layers (111, 111a, and 111b), to the light-emitting layers (113, 113a, and 113b). Note that the hole-transport layers (112, 112a, and 112b) are each a layer containing a hole-transport material. Thus, for the hole-transport layers (112, 112a, and 112b), a hole-transport material that can be used for the hole-injection layers (111, 111a, and 111b) can be used.

Note that in the light-emitting device of one embodiment of the present invention, the organic compound used for the hole-transport layers (112, 112a, and 112b) can also be used for the light-emitting layers (113, 113a, 113b, and 113c). The use of the same organic compound for the hole-transport layers (112, 112a, and 112b) and the light-emitting layers (113, 113a, 113b, and 113c) is preferable, in which case holes can be efficiently transported from the hole-transport layers (112, 112a, and 112b) to the light-emitting layers (113, 113a, 113b, and 113c).

### <Light-Emitting Layer>

The light-emitting layers (113, 113a, 113b, and 113c) are each a layer containing a light-emitting substance. Note that as the light-emitting substance that can be used for the light-emitting layers (113, 113a, 113b, and 113c), a substance that exhibits an emission color of blue, violet, bluish violet, green, yellowish green, yellow, orange, red, or the like can be used as appropriate. In the case where a plurality of light-emitting layers are provided, different light-emitting substances are used for the light-emitting layers; thus, different emission colors can be exhibited (for example, complementary emission colors are combined to obtain white light emission). Furthermore, one light-emitting layer may have a stacked-layer structure containing different light-emitting substances.

The light-emitting layers (113, 113a, 113b, and 113c) may each contain one or more kinds of organic compounds (a host material and the like) in addition to a light-emitting substance (a guest material).

In the case where a plurality of host materials are used in the light-emitting layers (113, 113a, 113b, and 113c), a second host material that is additionally used is preferably a substance having a larger energy gap than those of a known guest material and a first host material. Preferably, the lowest singlet excitation energy level (S1 level) of the second host material is higher than the S1 level of the first host material, and the lowest triplet excitation energy level (T1 level) of the second host material is higher than the T1 level of the guest material. Furthermore, the lowest triplet excitation energy level (T1 level) of the second host material is preferably higher than the T1 level of the first host material. With such a structure, an exciplex can be formed by the two kinds of host materials. To form an exciplex efficiently, it is particularly preferable to combine a compound that easily accepts holes (a hole-transport material) and a compound that easily accepts electrons (an electron-transport material). With this structure, high efficiency, low voltage, and a long lifetime can be achieved at the same time.

As an organic compound used as the above host material (including the first host material and the second host material), organic compounds such as the hole-transport materials that can be used for the hole-transport layers (112, 112a, and 112b) described above and electron-transport materials that can be used for electron-transport layers (114, 114a, and 114b) described later can be used as long as they satisfy requirements for the host material used in the light-emitting layer. Another example is an exciplex formed by a plurality of kinds of organic compounds (the first host material and the second host material). An exciplex (also referred to as Exciplex) whose excited state is formed by a plurality of kinds of organic compounds has an extremely small difference between the S1 level and the T1 level and functions as a TADF material capable of converting triplet excitation energy into singlet excitation energy. As a combination of the plurality of kinds of organic compounds forming an exciplex, for example, it is preferable that one have a π-electron deficient heteroaromatic ring and the other have a π-electron rich heteroaromatic ring. A phosphorescent substance such as an iridium-, rhodium-, or platinum-based organometallic complex or a metal complex may be used as one of the combination forming an exciplex. The organic compound described in Embodiment 1 has an electron-transport property and thus can be efficiently used as the first host material. Furthermore, since the organic compound has a hole-transport property, it can be used as the second host material.

The light-emitting substance that can be used in the light-emitting layers (113, 113a, 113b, and 113c) is not particularly limited, and a light-emitting substance that converts singlet excitation energy into light emission in the visible light range or a light-emitting substance that converts triplet excitation energy into light emission in the visible light range can be used.

<<Light-Emitting Substance that Converts Singlet Excitation Energy into Light>>

The following substances emitting fluorescent light (fluorescent substances) are given as the light-emitting substance that can be used for the light-emitting layers (113, 113a, 113b, and 113c) and convert singlet excitation energy into light emission. The examples include a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-diphenyl-N,N-bis[4-(9-phenyl-9H-fluoren-9-yl)phenyl]pyrene-1,6-diamine (abbreviation: 1,6FLPAPrn), N,N-bis(dibenzofuran-2-yl)-N,N-diphenylpyrene-1,6-diamine (abbreviation: 1,6FrAPrn), N,N-bis(dibenzothiophen-2-yl)-N,N-diphenylpyrene-1,6-diamine (abbreviation: 1,6ThAPrn), N,N-(pyrene-1,6-diyl)bis[(N-phenylbenzo[b]naphtho[1,2-d]furan)-6-amine](abbreviation: 1,6BnfAPrn), N,N-(pyrene-1,6-diyl)bis[(N-phenylbenzo[b]naphtho[1,2-d]furan)-8-amine](abbreviation: 1,6BnfAPrn-02), and N,N-(pyrene-1,6-diyl)bis[(6,N-diphenylbenzo[b]naphtho[1,2-d]furan)-8-amine](abbreviation: 1,6BnfAPrn-03).

In addition, it is possible to use 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N′-(2-tert-butylanthracene-9,10-diyldi-4,1-phenylene)bis[N,N,N-triphenyl-1,4-phenylenediamine](abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N,N-triphenyl-1,4-phenylenediamine (abbreviation: 2DPAPPA), or the like.

It is also possible to use, for example, N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N,N-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]4H-pyran4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, pyrenediamine compounds such as 1,6FLPAPrn, 1,6mMemFLPAPrn, and 1,6BnfAPrn-03 can be used, for example.

<<Light-Emitting Substance that Converts Triplet Excitation Energy into Light Emission>>

Next, as examples of the light-emitting substance that converts triplet excitation energy into light emission and can be used for the light-emitting layer 113, a substance that emits phosphorescent light (a phosphorescent substance) and a thermally activated delayed fluorescent (TADF) material that exhibits thermally activated delayed fluorescence are given.

A phosphorescent substance refers to a compound that exhibits phosphorescence but does not exhibit fluorescence at a temperature higher than or equal to low temperatures (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (a platinum complex), a rare earth metal complex, or the like. Specifically, a transition metal element is preferable and it is particularly preferable that a platinum group element (ruthenium (Ru), rhodium (Rh), palladium (Pd), osmium (Os), iridium (Ir), or platinum (Pt)), especially iridium, be contained, in which case the transition probability relating to direct transition between the singlet ground state and the triplet excited state can be increased.

<<Phosphorescent Substance (from 450 nm to 570 nm: Blue or Green)>>

As a phosphorescent substance that exhibits blue or green and whose emission spectrum has a peak wavelength at greater than or equal to 450 nm and less than or equal to 570 nm, the following substances are given.

The examples include: organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III)(abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III)(abbreviation: [Ir(Prptz1-Me)₃]); organometallic complexes having an imidazole ring, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a ligand is a phenylpyridine derivative having an electron-withdrawing group such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

<<Phosphorescent Substance (from 495 nm to 590 nm: Green or Yellow)>>

As a phosphorescent substance that exhibits green or yellow and whose emission spectrum has a peak wavelength at greater than or equal to 495 nm and less than or equal to 590 nm, the following substances are given.

The examples include: organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis(4,6-dimethyl-2-[6-(2,6-dimethylphenyl)-4-pyrimidinyl-κN³]phenyl-κC)iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C²)iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d3-methyl-8-(2-pyridinyl-κN)benzofuro[2,3-b]pyridine-κC]bis[2-(5-d3-methyl-2-pyridinyl-κN^{2′})phenyl-κC]iridium(III) (abbreviation: Ir(5mppy-d3)₂(mbfpypy-d3)), [2-(methyl-d3)-8-[4-(1-methylethyl-1-d)-2-pyridinyl-κN]benzofuro[2,3-b]pyridine-7-yl-κC]bis[5-(methyl-d3)-2-[5-(methyl-d3)-2-pyridinyl-κN]phenyl-κC]iridium(III) (abbreviation: Ir(5mtpy-d6)₂(mbfpypy-iPr-d4)), [2-d3-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d3)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); organometallic complexes such as bis(2,4-diphenyl-1,3-oxazolato-N,C²)iridium(III) acetylacetonate (abbreviation: [Ir(dpo)₂(acac)]), bis(2-[4′-(perfluorophenyl)phenyl]pyridinato-N,C^{2′})iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and rare earth metal complexes such as tris(acetylacetonato)(monophenanthroline)terbium(III) (abbreviation: [Tb(acac)₃(Phen)]).

<<Phosphorescent Substance (from 570 nm to 750 nm: Yellow or Red)>>

As a phosphorescent material that exhibits yellow or red and whose emission spectrum has a peak wavelength at greater than or equal to 570 nm and less than or equal to 750 nm, the following substances are given.

The examples include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III)(abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis[2-(5-(2,6-dimethylphenyl)-3-(3,5-dimethylphenyl)-2-pyrazinyl-κN)-4,6-dimethylphenyl-κC](2,2′,6,6′-tetramethyl-3,5-heptadionato-κ2O,O′)iridium(III) (abbreviation: [Ir(dmdppr-dmp)₂(dpm)]), (acetylacetonato)bis[2-methyl-3-phenylquinoxalinato-N,C^{2′}]iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); platinum complexes such as 2,3,7,8,12,13,17,18-octaethyl-21H,23H-porphyrinplatinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### <<TADF Material>>

Any of materials shown below can be used as the TADF material. The TADF material refers to a material that has a small difference (preferably, less than or equal to 0.2 eV) between the S1 level and the T1 level, can up-convert a triplet excited state into a singlet excited state (reverse intersystem crossing) using a little thermal energy, and efficiently exhibits light emission (fluorescence) from the singlet excited state. The thermally activated delayed fluorescence is efficiently obtained under the condition where the difference in energy between the triplet excited energy level and the singlet excited energy level is greater than or equal to 0 eV and less than or equal to 0.2 eV, preferably greater than or equal to 0 eV and less than or equal to 0.1 eV. Delayed fluorescence by the TADF material refers to light emission having a spectrum similar to that of normal fluorescence and an extremely long lifetime. The lifetime is 1×10⁻⁶ seconds or longer, or 1×10⁻³ seconds or longer. The organic compounds described in Embodiment 1 can also be used.

Note that the TADF material can be also used as an electron-transport material, a hole-transport material, or a host material.

Examples of the TADF material include fullerene, a derivative thereof, an acridine derivative such as proflavine, and eosin. Other examples include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

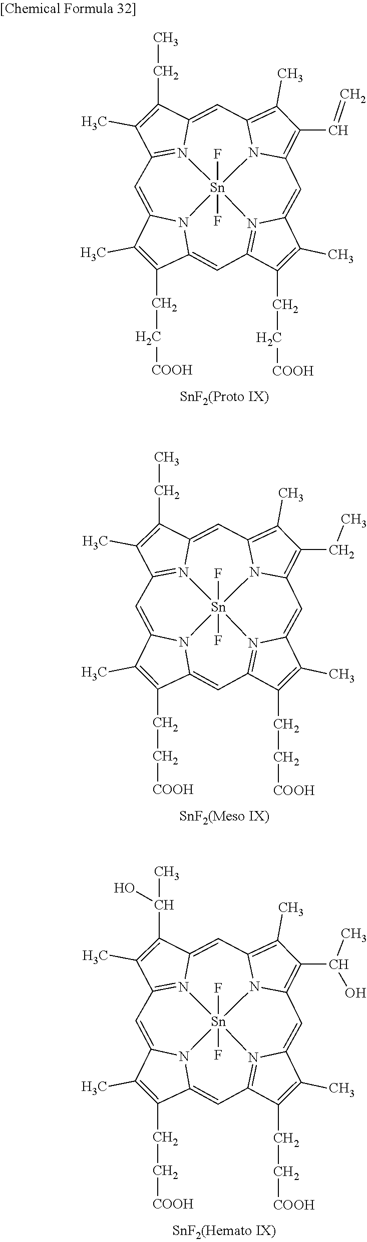

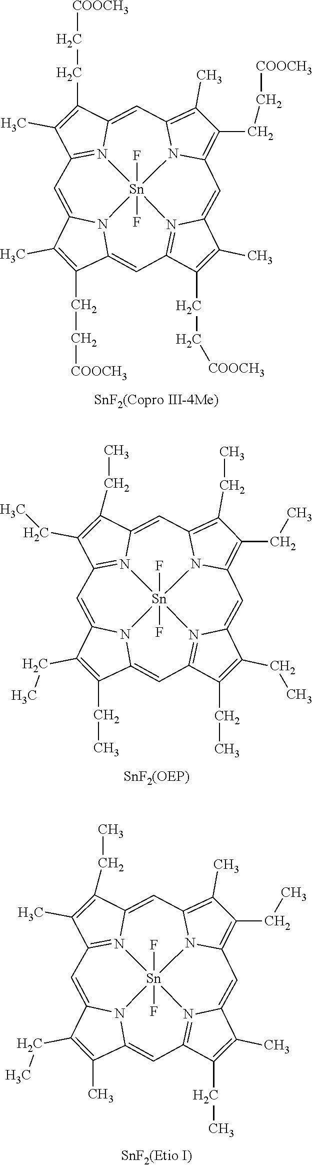

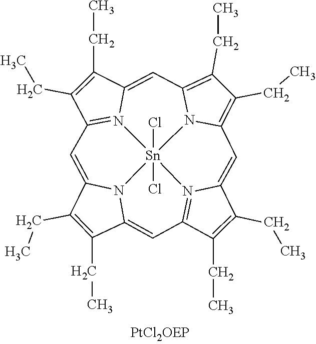

Alternatively, a heteroaromatic compound having a π-electron rich heteroaromatic compound and a π-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(1OH-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DNMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuiro[3,2-4]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), and 9-[3-(4,6-diphenyl-1,3,5-triazin-2-yl)phenyl]-9′-phenyl-2,3′-bi-9H-carbazole (abbreviation: mPCCzPTzn-02), may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are improved and the energy difference between the singlet excited state and the triplet excited state becomes small. As the TADF material, a TADF material (TADF 100) in which a singlet excited state and a triplet excited state is in a thermal equilibrium state may be used. Since such a TADF material enables a short emission lifetime (excitation lifetime), a decrease in efficiency of a light-emitting device in a high-luminance region can be inhibited.

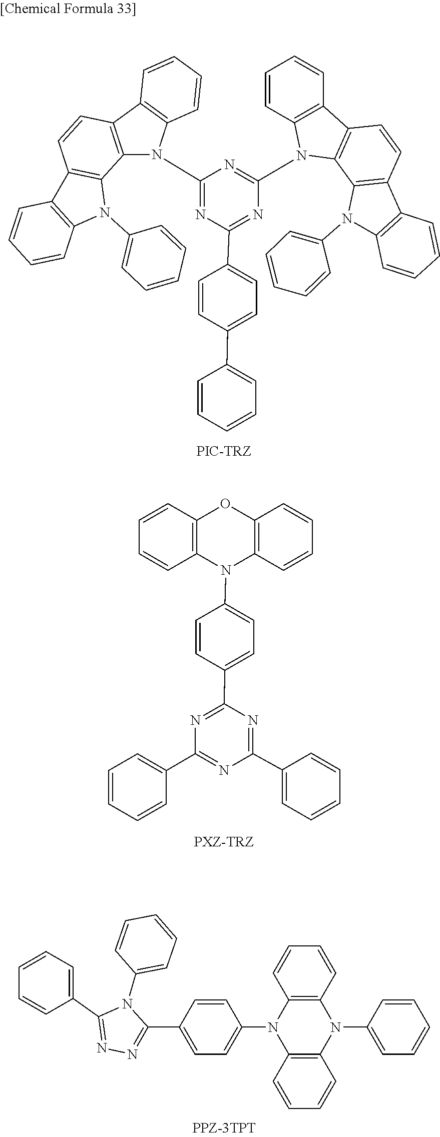 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1]

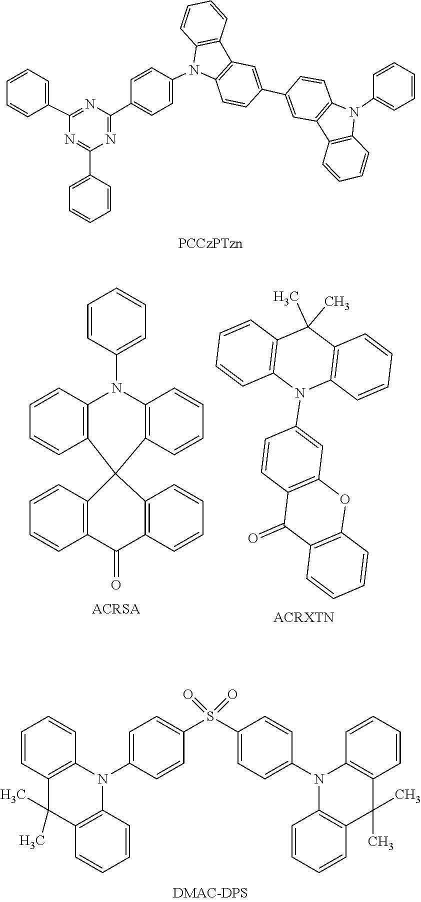

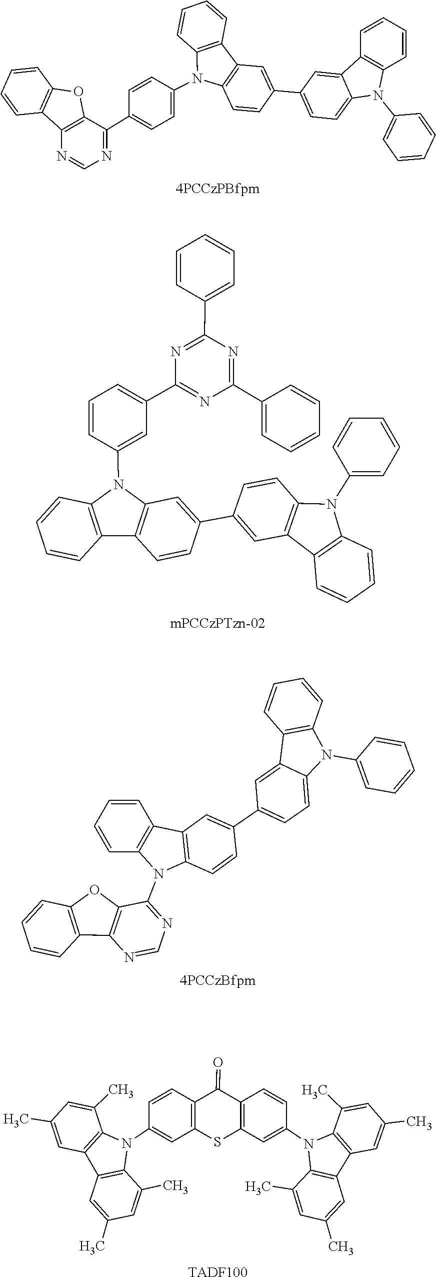

As the material having a function of converting triplet excitation energy into light emission, a nanostructure of a transition metal compound having a perovskite structure is also given in addition to the above. In particular, a nanostructure of a metal-halide perovskite material is preferable. The nanostructure is preferably a nanoparticle or a nanorod.

As the organic compounds (the host material and the like) used in combination with the above-described light-emitting substance (the guest material) in the light-emitting layers (113, 113a, 113b, and 113c), one or more kinds of substances having a larger energy gap than the light-emitting substance (the guest material) may be selected to be used.

### <<Host Material for Fluorescent Light Emission>>

In the case where the light-emitting substance used in the light-emitting layers (113, 113a, 113b, and 113c) is a fluorescent substance, an organic compound (a host material) used in combination with the light-emitting substance is preferably an organic compound that has a high energy level in a singlet excited state and has a low energy level in a triplet excited state or an organic compound having a high fluorescence quantum yield. Therefore, the hole-transport material (described above) and the electron-transport material (described below) in this embodiment, for example, can be used as long as it is an organic compound that satisfies such a condition. The organic compounds described in Embodiment 1 can also be used.

In terms of a preferable combination with the light-emitting substance (the fluorescent substance), examples of the organic compound (the host material) include condensed polycyclic aromatic compounds such as an anthracene derivative, a tetracene derivative, a phenanthrene derivative, a pyrene derivative, a chrysene derivative, and a dibenzo[g,p]chrysene derivative, although some of them overlap with the above specific examples.

Note that specific examples of the organic compound (the host material) preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)-phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N″,N″,N″,N′″,N′″-octaphenyldibenzo[gp]chrysene-2,7,10,15-tetraamine(abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)-biphenyl-4′-yl}-anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: α,β-ADN), 2-(10-phenylanthracen-9-yl)dibenzofuran, 2-(10-phenyl-9-anthracenyl)-benzo[b]naphtho[2,3-d]furan (abbreviation: Bnf(II)PhA), 9-(1-naphthyl)-10-[4-(2-naphthyl)phenyl]anthracene (abbreviation: αN-βNPAnth), 2,9-di(1-naphthyl)-10-phenylanthracene (abbreviation: 2αN-αNPhA), 9-(1-naphthyl)-10-[3-(1-naphthyl)phenyl]anthracene (abbreviation: αN-mαNPAnth), 9-(2-naphthyl)-10-[3-(1-naphthyl)phenyl]anthracene (abbreviation: βN-mαNPAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: ON-βNPAnth), 2-(1-naphthyl)-9-(2-naphthyl)-10-phenylanthracene (abbreviation: 2αN-βNPhA), 9-(2-naphthyl)-10-[3-(2-naphthyl)phenyl]anthracene (abbreviation: βN-mβNPAnth), 1-[4-(10-[1,1′-biphenyl]-4-yl-9-anthracenyl)phenyl]-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, 5,12-bis(biphenyl-2-yl)tetracene, and the like.

### <<Host Material for Phosphorescent Light>>

In the case where the light-emitting substance used for the light-emitting layers (113, 113a, 113b, and 113c) is a phosphorescent substance, an organic compound having triplet excitation energy (energy difference between a ground state and a triplet excited state) which is higher than that of the light-emitting substance is preferably selected as the organic compound (the host material) used in combination with the light-emitting substance. Note that in the case where a plurality of organic compounds (e.g., a first host material and a second host material (or an assist material)) are used in combination with a light-emitting substance in order to form an exciplex, the plurality of organic compounds are preferably mixed with a phosphorescent substance. The organic compounds described in Embodiment 1 can be used.

Such a structure makes it possible to efficiently obtain light emission utilizing ExTET (Exciplex-Triplet Energy Transfer), which is energy transfer from an exciplex to a light-emitting substance. Note that a combination of the plurality of organic compounds that easily forms an exciplex is preferably employed, and it is particularly preferable to combine a compound that easily accepts holes (a hole-transport material) and a compound that easily accepts electrons (an electron-transport material).

In terms of a preferable combination with the light-emitting substance (phosphorescent substance), examples of the organic compounds (the host material and the assist material) include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- or aluminum-based metal complexes, although some of them overlap with the above specific examples.

Among the above organic compounds, specific examples of the aromatic amine and the carbazole derivative, which are organic compounds having a high hole-transport property, are the same as the specific examples of the hole-transport materials described above. Any of these is preferable as the host material.

Among the above organic compounds, specific examples of the dibenzothiophene derivative and the dibenzofuran derivative, which are organic compounds having a high hole-transport property, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri(dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Any of these is preferable as the host material.

In addition, a metal complex having an oxazole-based or thiazole-based ligand, such as bis[2-(2-benzoxazolyl)phenolato]zinc(II) (abbreviation: ZnPBO) or bis[2-(2-benzothiazolyl)phenolato]zinc(II) (abbreviation: ZnBTZ), or the like is given as a preferable example of the host material.

Among the above organic compounds, specific examples of the oxadiazole derivative, the triazole derivative, the benzimidazole derivative, the quinoxaline derivative, the dibenzoquinoxaline derivative, the quinazoline derivative, the phenanthroline derivative, and the like, which are organic compounds having a high electron-transport property, include an organic compound including a heteroaromatic ring having a polyazole ring such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs); an organic compound including a heteroaromatic ring having a pyridine ring such as bathophenanthroline (abbreviation: Bphen), bathocuproine (abbreviation: BCP), 2,9-di(naphthalen-2-yl)-4,7-diphenyl-1,10-phenanthroline (abbreviation: NBphen), 2,2-(1,3-phenylene)bis[9-phenyl-1,10-phenanthroline](abbreviation: mPPhen2P), and 2-phenyl-9-[4-[4-(9-phenyl-1,10-phenanthrolin-2-yl)phenyl]phenyl]-1,10-phenanthroline (abbreviation: PPhen2BP) (abbreviation: PPhen2BP); 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 2-{4-[9,10-di(2-naphthyl)-2-anthryl]phenyl}-1-phenyl-1H-benzimidazole (abbreviation: ZADN), and 2-[4′-(9-phenyl-9H-carbazol-3-yl)-3,1′-biphenyl-1-yl]dibenzo[f,h]quinoxaline (abbreviation: 2mpPCBPDBq). Any of these is preferable as the host material.

Among the above organic compounds, specific examples of the pyridine derivative, the diazine derivative (including the pyrimidine derivative, the pyrazine derivative, and the pyridazine derivative), the triazine derivative, the furodiazine derivative, which are organic compounds having a high electron-transport property, include an organic compound including a heteroaromatic ring having a diazine ring such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)-1,1′-biphenyl-3-yl]-4,6-diphenyl-1,3,5-triazine (abbreviation: mFBPTzn), 8-(1,1′-biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′: 4,5]furo[2,3-b]pyrazine (abbreviation: 9mDBtBPNfpr), 9-[3′-(dibenzothiophen-4-yl)biphenyl-4-yl]naphtho[1′,2′: 4,5]furo[2,3-b]pyrazine (abbreviation: 9pmDBtBPNfpr), 11-[3′-(dibenzothiophen-4-yl)biphenyl-3-yl]phenanthro[9′,10′:4,5]furo[2,3-b]pyrazine (abbreviation: 11mDBtBPPnfpr), 11-[3′-(dibenzothiophen-4-yl)biphenyl-4-yl]phenanthro[9′,10′:4,5]furo[2,3-b]pyrazine, 11-[(3′-(9H-carbazol-9-yl)biphenyl-3-yl]phenanthro[9′,10′:4,5]furo[2,3-b]pyrazine, 12-(9′-phenyl-3,3′-bi-9H-carbazol-9-yl)phenanthro[9′,10′:4,5]furo[2,3-b]pyrazine (abbreviation: 12PCCzPnfpr), 9-[(3′-9-phenyl-9H-carbazol-3-yl)biphenyl-4-yl]naphtho[1′,2′:4,5]furo[2,3-b]pyrazine (abbreviation: 9pmPCBPNfpr), 9-(9′-phenyl-3,3′-bi-9H-carbazol-9-yl)naphtho[1′,2′:4,5]furo[2,3-b]pyrazine (abbreviation: 9PCCzNfpr), 10-(9′-phenyl-3,3′-bi-9H-carbazol-9-yl)naphtho[1′,2′:4,5]furo[2,3-b]pyrazine (abbreviation: 10PCCzNfpr), 9-[3′-(6-phenylbenzo[b]naphtho[1,2-d]furan-8-yl)biphenyl-3-yl]naphtho[1′,2′:4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′:4,5]furo[2,3-b]pyrazine (abbreviation: 9mFDBtPNfpr), 9-[3′-(6-phenyldibenzothiophen-4-yl)biphenyl-3-yl]naphtho[1′,2′:4,5]furo[2,3-b]pyrazine (abbreviation: 9mDBtBPNfpr-02), 9-[3-(9′-phenyl-3,3′-bi-9H-carbazol-9-yl)phenyl]naphtho[1′,2′:4,5]furo[2,3-b]pyrazine (abbreviation: 9mPCCzPNfpr), 9-{(3′-[2,8-diphenyldibenzothiophen-4-yl)biphenyl-3-yl}naphtho[1′,2′:4,5]furo[2,3-b]pyrazine, 11-{(3′-[2,8-diphenyldibenzothiophen-4-yl)biphenyl-3-yl]phenanthro[9′,10′:4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)-1,1′-biphenyl-3-yl]-4,6-diphenyl-1,3,5-triazine (abbreviation: mTpBPTzn), 2-[(1,1′-biphenyl)-4-yl]-4-phenyl-6-[9,9′-spirobi(9H-fluoren)-2-yl]-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-[1,1′-biphenyl]-3-yl-4-phenyl-6-(8-[1,1′:4′,1″-terphenyl]-4-yl-1-dibenzofuranyl)-1,3,5-triazine (abbreviation: mBP-TPDBfTzn), 6-(1,1′-biphenyl-3-yl)-4-[3,5-bis(9H-carbazol-9-yl)phenyl]-2-phenylpyrimidine (abbreviation: 6mBP-4Cz2PPm), and 4-[3,5-bis(9H-carbazol-9-yl)phenyl]-2-phenyl-6-(1,1′-biphenyl-4-yl)pyrimidine (abbreviation: 6BP-4Cz2PPm). Any of these is preferable as the host material.

Among the above organic compounds, specific examples of the metal complexes, which are organic compounds having a high electron-transport property, include zinc- and aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq), and metal complexes having a quinoline ring or a benzoquinoline ring. Any of these is preferable as the host material.

In addition, a high molecular compound such as poly(2,5-pyridinediyl) (abbreviation: PPy), poly[(9,9-dihexylfluorene-2,7-diyl)-co-(pyridine-3,5-diyl)](abbreviation: PF-Py), or poly[(9,9-dioctylfluorene-2,7-diyl)-co-(2,2′-bipyridine-6,6′-diyl)](abbreviation: PF-BPy) or the like is also preferable as the host material.

Furthermore, an organic compound having a bipolar property, i.e., both a high hole-transport property and a high electron-transport property, and including a diazine ring such as 9-phenyl-9′-(4-phenyl-2-quinazolinyl)-3,3′-bi-9H-carbazole (abbreviation: PCCzQz), 2-[4′-(9-phenyl-9H-carbazol-3-yl)-3,1′-biphenyl-1-yl]dibenzo[f,h]quinoxaline (abbreviation: 2mpPCBPDBq), 5-[3-(4,6-diphenyl-1,3,5-triazin-2yl)phenyl]-7,7-dimethyl-5H,7H-indeno[2,1-b]carbazole (abbreviation: mINc(II)PTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), or 7-[4-(9-phenyl-9H-carbazol-2-yl)quinazolin-2-yl]-7H-dibenzo[c,g]carbazole (abbreviation: PC-cgDBCzQz) can be used as the host material.

### <Electron-Transport Layer>

The electron-transport layers (114, 114a, and 114b) are each a layer that transports the electrons, which are injected from the second electrode 102 and charge-generation layers (106, 106a, and 106b) by the electron-injection layers (115, 115a, and 115b) to be described later, to the light-emitting layers (113, 113a, and 113b). Note that the heat resistance of the light-emitting device of one embodiment of the present invention can be improved by including the stacked electron-transport layers. It is preferable that the electron-transport materials used in the electron-transport layers (114, 114a, and 114b) be substances with an electron mobility higher than or equal to 1×10⁻⁶ cm²/Vs in the case where the square root of the electric field strength [V/cm] is 600. Note that other substances can also be used as long as they have an electron-transport property higher than a hole-transport property. Each of the electron-transport layers (114, 114a, and 114b) functions even in the form of a single layer but may have a stacked-layer structure of two or more layers. Note that since the above-described mixed material has heat resistance, performing a photolithography step over the electron-transport layer including such a material can inhibit the influence of a thermal process on the device characteristics.

### <<Electron-Transport Material>>

As the electron-transport material that can be used for the electron-transport layers (114, 114a, and 114b), an organic compound with a high electron-transport property can be used; for example, a heteroaromatic compound can be used. The heteroaromatic compound refers to a cyclic compound containing at least two different kinds of elements in a ring. Examples of cyclic structures include a three-membered ring, a four-membered ring, a five-membered ring, a six-membered ring, and the like, among which a five-membered ring and a six-membered ring are particularly preferable; the elements contained in the heteroaromatic compound are preferably one or more of nitrogen, oxygen, sulfur, and the like, as well as carbon. In particular, a heteroaromatic compound containing nitrogen (a nitrogen-containing heteroaromatic compound) is preferable, and any of materials having a high electron-transport property (electron-transport materials), such as a nitrogen-containing heteroaromatic compound and a π-electron deficient heteroaromatic compound including the nitrogen-containing heteroaromatic compound, is preferably used. The compound in Embodiment 1 has an electron-transport property and thus can be used as an electron-transport material.

Note that the electron-transport material can be different from the materials used for the light-emitting layer. Not all excitons formed by recombination of carriers in the light-emitting layer can contribute to light emission and some excitons might be diffused into a layer in contact with the light-emitting layer or a layer in the vicinity of the light-emitting layer. In order to avoid this phenomenon, the energy level (the lowest singlet excitation energy level or the lowest triplet excitation energy level) of a material used for the layer in contact with the light-emitting layer or the layer in the vicinity of the light-emitting layer is preferably higher than that of a material used for the light-emitting layer. Therefore, when a material different from the material of the light-emitting layer is used as the electron-transport material, a light-emitting device with high efficiency can be obtained.

The heteroaromatic compound is an organic compound including at least one heteroaromatic ring.

Note that the heteroaromatic ring includes any one of a pyridine ring, a diazine ring, a triazine ring, a polyazole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having a polyazole ring includes a heteroaromatic ring having an imidazole ring, a triazole ring, or an oxadiazole ring.

The heteroaromatic ring includes a fused heteroaromatic ring having a fused ring structure. Examples of the fused heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of a heteroaromatic compound including carbon and one or more of nitrogen, oxygen, sulfur, and the like and having a five-membered ring structure include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of a heteroaromatic compound including carbon and one or more of nitrogen, oxygen, sulfur, and the like and having a six-membered ring structure include a heteroaromatic compound having a heteroaromatic ring such as a pyridine ring, a diazine ring (a pyrimidine ring, a pyrazine ring, a pyridazine ring, or the like), a triazine ring, or a polyazole ring. Other examples include a heteroaromatic compound having a bipyridine structure and a heteroaromatic compound having a terpyridine structure, which are included in heteroaromatic compounds in which pyridine rings are connected.

Examples of the heteroaromatic compound having a fused ring structure including the above six-membered ring structure as a part include a heteroaromatic compound having a fused heteroaromatic ring such as a quinoline ring, a benzoquinoline ring, a quinoxaline ring, a dibenzoquinoxaline ring, a phenanthroline ring, a furodiazine ring (including a structure in which an aromatic ring is fused to the furan ring of a furodiazine ring), or a benzimidazole ring.

Specific examples of the above-described heteroaromatic compound having a five-membered ring structure (e.g., a polyazole ring (including an imidazole ring, a triazole ring, an oxadiazole ring), an oxazole ring, a thiazole ring, or a benzimidazole ring) include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl)stilbene (abbreviation: BzOs).

Specific examples of the above-described heteroaromatic compound having a six-membered ring structure (including a heteroaromatic ring having a pyridine ring, a diazine ring, a triazine ring, or the like) include a heteroaromatic compound including a heteroaromatic ring having a pyridine ring, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB), a heteroaromatic compound including a heteroaromatic ring having a triazine ring, such as 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)-1,1′-biphenyl-3-yl]-4,6-diphenyl-1,3,5-triazine (abbreviation: mTpBPTzn), 2-[(1,1′-biphenyl)-4-yl]-4-phenyl-6-[9,9′-spirobi(9H-fluoren)-2-yl]-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-[1,1′-biphenyl]-3-yl-4-phenyl-6-(8-[1,1′:4′,1″-terphenyl]-4-yl-1-dibenzofuranyl)-1,3,5-triazine (abbreviation: mBP-TPDBfTzn), 2-{3-[3-(dibenzothiophen-4-yl)phenyl]phenyl}-4,6-diphenyl-1,3,5-triazine (abbreviation: mDBtBPTzn), or mFBPTzn, and a heteroaromatic compound including a heteroaromatic ring having a diazine (pyrimidine) ring, such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 4,6mCzBP2Pm, 6-(1,1′-biphenyl-3-yl)-4-[3,5-bis(9H-carbazol-9-yl)phenyl)-2-phenylpyrimidine (abbreviation: 6mBP-4Cz2PPm), 4-[3,5-bis(9H-carbazol-9-yl)phenyl]-2-phenyl-6-(1,1′-biphenyl-4-yl)pyrimidine (abbreviation: 6BP-4Cz2PPm), 4-[3-(dibenzothiophen-4-yl)phenyl]-8-(naphthalen-2-yl)-[1]benzofuro[3,2-d]pyrimidine (abbreviation: 80N-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)(1,1′-biphenyl-3-yl)]naphtho[1′,2′: 4,5]furo[3,2-d]pyrimidine (abbreviation: 8mDBtBPNfpm), or 8-[(2,2′-binaphthalen)-6-yl]-4-[3-(dibenzothiophen-4-yl)phenyl]-[1]benzofuro[3,2-d]pyrimidine (abbreviation: 8(βN2)-4mDBtPBfpm). Note that the above aromatic compounds including a heteroaromatic ring include a heteroaromatic compound having a fused heteroaromatic ring.

Other examples include a heteroaromatic compound including a heteroaromatic ring having a diazine (pyrimidine) ring, such as 2,2′-(pyridine-2,6-diyl)bis(4-phenylbenzo[h]quinazoline) (abbreviation: 2,6(P-Bqn)2Py), 2,2′-(2,2′-bipyridine-6,6′-diyl)bis(4-phenylbenzo[h]quinazoline)(abbreviation: 6,6′(P-Bqn)2BPy), 2,2′-(pyridine-2,6-diyl)bis(4-[4-(2-naphthyl)phenyl]-6-phenylpyrimidine) (abbreviation: 2,6(NP-PPm)2Py), or 6-(1,1′-biphenyl-3-yl)-4-[3,5-bis(9H-carbazol-9-yl)phenyl]-2-phenylpyrimidine (abbreviation: 6mBP-4Cz2PPm), and a heteroaromatic compound including a heteroaromatic ring having a triazine ring, such as 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tz), or 2-[3-(2,6-dimethyl-3-pyridyl)-5-(9-phenanthryl)phenyl]-4,6-diphenyl-1,3,5-triazine (abbreviation: mPn-mDMePyPTzn).

Specific examples of the above-described heteroaromatic compound having a fused ring structure including the six-membered ring structure as a part (a heteroaromatic compound having a fused ring structure) include a heteroaromatic compound having a quinoxaline ring, such as bathophenanthroline (abbreviation: BPhen), bathocuproine (abbreviation: BCP), 2,9-di(naphthalen-2-yl)-4,7-diphenyl-1,10-phenanthroline (abbreviation: NBphen), 2,2′-(1,3-phenylene)bis[9-phenyl-1,10-phenanthroline](abbreviation: mPPhen2P), 2-phenyl-9-[4-[4-diylbis(9-phenyl-1,10-phenanthrolin-2-yl)phenyl]phenyl]-1,10-phenanthroline (abbreviation: PPhen2BP), 2,2′-(pyridine-2,6-diyl)bis(4-phenylbenzo[h]quinazoline) (abbreviation: 2,6(P-Bqn)2Py), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), or 2mpPCBPDBq.

For the electron-transport layers (114, 114a, and 114b), any of the metal complexes given below as well as the heteroaromatic compounds given above can be used. Examples of the metal complexes include a metal complex having a quinoline ring or a benzoquinoline ring, such as tris(8-quinolinolato)aluminum(III) (abbreviation: Alq₃), Almq₃, 8-quinolinolatolithium(I) (abbreviation: Liq), BeBq₂, bis(2-methyl-8-quinolinolato)(4-phenylphenolato)aluminum(III) (abbreviation: BAlq), or bis(8-quinolinolato)zinc(II) (abbreviation: Znq), and a metal complex having an oxazole ring or a thiazole ring, such as bis[2-(2-benzoxazolyl)phenolato]zinc(II) (abbreviation: ZnPBO) or bis[2-(2-benzothiazolyl)phenolato]zinc(II) (abbreviation: ZnBTZ).

A high molecular compound such as poly(2,5-pyridinediyl) (abbreviation: PPy), poly[(9,9-dihexylfluorene-2,7-diyl)-co-(pyridine-3,5-diyl)](abbreviation: PF-Py), or poly[(9,9-dioctylfluorene-2,7-diyl)-co-(2,2′-bipyridine-6,6′-diyl)](abbreviation: PF-BPy) can also be used as an electron-transport material.

The electron-transport layer (114, 114a, or 114b) is not limited to a single layer, and may be a stack of two or more layers each made of any of the above substances.

### <Electron-Injection Layer>

The electron-injection layers (115, 115a, and 115b) are each a layer containing a substance having a high electron-injection property. The electron-injection layers (115, 115a, and 115b) are each a layer for increasing the efficiency of electron injection from the second electrode 102 and are each preferably formed using a material whose LUMO level value has a small difference (0.5 eV or less) from the work function value of the material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), Liq, 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), lithium oxide (LiOₓ), or cesium carbonate. A rare earth metal or a rare earth metal compound such as erbium fluoride (ErF₃) or ytterbium (Yb) can also be used. Note that to form the electron-injection layers (115, 115a, and 115b), a plurality of kinds of the above-described materials may be mixed or a plurality of kinds of the above-described materials may be stacked. Electride may also be used for the electron-injection layers (115, 115a, and 115b). Examples of the electride include a substance in which electrons are added at high concentration to a mixed oxide of calcium and aluminum. Note that any of the substances used in the electron-transport layers (114, 114a, and 114b), which are given above, can also be used.

A mixed material in which an organic compound and an electron donor (donor) are mixed may also be used in the electron-injection layers (115, 115a, and 115b). Such a mixed material is excellent in an electron-injection property and an electron-transport property because electrons are generated in the organic compound by the electron donor. In this case, the organic compound is preferably a material excellent in transporting the generated electrons; specifically, for example, the above-mentioned electron-transport materials (metal complexes, heteroaromatic compounds, and the like) used in the electron-transport layers (114, 114a, and 114b) can be used. Any substance showing an electron-donating property with respect to the organic compound can serve as an electron donor. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. A Lewis base such as magnesium oxide can also be used. An organic compound such as tetrathiafulvalene (abbreviation: TTF) can also be used. Alternatively, a stack of these materials may be used.

Moreover, a mixed material in which an organic compound and a metal are mixed may also be used in the electron-injection layers (115, 115a, and 115b). The organic compound used here preferably has a LUMO level higher than or equal to −3.6 eV and lower than or equal to −2.3 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferable examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, and the like), a triazine ring, a bipyridine ring, or a terpyridine ring), and a heteroaromatic compound having a fused ring structure including a six-membered ring structure as a part (e.g., a quinoline ring, a benzoquinoline ring, a quinoxaline ring, a dibenzoquinoxaline ring, or a phenanthroline ring). Since the materials are specifically described above, description thereof is omitted here.

As the metal used for the above mixed material, a transition metal that belongs to Group 5, Group 7, Group 9, or Group 11 in the periodic table or a material that belongs to Group 13 is preferably used, and Ag, Cu, Al, In, and the like can be given as examples. In this case, the organic compound forms a singly occupied molecular orbital (SOMO) with the transition metal.

To amplify light obtained from the light-emitting layer 113b, for example, the optical path length between the second electrode 102 and the light-emitting layer 113b is preferably less than one fourth of the wavelength λ of light emitted from the light-emitting layer 113b. In that case, the optical path length can be adjusted by changing the thickness of the electron-transport layer 114b or the electron-injection layer 115b.

When the charge-generation layer 106 is provided between the two EL layers (103a and 103b) as in the light-emitting device in FIG. 1D, a structure in which a plurality of EL layers are stacked between a pair of electrodes (the structure is also referred to as a tandem structure) can be obtained.

### <Charge-Generation Layer>

The charge-generation layer 106 has a function of injecting electrons into the EL layer 103a and injecting holes into the EL layer 103b when a voltage is applied between the first electrode (anode) 101 and the second electrode (cathode) 102. The charge-generation layer 106 may have either a structure in which an electron acceptor (acceptor) is added to a hole-transport material (also referred to as a P-type layer) or a structure in which an electron donor (donor) is added to an electron-transport material (also referred to as an electron-injection buffer layer). Alternatively, both of these structures may be stacked. Furthermore, an electron-relay layer may be provided between the P-type layer and the electron-injection buffer layer. Note that forming the charge-generation layer 106 with the use of any of the above materials can inhibit an increase in driving voltage caused by the stack of the EL layers.

In the case where the charge-generation layer 106 has a structure in which an electron acceptor is added to a hole-transport material that is an organic compound (P-type layer), any of the materials described in this embodiment can be used as the hole-transport material. As examples of the electron acceptor, 7,7,8,8-tetracyano-2,3,5,6-tetrafluoroquinodimethane (abbreviation: F₄-TCNQ), chloranil, and the like can be given. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of the P-type layer or a stack of single films containing the respective materials may be used.

In the case where the charge-generation layer 106 has a structure in which an electron donor is added to an electron-transport material (electron-injection buffer layer), any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An organic compound such as tetrathianaphthacene may be used as the electron donor.

When an electron-relay layer is provided between a P-type layer and an electron-injection buffer layer in the charge-generation layer 106, the electron-relay layer contains at least a substance having an electron-transport property and has a function of preventing an interaction between the electron-injection buffer layer and the P-type layer and transferring electrons smoothly. The LUMO level of the substance having an electron-transport property in the electron-relay layer is preferably between the LUMO level of the acceptor substance in the P-type layer and the LUMO level of the substance having an electron-transport property in the electron-transport layer in contact with the charge-generation layer 106. A specific energy level of the LUMO level of the substance having an electron-transport property in the electron-relay layer is preferably higher than or equal to −5.0 eV, further preferably higher than or equal to −5.0 eV and lower than or equal to −3.0 eV. Note that as the substance having an electron-transport property in the electron-relay layer, a phthalocyanine-based material or a metal complex having a metal-oxygen bond and an aromatic ligand is preferably used.

Although FIG. 1D illustrates the structure in which two EL layers 103 are stacked, three or more EL layers may be stacked with charge-generation layers each provided between different EL layers.

### <Cap Layer>

Although not illustrated in FIG. 1A to FIG. 1E, a cap layer may be provided over the second electrode 102 of the light-emitting device. For example, a material with a high refractive index can be used for the cap layer. When the cap layer is provided over the second electrode 102, extraction efficiency of light emitted from the second electrode 102 can be improved.

Specific examples of a material that can be used for the cap layer include 5,5′-diphenyl-2,2′-di-5H-[1]benzothieno[3,2-c]carbazole (abbreviation: BisBTc) and 4,4′,4″-(benzene-1,3,5-triyl)tri(dibenzothiophene) (abbreviation: DBT3P-II). The organic compounds described in Embodiment 1 can also be used.

### <Substrate>

The light-emitting device described in this embodiment can be formed over a variety of substrates. Note that the type of substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, and a base material film including a fibrous material.

Examples of the glass substrate include barium borosilicate glass, aluminoborosilicate glass, and soda lime glass. Examples of the flexible substrate, the attachment film, and the base material film include plastics typified by polyethylene terephthalate (PET), polyethylene naphthalate (PEN), and polyether sulfone (PES), a synthetic resin such as acrylic resin, polypropylene, polyester, polyvinyl fluoride, polyvinyl chloride, polyamide, polyimide, aramid, epoxy resin, an inorganic vapor deposition film, and paper.

For fabrication of the light-emitting device described in this embodiment, a vapor phase method such as an evaporation method or a liquid phase method such as a spin coating method and an ink-jet method can be used. In the case of using an evaporation method, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, layers having a variety of functions (the hole-injection layer 111, the hole-transport layer 112, the light-emitting layer 113, the electron-transport layer 114, and the electron-injection layer 115) included in the EL layer of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, a screen printing (stencil) method, an offset printing (planography) method, a flexography (relief printing) method, a gravure printing method, or a micro-contact printing method), or the like.

In the case where a film formation method such as the coating method or the printing method is employed, a high molecular compound (e.g., an oligomer, a dendrimer, or a polymer), a middle molecular compound (a compound between a low molecular compound and a high molecular compound with a molecular weight of greater than or equal to 400 and less than or equal to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

Note that in this specification and the like, the term “layer” and the term “film” can be interchanged with each other as appropriate.

The structures described in this embodiment can be used in an appropriate combination with any of the structures described in the other embodiments.

### Embodiment 3

In this embodiment, a light-emitting and light-receiving apparatus 700 will be described in order to describe specific structure examples and an example of a manufacturing method of a light-emitting and light-receiving apparatus of one embodiment of the present invention. The light-emitting and light-receiving apparatus 700 includes a light-emitting device and thus can be regarded as a light-emitting apparatus; includes a light-receiving device and thus can be regarded as a light-receiving apparatus; and can be used in a display portion in an electronic apparatus and thus can be regarded as a display panel or a display apparatus.

### <Structure Example of Light-Emitting and Light-Receiving Apparatus 700>

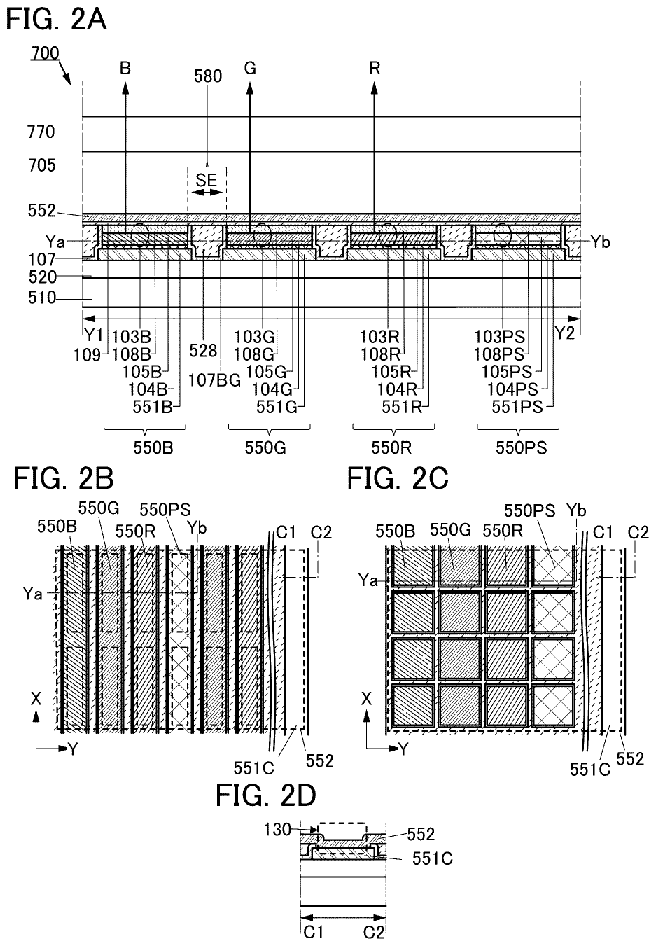

A light-emitting and light-receiving apparatus 700 illustrated in FIG. 2A includes a light-emitting device 550B, a light-emitting device 550G, a light-emitting device 550R, and a light-receiving device 550PS. The light-emitting device 550B, the light-emitting device 550G, the light-emitting device 550R, and the light-receiving device 550PS are formed over a functional layer 520 provided over a first substrate 510. The functional layer 520 includes driver circuits such as a gate driver and a source driver that are composed of a plurality of transistors and wirings that electrically connect these circuits. Note that these driver circuits are electrically connected to the light-emitting device 550B, the light-emitting device 550G, the light-emitting device 550R, and the light-receiving device 550PS, for example, and can drive them. The light-emitting and light-receiving apparatus 700 includes an insulating layer 705 over the functional layer 520 and the devices (the light-emitting devices and the light-receiving device), and the insulating layer 705 has a function of bonding a second substrate 770 and the functional layer 520.

The light-emitting device 550B, the light-emitting device 550G, and the light-emitting device 550R include the device structure described in Embodiment 2, and the light-receiving device 550PS has a device structure described later in Embodiment 8. Note that although in this embodiment, the case where the devices (a plurality of light-emitting devices and a light-receiving device) are formed separately is described, part of an EL layer of a light-emitting device (a hole-injection layer, a hole-transport layer, or an electron-transport layer) and part of an active layer of a light-receiving device (a first transport layer or a second transport layer) may be formed using the same material at the same time in the manufacturing process. The details will be described in Embodiment 8.

In this specification and the like, a structure in which light-emitting layers in light-emitting devices of different colors (e.g., blue (B), green (G), and red (R)) and light-receiving layers in light-receiving devices are separately formed or separately patterned may be referred to as an SBS (Side By Side) structure. Note that in the light-emitting and light-receiving apparatus 700 illustrated in FIG. 2A, the light-emitting device 550B, the light-emitting device 550G, the light-emitting device 550R, and the light-receiving device 550PS are arranged in this order; however, one embodiment of the present invention is not limited thereto. For example, in the light-emitting and light-receiving apparatus 700, the light-emitting device 550R, the light-emitting device 550G, the light-emitting device 550B, and the light-receiving device 550PS may be arranged in this order.

As illustrated in FIG. 2A, the light-emitting device 550B includes an electrode 551B, an electrode 552, and the EL layer 103B. The light-emitting device 550G includes an electrode 551G, the electrode 552, and an EL layer 103G. The light-emitting device 550R includes an electrode 551R, the electrode 552, and an EL layer 103R. The light-receiving device 550PS includes an electrode 551PS, the electrode 552, and a light-receiving layer 103PS. Note that a specific structure of each layer of the light-receiving device is as described in Embodiment 8. A specific structure of each layer of the light-emitting device is as described in Embodiment 2. The EL layer 103B, the EL layer 103G, and the EL layer 103R each have a stacked-layer structure of layers having different functions including light-emitting layers (105B, 105G, and 105R). The light-receiving layer 103PS has a stacked-layer structure of layers having different functions including an active layer 105PS. FIG. 2A illustrates a case where the EL layer 103B includes a hole-injection/transport layer 104B, the light-emitting layer 105B, an electron-transport layer 108B, and an electron-injection layer 109; the EL layer 103G includes a hole-injection/transport layer 104G, the light-emitting layer 105G, an electron-transport layer 108G, and the electron-injection layer 109; the EL layer 103R includes a hole-injection/transport layer 104R, the light-emitting layer 105R, an electron-transport layer 108R, and the electron-injection layer 109; and the light-receiving layer 103PS includes a first transport layer 104PS, the active layer 105PS, a second transport layer 108PS, and the electron-injection layer 109. However, the present invention is not limited thereto. Note that the hole-injection/transport layers (104B, 104G, and 104R) represent the layer having the functions of the hole-injection layer and the hole-transport layer described in Embodiment 2 and may have a stacked-layer structure.

Note that the electron-transport layers (108B, 108G, and 108R) and the second transport layer 108PS may have a function of blocking holes moving from the anode side to the cathode side through the EL layers (103B, 103G, and 103R) and the light-receiving layer 103PS of the light-receiving device. The electron-injection layer 109 may have a stacked-layer structure in which some or all of layers are formed using different materials.

As illustrated in FIG. 2A, an insulating layer 107 may be formed on side surfaces (or end portions) of the hole-injection/transport layers (104B, 104G, and 104R), the light-emitting layers (105B, 105G, and 105R), and the electron-transport layers (108B, 108G, and 108R) included in the EL layers (103B, 103G, and 103R), and side surfaces (or end portions) of the first transport layer 104PS, the active layer 105PS, and the second transport layer 108PS included in the light-receiving layer 103PS. The insulating layer 107 is formed in contact with the side surfaces (or the end portions) of the EL layers (103B, 103G, and 103R) and the light-receiving layer 103PS. This can inhibit entry of oxygen, moisture, or constituent elements thereof into the inside through the side surfaces of the EL layers (103B, 103G, and 103R) and the light-receiving layer 103PS. For the insulating layer 107, aluminum oxide, magnesium oxide, hafnium oxide, gallium oxide, indium gallium zinc oxide, silicon nitride, silicon nitride oxide, or the like can be used, for example. Some of the above-described materials may be stacked to form the insulating layer 107. The insulating layer 107 can be formed by a sputtering method, a CVD method, an MBE method, a PLD method, an ALD method, or the like and is formed preferably by an ALD method, which enables favorable coverage. Note that the insulating layer 107 continuously covers the side surfaces (or the end portions) of parts of the EL layers (103B, 103G, and 103R) and parts of the light-receiving layer 103PS of adjacent devices. For example, in FIG. 2A, the side surfaces of part of the EL layer 103B of the light-emitting device 550B and part of the EL layer 103G of the light-emitting device 550G are covered with an insulating layer 107. In a region covered with the insulating layer 107, a partition wall 528 formed using an insulating material is preferably formed, as illustrated in FIG. 2A.

In addition, the electron-injection layer 109 is formed over the electron-transport layers (108B, 108G, and 108R) that are parts of the EL layers (103B, 103G, and 103R), the second transport layer 108PS that is part of the light-receiving layer 103PS, and the insulating layers 107. Note that the electron-injection layer 109 may have a stacked-layer structure of two or more layers (for example, stacked layers having different electric resistances).

The electrode 552 is formed over the electron-injection layer 109. Note that the electrodes (551B, 551G, and 551R) and the electrode 552 have overlapping regions. The light-emitting layer 105B is provided between the electrode 551B and the electrode 552, the light-emitting layer 105G is provided between the electrode 551G and the electrode 552, the light-emitting layer 105R is provided between the electrode 551R and the electrode 552, and the light-receiving layer 103PS is provided between the electrode 551PS and the electrode 552.

The EL layers (103B, 103G, and 103R) illustrated in FIG. 2A each have a structure similar to that of the EL layer 103 described in Embodiment 2. The light-receiving layer 103PS has a structure similar to that of a light-receiving layer to be described later in Embodiment 8. The light-emitting layer 105B can emit blue light, the light-emitting layer 105G can emit green light, and the light-emitting layer 105R can emit red light, for example.

The partition walls 528 are provided in regions surrounded by the electron-injection layer 109 and the insulating layer 107. As illustrated in FIG. 2A, the partition walls 528 are in contact with the side surfaces (or the end portions) of the electrodes (551B, 551G, 551R, and 551PS) and parts of the EL layers (103B, 103G, and 103R) of the light-emitting devices and parts of the light-receiving layer 103PS through the insulating layer 107.

In each of the EL layers and the light-receiving layer, particularly the hole-injection layer, which is included in the hole-transport region between the anode and the light-emitting layer and the hole-transport region between the anode and the active layer, often has high conductivity; therefore, a hole-injection layer formed as a layer shared by adjacent devices might cause crosstalk. Thus, as described in this structure example, the partition walls 528 formed using an insulating material are provided between the EL layers and between the EL layer and the light-receiving layer, which can inhibit occurrence of crosstalk between adjacent devices.

In the manufacturing method described in this embodiment, side surfaces (or end portions) of the EL layer and the light-receiving layer are exposed in the patterning step. This may promote deterioration of the EL layer and the light-receiving layer by allowing the entry of oxygen, water, or the like through the side surfaces (or the end portions) of the EL layer and the light-receiving layer. Therefore, providing the partition wall 528 can inhibit the deterioration of the EL layer and the light-receiving layer in the manufacturing process.

Providing the partition wall 528 can flatten a depressed portion formed between adjacent devices. When the depressed portion is reduced, disconnection of the electrode 552 formed over the EL layers and the light-receiving layer can be inhibited. As an insulating material used for forming the partition wall 528, an organic material such as an acrylic resin, a polyimide resin, an epoxy resin, an imide resin, a polyamide resin, a polyimide-amide resin, a silicone resin, a siloxane resin, a benzocyclobutene-based resin, a phenol resin, or precursors of these resins can be used, for example. An organic material such as polyvinyl alcohol (PVA), polyvinyl butyral, polyvinylpyrrolidone, polyethylene glycol, polyglycerin, pullulan, water-soluble cellulose, or an alcohol-soluble polyamide resin can also be used. A photosensitive resin such as a photoresist can also be used. Note that as the photosensitive resin, a positive material or a negative material can be used.

With the photosensitive resin, the partition wall 528 can be fabricated only by light exposure and development steps. The partition wall 528 may be formed using a negative photosensitive resin (e.g., a resist material). In the case where an insulating layer containing an organic material is used as the partition wall 528, a material absorbing visible light is suitably used. When a material that absorbs visible light is used for the partition wall 528, light emitted from the EL layer can be absorbed by the partition wall 528, so that light that might leak to the adjacent EL layer and the adjacent light-receiving layer (stray light) can be inhibited. Thus, a display panel having high display quality can be provided.

For example, the difference between the top-surface level of the partition wall 528 and the top-surface level of any of the EL layer 103B, the EL layer 103G, the EL layer 103R, and the light-receiving layer 103PS is preferably 0.5 times or less, further preferably 0.3 times or less the thickness of the partition wall 528. The partition wall 528 may be provided such that the top-surface level of any of the EL layer 103B, the EL layer 103G, the EL layer 103R, and the light-receiving layer 103PS is higher than the top-surface level of the partition wall 528, for example. Alternatively, the partition wall 528 may be provided such that the top-surface level of the partition wall 528 is higher than the top-surface level of each of the EL layer 103B, the EL layer 103G, the EL layer 103R, and the light-receiving layer 103PS, for example.

When electrical continuity is established between the EL layer 103B, the EL layer 103G, the EL layer 103R, and the light-receiving layer 103PS in a light-emitting and light-receiving apparatus (display panel) with a high definition exceeding 1000 ppi, a crosstalk phenomenon occurs, resulting in a narrower color gamut of the light-emitting and light-receiving apparatus. Providing the partition wall 528 in a high-definition display panel with more than 1000 ppi, preferably more than 2000 ppi, or further preferably in an ultrahigh-definition display panel with more than 5000 ppi allows the display panel to express vivid colors.

FIG. 2B and FIG. 2C are each a schematic top view of the light-emitting and light-receiving apparatus 700 taken along the dashed-dotted line Ya-Yb in the cross-sectional view of FIG. 2A. The light-emitting devices 550B, the light-emitting devices 550G, and the light-emitting devices 550R are arranged in a matrix. FIG. 2B illustrates what is called stripe arrangement, in which the light-emitting devices of the same color are arranged in X-direction. FIG. 2C illustrates a structure in which the light-emitting devices of the same color are arranged in the X-direction and separated by patterning for each pixel. Note that the arrangement method of the light-emitting devices is not limited thereto; another arrangement method such as delta arrangement, zigzag arrangement, PenTile arrangement, or diamond arrangement may also be used.

Each of the EL layers (103B, 103G, and 103R) and the light-receiving layer 103PS are processed to be separated by patterning using a photolithography method; hence, a high-definition light-emitting and light-receiving apparatus (display panel) can be fabricated. End portions (side surfaces) of layers of the EL layer processed by patterning using a photolithography method have substantially one surface (or are positioned on substantially the same plane). In addition, the side surfaces (end portions) of the layers of the light-receiving layer processed by patterning using a photolithography method have substantially the same surface (or are positioned on substantially the same plane). In this case, the width (SE) of the space 580 between the EL layers and between the EL layer and the light-receiving layer is preferably 5 μm or less, further preferably 1 μm or less.

In the EL layer, particularly the hole-injection layer, which is included in the hole-transport region between the anode and the light-emitting layer, often has high conductivity; therefore, a hole-injection layer formed as a layer shared by adjacent light-emitting devices might cause crosstalk. Therefore, processing the EL layers to be separated by patterning using a photolithography method as shown in this structure example can suppress occurrence of crosstalk between adjacent light-emitting devices.

FIG. 2D is a schematic cross-sectional view taken along the dashed-dotted line C1-C2 in FIG. 2B and FIG. 2C. FIG. 2D illustrates a connection portion 130 where the connection electrode 551C and the electrode 552 are electrically connected. In the connection portion 130, the electrode 552 is provided over and in contact with the connection electrode 551C. In addition, the partition wall 528 is provided to cover the end portion of the connection electrode 551C.

### <Manufacturing Method Example of Light-Emitting and Light-Receiving Apparatus>

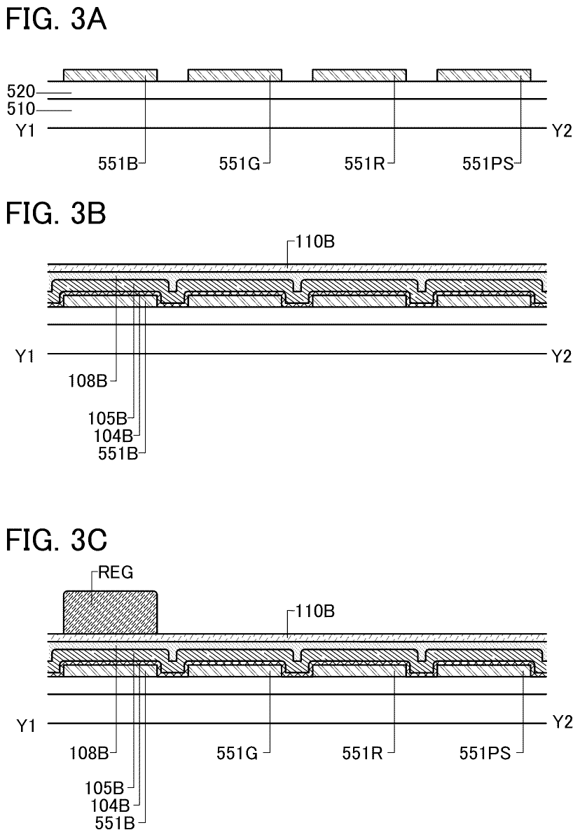

The electrode 551B, the electrode 551G, the electrode 551R, and the electrode 551PS are formed as illustrated in FIG. 3A. For example, a conductive film is formed over the functional layer 520 over the first substrate 510 and processed into predetermined shapes by a photolithography method.

The conductive film can be formed by a sputtering method, a chemical vapor deposition (CVD) method, a molecular beam epitaxy (MBE) method, a vacuum evaporation method, a pulsed laser deposition (PLD) method, an atomic layer deposition (ALD) method, or the like. Examples of the CVD method include a plasma-enhanced chemical vapor deposition (PECVD: Plasma Enhanced CVD) method and a thermal CVD method. As an example of the thermal CVD method, a metal organic chemical vapor deposition (MOCVD: Metal Organic CVD) method can be given.

The conductive film may be processed by a nanoimprinting method, a sandblasting method, a lift-off method, or the like as well as a photolithography method described above. Alternatively, island-shaped thin films may be directly formed by a film formation method using a shielding mask such as a metal mask.

There are two typical processing methods using a photolithography method. In one of the methods, a resist mask is formed over a thin film that is to be processed, the thin film is processed by etching or the like, and then the resist mask is removed. In the other method, a photosensitive thin film is formed and then processed into a desired shape by light exposure and development. The former method involves heat treatment steps such as heating after resist application (PAB: Pre Applied Bake) and heating after light exposure (PEB: Post Exposure Bake). In one embodiment of the present invention, a lithography method is used not only for processing of a conductive film but also for processing of a thin film used for formation of an EL layer (a film made of an organic compound or a film partly including an organic compound).

As light for light exposure in a photolithography method, it is possible to use the i-line (wavelength: 365 nm), the g-line (wavelength: 436 nm), the h-line (wavelength: 405 nm), or light in which the i-line, the g-line, and the h-line are mixed. Alternatively, ultraviolet light, KrF laser light, ArF laser light, or the like can be used. Light exposure may be performed by liquid immersion light exposure technique. As the light for light exposure, extreme ultraviolet (EUV) light or X-rays may also be used. Instead of the light for light exposure, an electron beam can be used. It is preferable to use extreme ultraviolet light, X-rays, or an electron beam because extremely minute processing can be performed. Note that a photomask is not needed when light exposure is performed by scanning with a beam such as an electron beam.

For etching of a thin film using a resist mask, a dry etching method, a wet etching method, a sandblast method, or the like can be used.

Subsequently, as illustrated in FIG. 3B, the hole-injection/transport layer 104B, the light-emitting layer 105B, and the electron-transport layer 108B are formed over the electrode 551B, the electrode 551G, the electrode 551R, and the electrode 551PS. Note that the hole-injection/transport layer 104B, the light-emitting layer 105B, and the electron-transport layer 108B can be formed using a vacuum evaporation method, for example. Furthermore, a sacrificial layer 110B is formed over the electron-transport layer 108B. For the formation of the hole-injection/transport layer 104B, the light-emitting layer 105B, and the electron-transport layer 108B, any of the materials described in Embodiment 2 can be used.

For the sacrificial layer 110B, it is preferable to use a film highly resistant to etching treatment performed on the hole-injection/transport layer 104B, the light-emitting layer 105B, and the electron-transport layer 108B, i.e., a film having high etching selectivity. The sacrificial layer 110B preferably has a stacked-layer structure of a first sacrificial layer and a second sacrificial layer which have different etching selectivities. Moreover, for the sacrificial layer 110B, it is possible to use a film that can be removed by a wet etching method less likely to cause damage to the EL layer 103B. In wet etching, oxalic acid or the like can be used as an etching material.

The sacrificial layer 110B can be formed using an inorganic film such as a metal film, an alloy film, a metal oxide film, a semiconductor film, or an inorganic insulating film, for example. The sacrificial layer 110B can be formed by any of a variety of film formation methods such as a sputtering method, an evaporation method, a CVD method, and an ALD method.

For the sacrificial layer 110B, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used. It is particularly preferable to use a low-melting-point material such as aluminum or silver.

The sacrificial layer 110B can be formed using a metal oxide such as indium gallium zinc oxide (In—Ga—Zn oxide, also referred to as IGZO). It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon can also be used, for example.

Note that an element M(Mis one or more kinds selected from aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used instead of gallium described above. In particular, M is preferably one or more kinds selected from gallium, aluminum, and yttrium.

For the sacrificial layer 110B, an inorganic insulating material such as aluminum oxide, hafnium oxide, or silicon oxide can be used.

The sacrificial layer 110B is preferably formed using a material that can be dissolved in a solvent chemically stable with respect to the electron-transport layer 108B, which is the uppermost layer. In particular, a material that will be dissolved in water or alcohol can be suitably used for the sacrificial layer 110B. In formation of the sacrificial layer 110B, it is preferable that application of such a material dissolved in a solvent such as water or alcohol be performed by a wet film formation method and followed by heat treatment for evaporating the solvent. At this time, the heat treatment is preferably performed in a reduced-pressure atmosphere, in which case the solvent can be removed at a low temperature in a short time and thermal damage to the hole-injection/transport layer 104B, the light-emitting layer 105B, and the electron-transport layer 108B can be reduced accordingly.

In the case where the sacrificial layer 110B having a stacked-layer structure is formed, the stacked-layer structure can include the first sacrificial layer formed using any of the above-described materials and the second sacrificial layer thereover.

The second sacrificial layer in that case is a film used as a hard mask for etching of the first sacrificial layer. In processing the second sacrificial layer, the first sacrificial layer is exposed. Thus, a combination of films having high etching selectivity therebetween is selected for the first sacrificial layer and the second sacrificial layer. Thus, a film that can be used for the second sacrificial layer can be selected in accordance with the etching conditions of the first sacrificial layer and the etching conditions of the second sacrificial layer.

For example, in the case where dry etching using a gas containing fluorine (also referred to as a fluorine-based gas) is performed for the etching of the second sacrificial layer, silicon, silicon nitride, silicon oxide, tungsten, titanium, molybdenum, tantalum, tantalum nitride, an alloy containing molybdenum and niobium, an alloy containing molybdenum and tungsten, or the like can be used for the second sacrificial layer. Here, a metal oxide film of IGZO, ITO, or the like is given as an example of a film having high etching selectivity (that is, enabling low etching rate) in dry etching using the fluorine-based gas, and such a film can be used as the first sacrificial layer.

Note that the material for the second sacrificial layer is not limited to the above and can be selected from a variety of materials in accordance with the etching conditions of the first sacrificial layer and the etching conditions of the second sacrificial layer. For example, any of the films that can be used for the first sacrificial layer can be selected.

As the second sacrificial layer, a nitride film can be used, for example. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used as the second sacrificial layer. Typically, a film of an oxide or an oxynitride such as silicon oxide, silicon oxynitride, aluminum oxide, aluminum oxynitride, hafnium oxide, or hafnium oxynitride can be used.

Next, as illustrated in FIG. 3C, a resist is applied onto the sacrificial layer 110B, and the resist having a desired shape (a resist mask REG) is formed by a photolithography method. Such a method involves heat treatment steps such as heating after resist application (PAB: Pre Applied Bake) and heating after light exposure (PEB: Post Exposure Bake). The PAB temperature reaches approximately 100° C. and the PEB temperature reaches approximately 120° C., for example. Therefore, the light-emitting device needs to be resistant to such treatment temperatures.

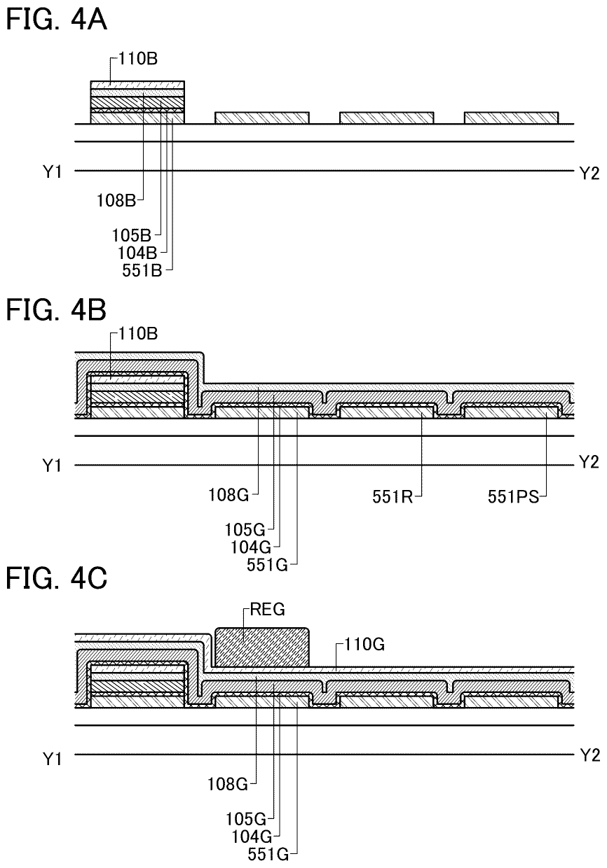

Next, part of the sacrificial layer 110B that is not covered with the resist mask REG is removed by etching using the obtained resist mask REG, the resist mask REG is removed, and then the hole-injection/transport layer 104B, the light-emitting layer 105B, and the electron-transport layer 108B that are not covered with the sacrificial layer 110B are partly removed by etching, so that the hole-injection/transport layer 104B, the light-emitting layer 105B, and the electron-transport layer 108B are processed to have side surfaces (or have their side surfaces exposed) over the electrode 551B or have belt-like shapes extending in the direction intersecting with the paper. Note that dry etching is preferably employed for the etching. In the case where the sacrificial layer 110B has the aforementioned stacked-layer structure of the first sacrificial layer and the second sacrificial layer, the hole-injection/transport layer 104B, the light-emitting layer 105B, and the electron-transport layer 108B may be processed into predetermined shapes in the following manner: part of the second sacrificial layer is etched with use of the resist mask REG, the resist mask REG is then removed, and part of the first sacrificial layer is etched with use of the second sacrificial layer as a mask. The shape illustrated in FIG. 4A is obtained through these etching treatment.

Subsequently, as illustrated in FIG. 4B, the hole-injection/transport layer 104G, the light-emitting layer 105G, and the electron-transport layer 108G are formed over the sacrificial layer 110B, the electrode 551G, the electrode 551R, and the electrode 551PS. As the materials for forming the hole-injection/transport layer 104G, the light-emitting layer 105G, and the electron-transport layer 108G, any of the materials described in Embodiment 2 can be used. Note that the hole-injection/transport layer 104G, the light-emitting layer 105G, and the electron-transport layer 108G can be formed by a vacuum evaporation method, for example.

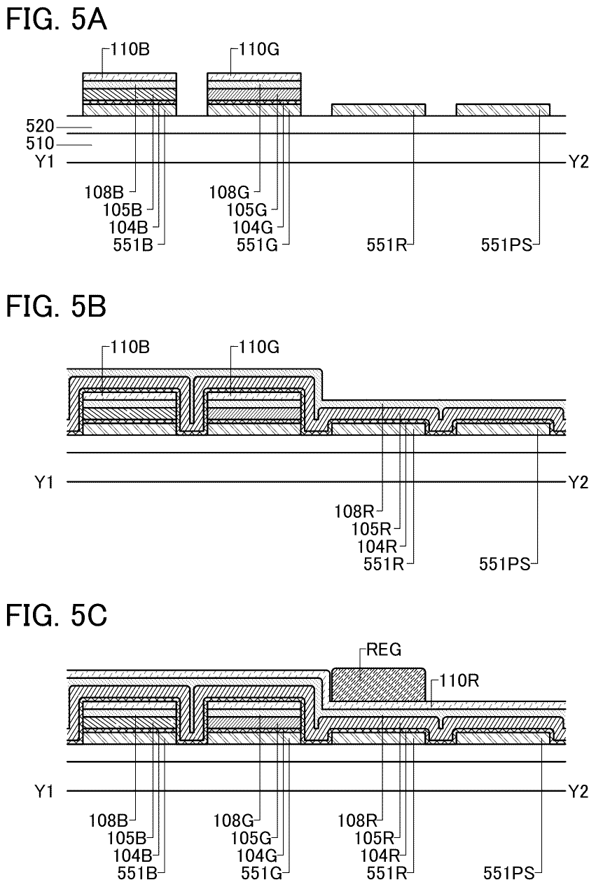

Next, as illustrated in FIG. 4C, the sacrificial layer 110G is formed over the electron-transport layer 108G, a resist is applied onto the sacrificial layer 110G, and the resist having a desired shape (the resist mask) is formed by a photolithography method. Part of the sacrificial layer 110G that is not covered with the obtained resist mask is removed by etching, the resist mask is then removed, and then the hole-injection/transport layer 104G, the light-emitting layer 105G, and the electron-transport layer 108G that are not covered with the sacrificial layer 110G are partly removed by etching. Thus, the hole-injection/transport layer 104G, the light-emitting layer 105G, and the electron-transport layer 108G are processed to have side surfaces (or have their side surfaces exposed) over the electrode 551G or have belt-like shapes extending in the direction intersecting with the paper. Note that dry etching is preferably employed for the etching. The sacrificial layer 110G can be formed using a material similar to that for the sacrificial layer 110B. In the case where the sacrificial layer 110G has the aforementioned stacked-layer structure of the first sacrificial layer and the second sacrificial layer, the hole-injection/transport layer 104G, the light-emitting layer 105G, and the electron-transport layer 108G may be processed into predetermined shapes in the following manner: part of the second sacrificial layer is etched with use of the resist mask, the resist mask is then removed, and then part of the first sacrificial layer is etched with use of the second sacrificial layer as a mask. The shape illustrated in FIG. 5A is obtained through these etching treatment.

Next, as illustrated in FIG. 5B, the hole-injection/transport layer 104R, the light-emitting layer 105R, and the electron-transport layer 108R are formed over the sacrificial layer 110B, the sacrificial layer 110G, the electrode 551R, and the electrode 551PS. For the formation of the hole-injection/transport layer 104R, the light-emitting layer 105R, and the electron-transport layer 108R, any of the materials described in Embodiment 2 can be used. Note that the hole-injection/transport layer 104R, the light-emitting layer 105R, and the electron-transport layer 108R can be formed by a vacuum evaporation method, for example.

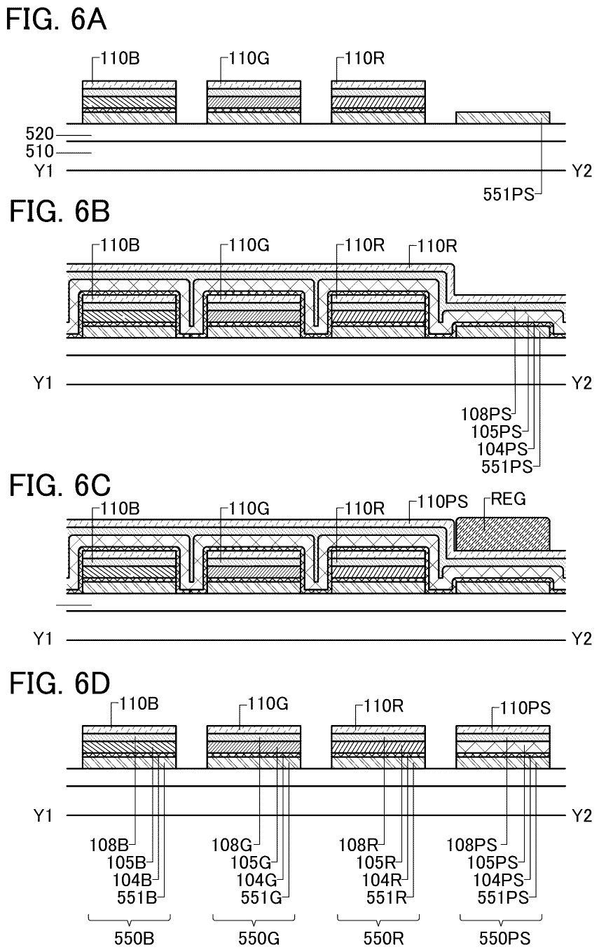

Next, as illustrated in FIG. 5C, the sacrificial layer 110R is formed over the electron-transport layer 108R, a resist is applied onto the sacrificial layer 110R, and the resist having a desired shape (the resist mask REG) is formed by a photolithography method. Part of the sacrificial layer 110R that is not covered with the obtained resist mask REG is removed by etching, the resist mask REG is then removed, and then the hole-injection/transport layer 104R, the light-emitting layer 105R, and the electron-transport layer 108R that are not covered with the sacrificial layer 110R are removed by etching. Thus, the hole-injection/transport layer 104R, the light-emitting layer 105R, and the electron-transport layer 108R are processed to have side surfaces (or have their side surfaces exposed) over the electrode 551R or have belt-like shapes extending in the direction intersecting with the paper. Note that dry etching is preferably employed for the etching. The sacrificial layer 110R can be formed using a material similar to that for the sacrificial layer 110B. In the case where the sacrificial layer 110R has the aforementioned stacked-layer structure of the first sacrificial layer and the second sacrificial layer, the hole-injection/transport layer 104R, the light-emitting layer 105R, and the electron-transport layer 108R may be processed into a predetermined shape in the following manner: part of the second sacrificial layer is etched with use of the resist mask REG, the resist mask REG is then removed, and part of the first sacrificial layer is etched with use of the second sacrificial layer as a mask. The shape illustrated in FIG. 6A is obtained through these etching treatment.

Next, as illustrated in FIG. 6B, the first transport layer 104PS, the active layer 105PS, and the second transport layer 108PS are formed over the sacrificial layer 110B, the sacrificial layer 110G, the sacrificial layer 110R, and the electrode 551PS. As a material for forming the first transport layer 104PS, for example, the material for the hole-injection layer and the hole-transport layer described in Embodiment 2 can be used. As a material for the active layer 105PS, a material described in Embodiment 8 can be used. Furthermore, as a material for forming the second transport layer 108PS, for example, the material for the electron-transport layer and the electron-injection layer described in Embodiment 2 can be used. Note that the first transport layer 104PS, the active layer 105PS, and the second transport layer 108PS can be formed by a vacuum evaporation method, for example.

Next, as illustrated in FIG. 6C, the sacrificial layer 110PS is formed over the second transport layer 108PS, a resist is applied onto the sacrificial layer 110PS, and the resist having a desired shape (the resist mask REG) is formed by a photolithography method. Part of the sacrificial layer 110PS that is not covered with the obtained resist mask REG is removed by etching, the resist mask REG is then removed, and the first transport layer 104PS, the active layer 105PS, and the second transport layer 108PS that are not covered with the sacrificial layer 110PS are partly removed by etching. Thus, the first transport layer 104PS, the active layer 105PS, and the second transport layer 108PS are processed to have side surfaces (or have their side surfaces exposed) over the electrode 551PS or have belt-like shapes extending in the direction intersecting with the paper. Note that dry etching is preferably employed for the etching. The sacrificial layer 110PS can be formed using a material similar to that for the sacrificial layer 110B. In the case where the sacrificial layer 110PS has the aforementioned stacked-layer structure of the first sacrificial layer and the second sacrificial layer, the first transport layer 104PS, the active layer 105PS, and the second transport layer 108PS may be processed into a predetermined shape in the following manner: part of the second sacrificial layer is etched using the resist mask REG, the resist mask REG is then removed, and part of the first sacrificial layer is etched using the second sacrificial layer as a mask. The shape illustrated in FIG. 6D is obtained through these etching treatment.

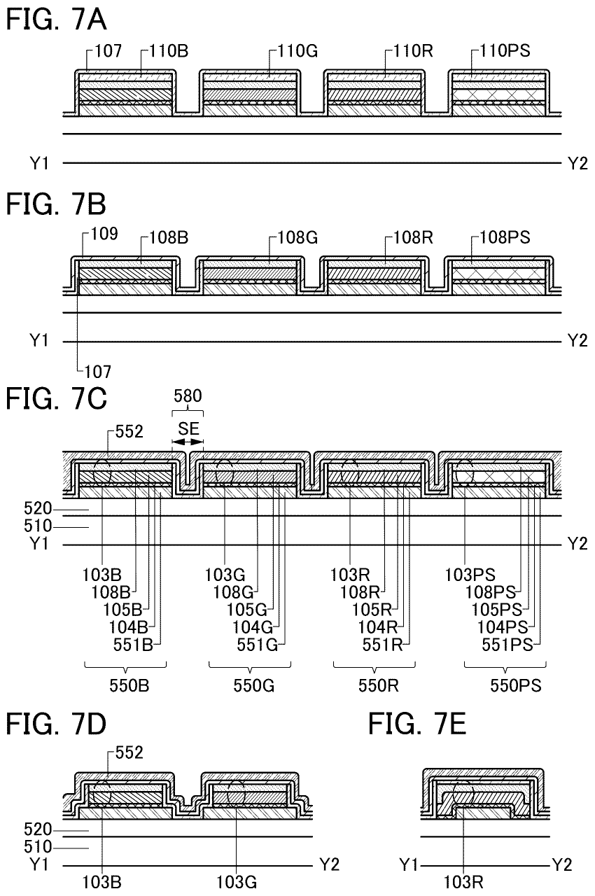

Next, as illustrated in FIG. 7A, the insulating layer 107 is formed over the sacrificial layer 110B, the sacrificial layer 110G, the sacrificial layer 110R, and the sacrificial layer 110PS.

For formation of the insulating layer 107, an ALD method can be used, for example. In this case, as illustrated in FIG. 7A, the insulating layer 107 is formed to be in contact with the side surfaces (end portions) of the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device. This can inhibit entry of oxygen, moisture, or constituent elements thereof into the inside through the side surfaces. As a material used for the insulating layer 107, aluminum oxide, magnesium oxide, hafnium oxide, gallium oxide, indium gallium zinc oxide, silicon nitride, silicon nitride oxide, or the like can be used, for example.

Next, as illustrated in FIG. 7B, part of the insulating layer 107 and the sacrificial layers (110B, 110G, 110R, and 110PS) are removed, and then the electron-injection layer 109 is formed over the insulating layer 107, the electron-transport layers (108B, 108G, and 108R), and the second transport layer 108PS. For forming the electron-injection layer 109, any of the materials described in Embodiment 2 can be used. The electron-injection layer 109 is formed by a vacuum evaporation method, for example. Note that the electron-injection layer 109 is in contact with the side surfaces (end portions) of the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device through the insulating layer 107.

Next, as illustrated in FIG. 7C, the electrode 552 is formed. The electrode 552 is formed by a vacuum evaporation method, for example. The electrode 552 is formed over the electron-injection layer 109. Note that the electrode 552 is in contact with the side surfaces (end portions) of the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device through the electron-injection layer 109 and the insulating layer 107. This can prevent electrical short circuits between the electrode 552 and each of the following layers: the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device.

Through the above steps, the EL layer 103B, the EL layer 103G, the EL layer 103R, and the light-receiving layer 103PS in the light-emitting device 550B, the light-emitting device 550G, the light-emitting device 550R, and the light-receiving device 550PS can be processed to be separated from each other.

The EL layers (the EL layer 103B, the EL layer 103G, and the EL layer 103R) and the light-receiving layer 103PS are processed to be separated by patterning using a photolithography method; hence, a high-definition light-emitting and light-receiving apparatus (display panel) can be fabricated. End portions (side surfaces) of layers of the EL layer processed by patterning using a photolithography method have substantially one surface (or are positioned on substantially the same plane). In addition, the side surfaces (end portions) of the layers of the light-receiving layer processed by patterning using a photolithography method have substantially the same surface (or are positioned on substantially the same plane).

The hole-injection/transport layers (104B, 104G, and 104R) of the EL layers and the first transport layer 104PS of the light-receiving layer often have high conductivity, and thus might cause crosstalk when formed as layers shared by adjacent devices. Therefore, processing the layers to be separated by patterning using a photolithography method as shown in this structure example can suppress occurrence of crosstalk between adjacent devices.

In this structure, the hole-injection/transport layers (104B, 104G, and 104R), the light-emitting layers (105B, 105G, and 105R), and the electron-transport layers (108B, 108G, and 108R) of the EL layers (103B, 103G, and 103R) included in the light-emitting devices and the first transport layer 104PS, the active layer 105PS, and the second transport layer 108PS of the light-receiving layer 103PS included in the light-receiving device are processed to be separated by patterning using a photolithography method; thus, the side surfaces (end portions) of the processed EL layers have substantially the same surface (or are positioned on substantially the same plane). In addition, the side surfaces (end portions) of the layers of the light-receiving layer processed by patterning using a photolithography method have substantially the same surface (or are positioned on substantially the same plane).

In addition, the hole-injection/transport layers (104B, 104G, and 104R), the light-emitting layers (105B 105G, and 105R), and the electron-transport layers (108B, 108G, and 108R) of the EL layers (103B, 103G, and 103R) included in the light-emitting devices and the first transport layer 104PS, the active layer 105PS, and the second transport layer 108PS of the light-receiving layer 103PS included in the light-receiving device are processed to be separated by patterning using a photolithography method. Thus, the space 580 is provided between the processed end portions (side surfaces) of adjacent light-emitting devices. In FIG. 7C, when the space 580 is denoted by a distance SE between the EL layers or between the EL layer and the light-receiving layer of the adjacent light-emitting devices, the aperture ratio can be increased and definition can be increased as the distance SE decreases. By contrast, as the distance SE increases, the effect of the difference in the fabrication process between the adjacent light-emitting devices becomes permissible, which leads to an increase in manufacturing yield. Since the light-emitting device and the light-receiving device fabricated according to this specification is suitable for a miniaturization process, the distance SE between the EL layers or between the EL layer and the light-receiving layer of the adjacent light-emitting devices can be longer than or equal to 0.5 μm and shorter than or equal to 5 μm, preferably longer than or equal to 1 μm and shorter than or equal to 3 μm, further preferably longer than or equal to 1 μm and shorter than or equal to 2.5 μm, and still further preferably longer than or equal to 1 μm and shorter than or equal to 2 μm. Typically, the distance SE is preferably longer than or equal to 1 μm and shorter than or equal to 2 μm (e.g., 1.5 μm or a neighborhood thereof).

In this specification and the like, a device formed using a metal mask or an FMM (fine metal mask, high-definition metal mask) may be referred to as a device having an MM (metal mask) structure. In this specification and the like, a device formed without using a metal mask or an FMM may be referred to as a device having an MML (metal maskless) structure. Since a light-emitting and light-receiving apparatus having the MML structure is manufactured without using a metal mask, the pixel arrangement, the pixel shape, and the like can be designed more flexibly than in a light-emitting and light-receiving apparatus having the FMM structure or the MM structure.

Note that an island-shaped EL layer of a light-emitting and light-receiving apparatus having an MML structure is formed not by patterning with use of a metal mask but by processing after formation of an EL layer. Accordingly, a light-emitting and light-receiving apparatus with a higher definition or a higher aperture ratio than a conventional one can be achieved. Moreover, EL layers can be formed separately for the respective colors, enabling the light-emitting and light-receiving apparatus to perform extremely clear display with high contrast and high display quality. Moreover, providing the sacrificial layer over the EL layer can reduce damage to the EL layer in the manufacturing process, resulting in an increase in the reliability of the light-emitting device.

In FIG. 2A and FIG. 7C, the widths of the EL layers (103B, 103G, and 103R) are substantially equal to the widths of the electrodes (551B, 551G, and 551R) in the light-emitting device 550B, the light-emitting device 550G, and the light-emitting device 550R, and the width of the light-receiving layer 103PS is substantially equal to the width of the electrode 551PS in the light-receiving device 550PS; however, one embodiment of the present invention is not limited thereto.

In the light-emitting device 550B, the light-emitting device 550G, and the light-emitting device 550R, the widths of the EL layers (103B, 103G, and 103R) may be smaller than the widths of the electrodes (551B, 551G, and 551R). In the light-receiving device 550PS, the width of the light-receiving layer 103PS may be smaller than the width of the electrode 551PS. FIG. 7D illustrates an example in which the widths of the EL layers (103B and 103G) are smaller than the widths of the electrodes (551B and 551G) in the light-emitting device 550B and the light-emitting device 550G.

In the light-emitting device 550B, the light-emitting device 550G, and the light-emitting device 550R, the widths of the EL layers (103B, 103G, and 103R) may be larger than the widths of the electrodes (551B, 551G, and 551R). In the light-receiving device 550PS, the width of the light-receiving layer 103PS may be larger than the width of the electrode 551PS. FIG. 7E illustrates an example in which the width of the EL layer 103R is larger than the width of the electrode 551R in the light-emitting device 550R.

The structures described in this embodiment can be used in an appropriate combination with any of the structures described in the other embodiments.

### Embodiment 4

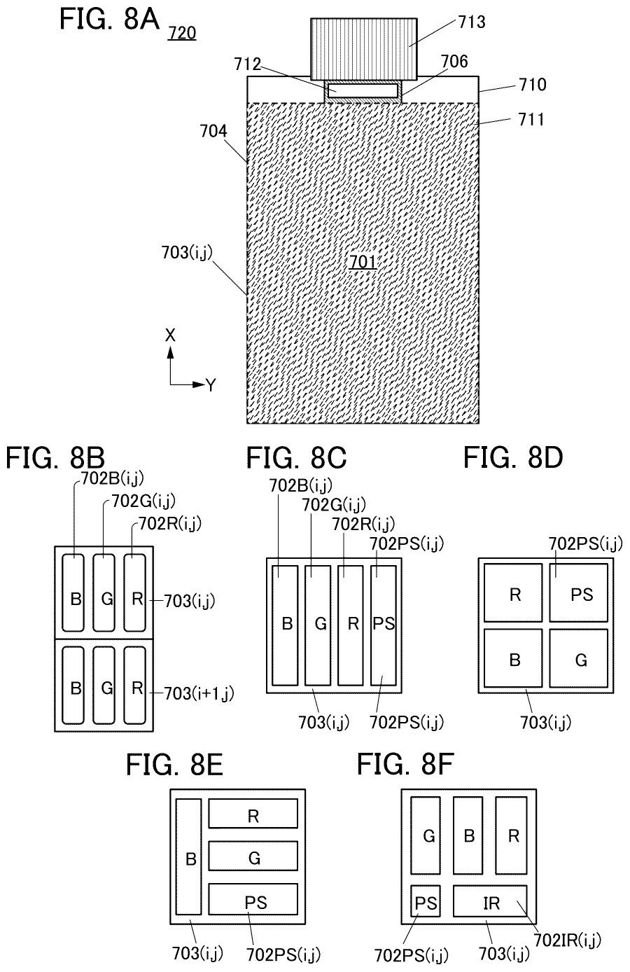

In this embodiment, an apparatus 720 will be described with reference to FIG. 8 to FIG. 10. The apparatus 720 illustrated in FIG. 8 to FIG. 10 includes any of the light-emitting devices described in Embodiment 2. Furthermore, the apparatus 720 described in this embodiment can be used in a display portion of an electronic apparatus or the like and thus can also be referred to as a display panel or a display apparatus. Moreover, when the apparatus includes the light-emitting device as a light source and a light-receiving device that can receive light from the light-emitting device, the apparatuses can also be referred to as a light-emitting and light-receiving apparatus. Note that the light-emitting apparatus, the display panel, the display apparatus, and the light-emitting and light-receiving apparatus each include at least a light-emitting device.

Furthermore, the light-emitting apparatus, the display panel, the display apparatus, and the light-emitting and light-receiving apparatus of this embodiment can each have a high definition or a large size. Accordingly, the light-emitting apparatus, the display panel, the display apparatus, and the light-emitting and light-receiving apparatus can be used for display portions of electronic apparatuses such as a digital camera, a digital video camera, a digital photo frame, a mobile phone, a portable game console, a smartphone, a wristwatch terminal, a tablet terminal, a portable information terminal, and an audio reproducing device, in addition to display portions of electronic apparatuses with a relatively large screen, such as a television device, a desktop or notebook personal computer, a monitor of a computer or the like, digital signage, and a large game machine such as a pachinko machine.

FIG. 8A is a top view of the apparatus 720 (including the light-emitting apparatus, the display panel, the display apparatus, and the light-emitting and light-receiving apparatus).

In FIG. 8A, the apparatus 720 has a structure in which a substrate 710 and a substrate 711 are bonded to each other. In addition, the apparatus 720 includes a display region 701, a circuit 704, a wiring 706, and the like. Note that the display region 701 includes a plurality of pixels. As illustrated in FIG. 8B, a pixel 703(i,j) illustrated in FIG. 8A has a pixel 703(i+1,j) adjacent to the pixel 703(i,j).

Furthermore, as illustrated in the example of FIG. 8A, the substrate 710 is provided with an IC (integrated circuit) 712 by a COG (Chip On Glass) method, a COF (Chip On Film) method, or the like in the apparatus 720. As the IC 712, an IC including a scan line driver circuit, a signal line driver circuit, or the like can be used, for example. FIG. 8A illustrates a structure where an IC including a signal line driver circuit is used as the IC 712, and a scan line driver circuit is used as the circuit 704.

The wiring 706 has a function of supplying signals and power to the display region 701 and the circuit 704. The signals and power are input to the wiring 706 from the outside through an FPC (Flexible Printed Circuit) 713 or to the wiring 706 from the IC 712. Note that the apparatus 720 is not necessarily provided with the IC. The IC may be mounted on the FPC by a COF method or the like.

FIG. 8B illustrates the pixel 703(i,j) and the pixel 703(i+1,j) of the display region 701. The pixel 703(i,j) can have a plurality of kinds of subpixels including light-emitting devices that emit light of different colors. In addition to the above, a plurality of subpixels including light-emitting devices that emit light of the same color may be included. In the case where a plurality of kinds of subpixels including light-emitting devices that emit different color light from each other are included in the pixel, three kinds of subpixels can be included, for example. The three subpixels can be of three colors of red (R), green (G), and blue (B) or of three colors of yellow (Y), cyan (C), and magenta (M), for example. Alternatively, the pixel can include four kinds of subpixels. As the four subpixels, subpixels of four colors of R, G, B, and white (W), subpixels of four colors of R, G, B, and Y, and the like can be given. Specifically, the pixel 703(i,j) can be composed of a subpixel 702B(i,j) displaying blue, a subpixel 702G(i,j) displaying green, and a subpixel 702R(i,j) displaying red.

The apparatus 720 includes not only a subpixel including a light-emitting device, but also a subpixel including a light-receiving device.

FIG. 8C to FIG. 8E illustrate various layout examples of the pixel 703(i,j) including a subpixel 702PS(i,j) including a light-receiving device. The pixel arrangement illustrated in FIG. 8C is stripe arrangement, and the pixel arrangement illustrated in FIG. 8D is matrix arrangement. In the pixel arrangement illustrated in FIG. 8E, three subpixels (the subpixel R, the subpixel G, and the subpixel S) are vertically arranged next to one subpixel (the subpixel B).

Furthermore, as illustrated in FIG. 8F, a subpixel 702IR(i,j) that emits infrared rays may be added to any of the above-described sets of subpixels to form the pixel 703(i,j). In the pixel arrangement illustrated in FIG. 8F, the three vertically long subpixel G, subpixel B, and subpixel R are arranged laterally, and the subpixel PS and the horizontally long subpixel IR are arranged laterally below the three subpixels. Note that the wavelength of light detected by the subpixel 702PS(i,j) is not particularly limited; however, the light-receiving device included in the subpixel 702PS(i,j) preferably has sensitivity to light emitted from the light-emitting device included in the subpixel 702R(i, j), the subpixel 702G(i, j), the subpixel 702G(i, j), or the subpixel 702G(i, j). The light-receiving device preferably detects one or more of light in blue, violet, bluish violet, green, yellowish green, yellow, orange, red, and infrared wavelength ranges, for example.

Note that the arrangement of subpixels is not limited to the structures illustrated in FIG. 8B to FIG. 8F and a variety of arrangement methods can be employed. The arrangement of subpixels may be stripe arrangement, S stripe arrangement, matrix arrangement, delta arrangement, Bayer arrangement, or PenTile arrangement, for example.

Examples of a top surface shape of the subpixel include polygons such as a triangle, a tetragon (including a rectangle and a square), and a pentagon; polygons with rounded corners; an ellipse; and a circle. Here, the top surface shape of the subpixel corresponds to a top surface shape of a light-emitting region of the light-emitting device.

In the case where a pixel includes a light-receiving device in addition to a light-emitting device, the pixel has a light-receiving function; thus, a touch or an approach of an object can be detected while an image is being displayed. For example, all the subpixels included in the light-emitting apparatus can display an image; alternatively, some of the subpixels can emit light as a light source, and the rest of the subpixels can display an image.

Note that the light-receiving area of the subpixel 702PS(i, j) is preferably smaller than the light-emitting areas of the other subpixels. A smaller light-receiving area leads to a narrower image-capturing range, inhibits a blur in a capturing result, and improves the resolution. Thus, by using the subpixel 702PS(i,j), high-definition or high-resolution image capturing is possible. For example, image capturing for personal authentication with the use of a fingerprint, a palm print, the iris, the shape of a blood vessel (including the shape of a vein and the shape of an artery), a face, or the like is possible by using the subpixel 702PS(i,j).

Moreover, the subpixel 702PS(i, j) can be used in a touch sensor (also referred to as a direct touch sensor), a near touch sensor (also referred to as a hover sensor, a hover touch sensor, a contactless sensor, or a touchless sensor), or the like. For example, the subpixel 702PS(i, j) preferably detects infrared light. Thus, a touch can be detected even in a dark place.

Here, the touch sensor or the near touch sensor can detect the approach or contact of an object (e.g., a finger, a hand, or a pen). The touch sensor can detect the object when the light-emitting and light-receiving apparatus and the object come in direct contact with each other. Furthermore, the near touch sensor can detect the object even when the object is not in contact with the light-emitting and light-receiving apparatus. For example, the display apparatus is preferably capable of detecting an object positioned in the range of 0.1 mm to 300 mm inclusive, further preferably 3 mm to 50 mm inclusive from the light-emitting and light-receiving apparatus. This structure enables the light-emitting and light-receiving apparatus to be operated without direct contact of an object, that is, enables the light-emitting and light-receiving apparatus to be operated in a contactless (touchless) manner. With the above-described structure, the light-emitting and light-receiving apparatus can be operated with a reduced risk of making the light-emitting and light-receiving apparatus dirty or damaging the light-emitting and light-receiving apparatus or without the object directly touching a dirt (e.g., dust, bacteria, or a virus) attached to the display apparatus.

For high-definition image capturing, the subpixels 702PS(i,j) are preferably provided in all pixels included in the light-emitting and light-receiving apparatus. Meanwhile, in the case where the subpixel 702PS(i, j) is used in a touch sensor, a near touch sensor, or the like, high accuracy is not required as compared to the case of capturing an image of a fingerprint or the like; accordingly, the subpixel 702PS(i, j) may be provided in some pixels in the light-emitting and light-receiving apparatus. When the number of the subpixels 702PS(i, j) included in the light-emitting and light-receiving apparatus is smaller than the number of the subpixels 702R(i,j) or the like, higher detection speed can be achieved.

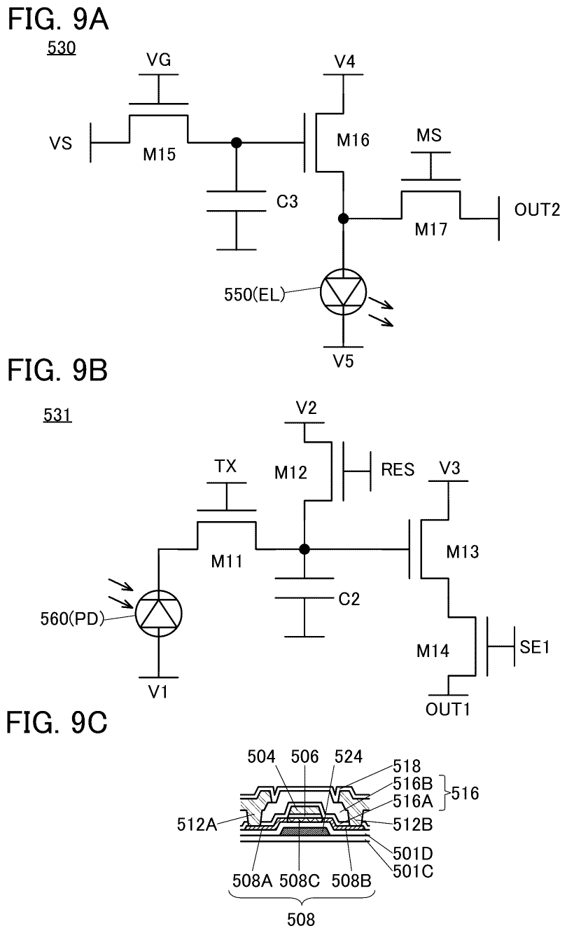

Next, an example of a pixel circuit of a subpixel including the light-emitting device is described with reference to FIG. 9A. A pixel circuit 530 illustrated in FIG. 9A includes a light-emitting device (EL) 550, a transistor M15, a transistor M16, a transistor M17, and a capacitor C3. Note that a light-emitting diode can be used as the light-emitting device 550. In particular, any of the light-emitting devices described in Embodiment 2 is preferably used as the light-emitting device 550.

In the transistor M15 illustrated in FIG. 9A, a gate is electrically connected to a wiring VG, one of a source and a drain is electrically connected to a wiring VS, and the other of the source and the drain is electrically connected to one electrode of the capacitor C3 and a gate of the transistor M16. One of a source and a drain of the transistor M16 is electrically connected to a wiring V4, and the other of the source and the drain of the transistor M16 is electrically connected to an anode of the light-emitting device 550 and one of a source and a drain of the transistor M17. A gate of the transistor M17 is electrically connected to a wiring MS, and the other of the source and the drain of the transistor M17 is electrically connected to a wiring OUT2. A cathode of the light-emitting device 550 is electrically connected to a wiring V5.

A constant potential is supplied to the wiring V4 and the wiring V5. In the light-emitting device 550, the anode side can have a high potential and the cathode side can have a lower potential than the anode side. The transistor M15 is controlled by a signal supplied to the wiring VG and functions as a selection transistor for controlling a selection state of the pixel circuit 530. The transistor M16 functions as a driving transistor that controls a current flowing through the light-emitting device 550 in accordance with a potential supplied to the gate of the transistor M16. When the transistor M15 is in a conduction state, a potential supplied to the wiring VS is supplied to the gate of the transistor M16, and the luminance of the light-emitting device 550 can be controlled in accordance with the potential. The transistor M17 is controlled by a signal supplied to the wiring MS and has a function of outputting a potential between the transistor M16 and the light-emitting device 550 to the outside through the wiring OUT2.

Here, transistors in which a metal oxide (an oxide semiconductor) is used in a semiconductor layer where a channel is formed are preferably used as the transistor M15, a transistor M12, the transistor M16, the transistor M17 included in the pixel circuit 530 illustrated in FIG. 9A, and a transistor M11, the transistor M12, and a transistor M14 included in the pixel circuit 531 illustrated in FIG. 9B.

A transistor using a metal oxide having a wider band gap and a lower carrier density than silicon achieves an extremely low off-state current. Therefore, owing to the low off-state current, charge accumulated in a capacitor that is connected in series with the transistor can be retained for a long time. Accordingly, it is particularly preferable to use transistors containing an oxide semiconductor as the transistor M11, the transistor M12, and the transistor M15 each of which is connected in series with a capacitor C2 or the capacitor C3. When the other transistors also include an oxide semiconductor, the manufacturing cost can be reduced.

Alternatively, transistors using silicon for a semiconductor in which a channel is formed can be used as the transistor M11 to the transistor M17. It is particularly preferable to use silicon with high crystallinity, such as single crystal silicon or polycrystalline silicon, because high field-effect mobility can be achieved and higher-speed operation can be performed.

Alternatively, a transistor using an oxide semiconductor may be used as one or more of the transistor M11 to the transistor M17, and transistors using silicon may be used as the other transistors.

Next, an example of a pixel circuit of a subpixel including a light-receiving device is described with reference to FIG. 9B. A pixel circuit 531 illustrated in FIG. 9B includes a light-receiving device (PD) 560, the transistor M11, the transistor M12, the transistor M13, the transistor M14, and the capacitor C2. Here, an example in which a photodiode is used as the light-receiving device (PD) 560 is illustrated.

In the light-receiving device (PD) 560 illustrated in FIG. 9B, an anode is electrically connected to a wiring V1, and a cathode is electrically connected to one of a source and a drain of the transistor M11. Agate of the transistor M11 is electrically connected to a wiring TX, and the other of the source and the drain of the transistor M11 is electrically connected to one electrode of the capacitor C2, one of a source and a drain of the transistor M12, and a gate of the transistor M13. Agate of the transistor M12 is electrically connected to a wiring RES, and the other of the source and the drain of the transistor M12 is electrically connected to a wiring V2. One of a source and a drain of the transistor M13 is electrically connected to a wiring V3, and the other of the source and the drain of the transistor M13 is electrically connected to one of a source and a drain of the transistor M14. A gate of the transistor M14 is electrically connected to a wiring SE1, and the other of the source and the drain of the transistor M14 is electrically connected to a wiring OUTL.

A constant potential is supplied to each of the wiring V1, the wiring V2, and the wiring V3. When the light-receiving device (PD) 560 is driven with a reverse bias, the wiring V2 is supplied with a potential higher than the potential of the wiring V1. The transistor M12 is controlled by a signal supplied to the wiring RES and has a function of resetting the potential of a node connected to the gate of the transistor M13 to a potential supplied to the wiring V2. The transistor M11 is controlled by a signal supplied to the wiring TX and has a function of controlling the timing at which the potential of the node changes, in accordance with a current flowing through the light-receiving device (PD) 560. The transistor M13 functions as an amplifier transistor for performing output corresponding to the potential of the node. The transistor M14 is controlled by a signal supplied to the wiring SE1 and functions as a selection transistor for making an external circuit connected to the wiring OUT1 read the output corresponding to the potential of the node.

Although n-channel transistors are shown as the transistors in FIG. 9A and FIG. 9B, p-channel transistors can alternatively be used.

The transistors included in the pixel circuit 530 and the transistors included in the pixel circuit 531 are preferably formed to be arranged over the same substrate. It is particularly preferable that the transistors included in the pixel circuit 530 and the transistors included in the pixel circuit 531 be periodically arranged in one region.

One or more layers including the transistor and/or the capacitor are preferably provided to overlap with the light-receiving device (PD) 560 or the light-emitting device (EL) 550. Thus, the effective area occupied by each pixel circuit can be reduced, and a high-definition light-receiving portion or display portion can be achieved.

FIG. 9C illustrates an example of a specific structure of a transistor that can be used in the pixel circuit described with reference to FIG. 9A and FIG. 9B. As the transistor, a bottom-gate transistor, a top-gate transistor, or the like can be used as appropriate.

The transistor illustrated in FIG. 9C includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C and has a function of a gate electrode.

The insulating film 506 includes a region positioned between the semiconductor film 508 and the conductive film 504. The insulating film 506 has a function of a first gate insulating film.

The conductive film 512A has one of a function of a source electrode and a function of a drain electrode, and the conductive film 512B has the other of the function of the source electrode and the function of the drain electrode.

A conductive film 524 can be used in the transistor. The conductive film 524 includes a region where the semiconductor film 508 is positioned between the conductive film 504 and the conductive film 524. The conductive film 524 has a function of a second gate electrode. An insulating film 501D is positioned between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For example, a material having a function of inhibiting diffusion of oxygen, hydrogen, water, an alkali metal, an alkaline earth metal, and the like is preferably used for the insulating film 518. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film with the same composition as the semiconductor film used in the transistor of the pixel circuit can be used in the driver circuit, for example.

The semiconductor film 508 preferably contains indium, M (M is one or more kinds selected from gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more kinds selected from aluminum, gallium, yttrium, and tin.

It is particularly preferable that an oxide containing indium (In), gallium (Ga), and zinc (Zn) (also referred to as IGZO) be used for the semiconductor film 508. Alternatively, it is preferable to use an oxide containing indium, tin, and zinc. Further alternatively, it is preferable to use an oxide containing indium, gallium, tin, and zinc. Alternatively, it is preferable to use an oxide containing indium (In), aluminum (Al), and zinc (Zn) (also referred to as IAZO). Further alternatively, it is preferable to use an oxide containing indium (In), aluminum (Al), gallium (Ga), and zinc (Zn) (also referred to as IAGZO).

In the case where the semiconductor film is an In-M-Zn oxide, the atomic proportion of In is preferably greater than or equal to the atomic proportion of M in the In-M-Zn oxide. Examples of the atomic ratio of the metal elements in such an In-M-Zn oxide include InM:Zn=1:1:1 or a composition in the neighborhood thereof, InMZn=1:1:1.2 or a composition in the neighborhood thereof, InM:Zn=1:3:2 or a composition in the neighborhood thereof, InM:Zn=1:3:4 or a composition in the neighborhood thereof, InMZn=2:1:3 or a composition in the neighborhood thereof, InM:Zn=3:1:2 or a composition in the neighborhood thereof, InM:Zn=4:2:3 or a composition in the neighborhood thereof, InMZn=4:2:4.1 or a composition in the neighborhood thereof, InM:Zn=5:1:3 or a composition in the neighborhood thereof, InM:Zn=5:1:6 or a composition in the neighborhood thereof, InMZn=5:1:7 or a composition in the neighborhood thereof, InM:Zn=5:1:8 or a composition in the neighborhood thereof, InM:Zn=6:1:6 or a composition in the neighborhood thereof, and InM:Zn=5:2:5 or a composition in the neighborhood thereof. Note that a composition in the neighborhood includes the range of ±30% of an intended atomic ratio.

For example, when the atomic ratio is described as In:Ga:Zn=4:2:3 or a composition in the neighborhood thereof, the case is included where the atomic ratio of Ga is greater than or equal to 1 and less than or equal to 3 and the atomic ratio of Zn is greater than or equal to 2 and less than or equal to 4 with the atomic ratio of In being 4. When the atomic ratio is described as In:Ga:Zn=5:1:6 or a composition in the neighborhood thereof, the case is included where the atomic ratio of Ga is greater than 0.1 and less than or equal to 2 and the atomic ratio of Zn is greater than or equal to 5 and less than or equal to 7 with the atomic ratio of In being 5. When the atomic ratio is described as In:Ga:Zn=1:1:1 or a composition in the neighborhood thereof, the case is included where the atomic ratio of Ga is greater than 0.1 and less than or equal to 2 and the atomic ratio of Zn is greater than 0.1 and less than or equal to 2 with the atomic ratio of In being 1.

There is no particular limitation on the crystallinity of a semiconductor material used for the transistors, and any of an amorphous semiconductor and a semiconductor having crystallinity (a microcrystalline semiconductor, a polycrystalline semiconductor, a single crystal semiconductor, or a semiconductor partly including crystal regions) may be used. A semiconductor having crystallinity is preferably used, in which case deterioration of the transistor characteristics can be inhibited.

The semiconductor layer of the transistor preferably includes a metal oxide (also referred to as an oxide semiconductor). As the oxide semiconductor having crystallinity, a CAAC (c-axis aligned crystalline)-OS, an nc (nanocrystalline)-OS, and the like are given.

Alternatively, a transistor using silicon in a channel formation region (a Si transistor) may be used. Examples of silicon include single crystal silicon (single crystal Si), polycrystalline silicon, and amorphous silicon. In particular, a transistor containing low-temperature polysilicon (LTPS) in its semiconductor layer (hereinafter also referred to as an LTPS transistor) can be used. The LTPS transistor has high field-effect mobility and favorable frequency characteristics.

With the use of a Si transistor such as an LTPS transistor, a circuit required to be driven at a high frequency (e.g., a source driver circuit) can be formed on the same substrate as the display portion. This allows simplification of an external circuit mounted on the light-emitting apparatus and a reduction in component cost and mounting cost.

An OS transistor has much higher field-effect mobility than a transistor using amorphous silicon. In addition, an OS transistor has an extremely low leakage current between a source and a drain in an off state (hereinafter also referred to as off-state current), and charge accumulated in a capacitor that is connected in series to the transistor can be retained for a long period. Furthermore, the power consumption of the light-emitting apparatus can be reduced with the OS transistor.

The off-state current value per micrometer of channel width of the OS transistor at room temperature can be lower than or equal to 1 aA (1×10⁻¹⁸ A), lower than or equal to 1 zA (1×10⁻²¹ A), or lower than or equal to 1 yA (1×10⁻²⁴ A). Note that the off-state current value per micrometer of channel width of a Si transistor at room temperature is higher than or equal to 1 fA (1×10⁻¹⁵ A) and lower than or equal to 1 pA (1×10⁻¹² A). In other words, the off-state current of an OS transistor is lower than that of a Si transistor by approximately ten orders of magnitude.

To increase the emission luminance of the light-emitting device included in a pixel circuit, it is necessary to increase the amount of current flowing through the light-emitting device. For that purpose, the source-drain voltage of the driving transistor included in the pixel circuit needs to be increased. Since an OS transistor has a higher withstand voltage between the source and the drain than a Si transistor, a high voltage can be applied between the source and the drain of the OS transistor. Thus, with use of an OS transistor as a driving transistor included in the pixel circuit, the amount of current flowing through the light-emitting device can be increased, resulting in an increase in emission luminance of the light-emitting device.

When a transistor operates in a saturation region, a change in source-drain current relative to a change in gate-source voltage can be smaller in an OS transistor than in a Si transistor. Accordingly, when an OS transistor is used as the driving transistor included in the pixel circuit, current flowing between the source and the drain can be set minutely by a change in gate-source voltage; hence, the amount of current flowing through the light-emitting device can be controlled. Accordingly, the number of gray levels in the pixel circuit can be increased.

Regarding saturation characteristics of current flowing when a transistor operates in a saturation region, even in the case where the source-drain voltage of an OS transistor increases gradually, more stable current (saturation current) can be made flow through an OS transistor than through a Si transistor. Thus, with use of an OS transistor as a driving transistor, current can be made flow stably through the light-emitting device, for example, even when a variation in current-voltage characteristics of the light-emitting device occurs. In other words, when the OS transistor operates in the saturation region, the source-drain current hardly changes with an increase in the source-drain voltage; hence, the emission luminance of the light-emitting device can be stable.

As described above, with use of an OS transistor as the driving transistor included in the pixel circuit, it is possible to achieve “inhibition of black floating”, “increase in emission luminance”, “increase in the number of gray levels”, “inhibition of variation in light-emitting devices”, and the like.

The semiconductor film used in the transistor of the driver circuit can be formed in the same step as the semiconductor film used in the transistor of the pixel circuit. The driver circuit can be formed over a substrate where the pixel circuit is formed. The number of components of an electronic apparatus can be reduced.

Silicon may be used for the semiconductor film 508. Examples of silicon include single crystal silicon, polycrystalline silicon, and amorphous silicon. In particular, a transistor containing low-temperature polysilicon (LTPS) in its semiconductor layer (hereinafter also referred to as an LTPS transistor) is preferably used. The LTPS transistor has high field-effect mobility and favorable frequency characteristics.

With the use of a transistor containing silicon, such as an LTPS transistor, a circuit required to be driven at a high frequency (e.g., a source driver circuit) can be formed on the same substrate as the display portion. This allows simplification of an external circuit mounted on the light-emitting apparatus and a reduction in component cost and mounting cost.

It is preferable to use a transistor containing a metal oxide (hereinafter also referred to as an oxide semiconductor) in its semiconductor where a channel is formed (hereinafter also referred to as an OS transistor) as at least one of the transistors included in the pixel circuit. An OS transistor has much higher field-effect mobility than a transistor using amorphous silicon. In addition, an OS transistor has an extremely low leakage current between a source and a drain in an off state (hereinafter also referred to as off-state current), and charge accumulated in a capacitor that is connected in series to the transistor can be retained for a long period. Furthermore, the power consumption of the light-emitting apparatus can be reduced with the OS transistor.

When LTPS transistors are used as some of the transistors included in the pixel circuit and OS transistors are used as the rest, the light-emitting apparatus can have low power consumption and high driving capability. As a favorable example, it is preferable that an OS transistor be used as a transistor functioning as a switch for controlling electrical continuity between wirings and an LTPS transistor be used as a transistor for controlling current. Note that a structure in which an LTPS transistor and an OS transistor are combined is referred to as LTPO in some cases. LTPO enables the display panel to have low power consumption and high driving capability.

For example, one transistor provided in the pixel circuit functions as a transistor for controlling current flowing through the light-emitting device and can also be referred to as a driving transistor. One of a source and a drain of the driving transistor is electrically connected to the pixel electrode of the light-emitting device. An LTPS transistor is preferably used as the driving transistor. Thus, current flowing through the light-emitting device in the pixel circuit can be increased.

In contrast, another transistor provided in the pixel circuit functions as a switch for controlling selection and non-selection of a pixel and can also be referred to as a selection transistor. A gate of the selection transistor is electrically connected to a gate line, and one of a source and a drain thereof is electrically connected to a source line (signal line). An OS transistor is preferably used as the selection transistor. Accordingly, the gray level of the pixel can be maintained even with an extremely low frame frequency (e.g., 1 fps or less); thus, power consumption can be reduced by stopping the driver in displaying a still image.

In the case of using an oxide semiconductor in a semiconductor film, the apparatus 720 includes a light-emitting device including an oxide semiconductor in its semiconductor film and having an MML (metal maskless) structure. With this structure, the leakage current that might flow through the transistor and the leakage current that might flow between adjacent light-emitting devices (also referred to as lateral leakage current, side leakage current, or the like) can be extremely low. With the structure, a viewer can notice any one or more of the image crispness, the image sharpness, a high chroma, and a high contrast ratio in an image displayed on the display apparatus. When the leakage current that might flow through the transistor and the lateral leakage current that might flow between light-emitting devices are extremely low, display with little leakage of light at the time of black display (what is called black floating) (such display is also referred to as deep black display) can be achieved.

In particular, in the case where a light-emitting device having an MML structure employs the above-described SBS structure, a layer provided between light-emitting devices (for example, also referred to as an organic layer or a common layer which is shared by the light-emitting devices) is divided; accordingly, display with no or extremely small lateral leakage can be achieved.

The structure of transistors used in a display panel may be selected as appropriate depending on the screen size of the display panel. For example, single crystal Si transistors can be used in the display panel with a screen diagonal greater than or equal to 0.1 inches and less than or equal to 3 inches. In addition, LTPS transistors can be used in the display panel with a screen diagonal greater than or equal to 0.1 inches and less than or equal to 30 inches, preferably greater than or equal to 1 inch and less than or equal to 30 inches. In addition, an LTPO structure (where an LTPS transistor and an OS transistor are used in combination) can be used in the display panel with a screen diagonal greater than or equal to 0.1 inches and less than or equal to 50 inches, preferably greater than or equal to 1 inch and less than or equal to 50 inches. In addition, OS transistors can be used in the display panel with a screen diagonal greater than or equal to 0.1 inches and less than or equal to 200 inches, preferably greater than or equal to 50 inches and less than or equal to 100 inches.

Note that with use of single crystal Si transistors, an increase in screen size is extremely difficult because of the size of a single crystal Si substrate. Furthermore, since a laser crystallization apparatus is used in the manufacturing process, LTPS transistors are unlikely to respond to an increase in screen size (typically to a screen diagonal greater than 30 inches). By contrast, since the manufacturing process does not necessarily require a laser crystallization apparatus or the like or can be performed at a relatively low process temperature (typically, lower than or equal to 450° C.), OS transistors can be used for a display panel with a relatively large area (typically, a screen diagonal greater than or equal to 50 inches and less than or equal to 100 inches). In addition, LTPO is applicable to a display panel with a size midway between the case of using LTPS transistors and the case of using OS transistors (typically, a diagonal size greater than or equal to 1 inch and less than or equal to 50 inches).

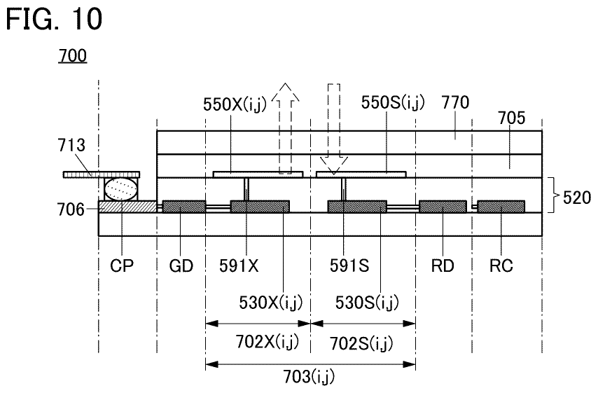

Next, a cross-sectional view of the light-emitting and light-receiving apparatus is shown. FIG. 10 is a cross-sectional view of the light-emitting and light-receiving apparatus illustrated in FIG. 8A.

FIG. 10 is a cross-sectional view of part of a region including the FPC 713 and the wiring 706, and part of the display region 701 including the pixel 703(i,j).

In FIG. 10, the light-emitting and light-receiving apparatus 700 includes the functional layer 520 between the first substrate 510 and the second substrate 770. The functional layer 520 includes, as well as the transistors (M11, M12, M13, M14, M15, M16, and M17), the capacitor (C2 and C3), and the like described with reference to FIG. 9, wirings (VS, VG, V1, V2, V3, V4, and V5) electrically connecting these components, for example. The structure of the functional layer 520 illustrated in FIG. 10 includes a pixel circuit 530X(i,j), a pixel circuit 530S(i,j), and the driver circuit GD; however, it is not limited thereto.

Each pixel circuit (e.g., the pixel circuit 530X(i,j) and the pixel circuit 530S(i,j) in FIG. 10) included in the functional layer 520 is electrically connected to light-emitting devices and light receiving devices (e.g., a light-emitting device 550X(i,j) and a light-receiving device 550S(i,j) in FIG. 10) formed over the functional layer 520. Specifically, the light-emitting device 550X(i,j) is electrically connected to the pixel circuit 530X(i, j) through a wiring 591X, and the light-receiving device 550S(i,j) is electrically connected to the pixel circuit 530S(ij) through a wiring 591S. The insulating layer 705 is provided over the functional layer 520, the light-emitting devices, and the light-receiving devices and the insulating layer 705 has a function of bonding the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting and light receiving apparatus of one embodiment of the present invention can be used as a touch panel.

Note that the structure described in this embodiment can be used in an appropriate combination with any of the structures described in the other embodiments.

### Embodiment 5

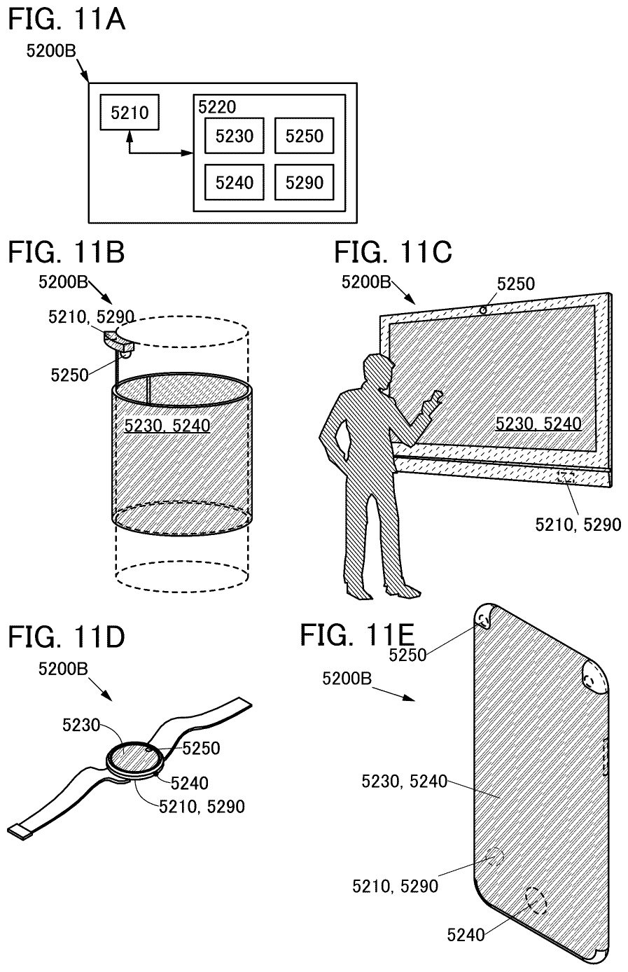

In this embodiment, electronic apparatuses of one embodiment of the present invention will be described with reference to FIG. 11A to FIG. 13B.

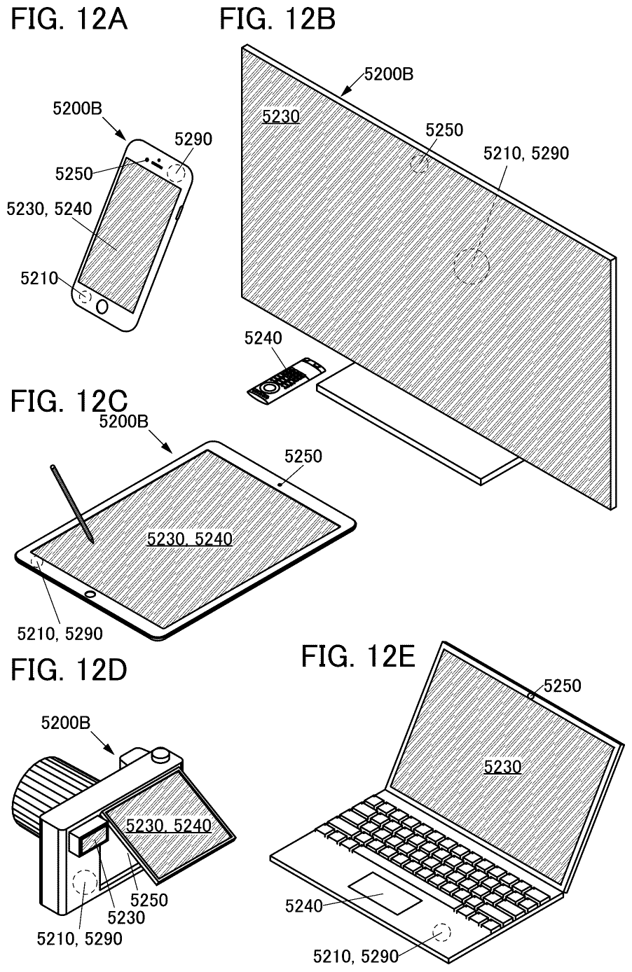

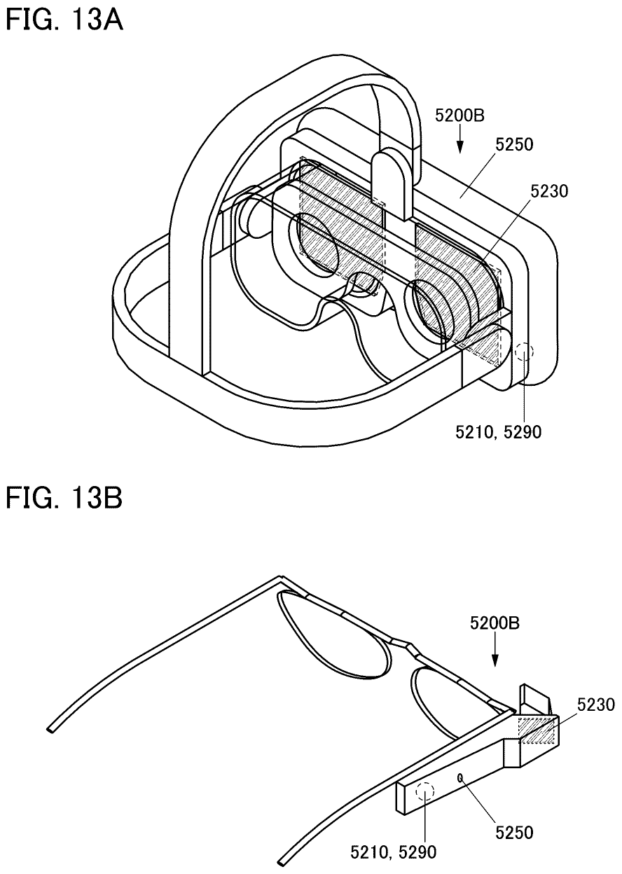

FIG. 11A to FIG. 13B are diagrams illustrating structures of electronic apparatuses of one embodiment of the present invention. FIG. 11A is a block diagram of the electronic apparatus and FIG. 11B to FIG. 11E are perspective views illustrating structures of the electronic apparatuses. FIG. 12A to FIG. 12E are perspective views illustrating structures of electronic apparatuses. FIG. 13A and FIG. 13B are perspective views illustrating structures of electronic apparatuses.

An electronic apparatus 5200B described in this embodiment includes an arithmetic device 5210 and an input/output device 5220 (see FIG. 11A).

The arithmetic device 5210 has a function of being supplied with operation data and has a function of supplying image data on the basis of the operation data.

The input/output device 5220 includes a display portion 5230, an input portion 5240, a detecting portion 5250, and a communication portion 5290 and has a function of supplying operation data and a function of being supplied with image data. The input/output device 5220 also has a function of supplying detection data, a function of supplying communication data, and a function of being supplied with communication data.

The input portion 5240 has a function of supplying operation data. For example, the input portion 5240 supplies operation data on the basis of operation by a user of the electronic apparatus 5200B.

Specifically, a keyboard, a hardware button, a pointing device, a touch sensor, an illuminance sensor, an imaging apparatus, an audio input device, an eye-gaze input device, an attitude detection device, or the like can be used as the input portion 5240.

The display portion 5230 includes a display panel and has a function of displaying image data. For example, the display panel described in Embodiment 3 can be used for the display portion 5230.

The detecting portion 5250 has a function of supplying detection data. For example, the detecting portion 5250 has a function of detecting a surrounding environment where the electronic apparatus is used and supplying detection data.

Specifically, an illuminance sensor, an imaging apparatus, an attitude detection device, a pressure sensor, a human motion sensor, or the like can be used as the detecting portion 5250.

The communication portion 5290 has a function of being supplied with communication data and a function of supplying communication data. For example, the communication portion 5290 has a function of being connected to another electronic apparatus or a communication network through wireless communication or wired communication. Specifically, the communication portion 5290 has a function of wireless local area network communication, telephone communication, near field communication, or the like.

FIG. 11B illustrates an electronic apparatus having an outer shape along a cylindrical column or the like. An example of such an electronic apparatus is digital signage. The display panel of one embodiment of the present invention can be used for the display portion 5230. Note that the electronic apparatus has a function of changing its display method in accordance with the illuminance of a usage environment. Furthermore, the electronic apparatus has a function of changing displayed content in response to detected existence of a person. Thus, for example, the electronic apparatus can be provided on a column of a building. The electronic apparatus can display advertising, guidance, or the like.

FIG. 11C illustrates an electronic apparatus having a function of generating image data on the basis of the path of a pointer used by the user. Examples of such an electronic apparatus include an electronic blackboard, an electronic bulletin board, and digital signage. Specifically, the display panel with a diagonal size of 20 inches or longer, preferably 40 inches or longer, and further preferably 55 inches or longer can be used. Alternatively, a plurality of display panels can be arranged and used as one display region. Alternatively, a plurality of display panels can be arranged and used as a multiscreen.

FIG. 11D illustrates an electronic apparatus that is capable of receiving data from another device and displaying the data on the display portion 5230. An example of such an electronic apparatus is a wearable electronic apparatus. Specifically, the electronic apparatus can display several options, or allow a user to choose some from the options and send a reply to the data transmitter. Alternatively, for example, the electronic apparatus has a function of changing its display method in accordance with the illuminance of a usage environment. Thus, the power consumption of the wearable electronic apparatus can be reduced, for example. Alternatively, an image can be displayed on the wearable electronic apparatus so that the wearable electronic apparatus can be suitably used even in an environment under strong external light, e.g., outdoors in fine weather, for example.

FIG. 11E illustrates an electronic apparatus including the display portion 5230 having a surface gently curved along a side surface of a housing. An example of such an electronic apparatus is a mobile phone. The display portion 5230 includes a display panel, and the display panel has a function of performing display on the front surface, the side surfaces, the top surface, and the rear surface, for example. Thus, for example, a mobile phone can display data not only on the front surface but also on the side surfaces, the top surface, and the rear surface.

FIG. 12A illustrates an electronic apparatus that is capable of receiving data via the Internet and displaying the data on the display portion 5230. An example of such an electronic apparatus is a smartphone. For example, a created message can be checked on the display portion 5230. The created message can be sent to another device. The electronic apparatus has a function of changing its display method in accordance with the illuminance of a usage environment, for example. Thus, the power consumption of a smartphone can be reduced. A smartphone can display an image so that the smartphone can be suitably used even in an environment under strong external light, e.g., outdoors in fine weather, for example.

FIG. 12B illustrates an electronic apparatus that can use a remote controller as the input portion 5240. An example of such an electronic apparatus is a television system. For example, the electronic apparatus that is capable of receiving data from a broadcast station or via the Internet and performing display on the display portion 5230. An image of a user can be taken using the detecting portion 5250. The image of the user can be transmitted. The electronic apparatus can acquire a viewing history of the user and provide it to a cloud service. The electronic apparatus can acquire recommendation data from a cloud service and display the data on the display portion 5230. A program or a moving image can be displayed on the basis of the recommendation data. The electronic apparatus has a function of changing its display method in accordance with the illuminance of a usage environment, for example. Accordingly, for example, the television system can display an image so that the television system can be suitably used even when irradiated with strong external light that enters a room in fine weather.

FIG. 12C illustrates an electronic apparatus that is capable of receiving educational materials via the Internet and displaying them on the display portion 5230. An example of such an electronic apparatus is a tablet computer. An assignment can be input with the input portion 5240 and sent via the Internet. A corrected assignment or the evaluation of the assignment can be obtained from a cloud service and displayed on the display portion 5230. Suitable educational materials can be selected on the basis of the evaluation and displayed.

For example, the display can be performed on the display portion 5230 using an image signal received from another electronic apparatus. When the electronic apparatus is placed on a stand or the like, the display portion 5230 can be used as a sub-display. Thus, for example, a tablet computer can display an image so that the tablet computer can be suitably used even in an environment under strong external light, e.g., outdoors in fine weather.

FIG. 12D illustrates an electronic apparatus including a plurality of display portions 5230. An example of such an electronic apparatus is a digital camera. For example, the display portion 5230 can display an image that the detecting portion 5250 is capturing. A captured image can be displayed on the detecting portion. A captured image can be decorated using the input portion 5240. A message can be attached to a captured image. A captured image can be transmitted via the Internet. The electronic apparatus has a function of changing its shooting conditions in accordance with the illuminance of a usage environment. Accordingly, for example, the digital camera can display an object so that an image is favorably viewed even in an environment under strong external light, e.g., outdoors in fine weather.

FIG. 12E illustrates an electronic apparatus in which the electronic apparatus of this embodiment is used as a master to control another electronic apparatus used as a slave. An example of such an electronic apparatus is a portable personal computer. As an example, part of image data can be displayed on the display portion 5230 and another part of image data can be displayed on a display portion of another electronic apparatus. Image signals can be supplied to another electronic apparatus. With the communication portion 5290, data to be written can be obtained from an input portion of another electronic apparatus. Thus, a large display region can be utilized by using the portable personal computer, for example.

FIG. 13A illustrates an electronic apparatus including the detecting portion 5250 that detects an acceleration or a direction. An example of such an electronic apparatus is a goggles-type electronic apparatus. The detecting portion 5250 can supply data on the position of the user or the direction in which the user faces. The electronic apparatus can generate image data for the right eye and image data for the left eye in accordance with the position of the user or the direction in which the user faces. The display portion 5230 includes a display region for the right eye and a display region for the left eye. Thus, a virtual reality image that gives the user a sense of immersion can be displayed on the goggles-type electronic apparatus, for example.

FIG. 13B illustrates an electronic apparatus including the detecting portion 5250 that detects an acceleration or a direction. An example of such an electronic apparatus is a glasses-type electronic apparatus. The detecting portion 5250 can supply data on the position of the user or the direction in which the user faces. The electronic apparatus can generate image data in accordance with the position of the user or the direction in which the user faces. Accordingly, the data can be shown together with a real-world scene, for example. An augmented reality image can be displayed on the glasses-type electronic apparatus.

Note that this embodiment can be combined with any of the other embodiments described in this specification as appropriate.

### Embodiment 6

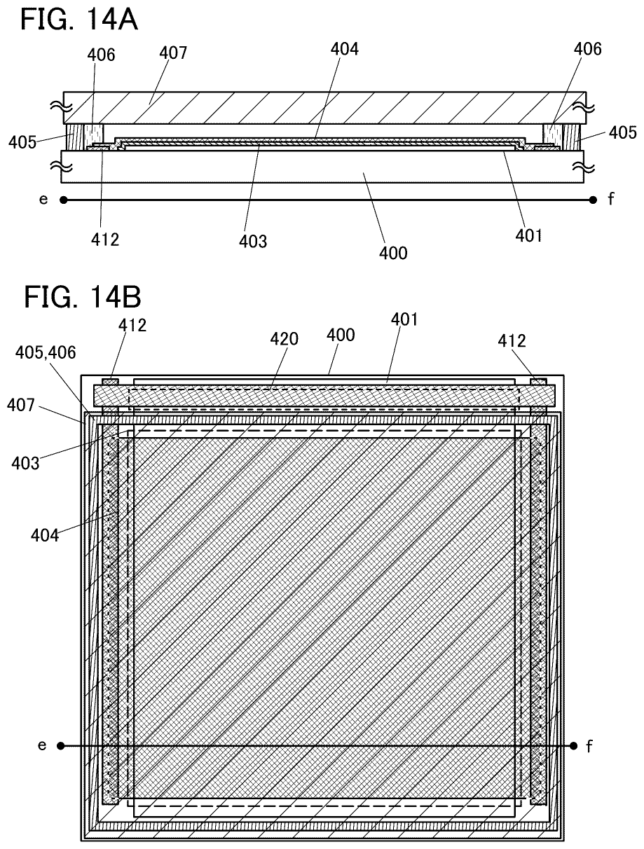

In this embodiment, a structure in which the light-emitting device described in Embodiment 2 is used for a lighting device will be described with reference to FIG. 14. FIG. 14A is a cross-sectional view taken along the line e-f in FIG. 14B which is a top view of a lighting device.

In the lighting device in this embodiment, a first electrode 401 is formed over a substrate 400 which is a support and has a light-transmitting property. The first electrode 401 corresponds to the first electrode 101 in Embodiment 2. In the case where light emission is extracted from the first electrode 401 side, the first electrode 401 is formed with a material having a light-transmitting property.

A pad 412 for supplying a voltage to a second electrode 404 is formed over the substrate 400.

An EL layer 403 is formed over the first electrode 401. The structure of the EL layer 403 corresponds to, for example, the structure of the EL layer 103 in Embodiment 2. Note that for these structures, the corresponding description can be referred to.

The second electrode 404 is formed to cover the EL layer 403. The second electrode 404 corresponds to the second electrode 102 in Embodiment 2. In the case where light-emission is extracted from the first electrode 401 side, the second electrode 404 is formed with a material having high reflectivity. The second electrode 404 is supplied with a voltage when connected to the pad 412.

As described above, the lighting device described in this embodiment includes a light-emitting device including the first electrode 401, the EL layer 403, and the second electrode 404. Since the light-emitting device is a light-emitting device with a high emission efficiency, the lighting device in this embodiment can be a lighting device with low power consumption.

The substrate 400 over which the light-emitting device having the above structure is formed is fixed to a sealing substrate 407 with sealants (405 and 406) and sealing is performed, whereby the lighting device is completed. It is possible to use only either the sealant 405 or the sealant 406. In addition, the inner sealant 406 (not illustrated in FIG. 14B) can be mixed with a desiccant, which enables moisture to be adsorbed, resulting in improved reliability.

When parts of the pad 412 and the first electrode 401 are provided to extend to the outside of the sealant 405 and the sealant 406, those can serve as external input terminals. An IC chip 420 mounted with a converter or the like may be provided over the external input terminals.

### Embodiment 7

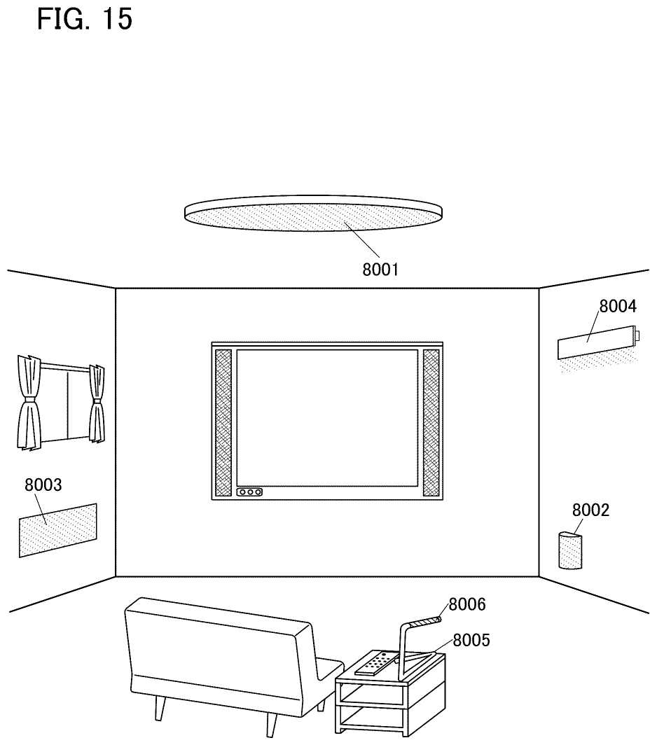

In this embodiment, application examples of lighting devices fabricated using the light-emitting apparatus of one embodiment of the present invention or the light-emitting device, which is part of the light-emitting apparatus, will be described with reference to FIG. 15.

A ceiling light 8001 can be used as an indoor lighting device. Examples of the ceiling light 8001 include a direct-mount light and an embedded light. Such lighting devices are fabricated using the light-emitting apparatus and a housing or a cover in combination. Other than that, application to a cord pendant light (light that is suspended from the ceiling by a cord) is also possible.

A foot light 8002 lights the floor so that safety on the floor can be improved. It can be effectively used in a bedroom, on a staircase, or in a passage, for example. In that case, the size or shape of the foot light can be changed in accordance with the area or structure of a room. The foot light can be a stationary lighting device made from the combination of the light-emitting apparatus and a support.

A sheet-like lighting 8003 is a thin sheet-like lighting device. The sheet-like lighting, which is attached to a wall when used, is space-saving and thus can be used for a wide variety of applications. Furthermore, the area of the sheet-like lighting can be easily increased. The sheet-like lighting can also be used on a wall or housing having a curved surface, for example.

In addition, a lighting device 8004 in which the light from a light source is controlled to be only in a desired direction can be used.

A desk lamp 8005 includes a light source 8006. As the light source 8006, the light-emitting apparatus of one embodiment of the present invention or the light-emitting device, which is part of the light-emitting apparatus, can be used.

In addition to the above examples, when the light-emitting apparatus of one embodiment of the present invention or the light-emitting device which, is part of the light-emitting apparatus, is used as a part of furniture in a room, a lighting device with functions of furniture can be obtained.

As described above, a variety of lighting devices that include the light-emitting apparatus can be obtained. Note that these lighting devices are also embodiments of the present invention.

The structures described in this embodiment can be used in an appropriate combination with any of the structures described in the other embodiments.

### Embodiment 8

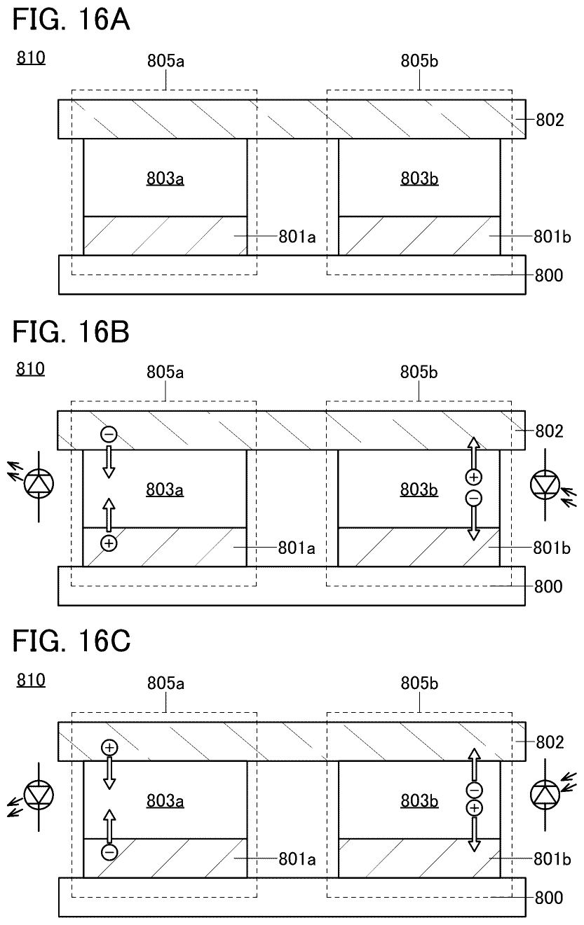

This embodiment will describe a light-emitting and light-receiving apparatus 810 with reference to FIG. 16, for description of a light-emitting device and a light-receiving device that can be used in a light-emitting apparatus of one embodiment of the present invention. The light-emitting and light-receiving apparatus 810 includes a light-emitting device and thus can be regarded as a light-emitting apparatus, includes a light-receiving device and thus can be regarded as a light-receiving apparatus, and can be used in a display portion in an electronic apparatus and thus can be regarded as a display panel or a display apparatus.

FIG. 16A is a schematic cross-sectional view illustrating a light-emitting device 805a and a light-receiving device 805b included in the light-emitting and light-receiving apparatus 810 of one embodiment of the present invention.

The light-emitting device 805a has a function of emitting light (hereinafter also referred to as a light-emitting function). The light-emitting device 805a includes an electrode 801a, an EL layer 803a, and an electrode 802. The light-emitting device 805a is preferably alight-emitting device utilizing organic EL (an organic EL device) described in Embodiment 2. The EL layer 803a interposed between the electrode 801a and the electrode 802 includes at least a light-emitting layer. The light-emitting layer contains a light-emitting substance. The EL layer 803a emits light when voltage is applied between the electrode 801a and the electrode 802. The EL layer 803a may include any of a variety of layers such as a hole-injection layer, a hole-transport layer, an electron-transport layer, an electron-injection layer, a carrier-blocking (hole-blocking or electron-blocking) layer, and a charge-generation layer, in addition to the light-emitting layer.

The light-receiving device 805b has a function of detecting light (hereinafter also referred to as a light-receiving function). For example, a pn or pin photodiode can be used as the light-receiving device 805b. The light-receiving device 805b includes an electrode 801b, a light-receiving layer 803b, and the electrode 802. The light-receiving layer 803b interposed between the electrode 801b and the electrode 802 includes at least an active layer. Note that for the light-receiving layer 803b, any of materials that are used for the variety of layers (e.g., the hole-injection layer, the hole-transport layer, the light-emitting layer, the electron-transport layer, the electron-injection layer, the carrier-blocking (hole-blocking or electron-blocking) layer, and the charge-generation layer) included in the above-described EL layer 803a can be used. The light-receiving device 805b functions as a photoelectric conversion device and generates charge on the basis of incident light on the light-receiving layer 803b, and the charge can be extracted as a current. At this time, voltage may be applied between the electrode 801b and the electrode 802. The amount of generated charge is determined depending on the amount of light incident on the light-receiving layer 803b.

The light-receiving device 805b has a function of detecting visible light. The light-receiving device 805b has sensitivity to visible light. The light-receiving device 805b further preferably has a function of detecting visible light and infrared light. The light-receiving device 805b preferably has sensitivity to visible light and infrared light.

In this specification and the like, a blue (B) wavelength range is greater than or equal to 400 nm and less than 490 nm, and blue (B) light has at least one emission spectrum peak in the wavelength range. A green (G) wavelength range is greater than or equal to 490 nm and less than 580 nm, and green (G) light has at least one emission spectrum peak in the wavelength range. A red (R) wavelength range is greater than or equal to 580 nm and less than 700 nm, and red (R) light has at least one emission spectrum peak in the wavelength range. In this specification and the like, a visible light wavelength is greater than or equal to 400 nm and less than 700 nm, and visible light has at least one emission spectrum peak in the wavelength range. An infrared (IR) wavelength range is greater than or equal to 700 nm and less than 900 nm, and infrared (IR) light has at least one emission spectrum peak in the wavelength range.

The active layer of the light-receiving device 805b contains a semiconductor. Examples of the semiconductor include an inorganic semiconductor such as silicon and an organic semiconductor including an organic compound. As the light-receiving device 805b, an organic semiconductor device (or an organic photodiode) including an organic semiconductor in the active layer is preferably used. An organic photodiode, which is easily made thin, lightweight, and large in area and has high flexibility in shape and design, can be employed for a variety of display apparatuses. With use of an organic semiconductor, the EL layer 803a included in the light-emitting device 805a and the light-receiving layer 803b included in the light-receiving device 805b can be formed by the same method (e.g., a vacuum evaporation method) with the same manufacturing apparatus, which is preferable. Note that the organic compound of one embodiment of the present invention can be used for the light-receiving layer 803b in the light-receiving device 805b.

In the display apparatus of one embodiment of the present invention, an organic EL device can be suitably used as the light-emitting device 805a and an organic photodiode can be suitably used as the light-receiving device 805b. The organic EL device and the organic photodiode can be formed over the same substrate. Thus, the organic photodiode can be incorporated in the display apparatus using the organic EL device. The display apparatus of one embodiment of the present invention has one or both of an image capturing function and a sensing function in addition to an image displaying function.

The electrode 801a and the electrode 801b are provided on the same plane. In FIG. 16A, the electrode 801a and the electrode 801b are provided over a substrate 800. The electrode 801a and the electrode 801b can be formed by processing a conductive film formed over the substrate 800 into island-like shapes, for example. In other words, the electrode 801a and the electrode 801b can be formed through the same process.

As the substrate 800, a substrate having heat resistance high enough to withstand the formation of the light-emitting device 805a and the light-receiving device 805b can be used. When an insulating substrate is used as the substrate 800, a glass substrate, a quartz substrate, a sapphire substrate, a ceramic substrate, an organic resin substrate, or the like can be used. Alternatively, a single crystal semiconductor substrate or a polycrystalline semiconductor substrate using silicon or silicon carbide as a material, a compound semiconductor substrate of silicon germanium or the like, or a semiconductor substrate such as an SOI substrate can be used.

As the substrate 800, it is particularly preferable to use the above-described insulating substrate or semiconductor substrate where a semiconductor circuit including a semiconductor element such as a transistor is formed. The semiconductor circuit preferably forms a pixel circuit, a gate line driver circuit (a gate driver), a source line driver circuit (a source driver), or the like. In addition to the above, an arithmetic circuit, a memory circuit, or the like may be formed.

The electrode 802 is formed of a layer shared by the light-emitting device 805a and the light-receiving device 805b. A conductive film transmitting visible light and infrared light is used as the electrode through which light exits or enters among these electrodes. A conductive film reflecting visible light and infrared light is preferably used as the electrode through which light neither exits nor enters.

The electrode 802 in the display apparatus of one embodiment of the present invention functions as one of the electrodes in each of the light-emitting device 805a and the light-receiving device 805b.

In FIG. 16B, the electrode 801a of the light-emitting device 805a has a potential higher than that of the electrode 802. In this case, the electrode 801a functions as an anode and the electrode 802 functions as a cathode in the light-emitting device 805a. The electrode 801b of the light-receiving device 805b has a potential lower than that of the electrode 802. For easy understanding of the flow direction of current, a circuit symbol of a light-emitting diode is shown on the left of the light-emitting device 805a and a circuit symbol of a photodiode is shown on the right of the light-receiving device 805b in FIG. 16B. The flow directions of carriers (electrons and holes) are also schematically indicated in each device by arrows.

In the structure illustrated in FIG. 16B, when a first potential is supplied to the electrode 801a through a first wiring, a second potential is supplied to the electrode 802 through a second wiring, and a third potential is supplied to the electrode 801b through a third wiring, the following relationship is satisfied: the first potential>the second potential>the third potential.

FIG. 16C illustrates the case where the electrode 801a of the light-emitting device 805a has a potential lower than that of the electrode 802. In this case, the electrode 801a functions as a cathode and the electrode 802 function as an anode in the light-emitting device 805a. The electrode 801b of the light-receiving device 805b has a potential lower than that of the electrode 802 and a potential higher than that of the electrode 801a. For easy understanding of the flow direction of current, FIG. 16C illustrates a circuit symbol of a light-emitting diode on the left of the light-emitting device 805a and a circuit symbol of a photodiode on the right of the light-receiving device 805b. The flow directions of carriers (electrons and holes) are schematically indicated in each device by arrows.

In the structure illustrated in FIG. 16C, when the first potential is supplied to the electrode 801a through the first wiring, the second potential is supplied to the electrode 802 through the second wiring, and the third potential is supplied to the electrode 801b through the third wiring, the following relationship is satisfied: the second potential>the third potential>the first potential.

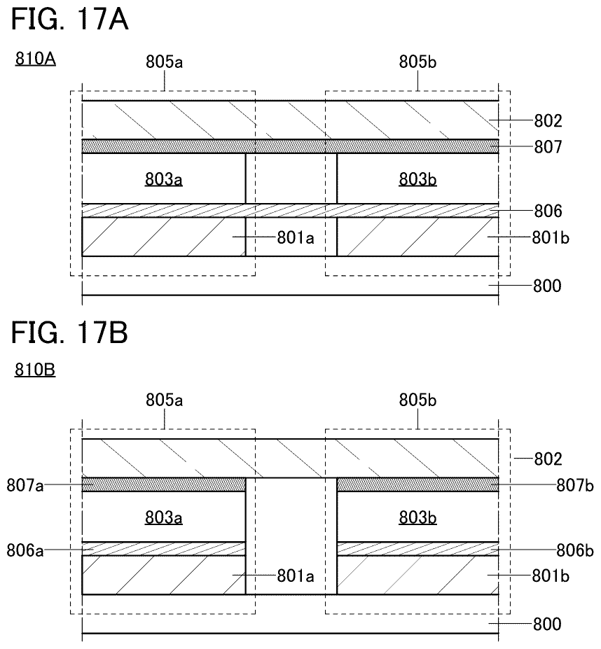

FIG. 17A illustrates a light-emitting and light-receiving apparatus 810A that is a variation example of the light-emitting and light-receiving apparatus 810. The light-emitting and light-receiving apparatus 810A is different from the light-emitting and light-receiving apparatus 810 in including a common layer 806 and a common layer 807. In the light-emitting device 805a, the common layer 806 and the common layer 807 function as part of the EL layer 803a. In the light-receiving device 805b, the common layer 806 and the common layer 807 function as part of the light-receiving layer 803b. The common layer 806 includes a hole-injection layer and a hole-transport layer, for example. The common layer 807 includes an electron-transport layer and an electron-injection layer, for example.

With the common layer 806 and the common layer 807, a light-receiving device can be incorporated without a significant increase in the number of times of separate formation of devices, whereby the light-emitting and light-receiving apparatus 810A can be manufactured with a high throughput.

FIG. 17B illustrates a light-emitting and light-receiving apparatus 810B that is a variation example of the light-emitting and light-receiving apparatus 810. The light-emitting and light-receiving apparatus 810B is different from the light-emitting and light-receiving apparatus 810 in that the EL layer 803a includes a layer 806a and a layer 807a and the light-receiving layer 803b includes a layer 806b and a layer 807b. The layer 806a and the layer 806b are formed using different materials, and each include a hole-injection layer and a hole-transport layer, for example. Note that the layer 806a and the layer 806b may be formed using the same material. The layer 807a and the layer 807b are formed using different materials, and each include an electron-transport layer and an electron-injection layer, for example. Note that the layer 807a and the layer 807b may be formed using the same material.

An optimum material for forming the light-emitting device 805a is selected for the layer 806a and the layer 807a and an optimum material for forming the light-receiving device 805b is selected for the layer 806b and the 807b, whereby the light-emitting device 805a and the light-receiving device 805b can have higher performance in the light-emitting and light-receiving apparatus 810B.

Note that the light-receiving devices 805b described in this embodiment can be arranged at a definition higher than or equal to 100 ppi, preferably higher than or equal to 200 ppi, more preferably higher than or equal to 300 ppi, further preferably higher than or equal to 400 ppi, still further preferably higher than or equal to 500 ppi and lower than or equal to 2000 ppi, lower than or equal to 1000 ppi, or lower than or equal to 600 ppi, for example. In particular, when the light-receiving devices 805b are arranged at a definition higher than or equal to 200 ppi and lower than or equal to 600 ppi, preferably higher than or equal to 300 ppi and lower than or equal to 600 ppi, the light-receiving devices can be suitably used for image capturing of a fingerprint. In the case where fingerprint authentication is performed with the display apparatus of one embodiment of the present invention, the increased definition of the light-receiving devices 805b enables, for example, highly accurate extraction of the minutiae of fingerprints; thus, the accuracy of the fingerprint authentication can be increased. The definition is preferably higher than or equal to 500 ppi, in which case the authentication conforms to the standard by the National Institute of Standards and Technology (NIST) or the like. On the assumption that the definition at which the light-receiving devices are arranged is 500 ppi, the size of each pixel is 50.8 μm, which indicates that the definition is adequate for image capturing of a fingerprint ridge distance (typically, greater than or equal to 300 μm and less than or equal to 500 μm).

The structures described in this embodiment can be used in an appropriate combination with any of the structures described in the other embodiments.

### Example 1

### Synthesis Example 1

In this synthesis example, a method of synthesizing 11-[4-(biphenyl-4-yl)-6-phenyl-1,3,5-triazin-2-yl]-11,12-dihydro-12-phenylindolo[2,3-a]carbazole-1,2,3,4,5,6,7,8,9,10-d10 (abbreviation: BP-Icz(II)Tzn-d10), which is the organic compound represented by Structural Formula (101) in Embodiment 1, will be specifically described.

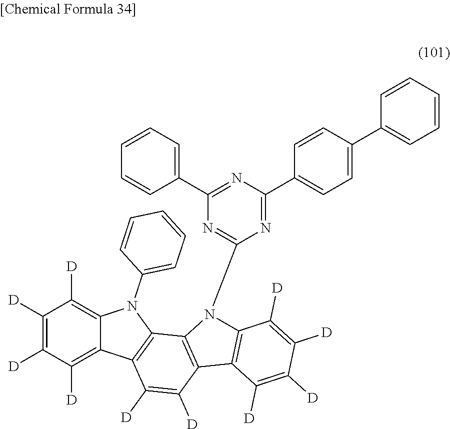 [SMILES: [2H]c1c([2H])c([2H])c2c(c1[2H])c1c([2H])c([2H])c3c4c([2H])c([2H])c([2H])c([2H])c4n(-c4nc(-c5ccccc5)nc(-c5ccc(-c6ccccc6)cc5)n4)c3c1n2-c1ccccc1]

### Step 1: Synthesis of 11,12-dihydro-11-phenylindolo[2,3-a]carbazole)-1,2,3,4,5,6,7,8,9,10-d10

Into a 200-mL three-neck flask were put 1.15 g (4.19 mmol) of molybdenum(V) pentachloride (abbreviation: MoCl₅) and 20 mL of deuterated toluene (abbreviation: toluene-d8), and 3.42 g (9.99 mmol) of 11,12-dihydro-11-phenylindolo[2,3-a]carbazole was added while the mixture was stirred. Next, this mixture was stirred under a nitrogen stream at 100° C. for 12 hours. After the reaction, toluene and 2 mol/L hydrochloric acid were added to this mixture and the aqueous layer and the organic layer were separated, and then the aqueous layer was subjected to extraction with toluene. The obtained organic layer was washed with a saturated aqueous solution of sodium hydrogen carbonate and a saturated aqueous solution of sodium chloride, and the organic layer was dried with magnesium sulfate. This mixture was separated by gravity filtration, and the filtrate was concentrated to give a solid.

The obtained solid was purified by silica gel column chromatography (toluene: hexane 5=1:1). The fraction was concentrated, whereby 1.80 g of a white solid of 11,12-dihydro-11-phenylindolo[2,3-a]carbazole-1,2,3,4,5,6,7,8,9,10-d10, which was a target substance, was obtained (yield: 52.6%). The synthesis scheme of Step 1 is shown in (A-1) below.

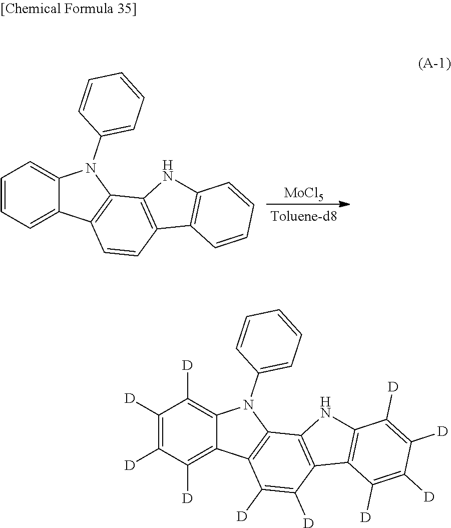

The obtained white solid was subjected to mass spectrometry; as a result, it was confirmed that the target substance, 11,12-dihydro-11-phenylindolo[2,3-a]carbazole-1,2,3,4,5,6,7,8,9,10-d10 (mass number 342) was obtained.

Furthermore, nuclear magnetic resonance spectroscopy (¹H-NMR) of a deuterated chloroform (abbreviation: CDCl₃) solution of the obtained white solid showed that the target substance, 11,12-dihydro-11-phenylindolo[2,3-a]carbazole)-1,2,3,4,5,6,7,8,9,10-d10 was obtained.

¹H-NMR (CDCl₃, 500 MHz): δ=7.49 (br, 1H), 7.64-7.75 (m, 5H)

### Step 2: Synthesis of BP-Icz(II)Tzn-d10

Into a 200-mL three-neck flask were added 1.80 g (5.25 mmol) of 11,12-dihydro-11-phenylindolo[2,3-a]carbazole-1,2,3,4,5,6,7,8,9,10-d10 obtained in Step 1, 2.62 g (7.61 mmol) of 2-([1,1-biphenyl]-4-yl)-4-chloro-6-phenyl-1,3,5-triazine, 1.21 g (12.6 mmol) of sodium-tert-butoxide (abbreviation: tBuONa), and 60 mL of xylene. Next, the air in the flask was replaced with nitrogen, and the mixture was degassed while being stirred under reduced pressure. Then, the mixture in the flask was heated at 90° C. under a nitrogen stream, 78 mg (0.15 mmol) of bis(tri-tert-butylphosphine)palladium(0) (abbreviation: Pd(t-Bu₃P)₂) was added, the temperature was raised to 110° C., and then the mixture was stirred for nine hours. After the reaction, water was added to the mixture and suction filtration was performed, and the obtained residue was washed with water and ethanol. The obtained residue was dissolved in heated toluene, followed by suction filtration through Celite and alumina. The filtrate was concentrated, and recrystallization with toluene and hexane and drying were performed to give 2.0 g of a pale yellow solid (yield: 58.7%). The synthesis scheme of Step 2 is shown in (A-2) below.

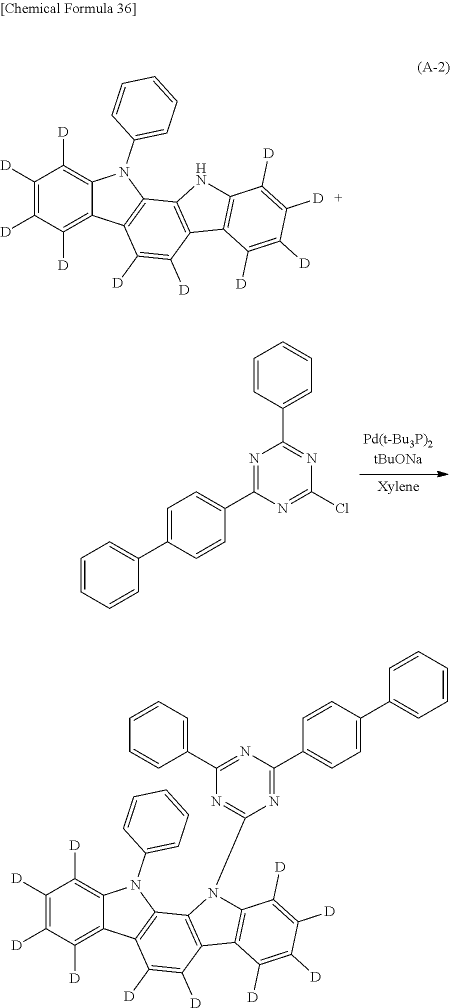

### <Purification by Sublimation>

By a train sublimation method, 1.97 g of the obtained pale yellow solid was purified by heating at 285° C. for 17 hours under a pressure of 2.75 Pa with an argon flow rate of 12 mL/min to give 1.47 g of a yellow solid (collection rate: 71%). The results of mass spectrometry indicate that BP-Icz(II)Tzn-d10 (mass number 649), which was a target substance, was obtained.

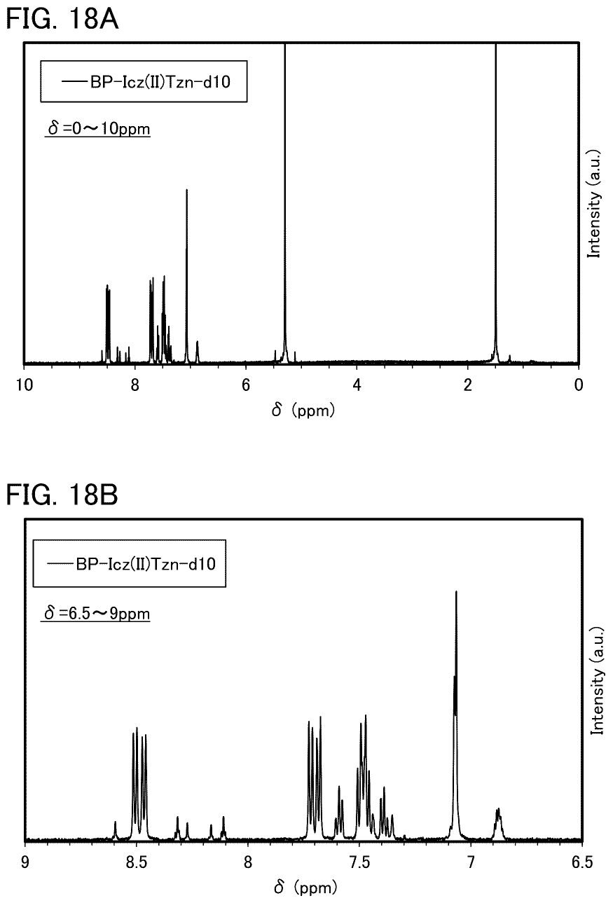

FIG. 18A and FIG. 18B show nuclear magnetic resonance spectroscopy (¹H-NMR) charts of BP-Icz(II)Tzn-d10 in a deuterated dichloromethane (abbreviation: CD₂Cl₂) solution after the purification by sublimation. The results show that BP-Icz(II)Tzn-d10 was obtained.

¹H-NMR (CD₂Cl₂, 500 MHz): δ=6.86-6.90 (m, 1H), 7.07-7.09 (m, 4H), 7.35-7.51 (m, 5H), 7.59 (t, J=6.8 Hz, 1H), 7.68 (d, J=8.5 Hz, 2H), 7.72 (d, J=8.0 Hz, 2H), 8.47 (d, J=8.5 Hz, 2H), 8.51 (d, J=8.0 Hz, 2H)

In FIG. 18B, fine signals are observed around δ=8.6 ppm, around 8.1 ppm to 8.4 ppm, and the like. They are assumed to be hydrogen that was not substituted by heavy hydrogen and remained in Synthesis Scheme (A-1). The deuteration rate of an indolocarbazole skeleton was estimated as follows.

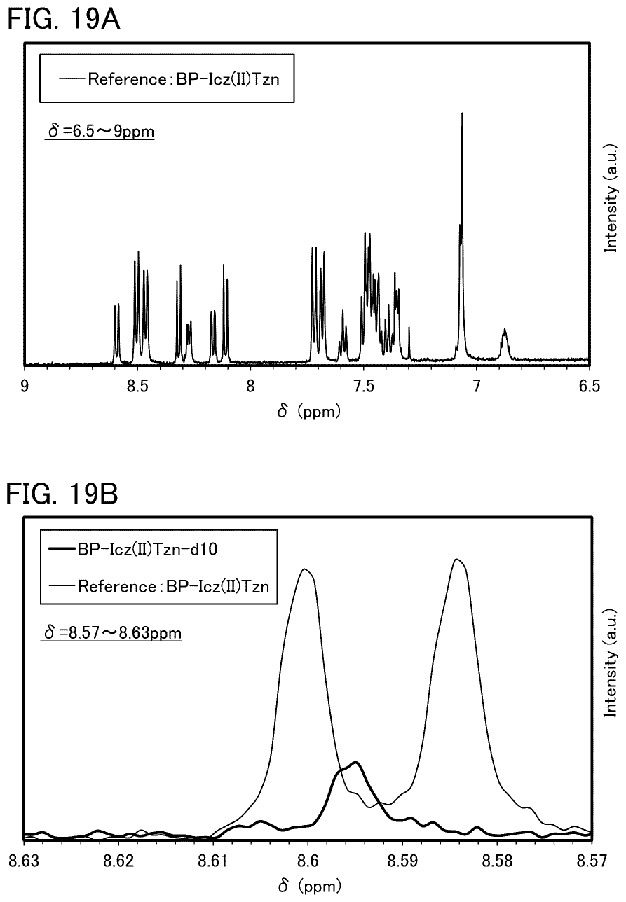

FIG. 19A is a ¹H-NMR chart of BP-Icz(II)Tzn, which is a non-deuterated substance of BP-Icz(II)Tzn-d10. FIG. 19B is an enlarged view showing around δ=8.6 ppm (δ=8.57 ppm to 8.63 ppm) for comparing BP-Icz(II)Tzn-d10 and BP-Icz(II)Tzn. From the signal area ratio at around δ=8.6 ppm, the deuteration rate of the indolocarbazole skeleton of BP-Icz(II)Tzn-d10 was estimated to be approximately 80% to 90%.

Note that BP-Icz(II)Tzn was formed by a known synthesis method. The molecular structure is described in Example 3.

### <Measurement of Emission Spectrum and Absorption Spectrum>

An ultraviolet-visible absorption spectrum and an emission spectrum of BP-Icz(II)Tzn-d10 in a dichloromethane solution were measured.

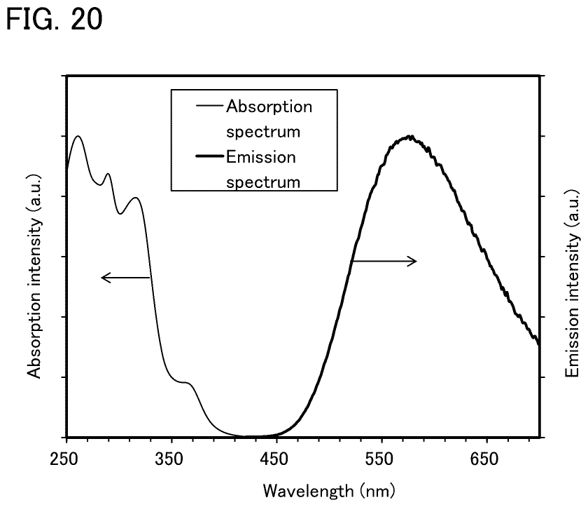

FIG. 20 is a graph showing wavelength dependence of absorption intensity and wavelength dependence of emission intensity. The ultraviolet-visible absorption spectrum in a solution state was obtained by subtraction of a measured absorption spectrum of a solvent (dichloromethane) alone in a quartz cell from a measured absorption spectrum of the solution of BP-Icz(II)Tzn-d10 in a quartz cell. Note that the measurement sample fabricated here (the solution in a quartz cell) is referred to as a light-emitting element, a light-emitting device, a light-emitting unit, or the like in some cases.

The ultraviolet-visible absorption spectrum of BP-Icz(II)Tzn-d10 in the dichloromethane solution has absorption intensity peaks at around 261 nm, 289 nm, 316 nm, and 360 nm (see FIG. 20). The emission spectrum has an emission intensity peak at around 579 nm (excitation light is 365 nm).

Note that the ultraviolet-visible absorption spectrum was measured with an ultraviolet-visible spectrophotometer (V-770DS, produced by JASCO Corporation). The emission spectrum was measured with a spectrofluorometer (FP-8600DS, produced by JASCO Corporation).

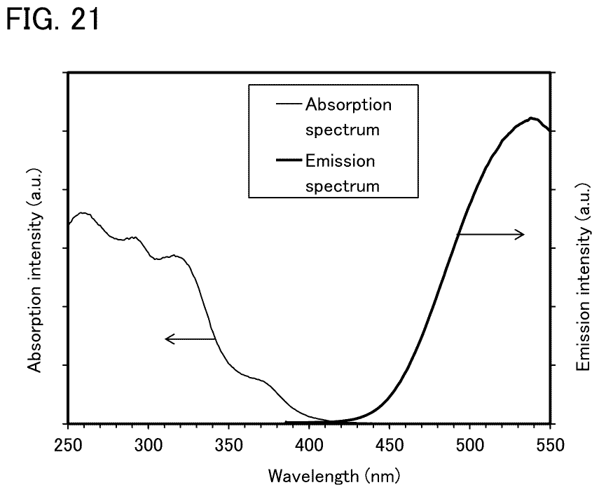

Next, FIG. 21 shows an absorption spectrum and an emission spectrum of a thin film. A solid thin film was formed over a quartz substrate by a vacuum evaporation method. The absorption spectrum of the thin film was obtained by subtraction of an absorption spectrum of a quartz substrate from an absorption spectrum of BP-Icz(II)Tzn-d10 deposited over a quartz substrate. Note that the measurement sample fabricated here (a state in which a thin film is formed over a substrate) is referred to as a light-emitting element, a light-emitting device, a light-emitting unit, or the like in some cases.

As shown in FIG. 21, the thin film of BP-Icz(II)Tzn-d10 has absorption peaks at around 262 nm, 294 nm, 374 nm, and 393 nm and an emission wavelength peak at 538 nm (excitation wavelength 375 nm). The results indicated that the organic compound of one embodiment of the present invention, BP-Icz(II)Tzn-d10, can be effectively used as a host material used in combination with a light-emitting substance or a light-emitting substance in the visible region.

The glass transition temperature (Tg) of BP-Icz(II)Tzn-d10 was measured. The Tg was measured with a differential scanning calorimeter (PYRIS 1 DSC produced by PerkinElmer Japan Co., Ltd.) while a powder was put on an aluminum cell. As a result, the Tg of BP-Icz(II)Tzn-d10 was 153° C.

The HOMO level and the LUMO level of BP-Icz(II)Tzn-d10 were calculated on the basis of cyclic voltammetry (CV) measurement. The calculation method is described below.

An electrochemical analyzer (model number: ALS model 600A or 600C, produced by BAS Inc.) was used as a measurement apparatus. To prepare a solution for the CV measurement, dehydrated dimethylformamide (DMF) (produced by Sigma-Aldrich Inc., 99.8%, catalog No. 22705-6) was used as a solvent, tetra-n-butylammonium perchlorate (n-Bu₄NClO₄) (produced by Tokyo Chemical Industry Co., Ltd., catalog No. T0836) as a supporting electrolyte was dissolved at a concentration of 100 mmol/L, and the object to be measured was dissolved at a concentration of 2 mmol/L.

A platinum electrode (PTE platinum electrode, produced by BAS Inc.) was used as a working electrode, another platinum electrode (Pt counter electrode for VC-3 (5 cm), produced by BAS Inc.) was used as an auxiliary electrode, and an Ag/Ag⁺ electrode (RE7 reference electrode for non-aqueous solvent, produced by BAS Inc.) was used as a reference electrode. Note that the measurement was performed at room temperature (20 to 25° C.).

The scan speed in the CV measurement was fixed to 0.1 V/see, and an oxidation potential Ea [V] and a reduction potential Ec [V] with respect to the reference electrode were measured. Ea is an intermediate potential of an oxidation-reduction wave, and Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Ea and LUMO level [eV]=−4.94−Ec.

Furthermore, CV measurement was repeated 100 times, and the oxidation-reduction wave in the hundredth cycle was compared with the oxidation-reduction wave in the first cycle to examine the electrical stability of the compound.

As a result, according to the measurement results of the oxidation potential Ea [V] of BP-Icz(II)Tzn-d10, the HOMO level was found to be −5.8 eV. By contrast, according to the measurement results of the reduction potential Ec [V], the LUMO level was found to be −2.99 eV. In addition, the results of repetitive measurement of the oxidation-reduction wave showed that when the waveform of the first cycle was compared with that of the hundredth cycle, 93% of the peak intensity and 89% of the peak intensity were maintained in the Ea measurement and the Ec measurement, respectively, which confirmed that BP-Icz(II)Tzn-d10 has extremely high resistance to oxidation and reduction. The results indicated that the organic compound of one embodiment of the present invention, BP-Icz(II)Tzn-d10, can be effectively used as a light-emitting substance, a hole-transport material, and an electron-transport material.

### <Measurement of Refractive Index>

The refractive index of BP-Icz(II)Tzn-d10 was measured by a spectroscopic ellipsometer (M-2000U, produced by J. A. Woollam Japan Corp.). For the measurement, a film in which BP-Icz(II)Tzn-d10 was deposited to a thickness of approximately 60 nm over a quartz substrate by a vacuum evaporation method was used. At a wavelength of 633 nm, the ordinary refractive index n Ordinary (no) was 1.79. This revealed that BP-Icz(II)Tzn-d10 was also able to be effectively used as a material of a cap layer provided over a cathode in a light-emitting apparatus. As a material of the cap layer, the refractive index is preferably higher than or equal to 1.75 and lower than or equal to 2.50.

In the case where BP-Icz(II)Tzn-d10 is used as a hole-transport material or an electron-transport material, the efficiency of the light-emitting device can be increased by reducing the refractive index. As a method of reducing the refractive index, an alkyl group is used as a substituent of BP-Icz(II)Tzn-d10, whereby the refractive index can be adjusted to be greater than or equal to 1.50 and less than or equal to 1.75.

### Example 2

### Synthesis Example 2

In this synthesis example, a method of synthesizing 11-[4-(biphenyl-4-yl)-6-phenyl-1,3,5-triazin-2-yl]-11,12-dihydro-12-(biphenyl-3-yl)-indolo[2,3-a]carbazole-1,2,3,4,5,6,7,8,9,10-d10 (abbreviation: BP-mBPIcz(II)Tzn-d10), which is the organic compound represented by Structural Formula (105) in Embodiment 1, will be specifically described.

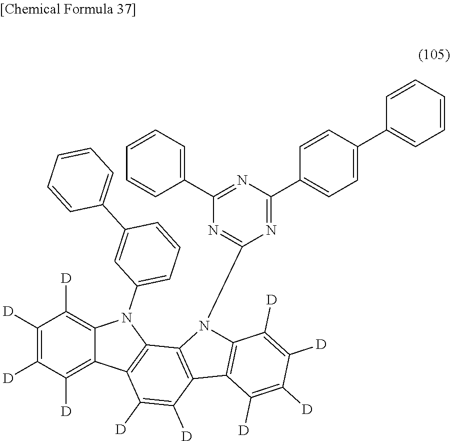 [SMILES: [2H]c1c([2H])c([2H])c2c(c1[2H])c1c([2H])c([2H])c3c4c([2H])c([2H])c([2H])c([2H])c4n(-c4nc(-c5ccccc5)nc(-c5ccc(-c6ccccc6)cc5)n4)c3c1n2-c1cccc(-c2ccccc2)c1]

### Step 1: Synthesis of 11,12-dihydro-11-(biphenyl-3-yl)-indolo[2,3-a]carbazole

Into a 200-mL three-neck flask were put 4.23 g (16.5 mmol) of 11,12-dihydroindolo[2,3-a]carbazole, 3.50 g (15.0 mmol) of 3-bromobiphenyl, 1.9 g (19.8 mmol) of sodium-tert-butoxide (abbreviation: tBuONa), 233 mg (0.57 mmol) of 2-dicyclohexylphosphino-2′,6′-dimethoxybiphenyl (abbreviation: S-Phos), and 75 mL of xylene. Next, the air in the flask was replaced with nitrogen, and the mixture was degassed while being stirred under reduced pressure. Then, the mixture in the flask was heated at 70° C. under a nitrogen stream; 103 mg (0.18 mmol) of bis(dibenzylideneacetone)palladium(0) (abbreviation: Pd(dba)₂) was added; the temperature was raised to 120° C. and the mixture was stirred for 13 hours; and the temperature was raised to 130° C. and the mixture was stirred for 3.5 hours. After the reaction, the mixture was subjected to suction filtration and washed with toluene. The obtained filtrate was concentrated and purified by silica gel column chromatography (toluene:hexane=1:1). The fraction was concentrated, whereby 3.9 g of a solid of 11,12-11-(biphenyl-3-yl)-dihydroindolo[2,3-a]carbazole, which was a target substance, was obtained (yield: 98%). The synthesis scheme of Step 1 is shown in (B-1) below.

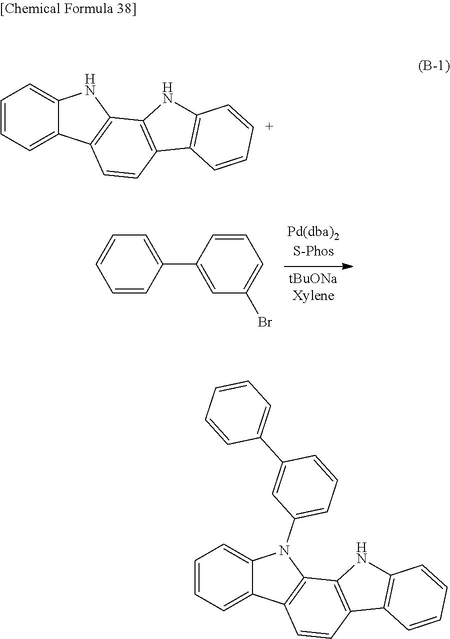 [SMILES: [C+]1=Cc2[nH]c3c(ccc4c5ccccc5[nH]c43)c2C=C1 ; Brc1cccc(-c2ccccc2)c1 ; c1ccc(-c2cccc(-n3c4ccccc4c4ccc5c6ccccc6[nH]c5c43)c2)cc1]

### Step 2: Synthesis of 11,12-dihydro-11-(biphenyl-3-yl)-indolo[2,3-a]carbazole-1,2,3,4,5,6,7,8,9,10-d10

Into a 200-mL three-neck flask were put 3.9 g (9.56 mmol) of 11,12-11-(biphenyl-3-yl)-dihydroindolo[2,3-a]carbazole which is obtained in Step 1 and 20 mL of deuterated toluene (abbreviation: toluene-d8), the air in the flask was replaced with nitrogen, and then 2.43 g (8.89 mmol) of molybdenum(V) pentachloride (abbreviation: MoCl₅) was added while the mixture was stirred. Next, this mixture was stirred under a nitrogen stream at 100° C. for four hours. After the reaction, toluene and 2 mol/L hydrochloric acid were added to this mixture and then the aqueous layer was subjected to extraction with toluene. The obtained organic layer was washed with a saturated aqueous solution of sodium hydrogen carbonate and a saturated aqueous solution of sodium chloride, and the organic layer was dried with magnesium sulfate. This mixture was separated by gravity filtration, and the filtrate was concentrated to give 2.2 g of a target solid, 11,12-dihydro-12-(biphenyl-3-yl)-indolo[2,3-a]carbazole-1,2,3,4,5,6,7,8,9,10-d10 (yield: 55%). The synthesis scheme of Step 2 is shown in (B-2) below.

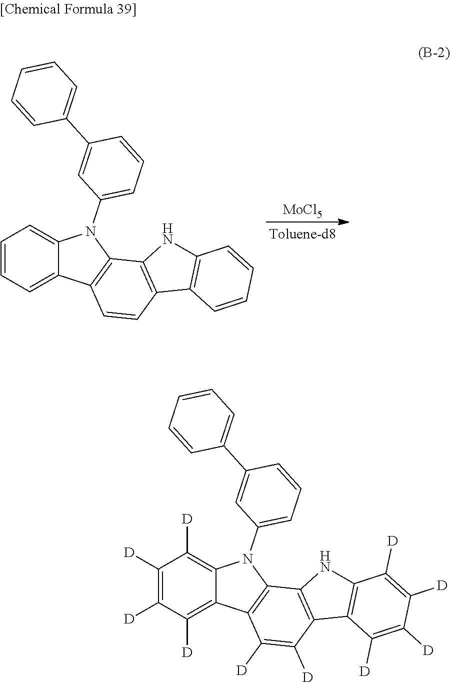 [SMILES: c1ccc(-c2cccc(-n3c4ccccc4c4ccc5c6ccccc6[nH]c5c43)c2)cc1 ; "MoCl5" ; [2H]c1c([2H])c([2H])c2c([nH]c3c2c([2H])c([2H])c2c4c([2H])c([2H])c([2H])c([2H])c4n(-c4cccc(-c5ccccc5)c4)c32)c1[2H]]

The obtained white solid was subjected to mass spectrometry; as a result, it was confirmed that the target substance, 11,12-dihydro-12-(biphenyl-3-yl)-indolo[2,3-a]carbazole-1,2,3,4,5,6,7,8,9,10-d10 (mass number 418) was obtained.

Furthermore, nuclear magnetic resonance spectroscopy (¹H-NMR) of a deuterated chloroform (abbreviation: CDCl₃) solution of the obtained white solid showed that the target substance, 11,12-dihydro-12-(biphenyl-3-yl)-indolo[2,3-a]carbazole-1,2,3,4,5,6,7,8,9,10-d10 was obtained.

¹H-NMR (CDCl₃, 500 MHz): δ=7.46-7.49 (m, 3H), 7.61 (br, 1H), 7.67-7.70 (m, 3H), 7.78-7.81 (m, 1H), 7.88 (d, J=7.5 Hz, 1H), 7.93 (s, 1H)

### Step 3: Synthesis of BP-mBPIcz(II)Tzn-d10

Into a 200-mL three-neck flask were put 2.02 g (4.82 mmol) of 11,12-dihydro-12-(biphenyl-3-yl)-indolo[2,3-a]carbazole-1,2,3,4,5,6,7,8,9,10-d, which was obtained in Step 2, 3.60 g (10.5 mmol) of 2-([1,1-biphenyl]-4-yl)-4-chloro-6-phenyl-1,3,5-triazine, 1.11 g (11.8 mmol) of sodium-tert-butoxide (abbreviation: tBuONa), and 60 mL of xylene. Next, this mixture was degassed while being stirred under reduced pressure, and the air in the flask was replaced with nitrogen. Next, the mixture in the flask was heated at 90° C. under a nitrogen stream, 80 mg (0.16 mmol) of bis(tri-tert-butylphosphine)palladium(0) (abbreviation: Pd(t-Bu₃P)₂) was added, the temperature was raised to 110° C., and then the mixture was stirred for five hours. After the reaction, water was added to the mixture, suction filtration was performed, ethyl acetate and water were added to the filtrate, and the aqueous layer was subjected to extraction. The obtained organic layer was washed with a saturated aqueous solution of sodium chloride and dried with magnesium sulfate. The resulting filtrate was concentrated, followed by purification by silica gel column chromatography (ethyl acetate:hexane=1:20). The obtained fraction was concentrated and dried, whereby 2.3 g of a pale yellow solid of BP-mBPIcz(II)Tzn-d10 was obtained (yield: 66%). The synthesis scheme of Step 3 is shown in (B-3) below.

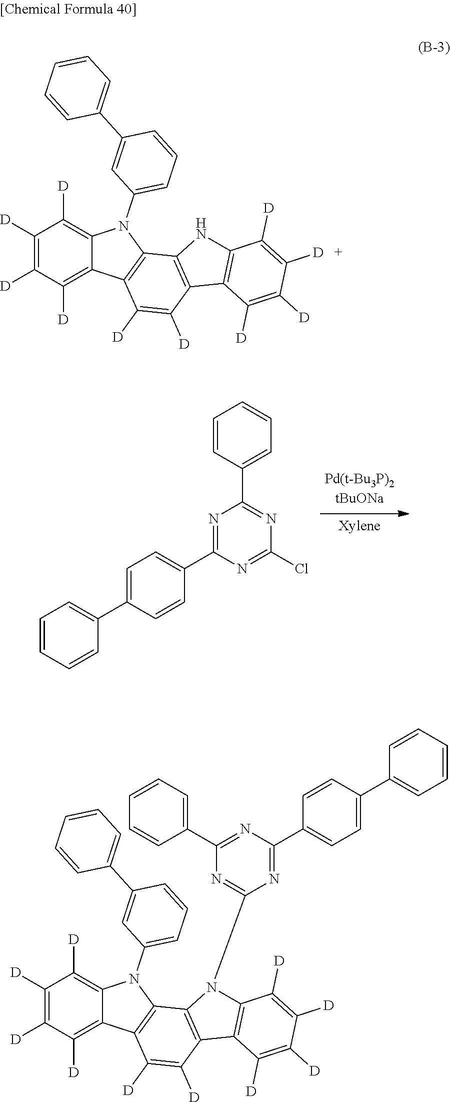

### <Purification by Sublimation>

By a train sublimation method, 2.28 g of the obtained pale yellow solid was purified by heating at 295° C. for 21 hours under a pressure of 3.07 Pa with an argon flow rate of 10 mL/min to give 1.99 g of a yellow solid (collection rate: 87%). The results of mass spectrometry indicate that BP-mBPIcz(II)Tzn-d10 (mass number 736), which was a target substance, was obtained.

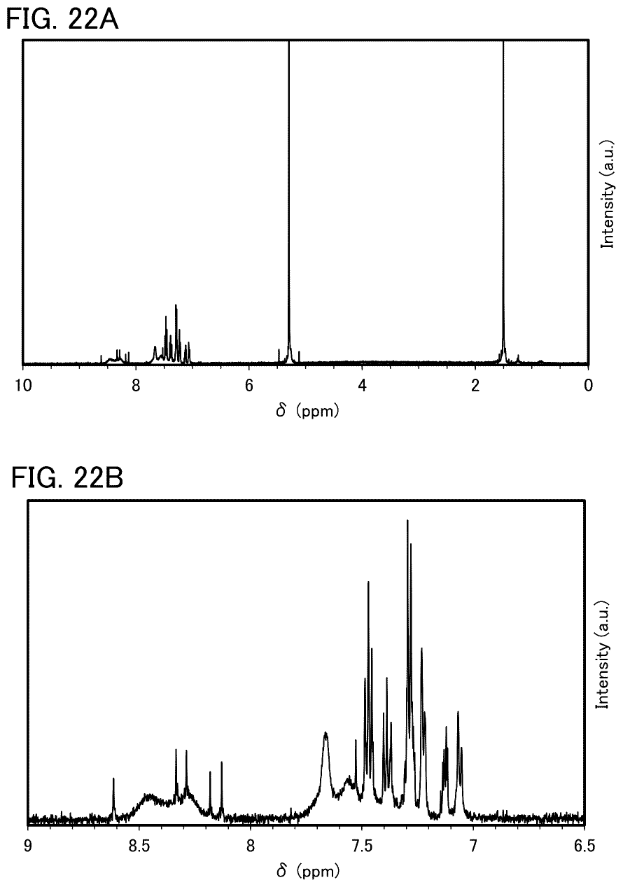

FIG. 22A and FIG. 22B show nuclear magnetic resonance spectroscopy (¹H-NMR) charts of BP-mBPIcz(II)Tzn-d10 in a deuterated dichloromethane (abbreviation: CD₂Cl₂) solution after the purification by sublimation. The results show that BP-mBPIcz(II)Tzn-d10 was obtained.

¹H-NMR (CD₂Cl₂, 500 MHz): S=7.05-7.15 (m, 3H), 7.55-7.66 (m, 16H), 8.29 (br, 2H), 8.48 (br, 2H)

When the deuteration rate of the indolocarbazole skeleton was estimated as in Example 1, the deuteration rate of the indolocarbazole skeleton was approximately 75% to 90%.

### <Measurement of Emission Spectrum and Absorption Spectrum>

An ultraviolet-visible absorption spectrum and an emission spectrum of BP-mBPIcz(II)Tzn-d10 in a dichloromethane solution were measured. The measurement method, the measurement device, and the like are similar to those in the other examples.

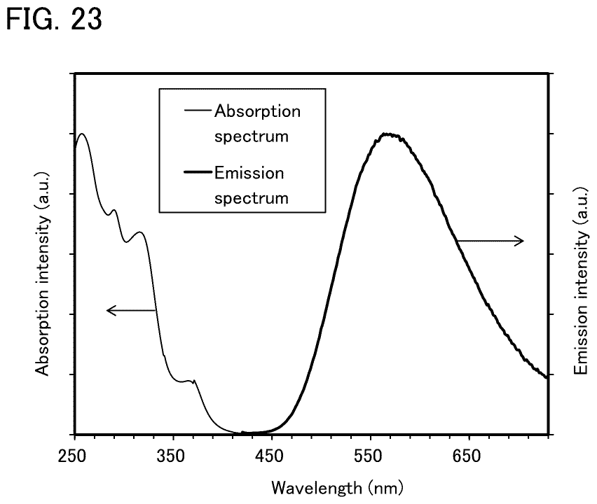

FIG. 23 is a graph showing wavelength dependence of absorption intensity and wavelength dependence of emission intensity. The ultraviolet-visible absorption spectrum of BP-mBPIcz(II)Tzn-d10 in the dichloromethane solution has absorption intensity peaks at around 256 nm, 289 nm, 315 nm, and 364 nm. The emission spectrum has an emission intensity peak at around 563 nm (excitation light is 371 nm).

The glass transition temperature (Tg) of BP-mBPIcz(II)Tzn-d10 was measured. The Tg was measured with a differential scanning calorimeter (PYRIS 1 DSC produced by PerkinElmer Japan Co., Ltd.) while a powder was put on an aluminum cell. As a result, the Tg of BP-mBPIcz(II)Tzn-d10 was 148° C.

### Example 3

In this example, a light-emitting device of one embodiment of the present invention described in the embodiment, and a comparative light-emitting device are described. Structural formulae of organic compounds used for the light-emitting device of one embodiment of the present invention and the comparative light-emitting device are shown below.

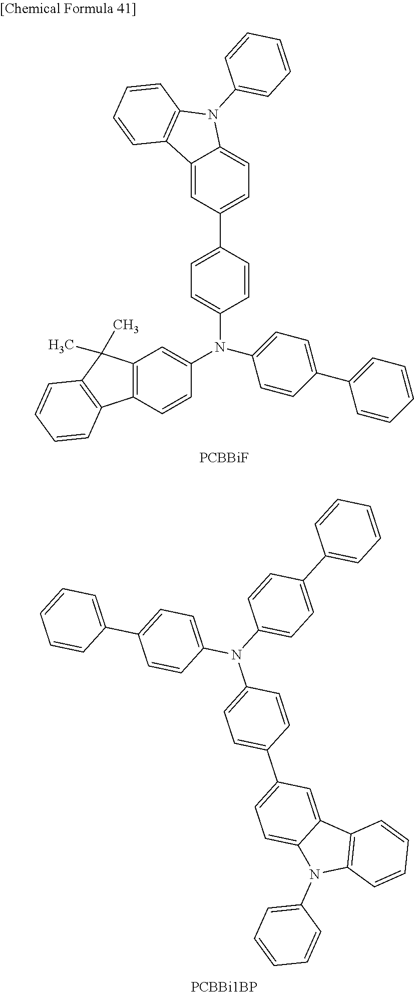

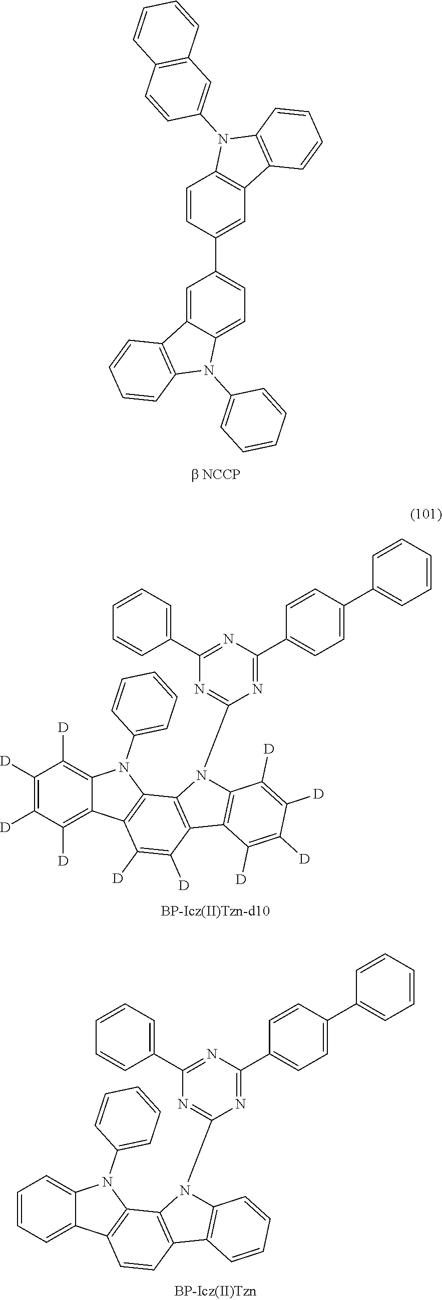 [SMILES: c1ccc(-n2c3ccccc3c3cc(-c4ccc5c(c4)c4ccccc4n5-c4ccc5ccccc5c4)ccc32)cc1 ; [2H]c1c([2H])c([2H])c2c(c1[2H])c1c([2H])c([2H])c3c4c([2H])c([2H])c([2H])c([2H])c4n(-c4nc(-c5ccccc5)nc(-c5ccc(-c6ccccc6)cc5)n4)c3c1n2-c1ccccc1 ; c1ccc(-c2ccc(-c3nc(-c4ccccc4)nc(-n4c5ccccc5c5ccc6c7ccccc7n(-c7ccccc7)c6c54)n3)cc2)cc1]

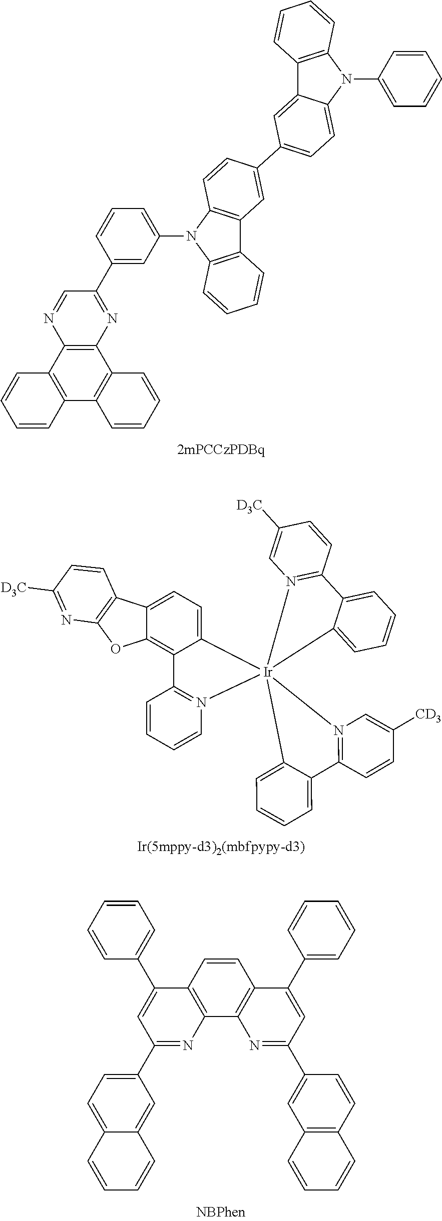

### (Fabrication Method of Light-Emitting Device 1)

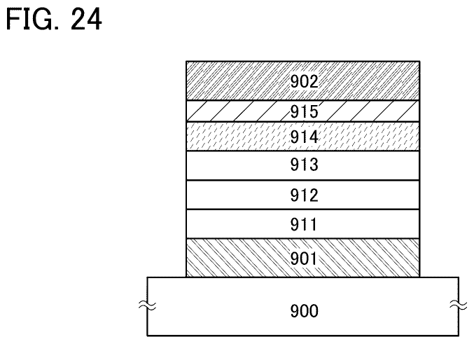

The light-emitting device 1 described in this example has a structure, as illustrated in FIG. 24, in which a hole-injection layer 911, a hole-transport layer 912, a light-emitting layer 913, an electron-transport layer 914, and an electron-injection layer 915 are stacked in this order over a first electrode 901 formed over a glass substrate 900, and a second electrode 902 is stacked over the electron-injection layer 915.

First, indium tin oxide containing silicon oxide (ITSO) was deposited over the glass substrate 900 by a sputtering method, so that the first electrode 901 was formed. Note that the thickness was 70 nm and the area of the electrode was 2 mm×2 mm.

Next, in pretreatment for forming the light-emitting device over the substrate, a surface of the substrate was washed with water and baked at 200° C. for an hour, and then UV ozone treatment was performed for 370 seconds. After that, the substrate was transferred into a vacuum evaporation apparatus where the pressure was reduced to approximately 10⁻⁴ Pa, vacuum baking was performed at 170° C. for 30 minutes in a heating chamber of the vacuum evaporation apparatus, and then the substrate was cooled down for approximately 30 minutes.

Next, the substrate over which the first electrode 901 was formed was fixed to a substrate holder provided in the vacuum evaporation apparatus such that the surface over which the first electrode 901 was formed faced downward. Over the first electrode 901, N-(1,1′-biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron-acceptor material with a molecular weight of 672 (OCHD-003) were deposited by co-evaporation using resistance heating to a thickness of 10 nm such that the weight ratio was 1:0.03 (=PCBBiF: OCHD-003), whereby the hole-injection layer 911 was formed.

Next, over the hole-injection layer 911, PCBBiF was deposited by evaporation to a thickness of 40 nm and then 4,4′-diphenyl-4″-(9-phenyl-9H-carbazol-3-yl)triphenylamine (abbreviation: PCBBi1BP) was deposited by evaporation to a thickness of 10 nm; whereby the hole-transport layer 912 was formed.

Then, over the hole-transport layer 912, BP-Icz(II)Tzn-d10 (Structural Formula (101)), which is an organic compound of one embodiment of the present invention whose synthesis method is described in Example 1, 9-(2-naphthyl)-9′-phenyl-9H,9′H-3,3′-bicarbazole (abbreviation: βNCCP), and [2-d3-methyl-8-(2-pyridinyl-κN)benzofuro[2,3-b]pyridine-κC]bis[2-(5-d3-methyl-2-pyridinyl-κN²)phenyl-κC]iridium(III) (abbreviation: Ir(5mppy-d3)₂(mbfpypy-d3)) were deposited by co-evaporation to a thickness of 40 nm such that the weight ratio of 0.5:0.5:0.1 (=BP-Icz(II)Tzn-d10: βNCCP: Ir(5mppy-d3)₂(mbfpypy-d3)), whereby the light-emitting layer 913 was formed.

After that, over the light-emitting layer 913, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 10 nm, and then, 2,9-di(naphthalen-2-yl)-4,7-diphenyl-1,10-phenanthroline (abbreviation: NBPhen) was deposited by evaporation to a thickness of 25 nm, whereby the electron-transport layer 914 was formed.

After the formation of the electron-transport layer 914, lithium fluoride (LiF) was deposited by evaporation to a thickness of 1 nm to form the electron-injection layer 915, and then aluminum (Al) was deposited by evaporation to a thickness of 200 nm to form the second electrode 902, whereby the light-emitting device 1 of this example was fabricated.

(Fabrication Method of Comparative Light-Emitting Device 2) A comparative light-emitting device 2 has a structure in which BP-Icz(II)Tzn-d10 used for the light-emitting layer 913 in the light-emitting device 1 is replaced with BP-Icz(II)Tzn. Specifically, the comparative light-emitting device 2 was fabricated in a manner similar to that for a comparative light-emitting device 1 except that the light-emitting layer 913 was formed to a thickness of 40 nm over the hole-transport layer 912 by co-evaporation of BP-Icz(II)Tzn, βNCCP, and Ir(5mppy-d3)₂(mbfpypy-d3) at a weight ratio of 0.5:0.5:0.1 (=BP-Icz(II)Tzn: βNCCP: Ir(5mppy-d3)₂(mbfpypy-d3)).

The element structures of the light-emitting device and the comparative light-emitting device are listed in the following table.

TABLE 1

| | | | Comparative |
|---|---|---|---|
| | Film | Light-emitting | light-emitting |
| | thickness | device 1 | device 2 |
| Second electrode | 200 nm | Al | |
| Electron-injection | 1 nm | LiF | |
| layer | | | |
| Electron-transport | 25 nm | NBPhen | |
| layer | 10 nm | 2mPCCzPDBq | |
| Light-emitting | 40 nm | BP-Icz(II)Tzn- | BP-Icz(II)Tzn:βNCCP:Ir(5mppy- |
| layer | | d10:βNCCP:Ir(5mppy- | d3)₂(mbfpypy-d3) |
| | | d3)₂(mbfpypy-d3) | (0.5:0.5:0.1) |
| | | (0.5:0.5:0.1) | |

| | | |
|---|---|---|
| Hole-transport | 10 nm | PCBBilBP |
| layer | 40 nm | PCBBiF |
| Hole-injection | 10 nm | PCBBiF:OCHD-003 |
| layer | | (1:0.03) |
| First electrode | 70 nm | ITSO |

The light-emitting device 1 and the comparative light-emitting device 2 were subjected to sealing with a glass substrate (a sealant was applied to surround the elements, followed by UV treatment and one-hour heat treatment at 80° C. at the time of sealing) in a glove box containing a nitrogen atmosphere so that the light-emitting devices were not exposed to the air. Then, the initial characteristics of the light-emitting devices were measured.

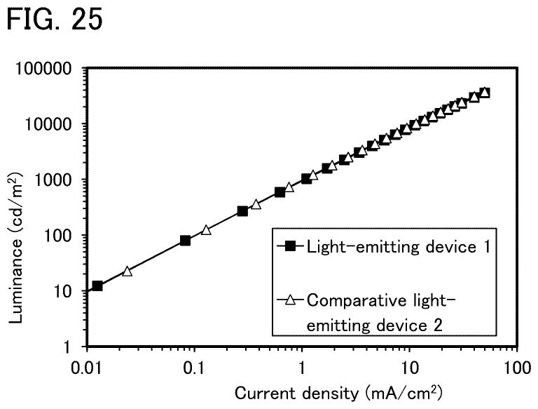

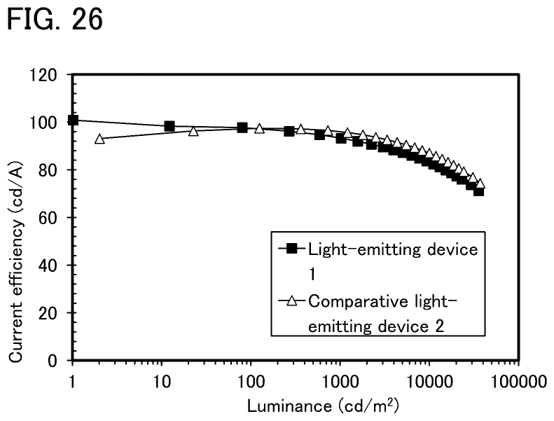

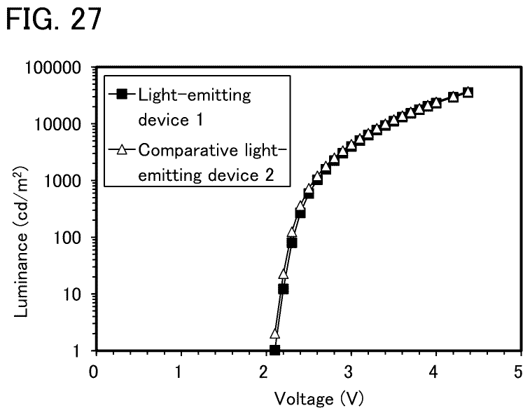

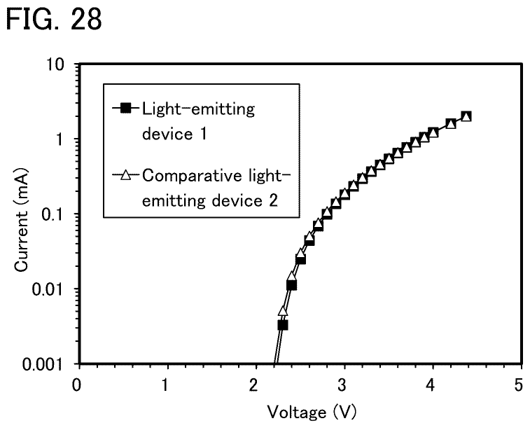

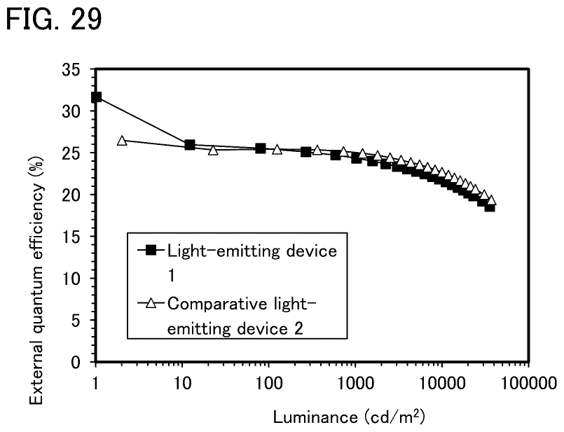

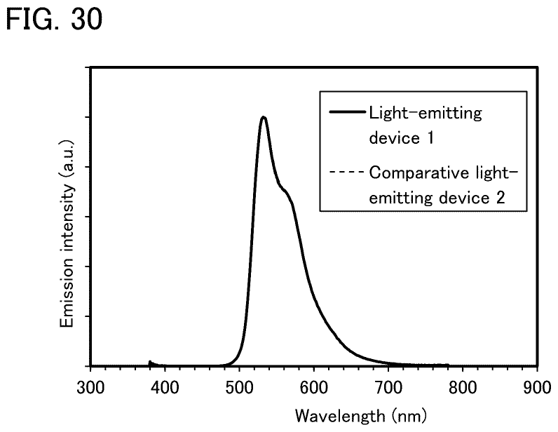

FIG. 25 shows the luminance-current density characteristics of the light-emitting device 1 and the comparative light-emitting device 2; FIG. 26 shows the current efficiency-luminance characteristics thereof, FIG. 27 shows the luminance-voltage characteristics thereof, FIG. 28 shows the current-voltage characteristics thereof, FIG. 29 shows the external quantum efficiency-luminance characteristics thereof, and FIG. 30 shows the emission spectra thereof. Table 2 shows main characteristics of the light-emitting devices at approximately 1000 cd/m². Note that the luminance, CIE chromaticity, and emission spectra were measured using a spectroradiometer (SR-UL1R, produced by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 2

| | | | Current | | | Current | External |
|---|---|---|---|---|---|---|---|
| | Voltage | Current | density | | | efficiency | quantum |
| | (V) | (mA) | (mA/cm²) | Chromaticity x | Chromaticity y | (cd/A) | efficiency (%) |
| Light-emitting | 2.6 | 0.044 | 1.10 | 0.370 | 0.609 | 93.2 | 24.3 |
| device 1 | | | | | | | |
| Comparative | 2.6 | 0.051 | 1.27 | 0.372 | 0.608 | 95.6 | 24.9 |
| light-emitting | | | | | | | |
| device 2 | | | | | | | |

It was found from FIG. 25 to FIG. 30 that the light-emitting device 1, which is the light-emitting device of one embodiment of the present invention, has emission efficiency comparable to that of the comparative light-emitting device 2.

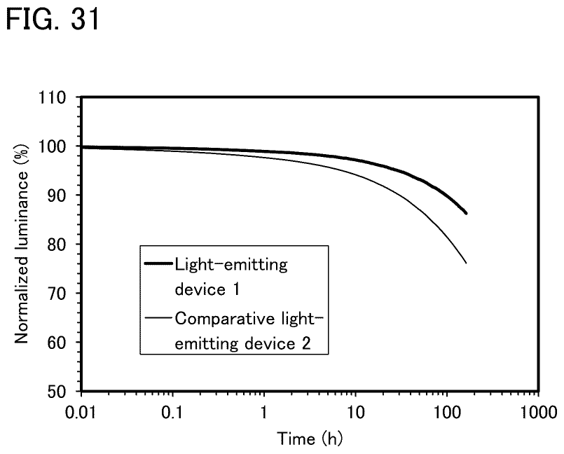

FIG. 31 shows changes in luminance over driving time when the light-emitting device 1 and the comparative light-emitting device 2 were driven at a constant current of 2 mA (50 mA/cm²). As shown in FIG. 31, the light-emitting device 1 had a longer lifetime than the comparative light-emitting device 2. Accordingly, it was found that the lifetime of the light-emitting device including BP-Icz(II)Tzn-d10, which is the organic compound of one embodiment of the present invention, is longer than that of the light-emitting device including BP-Icz(II)Tzn. That is, it was found that deuteration of an indolocarbazole skeleton included in BP-Icz(II)Tzn can prevent dissociation of a carbon-hydrogen bond, whereby a light-emitting device including the organic compound can have a longer lifetime.

Next, fabricated were a light-emitting device 1-a, a light-emitting device 1-b, and a light-emitting device 1-c in which the weight ratio of BP-Icz(II)Tzn-d10 to βNCCP in the light-emitting layer 913 of the light-emitting device 1 is changed to 0.3:0.7, 0.4:0.6, and 0.6:0.4, respectively. The weight ratios between BP-Icz(II)Tzn-d10, βNCCP, and Ir(5mppy-d3)₂(mbfpypy-d3) in the light-emitting device 1, the light-emitting device 1-a, the light-emitting device 1-b, and the light-emitting device 1-c are listed in the following table.

TABLE 3

| | Light-emitting | Light-emitting | Light-emitting | Light-emitting |
|---|---|---|---|---|
| | device 1-a | device 1-b | device 1 | device 1-c |
| BP-Icz(II)Tzn- | 0.3:0.7:0.1 | 0.4:0.6:0.1 | 0.5:0.5:0.1 | 0.6:0.4:0.1 |
| d10:βNCCP:Ir(5mppy- | | | | |
| d3)₂(mbfpypy-d3) | | | | |

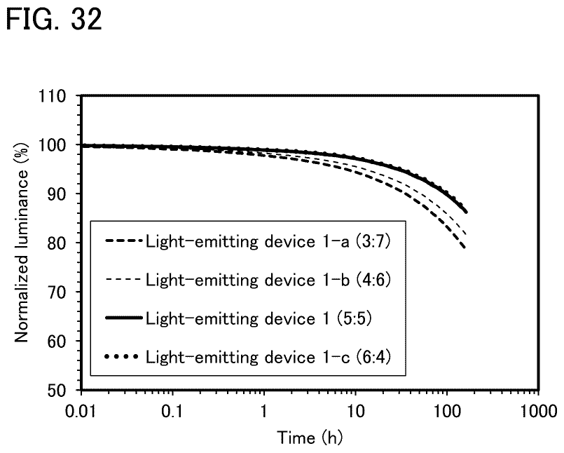

FIG. 32 shows changes in luminance over driving time when the light-emitting device 1, the light-emitting device 1-a, the light-emitting device 1-b, and the light-emitting device 1-c were driven at a constant current of 2 mA (50 mA/cm²). As shown in FIG. 32, the light-emitting device has a longer lifetime as the proportion of BP-Icz(II)Tzn-d10 in the light-emitting layer 913 is higher.

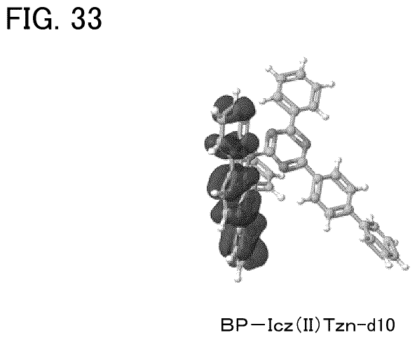

Here, HOMO orbital distribution of BP-Icz(II)Tzn-d10 was analyzed. The vibration (spin density) in the most stable structure where the singlet ground state (S₀) level of the compound is the lowest was analyzed by a calculation method. A density functional theory (DFT) method was used as the calculation method. The total energy calculated by the DFT is represented as the sum of potential energy, electrostatic energy between electrons, electronic kinetic energy, and exchange-correlation energy including all the complicated interactions between electrons. Also in the DFT, exchange-correlation interaction is approximated by a functional (a function of another function) of one electron potential represented in terms of electron density to enable high-speed calculations. Here, B3LYP which was a hybrid functional was used to specify the weight of each parameter related to exchange-correlation energy. As a basis function, 6-311G (d,p) was used. Gaussian 09 was used as a computational program. FIG. 33 shows the results.

In FIG. 33, a shadow in the molecule shows HOMO distribution of BP-Icz(II)Tzn-d10. FIG. 33 shows that HOMO distribution was observed over the indolocarbazole skeleton.

Specifically, when BP-Icz(II)Tzn-d10 receives a hole, the indolocarbazole skeleton where the HOMO is distributed probably receives a hole. That is, the use of BP-Icz(II)Tzn-d10 for the light-emitting device can probably prevent dissociation of a carbon-hydrogen bond in the indolocarbazole skeleton.

Although βNCCP used for the light-emitting layer 913 is also a hole-transport material and has a function of receiving a hole, receiving a hole might cause deterioration. By increasing the proportion of BP-Icz(II)Tzn-d10 and decreasing the proportion of βNCCP in the light-emitting layer 913, the amount of holes that BP-Icz(II)Tzn-d10, which is less likely to deteriorate, receives is increased; thus, deterioration of βNCCP can be prevented. Accordingly, as shown in FIG. 32, the light-emitting device has a longer lifetime as the proportion of BP-Icz(II)Tzn-d10 in the light-emitting layer 913 is higher.

### Example 4

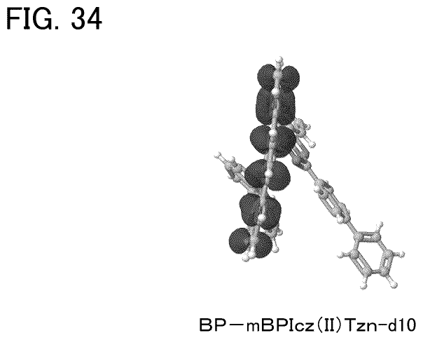

As for BP-mBPIcz(II)Tzn-d10 represented by Structural Formula (105), HOMO orbital distribution was analyzed as in Example 3. FIG. 34 shows the results.

In FIG. 34, a shadow in the molecule shows HOMO distribution of BP-mBPIcz(II)Tzn-d10. FIG. 34 shows that HOMO distribution was observed over the indolocarbazole skeleton.

From the calculation results, when BP-mBPIcz(II)Tzn-d10 receives a hole, the indolocarbazole skeleton where the HOMO is distributed probably receives a hole as in Example 3. That is, the use of BP-mBPIcz(II)Tzn-d10 for the light-emitting device can prevent dissociation of a carbon-hydrogen bond in the indolocarbazole skeleton and can increase the light emission lifetime. Furthermore, the lifetime of the light-emitting device can be longer as the proportion of BP-mBPIcz(II)Tzn-d10 is higher.

### Example 5

In this example, a light-emitting device, which is one embodiment of the present invention described in the embodiment, and a comparative light-emitting device are described. Structural formulae of organic compounds used for the light-emitting device of one embodiment of the present invention and the comparative light-emitting device are shown below.

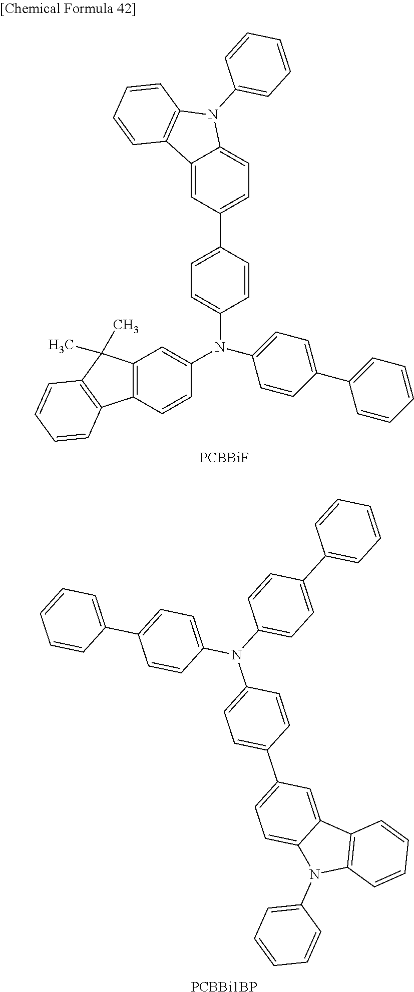

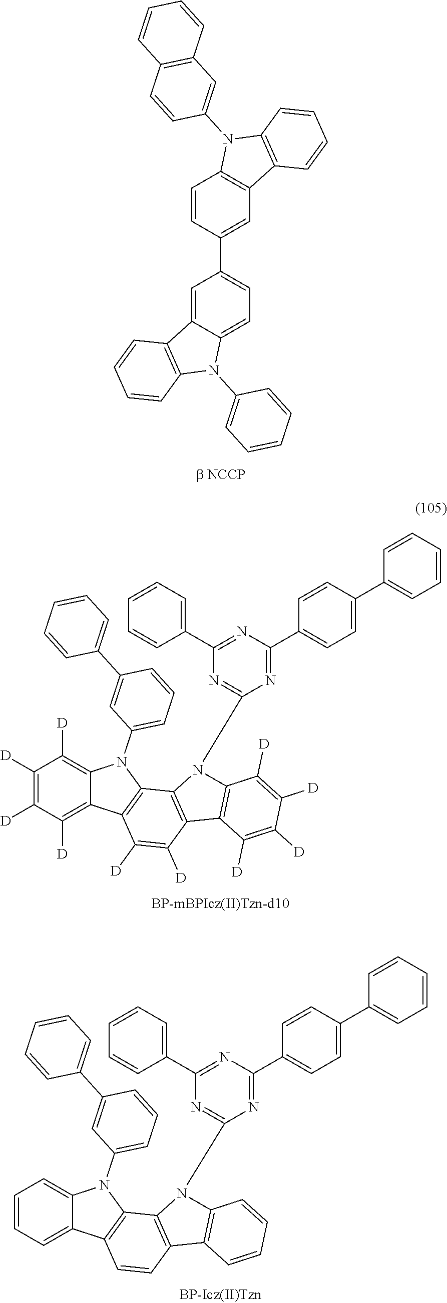 [SMILES: c1ccc(-n2c3ccccc3c3cc(-c4ccc5c(c4)c4ccccc4n5-c4ccc5ccccc5c4)ccc32)cc1 ; [2H]c1c([2H])c([2H])c2c(c1[2H])c1c([2H])c([2H])c3c4c([2H])c([2H])c([2H])c([2H])c4n(-c4nc(-c5ccccc5)nc(-c5ccc(-c6ccccc6)cc5)n4)c3c1n2-c1cccc(-c2ccccc2)c1 ; c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1]

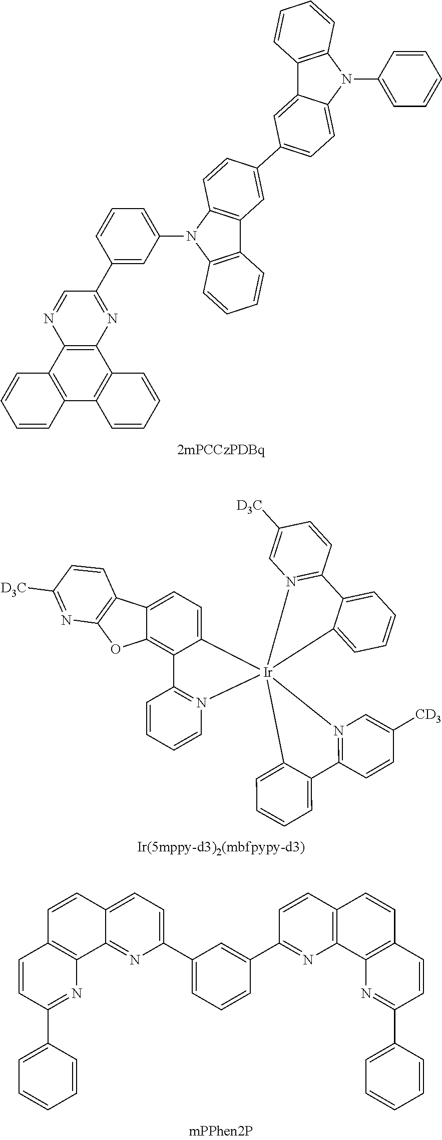

### (Fabrication Method of Light-Emitting Device 3)

A light-emitting device 3 has a structure in which BP-Icz(II)Tzn-d10 (Structural Formula (105)) used for the light-emitting layer 913 in the light-emitting device 1 described in Example 3 is replaced with BP-mBPIcz(II)Tzn-d10 of one embodiment of the present invention whose synthesis method is described in Example 2, NBPhen used for the electron-transport layer 914 is replaced with 2,2′-(1,3-phenylene)bis[9-phenyl-1,10-phenanthroline](abbreviation: mPPhen2P), and the thickness of the electron-transport layer 914 is set to 20 nm. Specifically, the light-emitting device 3 was fabricated in a manner similar to that for the light-emitting device 1 except that the light-emitting layer 913 was formed to a thickness of 40 nm over the hole-transport layer 912 by co-evaporation of BP-mBPIcz(II)Tzn-d10, βNCCP, and Ir(5mppy-d3)₂(mbfpypy-d3) at a weight ratio of 0.5:0.5:0.1 (=BP-Icz(II)Tzn: βNCCP: Ir(5mppy-d3)₂(mbfpypy-d3)) and that 2mPCCzPDBq was deposited to a thickness of 10 nm over the light-emitting layer 913 and then mPPhen2P was deposited to a thickness of 20 nm to form the electron-transport layer 914.

### (Fabrication Method of Comparative Light-Emitting Device 4)

A light-emitting device 4 has a structure in which BP-mBPIcz(II)Tzn-d10 used for the light-emitting layer 913 in the light-emitting device 3 is replaced with 11-[4-(biphenylyl-4-yl)-6-phenyl-1,3,5-triazine-2-yl]-11,12-dihydro-12-(biphenylyl-3-yl)-indolo[2,3-a]carbazole (abbreviation: BP-mBPIcz(II)Tzn). Specifically, the light-emitting device 4 was fabricated in a manner similar to that for the light-emitting device 3 except that the light-emitting layer 913 was formed to a thickness of 40 nm over the hole-transport layer 912 by co-evaporation of BP-mBPIcz(II)Tzn, βNCCP, and Ir(5mppy-d3)₂(mbfpypy-d3) at a weight ratio of 0.5:0.5:0.1 (=BP-PIcz(II)Tzn: βNCCP: Ir(5mppy-d3)₂(mbfpypy-d3)).

The element structures of the light-emitting device 3 and the comparative light-emitting device 4 are listed in the following table.

TABLE 4

| | | | Comparative |
|---|---|---|---|
| | Film | Light-emitting | light-emitting |
| | thickness | device 3 | device 4 |
| Second electrode | 200 nm | Al | |
| Electron-injection | 1 nm | LiF | |
| layer | | | |
| Electron-transport | 20 nm | mPPhen2P | |
| layer | 10 nm | 2mPCCzPDBq | |
| Light-emitting | 40 nm | BP-mBPIcz(II)Tzn- | BP-mBPIcz(II)Tzn:βNCCP:Ir(5mppy- |
| layer | | d10:βNCCP:Ir(5mppy- | d3)₂(mbfpypy-d3) |
| | | d3)₂(mbfpypy-d3) | (0.5:0.5:0.1) |
| | | (0.5:0.5:0.1) | |

| | | |
|---|---|---|
| Hole-transport | 10 nm | PCBBilBP |
| layer | 40 nm | PCBBiF |
| Hole-injection | 10 nm | PCBBiF:OCHD-003 |
| layer | | (1:0.03) |
| First electrode | 70 nm | ITSO |

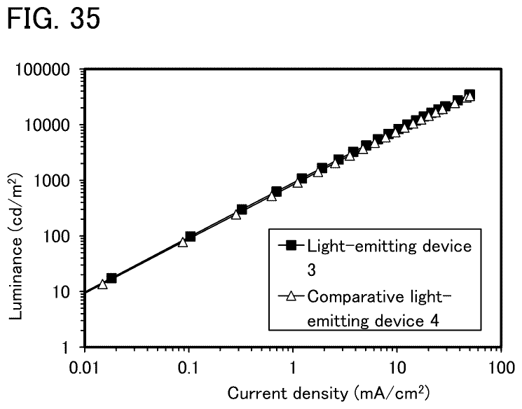

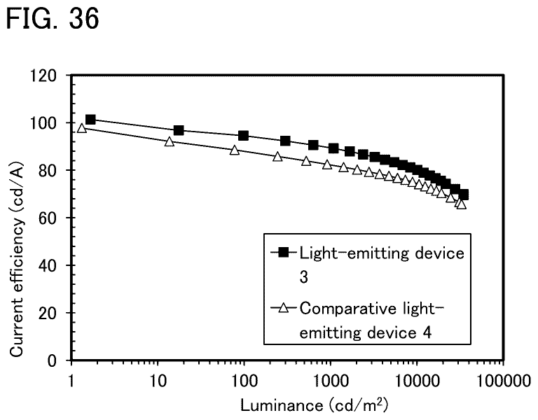

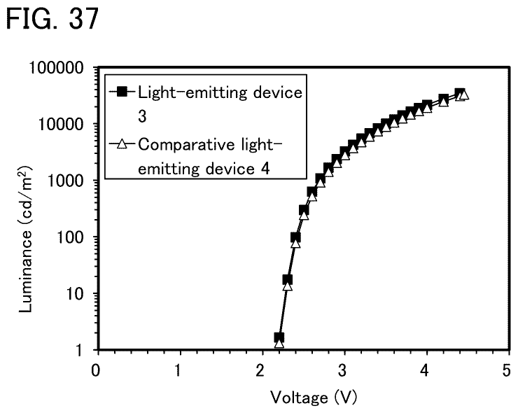

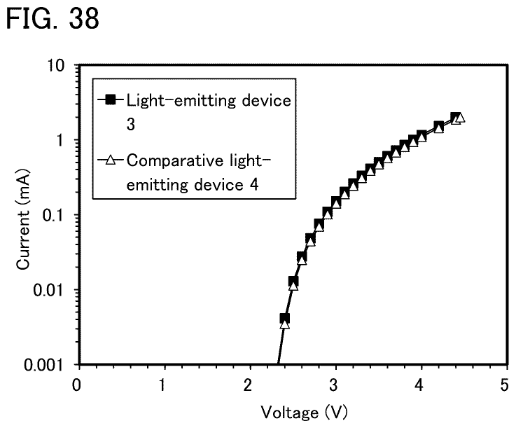

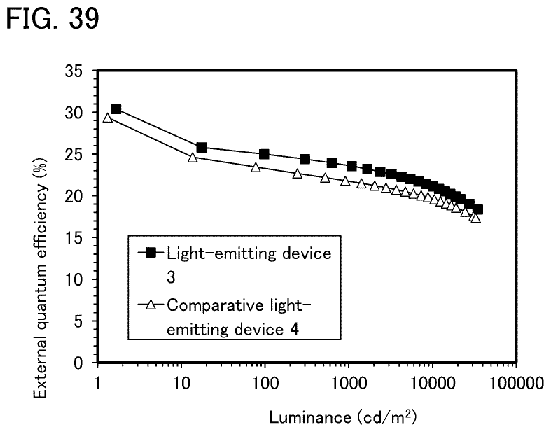

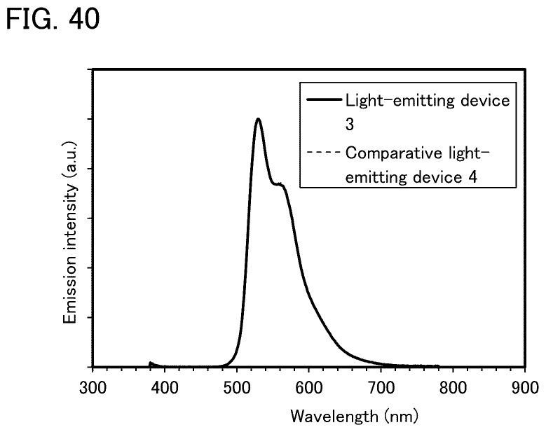

The light-emitting device 3 and the comparative light-emitting device 4 were sealed with a glass substrate in a manner similar to that of the light-emitting device 1 and the comparative light-emitting device 3, and then the initial characteristics of these light-emitting devices were measured. FIG. 35 shows the luminance-current density characteristics of the light-emitting device 3 and the comparative light-emitting device 4; FIG. 36 shows the current efficiency-luminance characteristics thereof; FIG. 37 shows the luminance-voltage characteristics thereof, FIG. 38 shows the current-voltage characteristics thereof, FIG. 39 shows the external quantum efficiency-luminance characteristics thereof, and FIG. 40 shows the emission spectra thereof. Table 2 shows main characteristics of the light-emitting devices at approximately 1000 cd/m². Note that the luminance, CIE chromaticity, and emission spectra were measured using a spectroradiometer (SR-UL1R, produced by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 5

| | | | Current | | | Current | External |
|---|---|---|---|---|---|---|---|
| | Voltage | Current | density | | | efficiency | quantum |
| | (V) | (mA) | (mA/cm²) | Chromaticity x | Chromaticity y | (cd/A) | efficiency (%) |
| Light-emitting | 2.7 | 0.049 | 1.22 | 0.371 | 0.607 | 89.1 | 23.6 |
| device 3 | | | | | | | |
| Comparative | 2.7 | 0.044 | 1.11 | 0.371 | 0.607 | 82.4 | 21.8 |
| light-emitting | | | | | | | |
| device 4 | | | | | | | |

It was found from FIG. 35 to FIG. 40 that the light-emitting device 3, which is the light-emitting device of one embodiment of the present invention, has higher emission efficiency than the comparative light-emitting device 4.

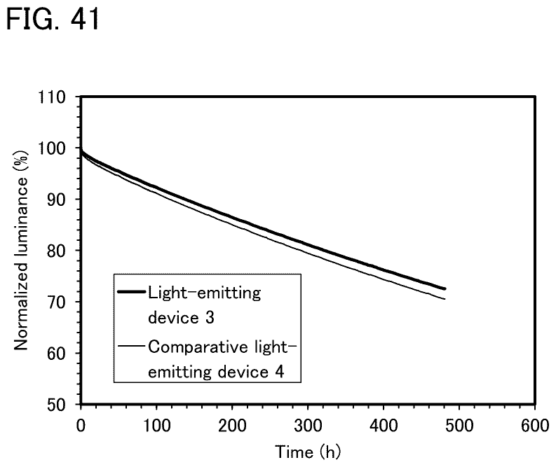

FIG. 41 shows changes in luminance over driving time when the light-emitting device 1 and the comparative light-emitting device 2 were driven at a constant current of 2 mA (50 mA/cm²). As shown in FIG. 41, the light-emitting device 3 had a longer lifetime than the comparative light-emitting device 4.

From the above results, it was found that the emission efficiency of the light-emitting device including BP-Icz(II)Tzn-d10, which is the organic compound of one embodiment of the present invention, is improved and the lifetime thereof is prolonged as compared with those of the light-emitting device including BP-mBPIcz(II)Tzn-d10. That is, it was found that deuteration of an indolocarbazole skeleton included in BP-mBPIcz(II)Tzn can prevent dissociation of a carbon-hydrogen bond, whereby a light-emitting device including the organic compound can have a longer lifetime.

### REFERENCE NUMERALS

GD: circuit, IR: subpixel, M11: transistor, M12: transistor, M13: transistor, M14: transistor, M15: transistor, M16: transistor, M17: transistor, MS: wiring, PS: subpixel, REG: resist mask, RES: wiring, SE1: wiring, SE: distance, Si: single crystal, TX: wiring, VG: wiring, VS: wiring, 100: light-emitting device, 101: first electrode, 102: second electrode, 103: EL layer, 103a: EL layer, 103b: EL layer, 103B: EL layer, 103G: EL layer, 103R: EL layer, 103PS: light-receiving layer, 104B: hole-injection/transport layer, 104G: hole-injection/transport layer, 104R: hole-injection/transport layer, 104PS: first transport layer, 105B: light-emitting layer, 105G: light-emitting layer, 105R: light-emitting layer, 105PS: active layer, 106: charge-generation layer, 106a: charge-generation layer, 106b: charge-generation layer, 107: insulating layer, 108B: electron-transport layer, 108G: electron-transport layer, 108R: electron-transport layer, 108PS: second transport layer, 109: electron-injection layer, 110B: sacrificial layer, 110G: sacrificial layer, 110R: sacrificial layer, 110PS: sacrificial layer, 111: hole-injection layer, 111a: hole-injection layer, 111b: hole-injection layer, 112: hole-transport layer, 112a: hole-transport layer, 112b: hole-transport layer, 113: light-emitting layer, 113a: light-emitting layer, 113b: light-emitting layer, 113c: light-emitting layer, 114: electron-transport layer, 114a: electron-transport layer, 114b: electron-transport layer, 115: electron-injection layer, 115a: electron-injection layer, 115b: electron-injection layer, 130: connection portion, 400: substrate, 401: first electrode, 403: EL layer, 404: second electrode, 405: sealing material, 406: sealing material, 407: sealing substrate, 412: pad, 420: IC chip, 501C: insulating film, 501D: insulating film, 504: conductive film, 506: insulating film, 508: semiconductor film, 508A: region, 508B: region, 508C: region, 510: first substrate, 512A: conductive film, 512B: conductive film, 516: insulating film, 516A: insulating film, 516B: insulating film, 518: insulating film, 520: functional layer, 524: conductive film, 528: partition wall, 530: pixel circuit, 530S: pixel circuit, 530X: pixel circuit, 531: pixel circuit, 550: light-emitting device, 550B: light-emitting device, 550G: light-emitting device, 550R: light-emitting device, 550PS: light-receiving device, 550X: light-emitting device, 550S: light-receiving device, 551B: electrode, 551C: connection electrode, 551G: electrode, 551R: electrode, 551PS: electrode, 552: electrode, 580: space, 591X: wiring, 591S: wiring, 700: light-emitting and light-receiving apparatus, 701: display region, 702B: subpixel, 702G: subpixel, 702PS: subpixel, 702R: subpixel, 702IR: subpixel, 703: pixel, 704: circuit, 705: insulating layer, 706: wiring, 710: substrate, 711: substrate, 712: IC, 713: FPC, 720: device, 770: substrate, 800: substrate, 801a: electrode, 801b: electrode, 802: electrode, 803a: EL layer, 803b: light-receiving layer, 805a: light-emitting device, 805b: light-receiving device, 810: light-emitting and light-receiving apparatus, 810A: light-emitting and light-receiving apparatus, 810B: light-emitting and light-receiving apparatus, 900: substrate, 901: first electrode, 902: second electrode, 911: hole-injection layer, 912: hole-transport layer, 913: light-emitting layer, 914: electron-transport layer, 915: electron-injection layer, 5200B: electronic apparatus, 5210: arithmetic device, 5220: input/output device, 5230: display portion, 5240: input portion, 5250: detecting portion, 5290: communication portion, 8001: ceiling light, 8002: foot light, 8003: sheet-like lighting, 8004: lighting device, 8005: desk lamp, 8006: light source

1. An organic compound represented by General Formula (G1): 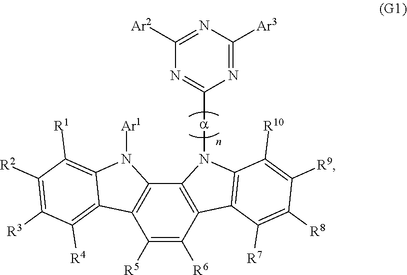
wherein:
in the General Formula (G1), each of R¹ to R¹⁰ independently represents any one of hydrogen, deuterium, a substituted or unsubstituted alkyl group having 1 to 6 carbon atoms, a substituted or unsubstituted aryl group having 6 to 30 carbon atoms, and a substituted or unsubstituted heteroaryl group having 2 to 30 carbon atoms,
at least one of R¹ to R¹⁰ represents deuterium,
each of Ar¹ to Ar³ independently represents any one of a substituted or unsubstituted aryl group having 6 to 30 carbon atoms and a substituted or unsubstituted heteroaryl group having 2 to 30 carbon atoms, and
α represents any one of a substituted or unsubstituted arylene group having 6 to 30 carbon atoms and a substituted or unsubstituted heteroarylene group having 2 to 30 carbon atoms;
n represents an integer of 0 to 2.

2. The organic compound according to claim 1, wherein n represents 1 or 2.

3. The organic compound according to claim 1,
wherein the arylene group having 6 to 30 carbon atoms and the heteroarylene group having 2 to 30 carbon atoms are represented by any one of Formulae (α-1) to (α-20):

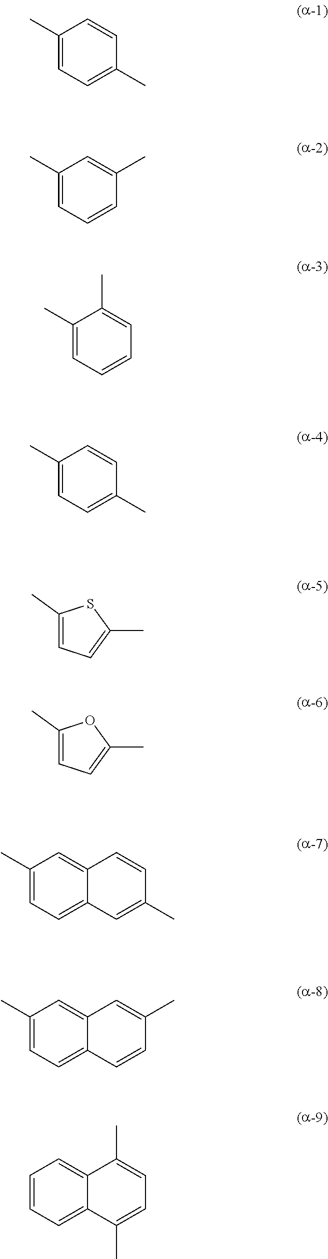

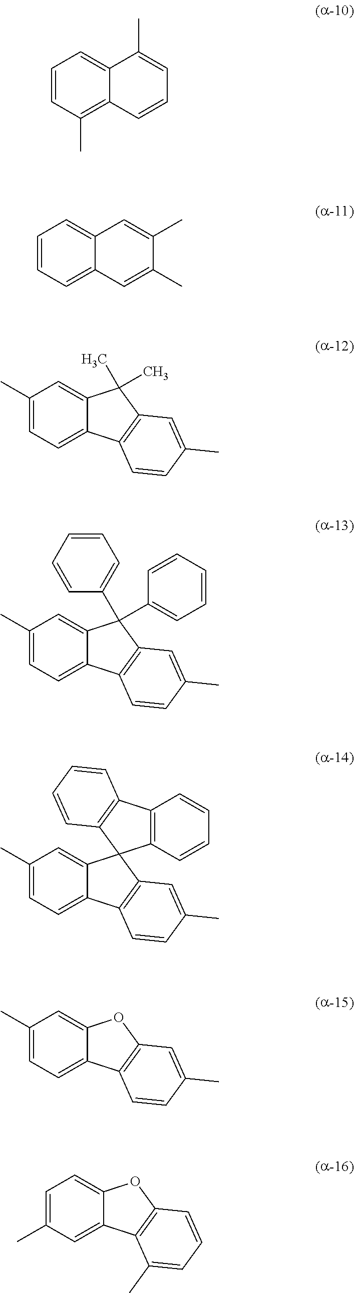

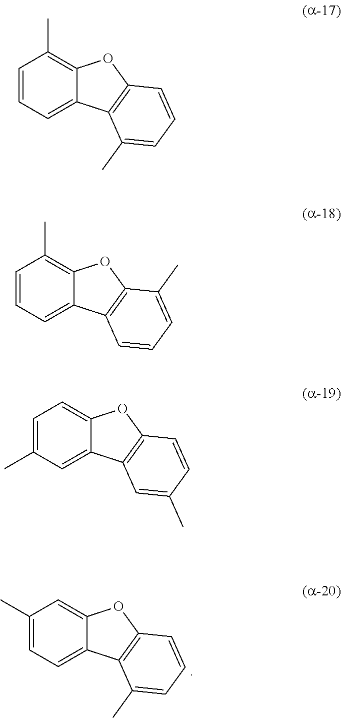

4. An organic compound represented by General Formula (G3): 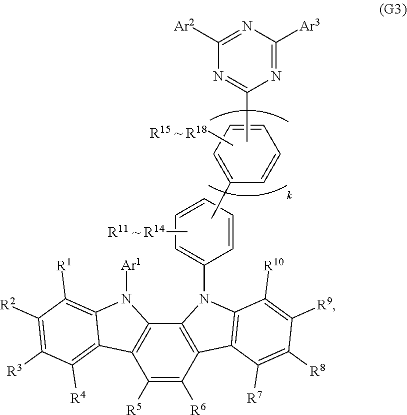
wherein:
in the General Formula (G3), each of R¹ to R¹⁰ independently represents any one of hydrogen, deuterium, a substituted or unsubstituted alkyl group having 1 to 6 carbon atoms, a substituted or unsubstituted aryl group having 6 to 30 carbon atoms, and a substituted or unsubstituted heteroaryl group having 2 to 30 carbon atoms,
at least one of R¹ to R¹⁰ represents deuterium,
each of Ar¹ to Ar³ independently represents a substituted or unsubstituted aryl group having 6 to 30 carbon atoms or a substituted or unsubstituted heteroaryl group having 2 to 30 carbon atoms;
each of R¹¹ to R¹⁸ independently represents any one of hydrogen, a substituted or unsubstituted alkyl group having 1 to 6 carbon atoms, a substituted or unsubstituted aryl group having 6 to 30 carbon atoms, and a substituted or unsubstituted heteroaryl group having 2 to 30 carbon atoms, and
k represents 0 or 1.

5. An organic compound represented by General Formula (G4):
[Chemical Formula 5] 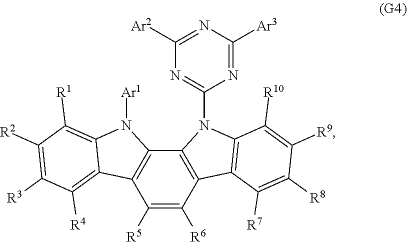
wherein:
in the General Formula (G4), each of R¹ to R¹⁰ independently represents any one of hydrogen, deuterium, a substituted or unsubstituted alkyl group having 1 to 6 carbon atoms, a substituted or unsubstituted aryl group having 6 to 30 carbon atoms, and a substituted or unsubstituted heteroaryl group having 2 to 30 carbon atoms,
at least one of R¹ to R¹⁰ represents deuterium, and
each of Ar¹ to Ar³ independently represents any one of a substituted or unsubstituted aryl group having 6 to 30 carbon atoms and a substituted or unsubstituted heteroaryl group having 2 to 30 carbon atoms.

6. An organic compound represented by General Formula (G5): 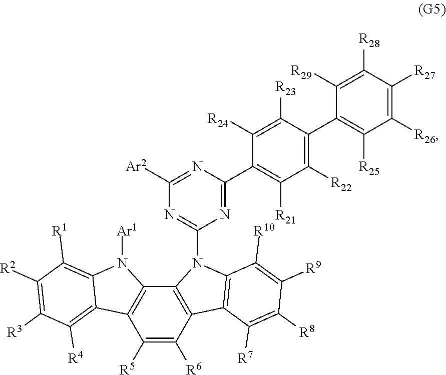
wherein:
in the General Formula (G5), each of R¹ to R¹⁰ independently represents any one of hydrogen, deuterium, a substituted or unsubstituted alkyl group having 1 to 6 carbon atoms, a substituted or unsubstituted aryl group having 6 to 30 carbon atoms, and a substituted or unsubstituted heteroaryl group having 2 to 30 carbon atoms,
at least one of R¹ to R¹⁰ represents deuterium,
each of Ar¹ and Ar² independently represents any one of a substituted or unsubstituted aryl group having 6 to 30 carbon atoms and a substituted or unsubstituted heteroaryl group having 2 to 30 carbon atoms, and
each of R²¹ to R²⁹ independently represents any one of hydrogen, a substituted or unsubstituted alkyl group having 1 to 6 carbon atoms, a substituted or unsubstituted aryl group having 6 to 30 carbon atoms, and a substituted or unsubstituted heteroaryl group having 2 to 30 carbon atoms.

7. The organic compound according to claim 1,
wherein the aryl group having 6 to 30 carbon atoms and the heteroaryl group having 2 to 30 carbon atoms are represented by any one of Formulae (Ar-1) to (Ar-80):

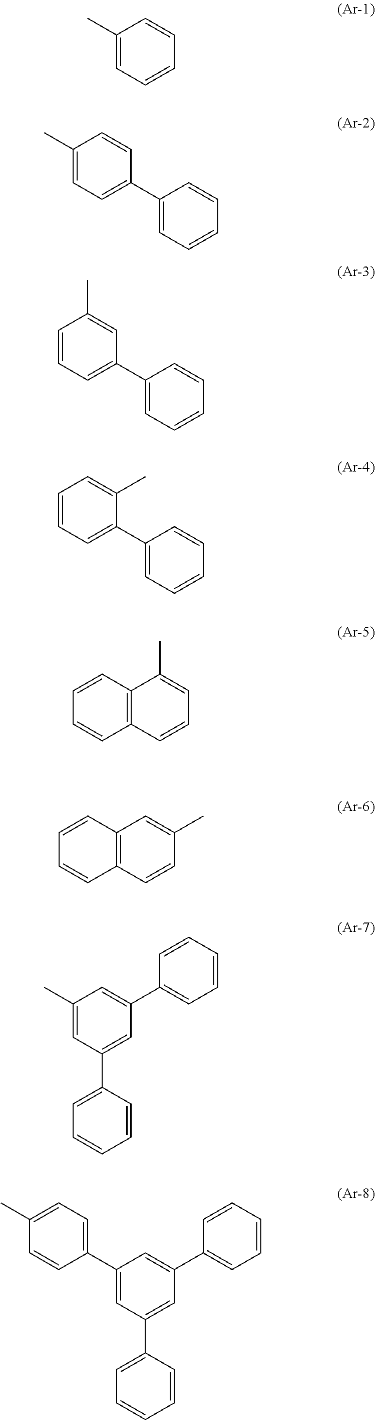

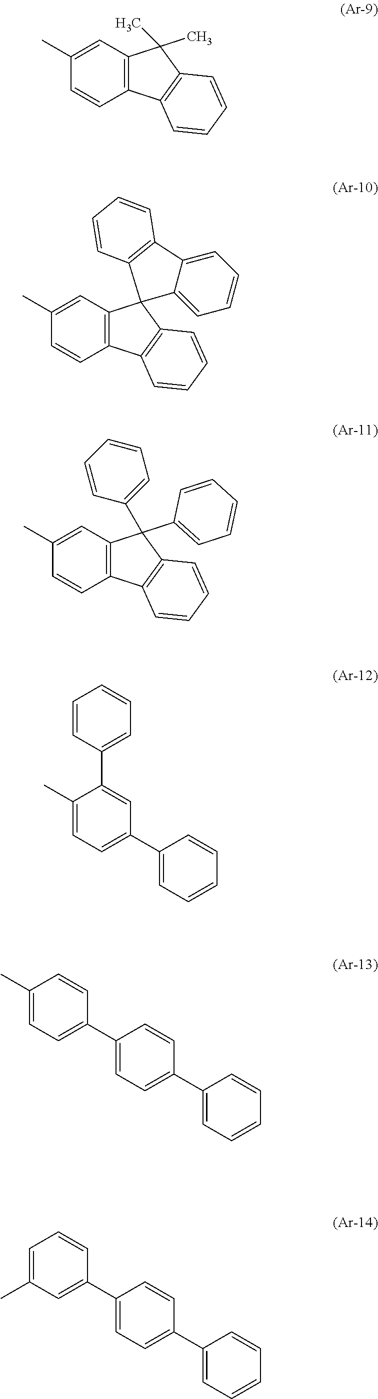

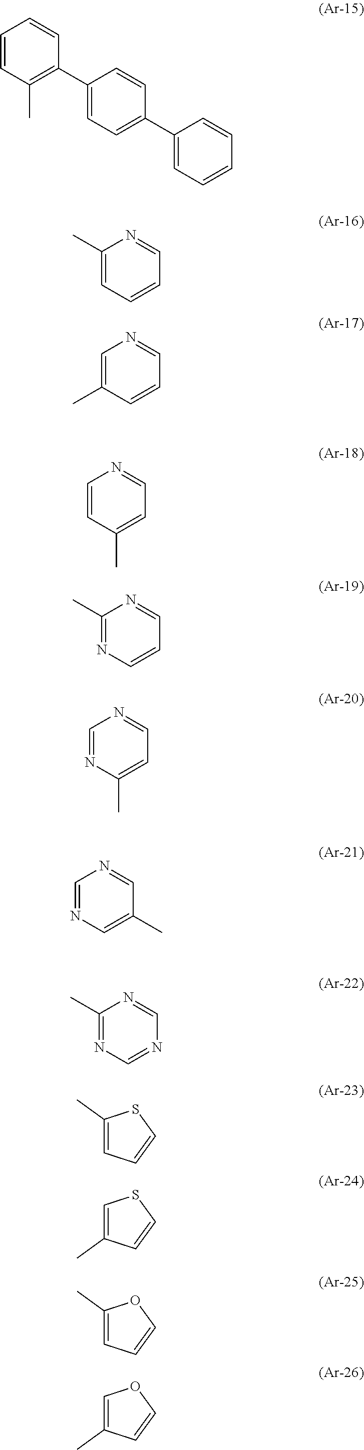

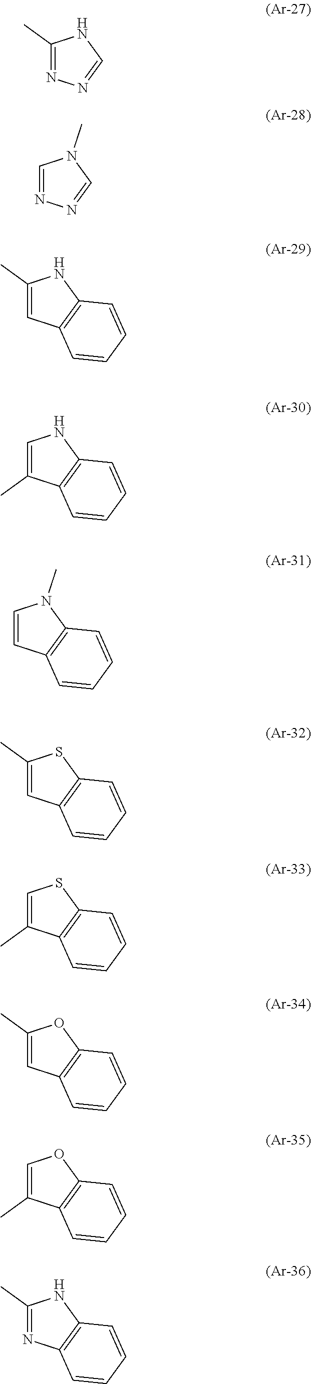

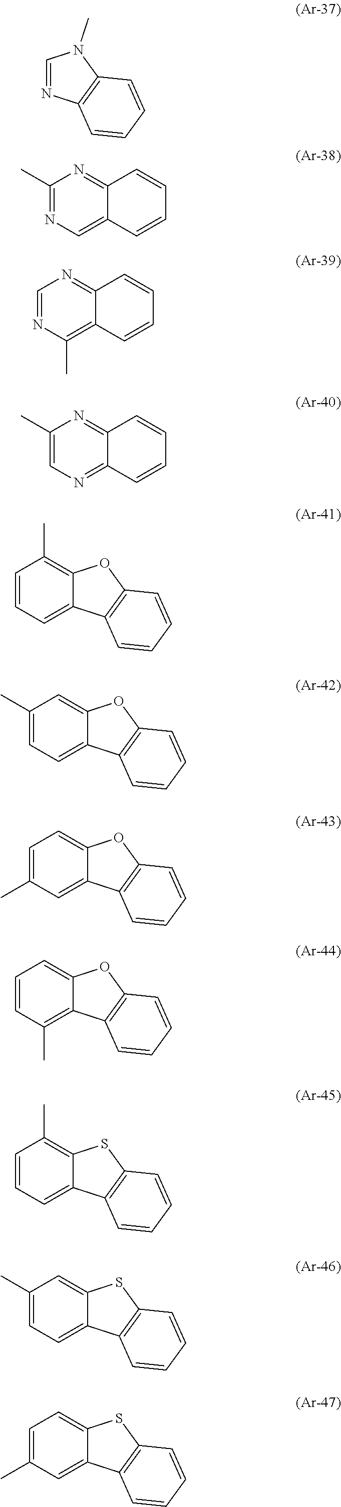

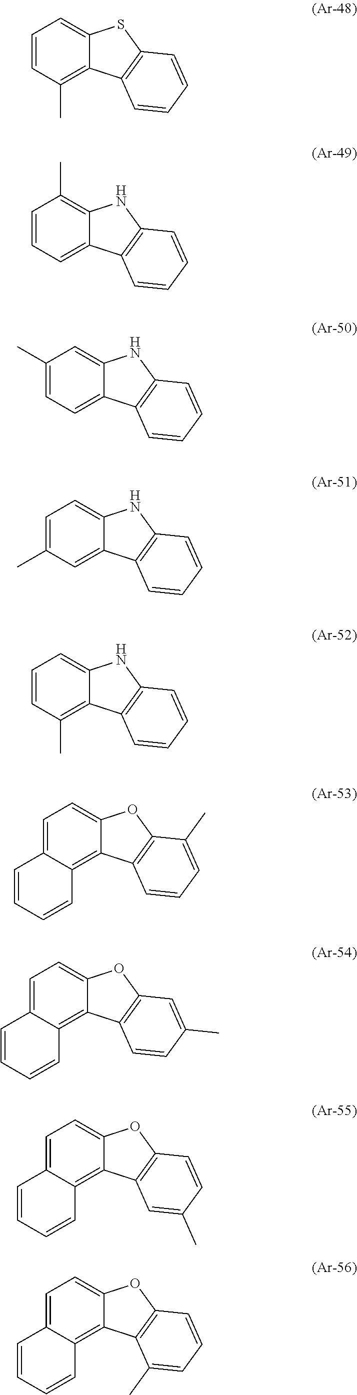

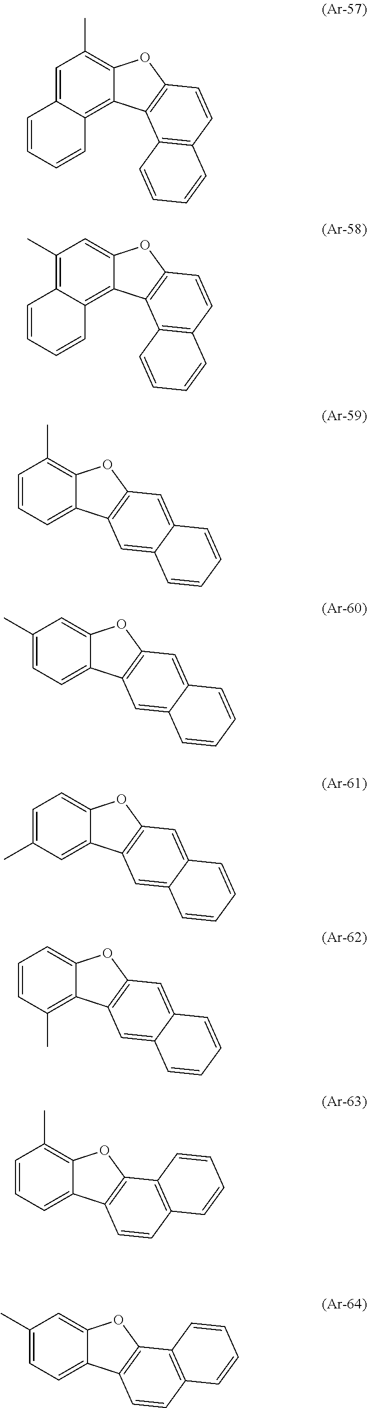

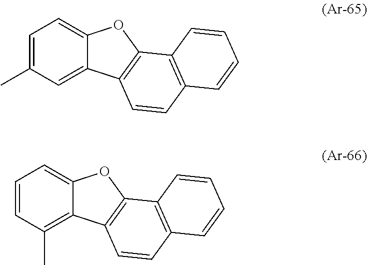 [SMILES: Cc1ccc2oc3c4ccccc4ccc3c2c1 ; Cc1cccc2oc3c4ccccc4ccc3c12]

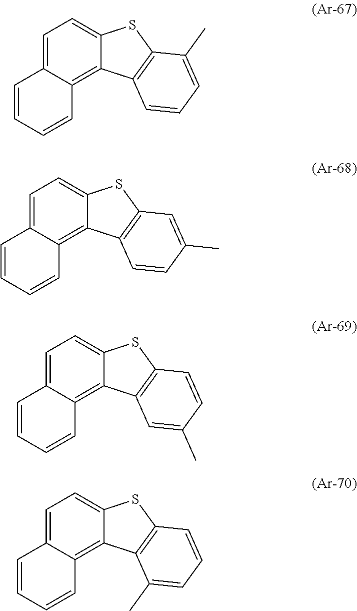

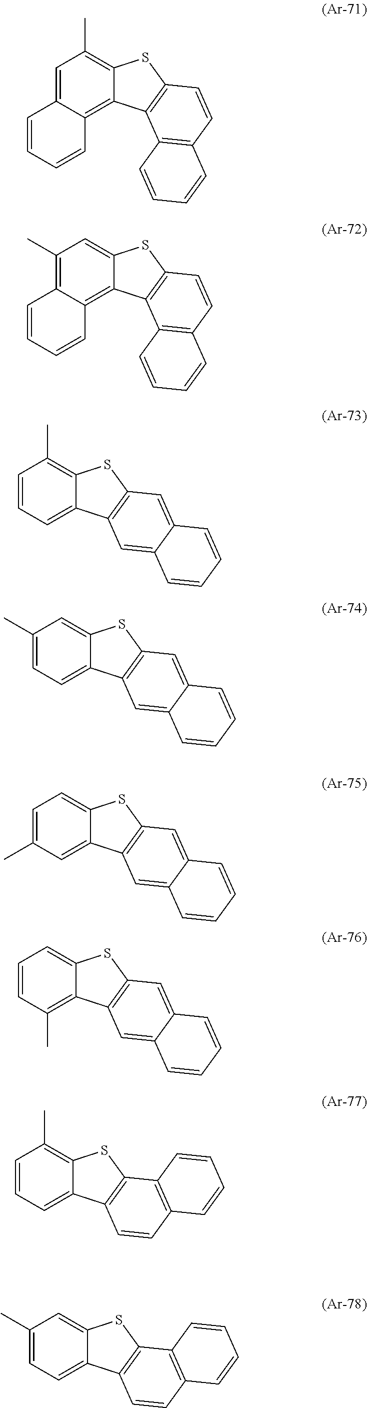

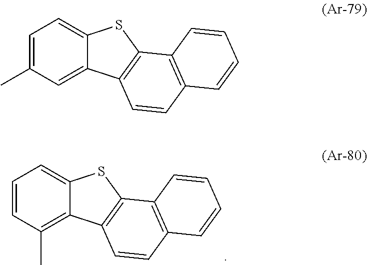 [SMILES: Cc1ccc2sc3c4ccccc4ccc3c2c1 ; Cc1cccc2sc3c4ccccc4ccc3c12]

8. The organic compound according to claim 1, wherein a plurality of or each of R¹ to R¹⁰ represent deuterium.

9. An organic compound represented by any one of Structural Formula (101) and (105):

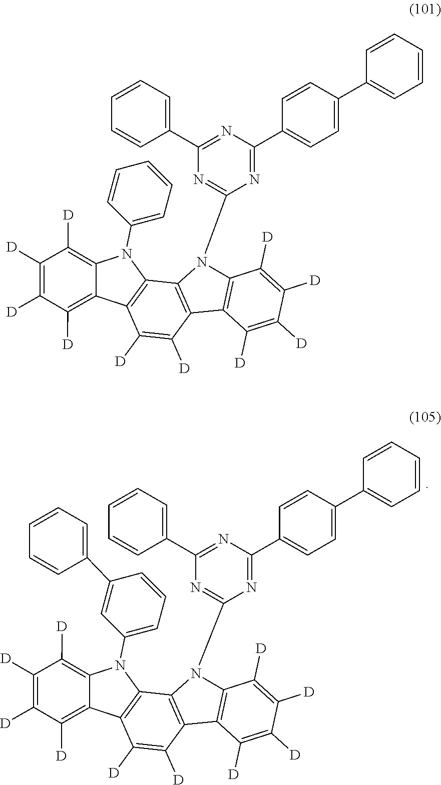 [SMILES: [2H]c1c([2H])c([2H])c2c(c1[2H])c1c([2H])c([2H])c3c4c([2H])c([2H])c([2H])c([2H])c4n(-c4nc(-c5ccccc5)nc(-c5ccc(-c6ccccc6)cc5)n4)c3c1n2-c1ccccc1 ; [2H]c1c([2H])c([2H])c2c(c1[2H])c1c([2H])c([2H])c3c4c([2H])c([2H])c([2H])c([2H])c4n(-c4nc(-c5ccccc5)nc(-c5ccc(-c6ccccc6)cc5)n4)c3c1n2-c1cccc(-c2ccccc2)c1]

10-14. (canceled)

15. The organic compound according to claim 4,
wherein the aryl group having 6 to 30 carbon atoms and the heteroaryl group having 2 to 30 carbon atoms are represented by any one of Formulae (Ar-1) to (Ar-80):

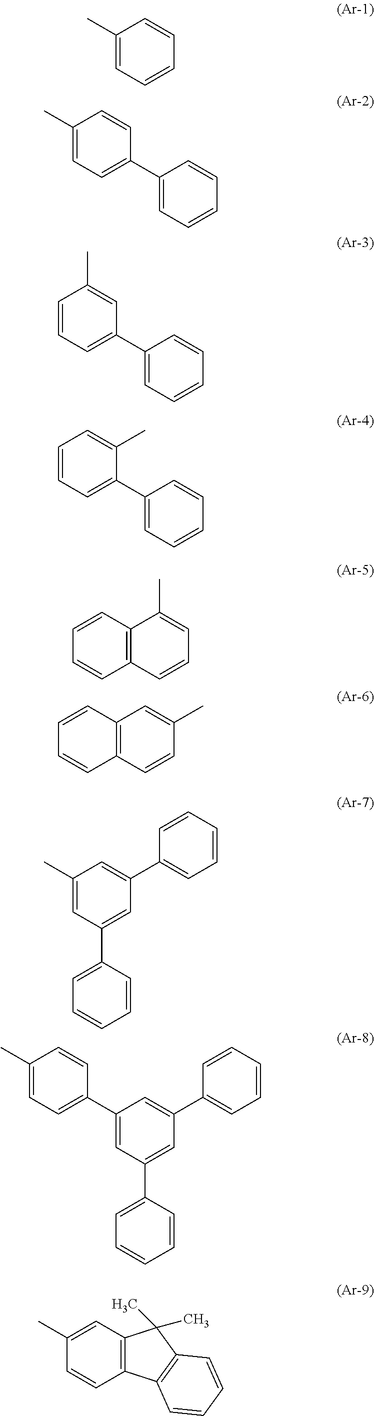

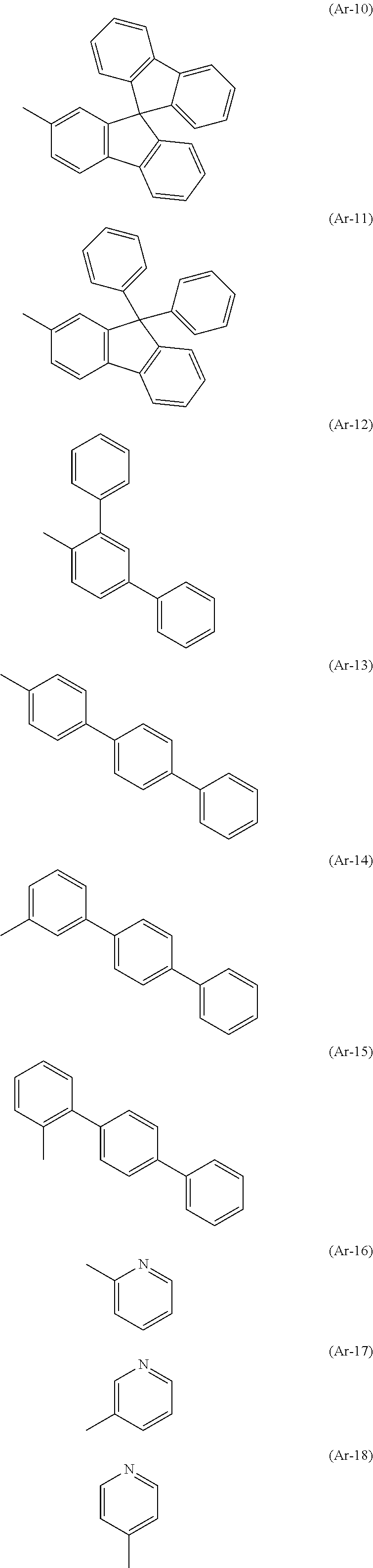

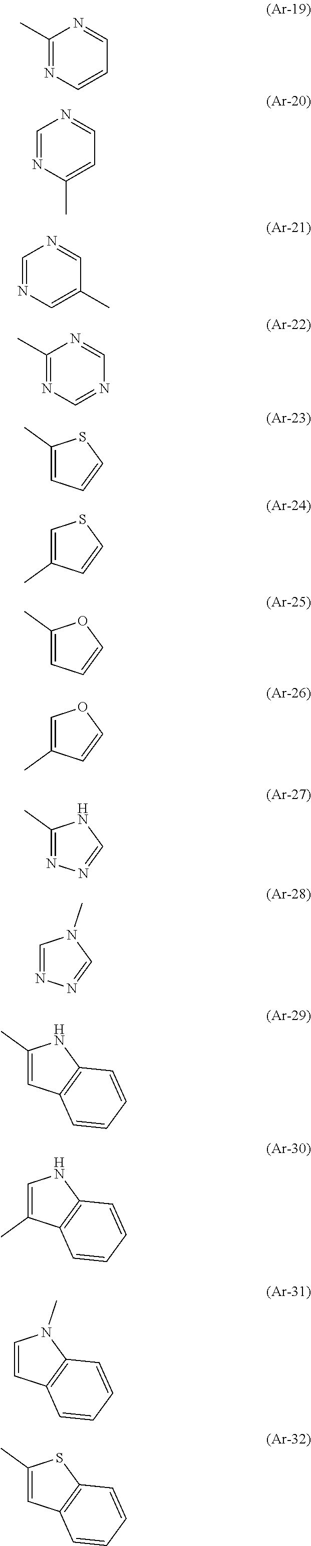

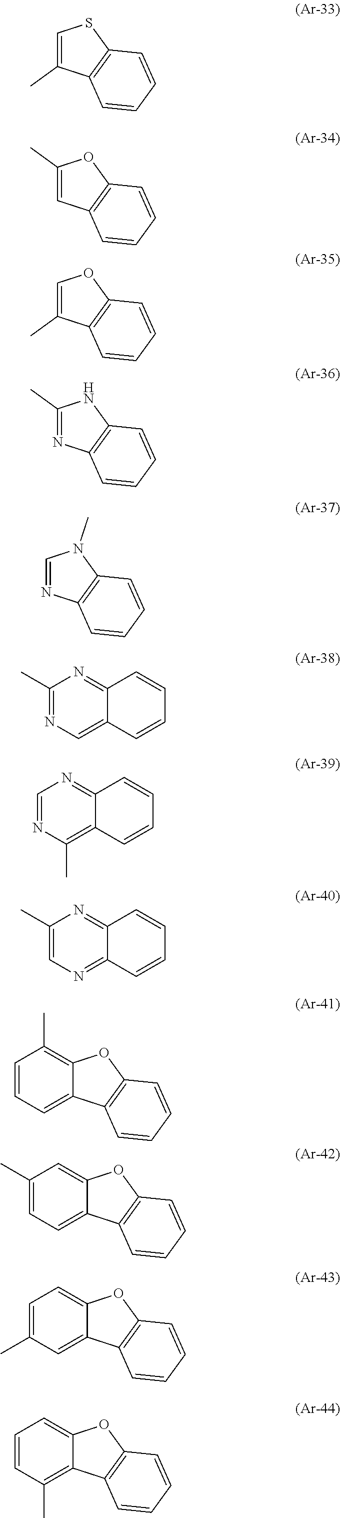

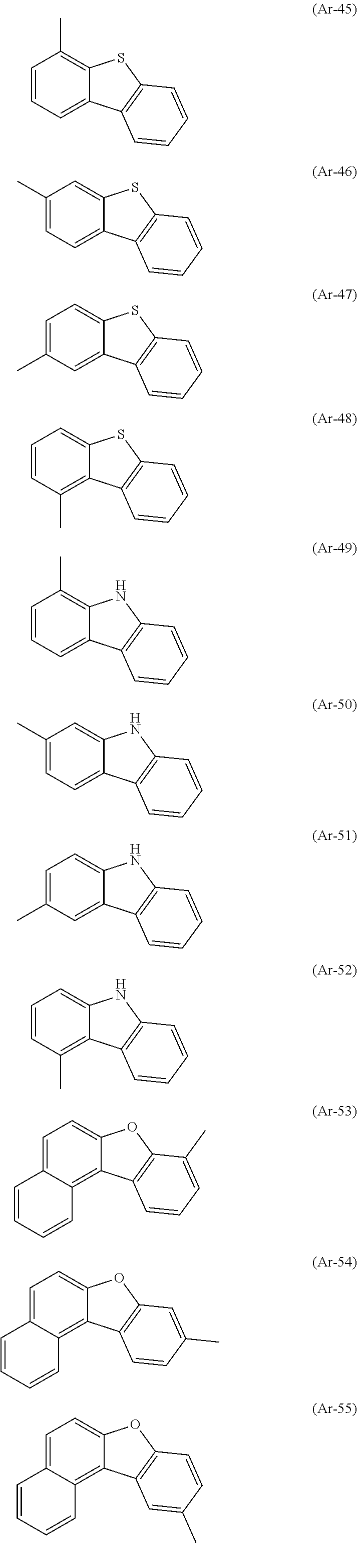

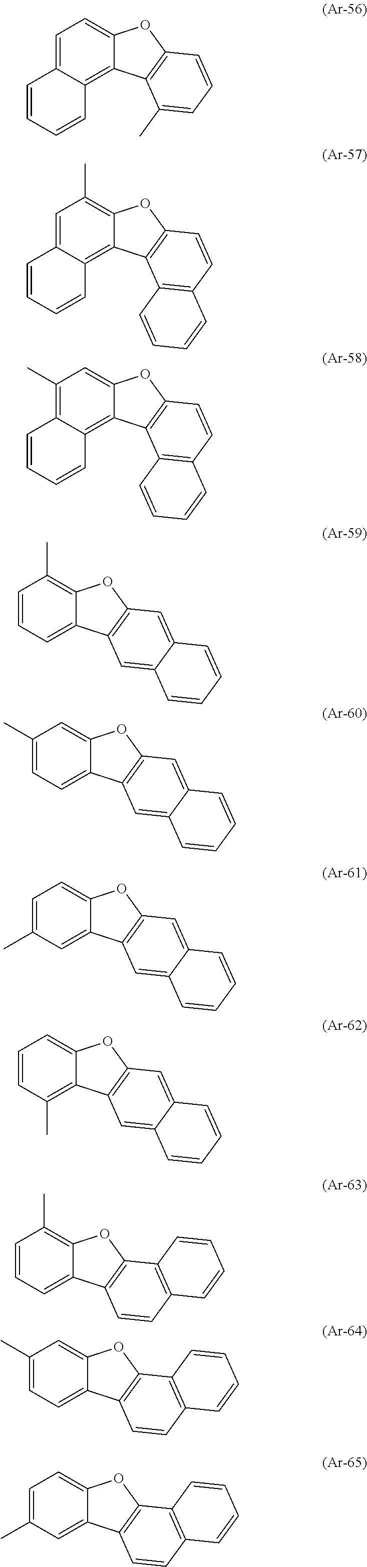

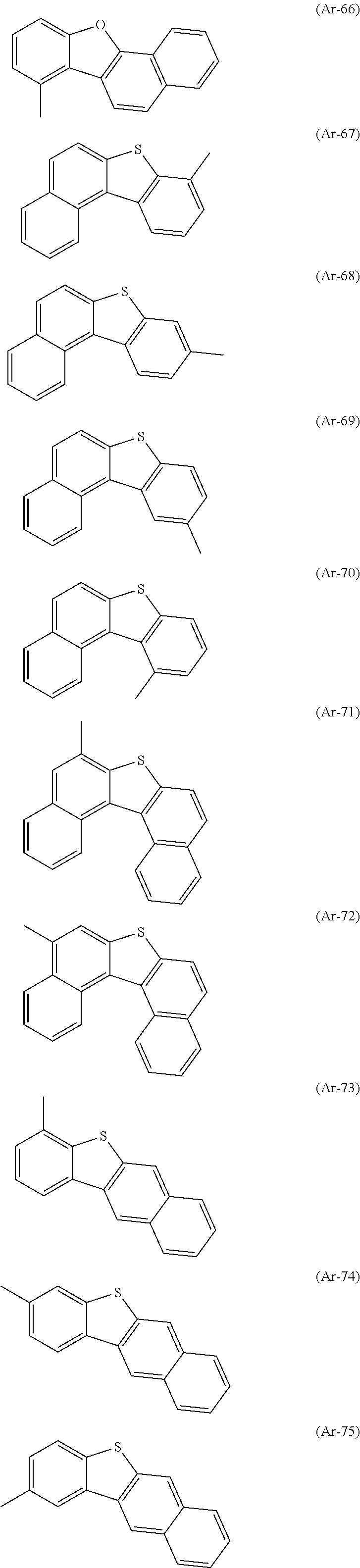

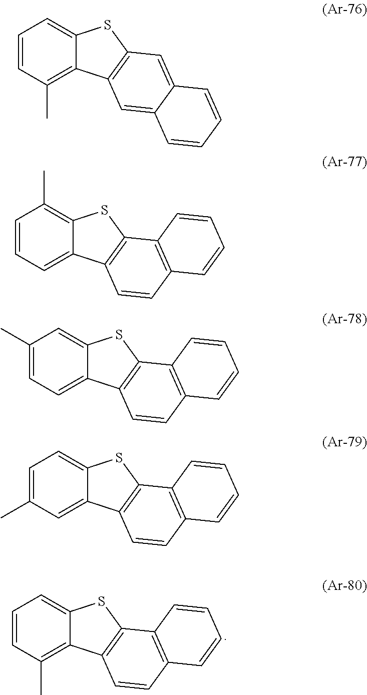

16. The organic compound according to claim 4, wherein a plurality of or each of R¹ to R¹⁰ represent deuterium.

17. The organic compound according to claim 5,
wherein the aryl group having 6 to 30 carbon atoms and the heteroaryl group having 2 to 30 carbon atoms are represented by any one of Formulae (Ar-1) to (Ar-80):

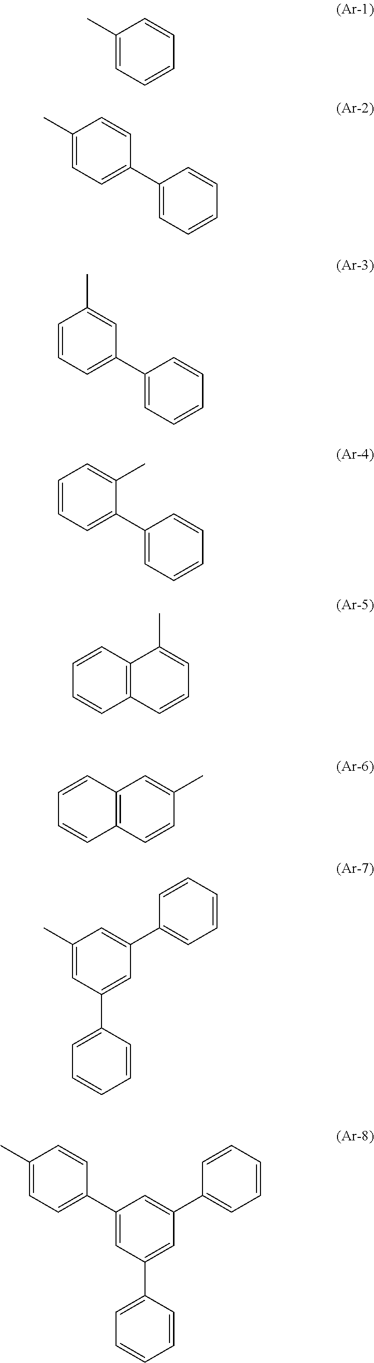

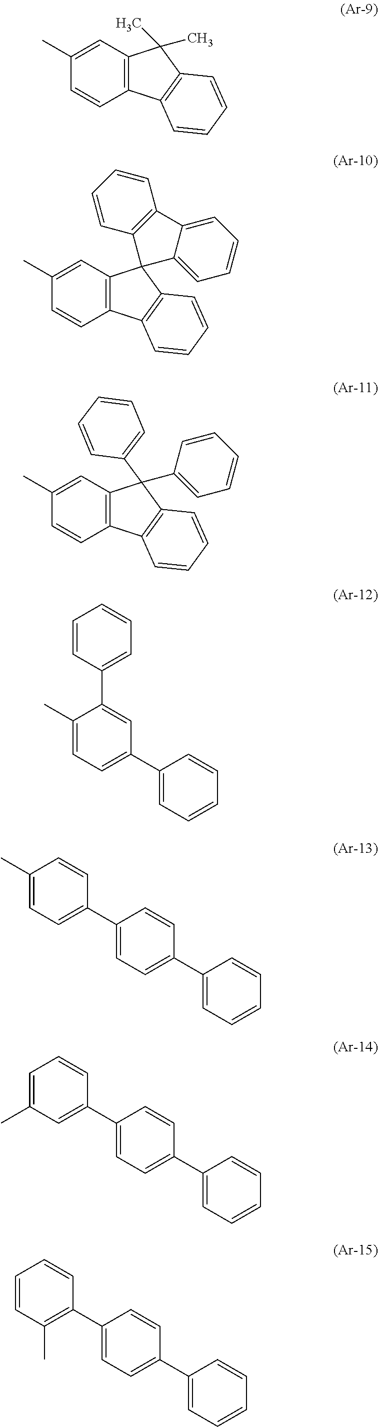

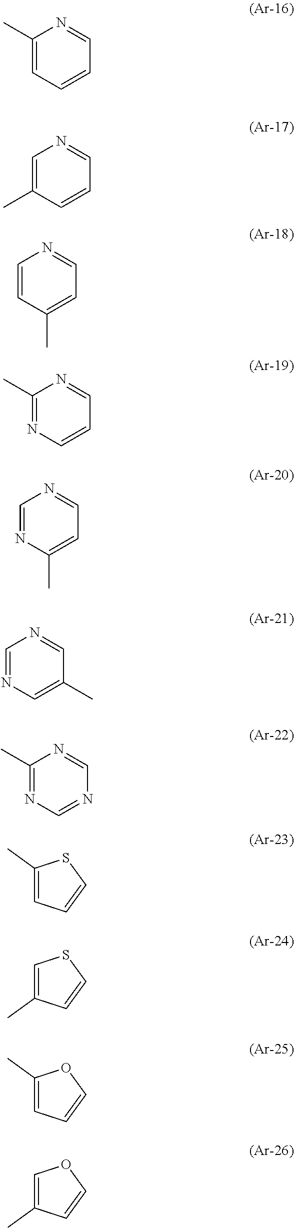

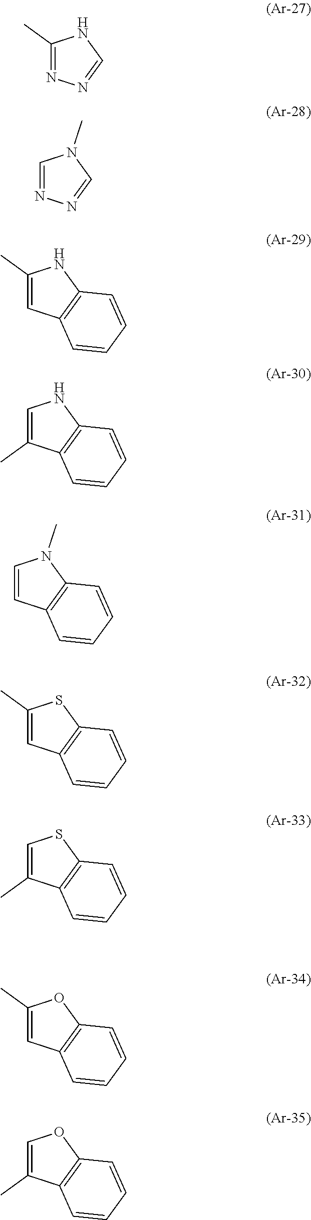

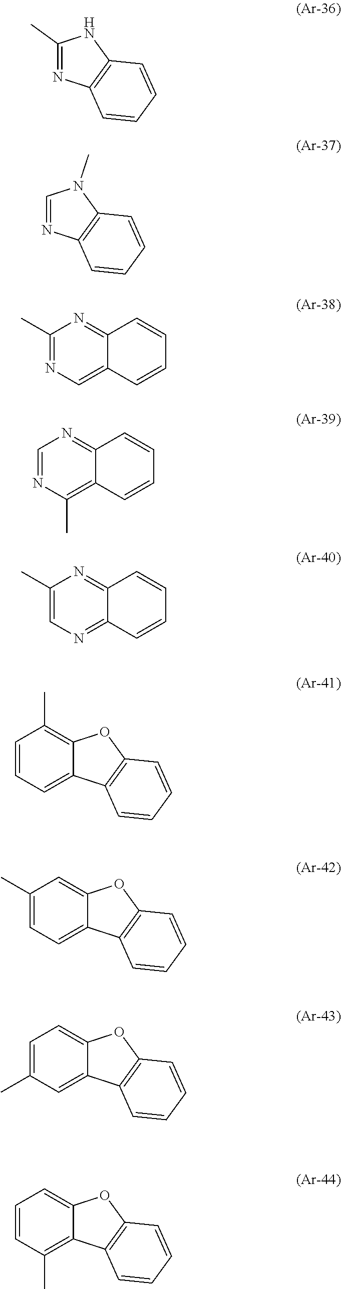

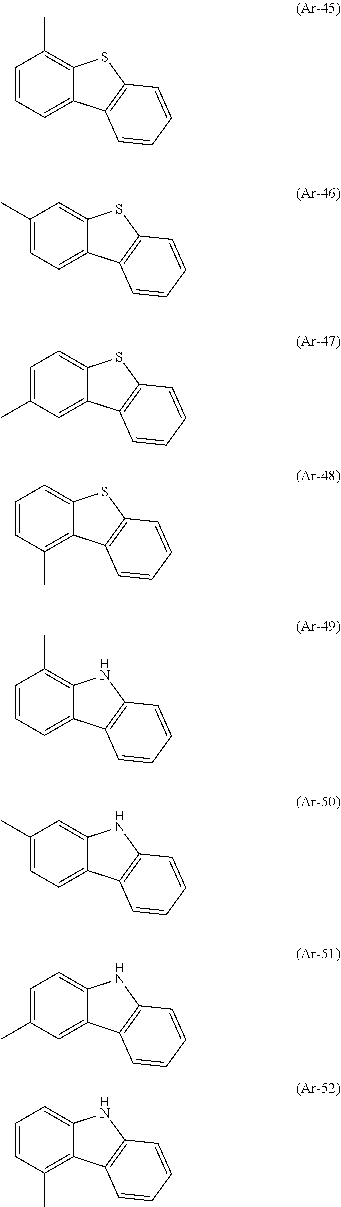

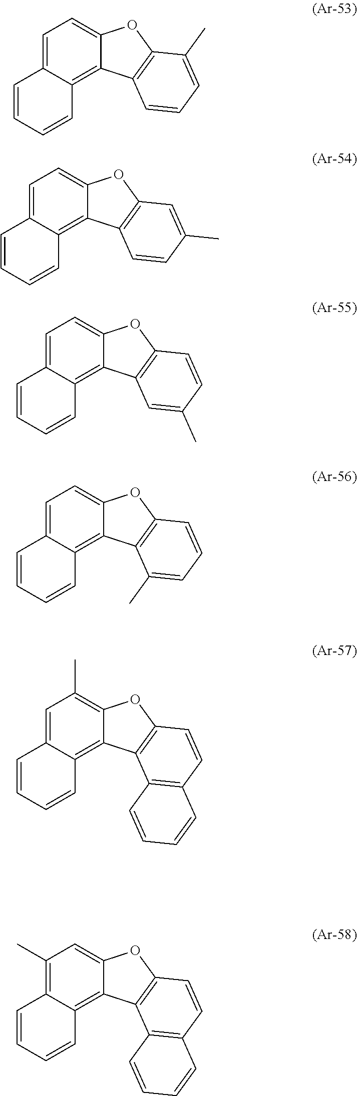

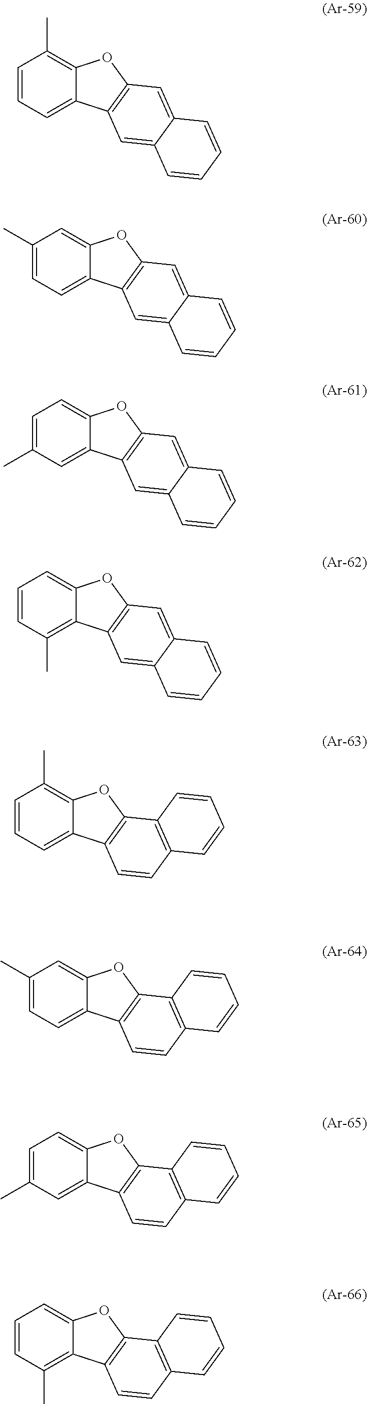

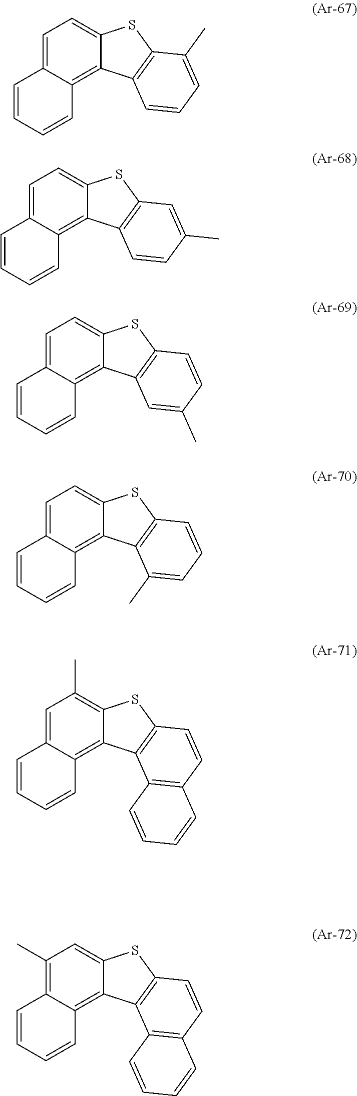

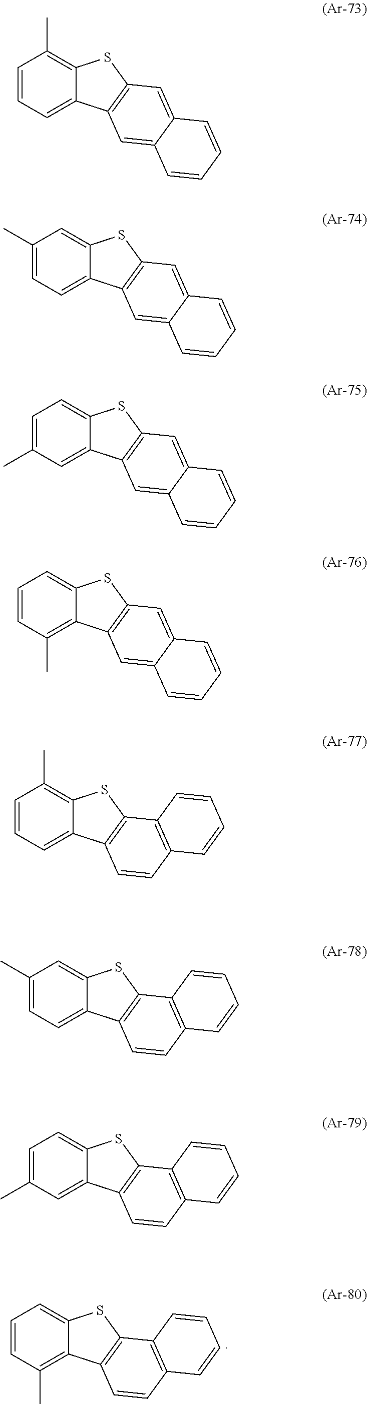

18. The organic compound according to claim 5, wherein a plurality of or each of R¹ to R¹⁰ represent deuterium.

19. The organic compound according to claim 6,
wherein the aryl group having 6 to 30 carbon atoms and the heteroaryl group having 2 to 30 carbon atoms are represented by any one of Formulae (Ar-1) to (Ar-80):

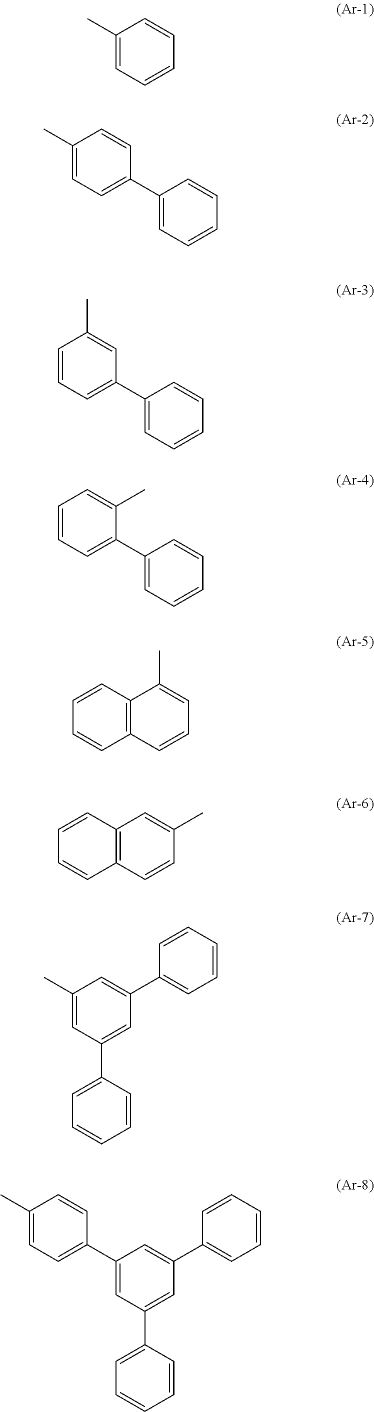

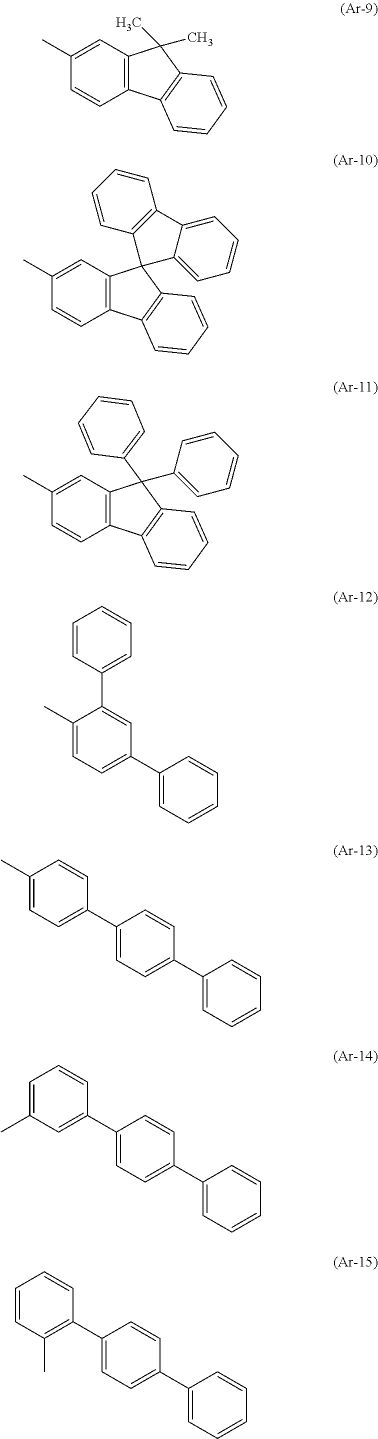

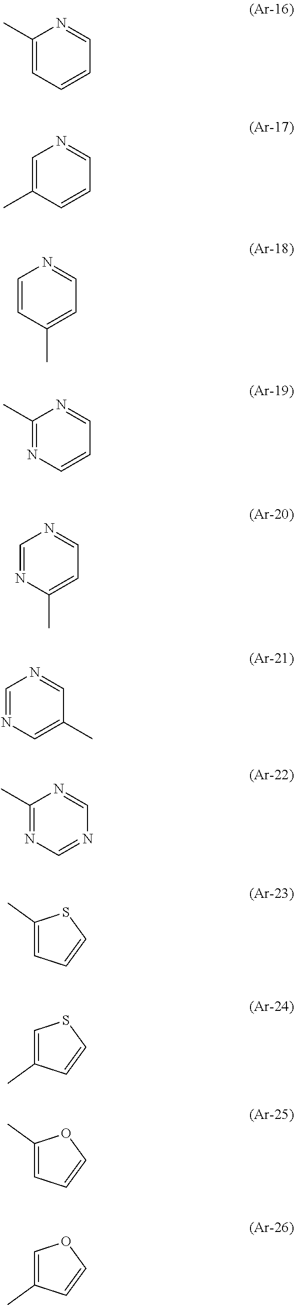

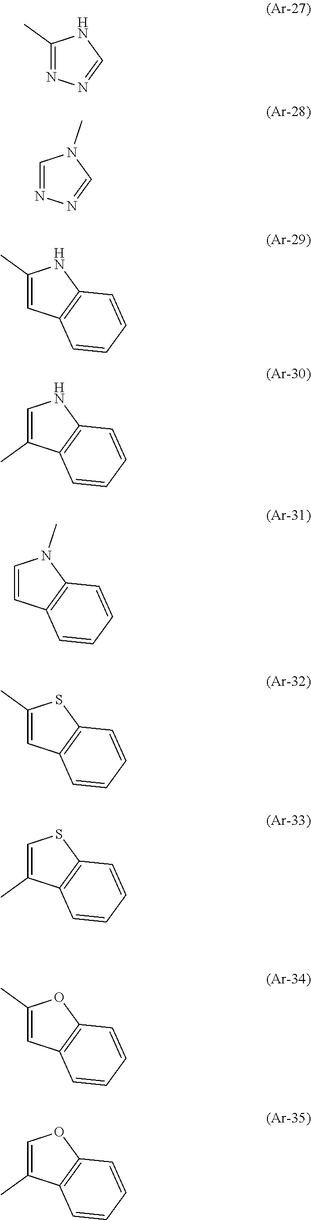

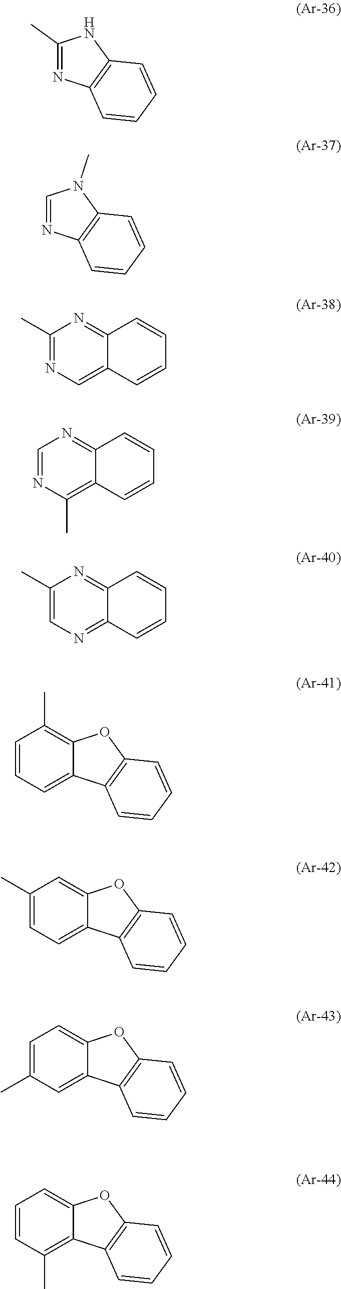

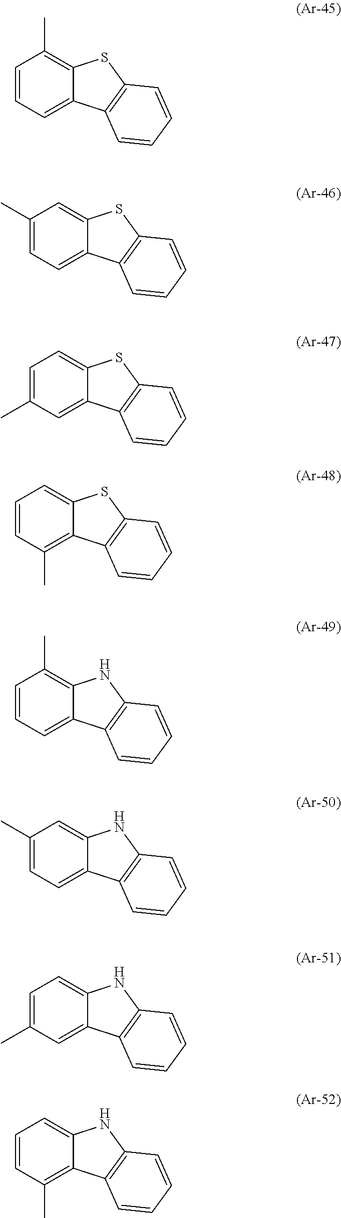

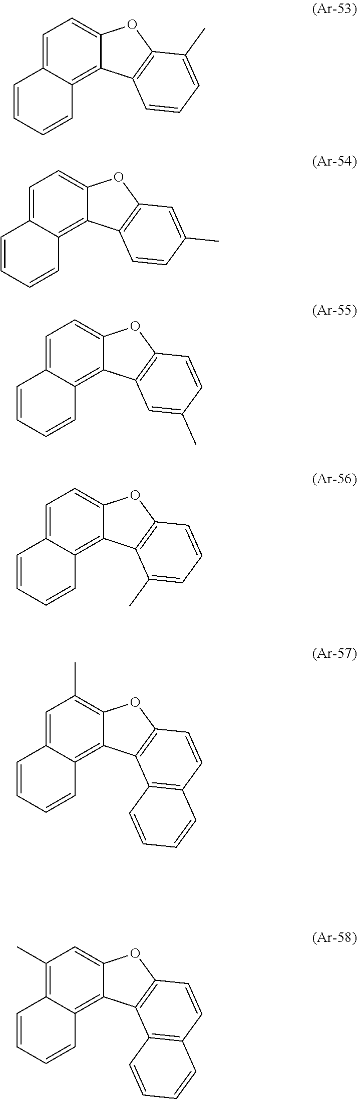

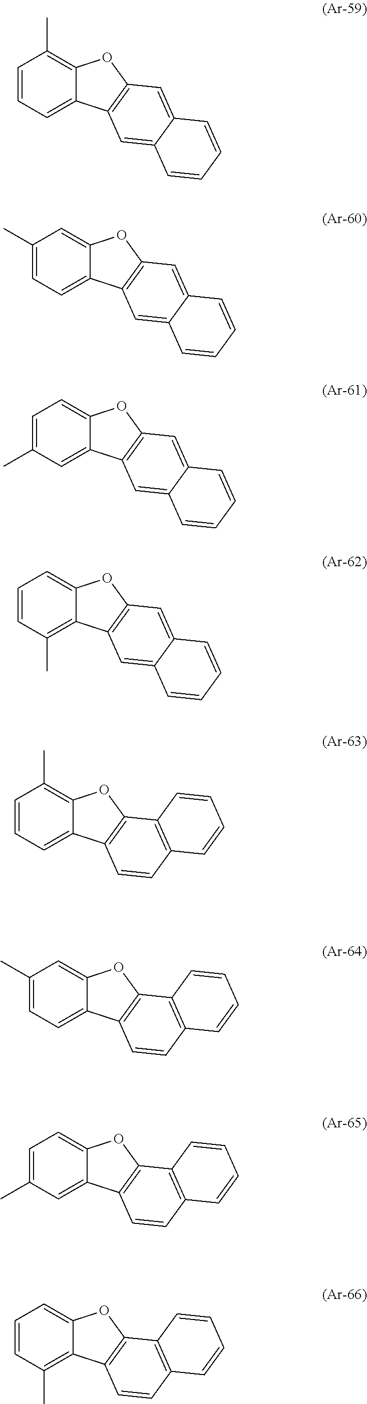

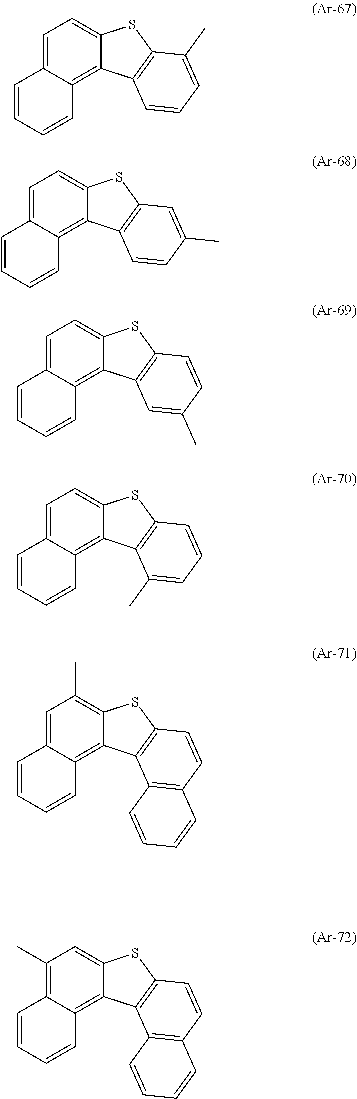

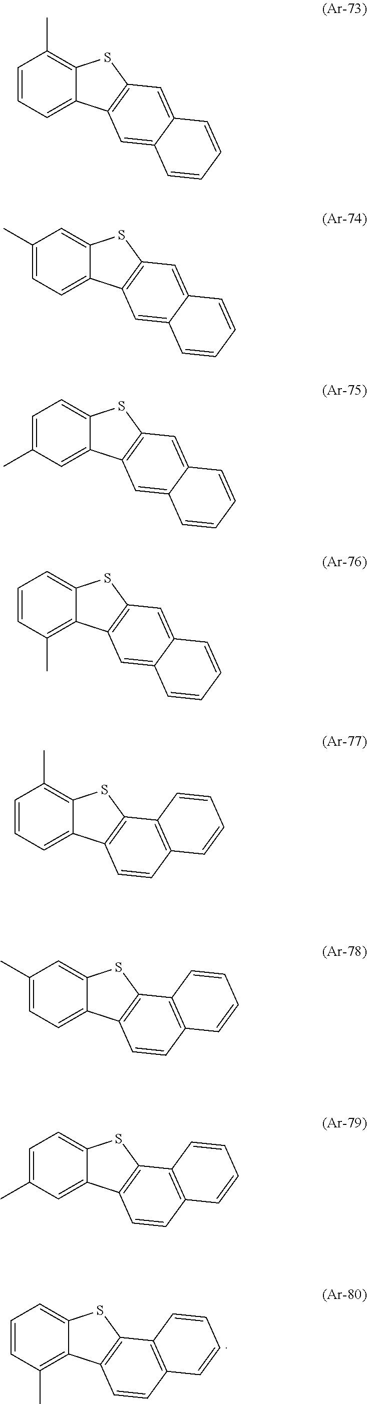

20. The organic compound according to claim 6, wherein a plurality of or each of R¹ to R¹⁰ represent deuterium.

21. The organic compound according to claim 1, wherein each of R¹ to R¹⁰ represent deuterium.

22. The organic compound according to claim 4, wherein each of R¹ to R¹⁰ represent deuterium.

23. The organic compound according to claim 5, wherein each of R¹ to R¹⁰ represent deuterium.

24. The organic compound according to claim 6, wherein each of R¹ to R¹⁰ represent deuterium.
